(19) United States
(12) Patent Application Publication
(10) Pub. No.: US 2011/0045100 A1
(43) Pub. Date: Feb. 24, 2011

(54) Antimalarial Quinolines and Methods of Use Thereof

(75) Inventors: Christian Wolf, Arlington, VA (US); Paul D. Roepe, Rockville, MD (US); Angel C. De Dios, Annandale, VA (US)

Correspondence Address: FOLEY HOAG, LLP;PATENT GROUP, WORLD TRADE CENTER WEST, 155 SEAPORT BLVD, BOSTON, MA, 02110, US

(73) Assignee: GEORGETOWN UNIVERSITY, WASHINGTON, DC (US)

(21) Appl. No.: 12/920,992

(22) PCT Filed: Mar. 5, 2009
(86) PCT No.: PCT/US09/36179

§ 371 Date: Nov. 3, 2010

Related U.S. Application Data
(60) Provisional application No. 61/033,941, filed on Mar. 5, 2008.
(60) Provisional application No. 61/115,256, filed on Nov. 17, 2008.

(51) Int. Cl. A61K 31/4706; C07D 215/46; C07D 401/12; C07D 215/36; C07D 265/36; C07D 215/233; A61K 31/47; A61K 31/4709; A61K 31/538; A61K 33/40; A61K 31/505; A61P 33/06

(52) U.S. Cl. 424/613; 546/163; 546/162; 546/153; 544/105; 514/313; 514/312; 514/230.5; 514/269

(57) ABSTRACT

One aspect of the invention relates to substitute quinolines with antimalarial activity, and compositions and kits comprising at least one of them. Another aspect of the invention relates to methods for the treatment or prevention or both of malaria comprising administering to a subject a therapeutically effective amount of such a compound. Importantly, a number of the compounds show excellent potency against both chloroquine-sensitive and chloroquine-resistant strains.

### RELATED APPLICATIONS

This application claims the benefit of priority to U.S. Provisional Patent Application Ser. No. 61/033,941, filed Mar. 5, 2008, and U.S. Provisional Patent Application Ser. No. 61/115,256, filed Nov. 17, 2008; both of which are hereby incorporated by reference in their entirety.

### GOVERNMENT SUPPORT

The invention was made with support provided by the National Institutes of Health (Grant RO1AI060792); therefore, the government has certain rights in the invention.

### BACKGROUND

Malaria remains the world's most widespread and devastating infectious disease, with approximately 300 million annual cases and more than 2 million casualties. Among the protozoan parasites of the genus Plasmodium causing malaria in humans, Plasmodium falciparum is the most lethal species. Since the discovery of the antimalarial potency of quinine and other cinchona alkaloids, a variety of agents exhibiting a 4-substituted quinoline pharmacophore has been introduced. In particular, chloroquine (CQ), mefloquine, sontoquine, and amodiaquine have proved to be among the most effective antimalarial drugs (FIG. 1). De, D.; Byers, L. D.; Krogstad, D. J. Antimalarials: synthesis of 4-aminoquinolines that circumvent drug resistance in malaria parasites. J. Heterocyclic Chem. 1997, 34, 315-320; O'Neill, P. M.; Bray, P. G.; Hawley, S. R.; Ward, S. A.; Park, B. K. 4-Aminoquinolines-past, present, and future: a chemical perspective. Pharmacol. Ther. 1998, 77, 29-58; and Dominguez, J. N. Chemotherapeutic agents against malaria: What next after chloroquine? Curr. Topics Med. Chem. 2002, 2, 1173-1185. Aminoquinolines are known to form a complex with ferriprotoporphyrin IX (FPIX), which is generated in the food vacuole of the intraerythrocytic malaria parasite as a result of proteolysis of host hemoglobin (Hb) which serves as a major source of amino acids during the protozoan life stages within the infected red blood cell. Free FPIX is cytotoxic to Plasmodium which therefore has developed a strategy to limit the amount of free FPIX, converting it into insoluble crystalline hemozoin. Pagola, S.; Stephens, P. W.; Bohle, D. S.; Kosar, A. D.; Madsen, S. K. The structure of malaria pigment β-haematin. Nature 2000, 404, 307-310. The drug-FPIX interactions inhibit conversion of hematin to hemozoin and hence its detoxification via crystallization, and the accumulation of significant concentrations of toxic FPIX-aminoquinoline adducts is believed to be ultimately responsible for killing the parasite. Ridley, R. G. Medical need, scientific opportunity and the drive for antimalarial drugs. Nature 2002, 415, 686-693; Leed, A.; DuBay, K.; Ursos, L. M.; Sears, D.; de Dios, A. C.; Roepe, P. D. Solution structures of antimalarial drug-heme complexes. Biochemistry 2002, 41, 10245-10255; Chong, C. R.; Sullivan, D. J. Jr Inhibition of heme crystal growth by antimalarials and other compounds: Implications for drug discovery. Biochem Pharmacol. 2003, 66, 2201-2212; de Dios, A. C.; Casabianca, L. B.; Kosar, A.; Roepe, P. D. Structure of the amodiaquine-FPIX μ-oxo dimer solution complex at atomic resolution. Inorg Chem. 2004, 43, 8078-8084; and de Dios, A. C.; Tycko, R.; Ursos, L. M. B.; Roepe, P. D. NMR Studies of Chloroquine—Ferriprotoporphyrin IX Complex J. Phys. Chem. A 2003, 107, 5821-5825. It is widely accepted that the 4-aminoquinoline pharmacophore plays a crucial role in the complexation to FPIX resulting in inhibition of hemozoin formation and parasite growth, while the presence of a basic amino group in the side chain is generally considered essential for trapping high concentrations of the drug in the acidic food vacuole of the parasite. Cheruku, S. R.; Maiti, S.; Dorn, A.; Scorneaux, B.; Bhattacharjee, A. K.; Ellis, W. Y.; Vennerstrom, J. L. Carbon isosteres of the 4-aminopyridine substructure of chloroquine: Effects on pKa, hematin binding, inhibition of hermozoin formation, and parasite growth. J. Med. Chem. 2003, 46, 3166-3169; and Egan, T. J.; Hunter, R.; Kaschula, C. H.; Marques, H. M.; Misplon, A.; Walden, J. Structure-function relationships in aminoquinolines: effect of amino and chloro groups on quinoline-hematin complex formation, inhibition of β-hematin formation, and antiplasmodial activity. J. Med. Chem. 2000, 43, 283-291.

To date, numerous isolates of P. falciparum have developed resistance against a majority of currently employed antimalarial drugs. In order to address the ever-increasing health impact of malaria, promising chloroquine resistant (CQR) reversal agents and new therapeutics including artemisinin and other endoperoxides have been introduced. Martin, S. K.; Oduola, A. M.; Milhous, W. K. Reversal of chloroquine resistance in Plasmodium falciparum by verapamil. Science 1987, 235, 899-901; Burgess, S. J.; Selzer, A.; Kelly, J. X.; Smilkstein, M. J.; Riscoe, M. K.; Peyton, D. H. A chloroquine-like molecule designed to reverse resistance in Plasmodium falciparum. J. Med. Chem. 2006, 49, 5623-5625; Weisman, J. L.; Liou, A. P.; Shelat, A. A.; Cohen, F. E.; Guy, R. K.; DeRisi, J. L. Searching for new antimalarial therapeutics amongst Known drugs. Chem. Biol. Drug Des. 2006, 67, 409-416; Tang, Y.; Dong, Y.; Wittlin, S.; Charman, S. A.; Chollet, J.; Chiu, F. C.; Charman, W. N.; Matile, H.; Urwyler, H.; Dorn, A.; Bajpai, S.; Wang, X.; Padmanilayam, M.; Karle, J. M.; Brun, R.; Vennerstrom, J. L. Weak base dispiro-1,2,4-trioxolanes: potent antimalarial ozonides. Bioorg. Med. Chem. Lett. 2007, 17, 1260-1265; Dong, Y.; Tang, Y.; Chollet, J.; Matile, H.; Wittlin, S.; Charman, S. A.; Charman, W. N.; Tomas, J. S.; Scheurer, C.; Snyder, C.; Scorneaux, B.; Bajpai, S.; Alexander, S. A.; Wang, X.; Padmanilayam, M.; Cheruku, S. R.; Brun, R.; Vennerstrom, J. L. Effect of functional group polarity on the antimalarial activity of Spiro and dispiro-1,2,4-trioxolanes. Bioorg. Med. Chem. 2006, 14, 6368-6382; Posner, G. H.; Paik, I. H.; Chang, W.; Borstnik, K.; Sinishtaj, S.; Rosenthal, A. S.; Shapiro, T. A. Malaria-Infected Mice Are Cured by a Single Dose of Novel Artemisinin Derivatives. J. Med. Chem. 2007, 50, 2516-2519; Paik, I. H.; Xie, S.; Shapiro, T. A.; Labonte, T.; Narducci Sarjeant, A. A.; Baege, A. C.; Posner, G. H. Second generation, orally active, antimalarial, artemisinin-derived trioxane dimers with high stability, efficacy, and anticancer activity. J. Med. Chem. 2006, 49, 2731-2734; Vennerstrom, L.; Arbe-Barnes, S.; Brun, R.; Charman, S. A.; Chiu, F. C.; Chollet, J.; Dong, Y.; Dorn, A.; Hunziker, D.; Matile, H.; McIntosh, K.; Padmanilayam, M.; Santo Tomas, J.; Scheurer, C.; Scorneaux, B.; Tang, Y.; Urwyler, H.; Wittlin, S.; Charman, W. N. Identification of an antimalarial synthetic trioxolane drug development candidate. Nature 2004, 430 (7002), 900-904; and O'Neill, P. M.; Posner, G. H. A medicinal chemistry perspective on artmeisinin and related endoperoxides. J. Med. Chem. 2004, 47, 2945-2964. However, the latter are less affordable in the most plagued tropical and subtropical regions and resistance to endoperoxide-derived antimalarials has already been reported. Jambou, R.; Legrand, E.; Niang, M.; Khim, N.; Lim, P.; Volney, B.; Ekala, M. T.; Bouchier, C.; Esterre, P.; Fandeur, T.; Mercereau-Puijalon, O. Resistance of Plasmodium falciparum field isolates to in-vitro artemether and point mutations of the SERCA-type PfATPase 6. Lancet 2005, 366, 1960-1963. Arguably, quinine, chloroquine and mefloquine are among the most successful antimalarial drugs ever used, and additional lead compounds with improved activity against CQR strains have been discovered via synthetic modifications of these structures. Delarue, S.; Girault, S.; Maes, L.; Debreu-Fontaine, M. A.; Labaeid, M.; Grellier, P.; Sergheraert, C. Synthesis and in vitro and in vivo antimalarial activity of new 4-anilinoquinolines. J. Med. Chem. 2001, 44, 2827-2833; O'Neill, P. M.; Willock, D. J.; Hawley, S. R.; Bray, P. G.; Storr, R. C.; Ward, S. A.; Park, B. K. Synthesis, antimalarial activity, and molecular modeling of tebuquine analogues. J. Med. Chem. 1997, 40, 437-448; and Madrid, P. B.; Liou, A. P.; DeRisi, J. L.; Guy, R. K. Incorporation of an intramolecular hydrogen-bonding motif in the side chain of 4-aminoquinolines enhances activity against drug-resistant P. falciparum. J. Med. Chem. 2006, 49, 4535-4543. Importantly, 4-aminoquinolines carrying an aliphatic side chain are often well tolerated and afford excellent activity-toxicity profiles. Riccio, E. S.; Lee, P. S.; Winegar, R. A.; Krogstad, D. J.; De, D.; Mirsalis, J. C. Genetic toxicology testing of the antimalarial drugs chloroquine and a new analog, AQ-13. Environ. Mol. Mutagen. 2001, 38, 69-79. The evident need for safe, effective and inexpensive antimalarials that are equally active against multiple species of Plasmodia, e.g., P. falciparum and P. vivax, has therefore directed increasing efforts to the design of new CQ analogues.

Since modification of the 7-chloroquinoline ring, i.e., incorporation of other electron-withdrawing or electron-donating substituents such as amino and methoxy groups into the various positions in the quinoline ring, have generally proved detrimental to the antimalarial activity, a systematic variation of the side chain structure and basicity seems to be more promising. Egan, T, J.; Hunter, R.; Kaschula, C. H.; Marques, H. M.; Misplon, A.; Walden J. C. Structure-function relationships in aminoquinolines: Effect of amino and chloro groups on quinoline-hematin complex formation, inhibition of β-hematin formation, and antiplasmodial activity. J. Med. Chem. 2000, 43, 283-291; and Kaschula, C. H.; Egan, T. J.; Hunter, R.; Basilico, N.; Parapani, S.; Tarameli, D.; Pasini, E.; Monti, D. Structure-activity relationships in 4-aminoquinoline antiplasmodials. The role of the group at the 7-position. J. Med. Chem. 2002, 45, 3531-3539. Although few comprehensive and methodical modifications of the CQ side chain have been reported to date, it has been established that both shortening and lengthening of the separation of the two aliphatic amino groups to either 2-3 or 10-12 carbon atoms as well as the incorporation of a phenol moiety can lead to increased activity against CQR strains. Ridley, R. G.; Hofheinz, H.; Matile, H.; Jacquet, C.; Dorn, A.; Masciadri, R.; Jolidon, S.; Richter, W. F.; Guenzi, A.; Girometta, M. A.; Urwyler, H.; Huber, W.; Thiathong, S.; Peters, W. 4-Aminoquinoline analogues of CQ with shortened side chains retain activity against CQ-resistant Plasmodium falciparum. Antimicrobial Chemother. 1996, 40, 1846-1854; De, D.; Krogstad, F. M.; Byers, L. D.; Krogstad, D. J. Structure-activity relationships for antiplasmodial activity among 7-substituted 4-aminoquinolines. J. Med. Chem. 1998, 41, 4918-4926; Madrid, P. B.; Liou, A. P.; DeRisi, J. L.; Guy, K. Incorporation of an intramolecular hydrogen bonding motif in the side chain of 4-aminoquinolines enhances activity against drug-resistant P. falciparum. J. Med. Chem. 2006, 49, 4535-4543; and Hawley, S. R.; Bray, P. G.; Park, B. K.; Ward, S. A. Amodiaquine accumulation in plasmodium falciparum as a possible explanation for its superior antimalarial activity over chloroquine. Mol. Biochem. Parasitol. 1996, 80, 15-25. Several studies revealed that introduction of a branched dialkylamino motif at the side chain terminus of CQ, e.g., replacement of the ethyl by isopropyl or tert-butyl groups, can furnish metabolically more stable antimalarials with enhanced life-time and retained activity against drug resistant strains of P. falciparum. Stocks, P. A.; Raynes, K. J.; Bray, P. G.; Park, B. K.; O'Neill, P. M.; Ward, S. A. Novel short chain chloroquine analogues retain activity against chloroquine resistant K1 Plasmodium falciparum. J. Med. Chem. 2002, 45, 4975-4983; and Madrid, P. B.; Wilson, N. T.; DeRisi, J. L.; Guy, R. K. Parallel synthesis and antimalarial screening of a 4-aminoquinoline library. J. Comb. Chem. 2004, 6, 437-442.

### SUMMARY

One aspect of the invention relates to substituted quinolines with antimalarial activity, and compositions and kits comprising at least one of them. Another aspect of the invention relates to methods for the treatment or prevention or both of malaria comprising administering to a subject a therapeutically effective amount of such a compound. Importantly, a number of the compounds show excellent potency against both chloroquine-sensitive and chloroquine-resistant strains.

### BRIEF DESCRIPTION OF THE FIGURES

FIG. 1 depicts structures of antimalarial quinolines.

FIG. 2 depicts structures of tribasic and dibasic 4-amino-7-chloroquinolines.

FIG. 3 depicts one route to the synthesis of 4-amino-7-chloroquinolines 4a-e and 5a-e.

FIG. 4 depicts one route to the synthesis of symmetrically branched 4-amino-7-chloroquinolines 6a,b and 7a,b.

FIG. 5 depicts one route to the synthesis of 1,3- and 1,4-diaminocyclohexane-derived chloroquinolines 16a,b and 17a,b.

FIG. 6 depicts a table showing antiplasmodial activity. The experimental IC₅₀'s are averages of two separate determinations each conducted in triplicate. The selectivity index (SI) is the ratio of the IC₅₀ for the resistant versus the sensitive strain (Dd2/HB3, 4^{th} column; FCB/GCO3, 7^{th} column).

FIG. 7 depicts a table showing activity of selected 4-amino-7-chloroquinolines of the invention versus GCO3 and FCB (results for two separate assays). The experimental IC₅₀'s were obtained from triplicate experiments.

FIG. 8 depicts a table showing activity of selected 4-amino-7-chloroquinolines of the invention versus HB3 and Dd2 (results for two separate assays). The experimental IC₅₀'s were obtained from triplicate experiments.

FIG. 9 depicts a table showing activity of selected 4-amino-7-chloroquinolines versus HB3, Dd2, GCO3 and FCB (results for two separate assays). The experimental IC₅₀'s were obtained from triplicate experiments.

FIG. 10 depicts synthetic routes to selected 4-amino-7-chloroquinolines.

FIG. 11 depicts the general structures of some 4-amino-7-chloroquinolyl-derived amides, sulfonamides, ureas and thioureas.

FIG. 12 depicts one approach to the synthesis of 4-amino-7-chloroquinolyl-derived sulfonamides 23-28, 31-34 and 35-38.

FIG. 13 depicts one approach to the preparation of 4-amino-7-chloroquinolyl-derived ureas and thioureas 39-56.

FIG. 14 depicts a table showing antiplasmodial activity of CQ-derived 4-amino-7-chloroquinolyl-derived sulfonamides, ureas and thioureas 39-56 against HB3 and Dd2. IC₅₀ values were obtained from an average of two separate determinations each performed in triplicate. Resistance Index, RI, is CQR-IC₅₀/CQS-IC₅₀.

FIG. 15 depicts one approach to the preparation of 4-amino-7-chloroquinolyl-derived amides 27-45.

FIG. 16 depicts a table showing antiplasmodial activity of CQ-derived 4-amino-7-chloroquinolyl-derived amides 27-45 against HB3 and Dd2. IC₅₀ values were obtained from an average of two separate determinations each performed in triplicate. Resistance Index, RI, is CQR-IC₅₀/CQS-IC₅₀.

FIG. 17 depicts depicts one approach to the preparation of 4-amino-7-chloroquinolyl-derived amides 46-54.

FIG. 18 depicts a table showing antiplasmodial activity of CQ-derived 4-amino-7-chloroquinolyl-derived amides 46-54 against HB3 and Dd2. IC₅₀ values were obtained from an average of two separate determinations each performed in triplicate. Resistance Index, RI, is CQR-IC₅₀/CQS-IC₅₀.

FIG. 19 depicts one approach to the synthesis of chloroquine derivatives 81-90.

FIG. 20 depicts one approach to the synthesis of CQ-derived ethers 91-95.

FIG. 21 depicts one approach to the synthesis of CQ-derived sulfides 97-100.

FIG. 22 depicts one approach to the introduction of α,ω-diaminoalkoxy branched sidechains to 7-chloroquinoline 101-103.

FIG. 23 depicts a table of IC₅₀ values for 4-amino-, 4-alkoxy- and 4-alkylthioquinoline derivatives 91-103. The selectivity index (SI) is the ratio of the IC₅₀ for a given drug shown by a CQ-resistant strain vs. IC₅₀ for the companion CQ-sensitive strain. Column 4 shows SI computed as Dd2 IC₅₀/HB3 IC₅₀, column 7 shows FCB IC₅₀/GCO3 IC₅₀, column 10 is K1/NF54, and column 13 is IndoI/Haiti 135.

FIG. 24 depicts a table showing calculated and measured pKa for representative compounds 88, 93, 98 and 102. SPARC is an online pKₐ approximation program developed at the University of Georgia (S. W. Karickhoff, L. A. Carreira and S. H. Hilal); pKₐ₁ or pKₐ₃ represent the pKₐ of side chain tertiary N and pKₐ₂ represents the pKₐ of quinolyl N; the pKa measurements represent an average of three determinations performed by acid/base titrations at room temperature; and “nd” denotes results not determined.

FIG. 25 depicts a table showing measured binding constants for monomeric (pH 3.9) and μ-oxo dimeric (pH 7.5) heme for representative compounds 88, 93, 98, and 102; “nd” denotes results not determined.

FIG. 26 depicts a table showing measured hemozoin (Hz) inhibition IC₅₀ for representative compounds 88, 93, 98, and 102.

FIG. 27 depicts a table showing computed Vacuolar Accumulation Ratios (VAR) for representative compounds 88, 93, 98, and 102. VAR is calculated using the Henderson-Hasselbach equation and knowing cytosolic pH=7.4, DV pH for CQR parasites=5.2, DV pH for CQS=5.6 and assuming: 1) that charged (protonated) drugs are essentially membrane impermeable; 2) net accumulation is not affected by binding to drug target. Although these are both simplifications, the calculated differences for (effectively) mono vs. diprotic drugs are orders of magnitude apart, whereas binding effects are expected to be (at most) several fold.

FIG. 28 depicts structures of drug-μ-oxo dimer complexes derived from distance geometry calculations using Fe(III)-drug (¹H) distance restraints from relaxation measurements. The drug molecules, on average, are approximately 3-4 Å above the plane of the porphyrin ring. Since the distance restraints are drawn from a single point (Fe(III)), the porphyrin plane's rotational orientation is not unequivocally defined (see FIG. 29). Within the limitations imposed by assumptions made in these calculations and the accuracy of the data, no significant differences in how these drug molecules interact with the μ-oxo dimer are found.

FIG. 29 depicts structures of drug-μ-oxo dimer complexes derived from distance geometry calculations using Fe(III)-drug (¹H) distance restraints from relaxation measurements, as in FIG. 28, from a top-down view. The relaxation rates of the alipathic protons are likewise enhanced by the addition of heme and as shown in these structures, the side chains do not extend away from Fe(III), but trace the perimeter of the porphyrin ring.

FIG. 30 depicts a suggested structure for a drug-μ-oxo dimer complex, in which the drug has a branched side chain. One of the branches is placed along the perimeter of the porphyrin ring, as seen in FIG. 29 and for previously solved CQ, QN, QD, and AQ structures, while the other branch extends away from the ring. In this arrangement, it is possible that this terminal amino group then forms an a hydrogen bonding pair with the propionate side chain of heme. A minimal distance (greater than 4 methylenes between terminal amino and the branch point) for both maximal π-π interaction and hydrogen bonding is defined in this structure.

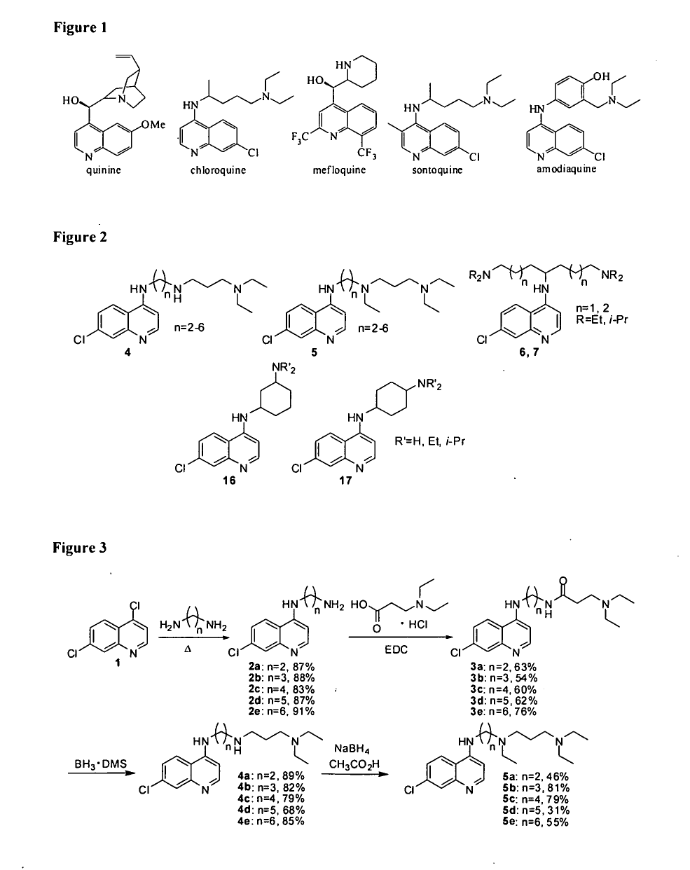

### DETAILED DESCRIPTION

One aspect of the invention relates to quinoline antimalarials (e.g., quinine, chloroquine, mefloquine, sontoquine and amodiaquine) in which the side chain has been systematically varied to provide affordable heme-targeted antimalarials that overcome the ever-increasing problem with worldwide drug resistance.

For example, one aspect of the invention relates to the preparation of a series of new heme-targeted antimalarials obtained by systematically varying both the structure and basicity of the side chain attached to the 7-chloro-4-aminoquinoline pharmacophore of chloroquine (CQ). All 18 compounds tested show potent antiplasmodial activity against 4 different strains in vitro and can be synthesized from readily available, inexpensive starting materials through a few high-yielding steps. Comparison with CQ revealed that 4b, 5a, 5b, 5d, 16a, 16b, 17a, and 17b afford clearly superior activity against the CQ resistant strain Dd2 and 4b, 5a, 5b, 5d, 17a, and 17b proved significantly more potent against FCB. In particular, the tribasic 4-aminoquinolines 5a and 5b carrying a short linear side chain with two additional aliphatic tertiary amino functions are highly potent antimalarials and equally effective against both CQS and CQR strains.

In addition, another aspect of the invention relates to the synthesis and in vitro antimalarial activities of 7-chloro-4-aminoquinolyl-derived sulfonamides 23-28 and 31-46, ureas 39-42, thioureas 43-46, and amides 27-54. Many of the CQ analogues disclosed herein showed submicromolar antimalarial activity versus HB3 (chloroquine sensitive) and Dd2 (chloroquine resistant strains of P. falciparum) and low resistance indices were obtained in most cases. Systematic variation of the side chain length and introduction of fluorinated aliphatic and aromatic termini revealed promising leads that overcome CQ resistance. In particular, sulfonamide 23 exhibits a short side chain with a terminal dansyl moiety combined high antiplasmodial potency with a low resistance index and showed IC₅₀'s of 17.5 nM and 22.7 nM against HB3 and Dd2 parasites.

Further, using predictions from recent solution and solid state heme-quinoline antimalarial complex structures, synthetic modifications of chloroquine (CQ), and current hypotheses for chloroquine resistance (CQR), CQ analogues have been designed and synthesized to systematically test key structure-function principles. These new compounds have been tested for activity vs. multiple chloroquine sensitive (CQS) and CQR malarial parasites using a recently developed high throughput SYBR Green I-based assay. In certain embodiments, the importance of aliphatic side chain length for a series of monoethyl and diethyl 4N CQ derivatives has been systematically probed. In other embodiments, the pKa of the critical quinolyl N have been altered by introducing alkylthio or alkoxy substituents into the 4 position, and also varied side chain length for these 4S and 4O CQ analogues. In yet other embodiments, an additional titratable amino group was introduced on the side chain of 4O analogues with promising CQR selectivity (meaning, higher activity for CQR vs. CQS malarial parasites) and increased activity while retaining improved selectivity. As described herein, the atomic resolution structures for complexes formed between representative 4N, 4S and 4O derivatives vs. μoxo dimeric heme were solved, the binding constants for monomeric vs. dimeric heme were measured, and the ability of the drugs to inhibit hemozoin (Hz) formation in vitro at different pH's were quantified. Taken together, the data provided additional insight for the design of CQ analogues with improved activity vs. CQR malarial parasites.

### 4-Amino-7-Chloroquinolines

It was hypothesized that incorporating an increasing number of basic amino groups along with systematic structural variations (length and branching) of the aliphatic side chain attached to the potent 4-amino-7-chloroquinoline pharmacophore would provide new candidates that overcome antimalarial drug resistance. For example, the introduction of a highly branched tether between the two amino functions in CQ as well as the replacement of the metabolically unstable terminal diethylamino group by an isopropyl analogue were expected to enhance the life-time of CQ analogues exhibiting retained activity against CQR strains. Herein, the synthesis and evaluation of the antimalarial activity of a series of novel 4-amino-7-chloroquinolines carrying either a branched or a linear side chain with two or three amino functions (FIG. 2) is disclosed.

As shown in FIG. 3, one synthetic approach towards these heme-targeted antimalarials involved inexpensive materials and high-yielding steps in most cases. Amination of 4,7-dichloroquinoline, 1, with commercially available α,ω-diaminoalkanes gave N-(7-chloro-4-quinolyl)-1,n-diaminoalkanes 2 in 83 to 91% yield. Coupling of 2 with N,N-diethylamino-3-propionic acid in the presence of 1-ethyl-3-(3-dimethylaminopropyl)carbodiimide hydrochloride (EDC) furnished amides 3 which were reduced with borane-dimethyl sulfide to the corresponding series of secondary amines 4. Finally, tertiary amines 5 were prepared by treatment of precursors 4 with sodium borohydride in glacial acetic acid.

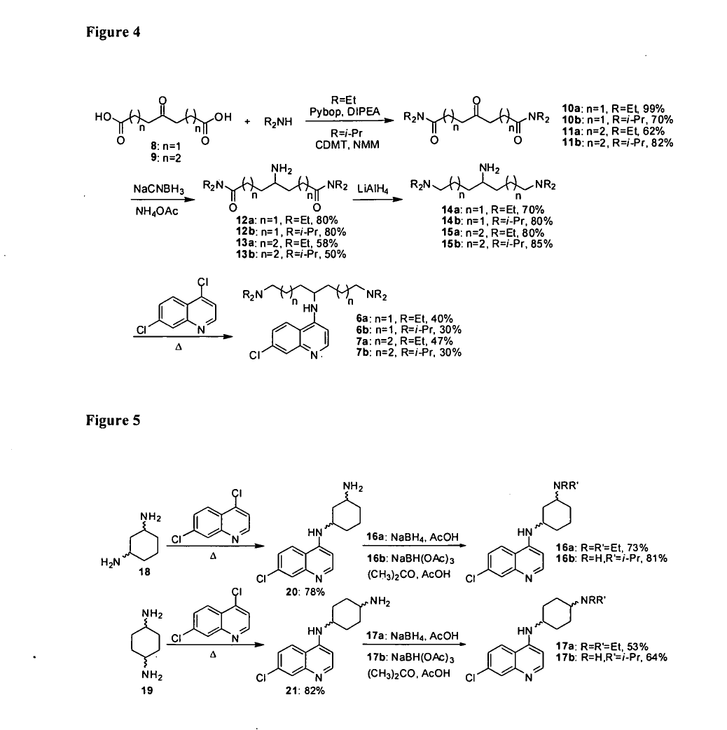

The symmetrically branched amines 6 and 7 were synthesized from 4-ketopimelic acid, 8, and 5-oxoazelaic acid, 9 (FIG. 4). Screening of different coupling conditions revealed that Pybop and CDMT allow efficient amide formation with diethylamine and diisopropylamine, respectively. The corresponding α,ω-diamides 10 and 11 were thus obtained in 62-99% yield. Reductive amination of the ketone group using ammonium acetate and sodium cyanoborohydride, and subsequent reduction of the terminal amides with lithium aluminium hydride gave triamines 14 and 15 in good yields. These amines were then employed in a carefully optimized nucleophilic aromatic substitution procedure using excess of 1 at high temperatures in a closed vessel to produce chloroquinolines 6 and 7. The 1,3- and 1,4-diaminocyclohexane-derived chloroquinolines 16a, 16b, 17a, and 17b were prepared from 4,7-dichloroquinoline and a mixture of the cis- and trans-isomers of diaminocyclohexanes 18 and 19. The nucleophilic halide displacements were followed by treatment of intermediates 20 and 21 with either NaBH₄ and acetic acid or acetone and NaBH(OAc)₃ (FIG. 5).

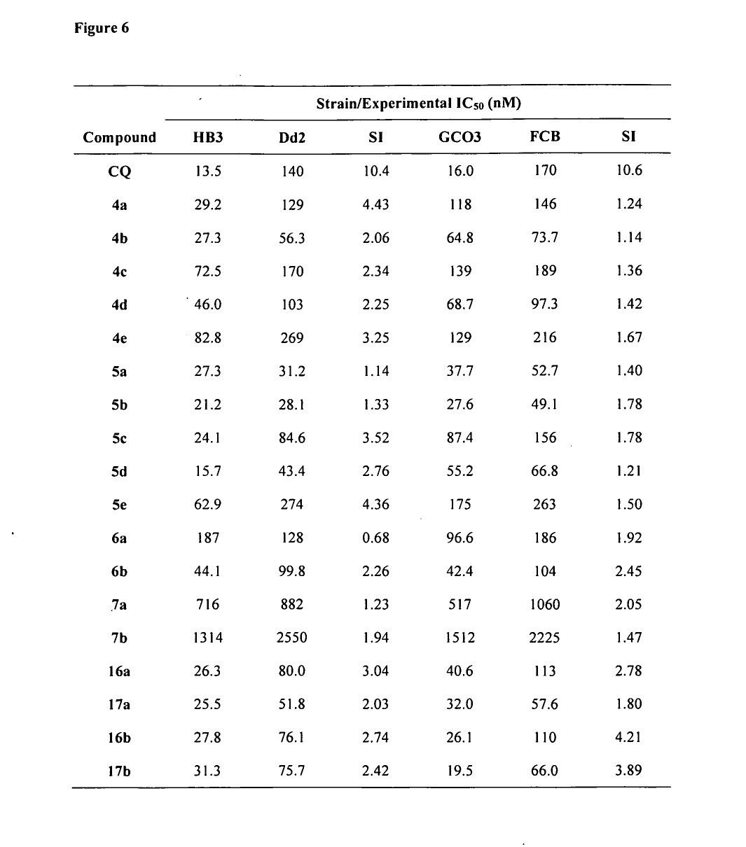

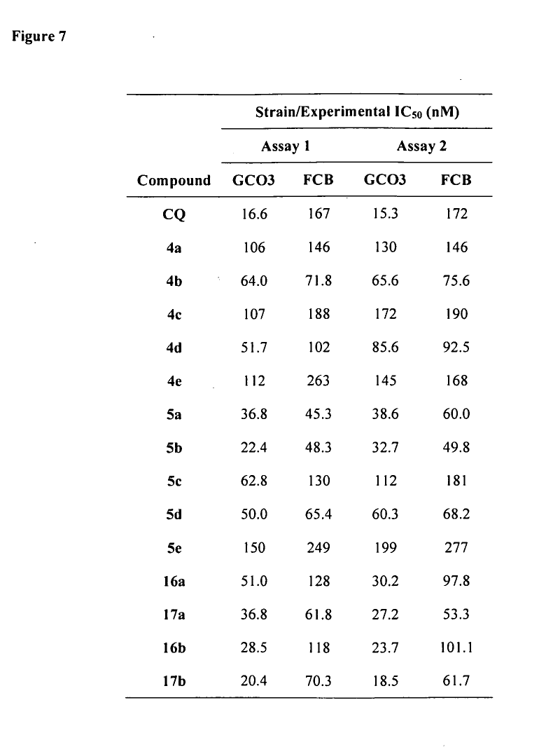

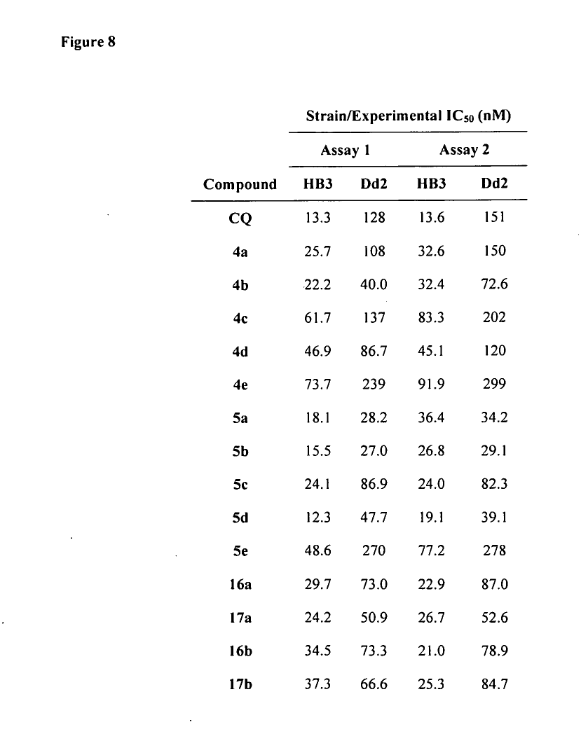

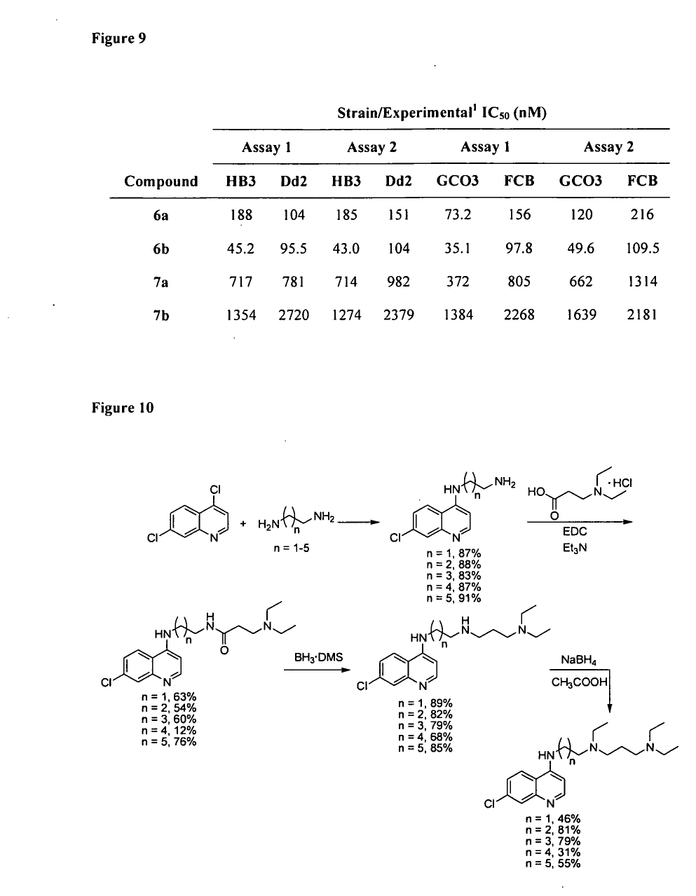

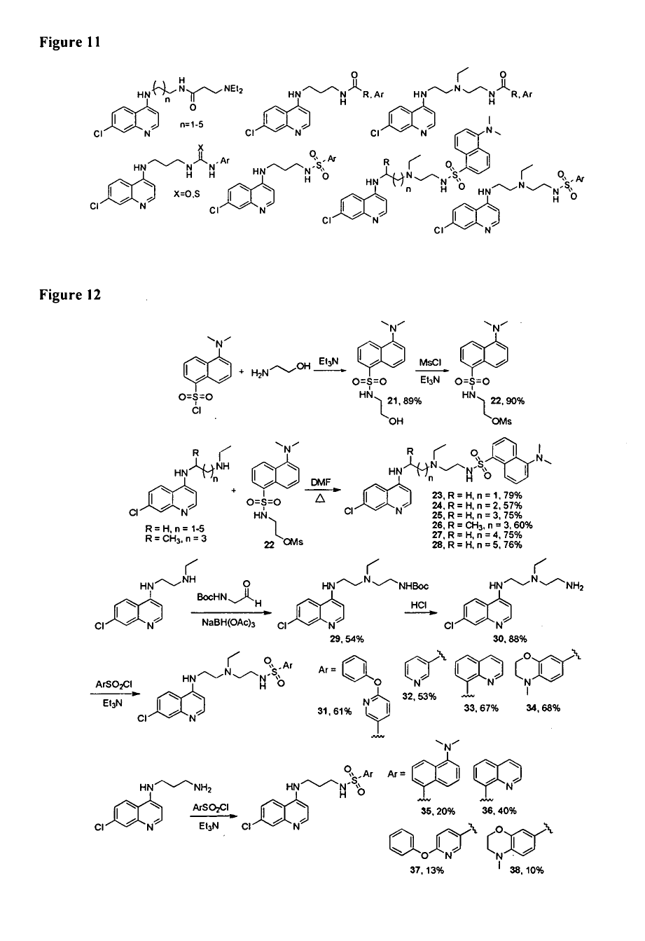

The antiplasmodial activity of tribasic compounds 4a-e, 5a-e, 6a, 6b, 7a, and 7b as well as the dibasic 1,3- and 1,4-diaminocyclohexane-derived chloroquinoline analogues 16a, 16b, 17a, and 17b was measured versus two CQS (HB3 and GCO3) and two CQR (Dd2 and FCB) strains using a standardized, inexpensive assay based on SYBR Green I intercalation that has recently been adopted and validated by several laboratories. Bennett T. N.; Paguio, M.; Gligorijevic, B.; Seudieu, C.; Kosar, A. D.; Davidson, E.; Roepe, P. D. Novel, rapid, and inexpensive cell-based quantification of antimalarial drug efficacy. Antimicrob. Agents Chemother. 2004, 48, 1807-1810; Smilkstein, M.; Sriwilaijaroen, N.; Kelly, J. X.; Wilairat, P.; Riscoe, M. Simple and inexpensive fluorescence-based technique for high-throughput antimalarial drug screening. Antimicrob. Agents Chemother. 2004, 48, 1803-1806; and Johnson, J. D.; Dennull, R. A.; Gerena, L.; Lopez-Sanchez, M.; Roncal, N. E.; Waters, N.C. Assessment and continued validation of the malaria SYBR green I-based fluorescence assay for use in malaria drug screening. Antimicrob. Agents Chemother. 2007, 51, 1926-1933. The IC₅₀ values were calculated from experiments carried out in triplicate and compared to CQ (FIG. 6). Many of the aminoquinolines prepared for this study showed antimalarial activity versus HB3 and GCO3 similar to that of CQ and we were pleased to find that several compounds were significantly more potent against the resistant strains Dd2 and FCB than CQ. Among the 18 compounds evaluated, 8 gave IC₅₀'s between 28.1 and 80.0 nM for Dd2 (CQ IC₅₀=140 nM) and 6 showed IC₅₀'s ranging from 49.1 to 73.7 nM for FCB (CQ IC₅₀=170 nM). Interestingly, the antimalarial activity of the linear tribasic aminoquinolines 4a-e and 5a-e proved to be generally superior over that of the tribasic compounds 6a, 6b, 7a, and 7b carrying a symmetrically branched side chain. Impressive antimalarial activity was also observed with the highly branched dibasic CQ analogues 16a, 16b, 17a, and 17b but these compounds possess an inherently higher selectivity index (SI, the ratio of the IC₅₀ for a resistant versus a sensitive strain) than the linear tribasic aminoquinolines. Noteworthy, diastereomeric mixtures of 16a and 17a have previously been prepared by Drake et al. and Jensen later reported higher antimalarial activity against certain CQS and CQR strains relative to chloroquine. Drake, N. L.; Creech, H. J.; Garman, J. A.; Haywood, S. T.; Peck, R. M.; van Hook, J. O.; Walton, E. Synthetic antimalrials. The preparation of certain 4-aminoquinolines. J. Am. Chem. Soc. 1946, 68, 1208-1213; Geary, T. G.; Divo, A. A.; Jensen, J. B. Activity of quinoline-containing antimalarials against chloroquine-sensitive and -resistant strains of Plasmodium falciparum in vitro. Trans. R. Soc. Trop. Med. Hyg. 1987, 81, 499-503; and Geary, T. G.; Jensen, J. B. Lack of cross-resistance to 4-aminoquinolines in chloroquine-resistant Plasmodium falciparum in vitro. J. Parasitol. 1983, 69, 97-105. The selectivity index provides a quantitative measure of the antimalarial activity against CQR strains relative to that against sensitive strains and thus indicates promising drug discovery leads. The selectivity index of CQ is about 10 whereas all compounds tested have SI's between 0.68 and 4.43. In this regard, it is important that 5a and 5b combine high antimalarial activity against HB3 and GCO3 with very low SI values between 1.14 and 1.78. These new heme-targeted antiplasmodials thus show activity versus CQS strains similar to that of CQ and, more importantly, they retain their potency against CQR strains. Comparison of the antimalarial activity of 5a and 5b with the results obtained for the tribasic aminoquinolines 4a and 4b suggests that the presence of a tertiary central amino group in this series is crucial for the activity against Dd2, GCO3 and FCB but not for HB3. Similarly, the potency of 5a and 5b versus Dd2, GCO3 and FCB diminishes when the chain length is increased. The impressive SI values of all compounds tested demonstrate that systematic variations of both the CQ side chain structure and basicity provide new venues to overcome antimalarial drug resistance. This can be combined with the introduction of a third basic amino function which should further favor accumulation of the drug within the acidic food vacuole of the parasite. However, the relatively high IC₅₀'s of 6a, 6b, 7a, and 7b reveal that basicity and structure of the side chain can not be optimized independently.

### 4-Amino-7-Chloroquinolyl Amides, Sulfonamides, Ureas and Thioureas

Sulfonamides including the protease inhibitor and antiretroviral fosamprenavir, the nonsteroidal anti-inflammatory drug celecoxib, and sumatriptan, which has been used to treat migraine headaches, have found widespread use as pharmaceuticals. Among the few examples of antimalarial sulfonamides reported to date, some exhibit remarkable potency. Ryckebusch, A.; Deprez-Poulain, R.; Debreu-Fontaine, M.-A.; Vandaele, R.; Mouray, E.; Grellier, P.; Sergheraert, C. Bioorg. Med. Chem. Lett. 2002, 12, 2595-2598; Krungkrai, J.; Scozzafava, A.; Reungprapavut, S.; Krungkrai, S. R.; Rattanajak, R.; Kamchongwongpaisan, S.; Supuran, C. T. Bioorg. Med. Chem. 2005, 13, 483-489; Klingenstein, R.; Melnyk, P.; Leliveld, S. R.; Ryckebusch, A.; Korth, C. J. Med. Chem. 2006, 49, 5300-5308; and Plouffe, D.; Brinker, A.; McNamara, C.; Henson, K.; Kato, N.; Kuhen, K.; Nagle, A.; Adrian, F.; Matzen, J. T.; Anderson, P.; Nam, T.-g.; Gray, N. S.; Chatterjee, A.; Janes, F.; Yan, S. F.; Trager, R.; Caldwell, J. S.; Schultz, P. G.; Zhou, Y.; Winzeler, E. A. Proc. Nat. Acad. Sci. 2008, 105, 9059-9064. Therfore, CQ-derived sulfonamides 23-28 and 31-38 (FIG. 12) were prepared. Following a literature procedure, 21 was synthesized in 89% yield from dansyl chloride and aminoethanol. Kim, T. W.; Park, J-H.; Hong, J-I. J. Chem. Soc., Perkin Trans. 2002, 923-927. Treatment of 21 with methanesulfonyl chloride gave the corresponding mesylate 2 in 90% yield which allowed formation of sulfonamides 23-28 from a series of N-(7-chloro-4-quinolyl)-1,n-diaminoalkanes. Reductive amination of N-(7-chloro-4-quinolyl)-N′-ethyl-1,2-diaminoethane in the presence of N-t-Boc-glycinal gave chloroquinoline 29 in 54% yield. Deprotection furnished 30 which was then converted to arylsulfonamides 31-34 in good yields. Using a similar approach, 35-38 were prepared from N-(7-chloro-4-quinolyl)-N′-propyl-1,3-diaminoethane and an arylsulfonyl chloride in a single step.

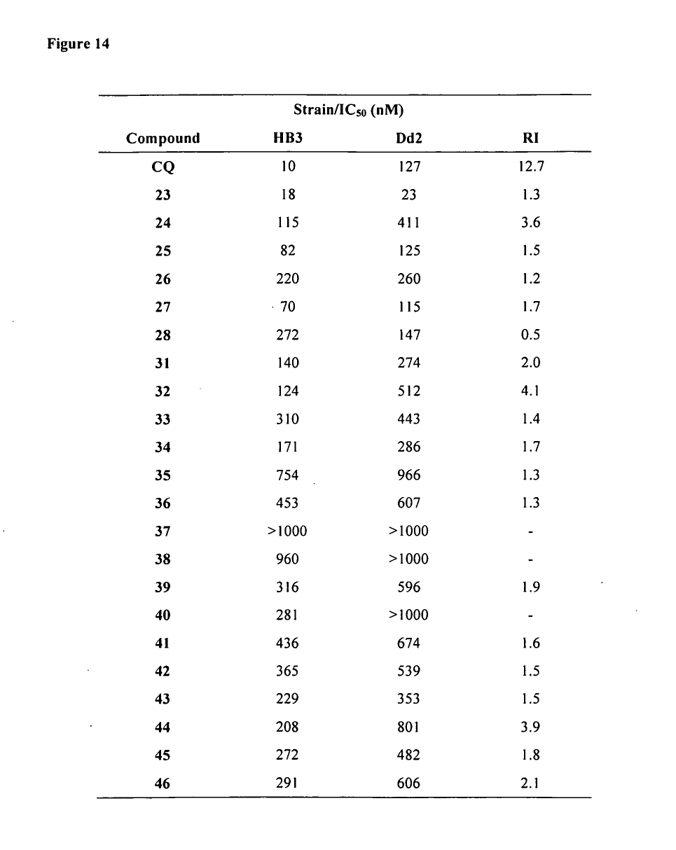

The antiplasmodial activity of these compounds was measured versus a CQS (HB3) and a CQR (Dd2) strain using a standardized, inexpensive assay based on SYBR Green I intercalation. Bennett T. N.; Paguio, M.; Gligorijevic, B.; Seudieu, C.; Kosar, A. D.; Davidson, E.; Roepe, P. D. Antimicrob. Agents Chemother. 2004, 48, 1807-1810; Smilkstein, M.; Sriwilaijaroen, N.; Kelly, J. X.; Wilairat, P.; Riscoe, M. Antimicrob. Agents Chemother. 2004, 48, 1803-1806; and Johnson, J. D.; Dennull, R. A.; Gerena, L.; Lopez-Sanchez, M.; Roncal, N. E.; Waters, N.C. Antimicrob. Agents Chemother. 2007, 51, 1926-1933. The IC₅₀ values were calculated from experiments carried out in triplicate and compared to CQ (FIG. 14). Sulfonamides 23-28 represent a series of CQ analogues with systematically varied side chain length and a dansyl unit attached to the diethylamino terminus. All compounds within this series showed antimalarial activity against both strains tested and a low resistance index (RI). The RI provides a quantitative measurement of the antiplasmodial activity against CQR strains relative to that against CQS strains and reveals promising drug discovery leads. We found that the RI for 23-28 range from 0.5 to 3.6 whereas the resistance index of CQ was determined as 11.8. Most remarkable in this series is that the short chain 7-chloro-4-aminoquinolyl sulfonamide 23 proved significantly more potent against the resistant strain Dd2 relative to CQ. Compound 23 gave IC₅₀'s of 17.5 and 22.7 nM against HB3 and Dd2, respectively. It thus retained its potency even when tested against a CQR strain. An increase in the chain length proved detrimental to the antimalarial activity. However, a maximum of the IC₅₀'s against the CQS and the CQR strains was obtained for compound 24 exhibiting three methylene units between the 4-aminoquinoline moiety and the tertiary amino function. Krogstad previously reported a somewhat similar trend for the antimalarial potency of CQ derivatives with varying side chain length against Indochina I, a CQR strain, but not against Haiti 135, a CQS strain. De, D.; Krogstad, F. M.; Cogswell, F. B.; Krogstad, D. J. Am. J. Trop. Med. Hyg. 1996, 55, 579-583. Interestingly, comparison of compounds 25 and 26 shows that introduction of a methyl group, which perfectly mimics the side chain of CQ, reduces the activity against both strains tested. Exchange of the 6-dimethylaminonaphthyl group in 23 by other aromatic groups furnished sulfonamides 31-34. These compounds gave similar RI values, ranging from 1.4 to 4.1, but showed lower antimalarial activity than 23, which indicates the significance of the terminal dansyl group. The basic tertiary amino function in the side chain is commonly believed to be crucial for the accumulation of the drug within the acidic food DV. The IC₅₀'s of sulfonamides 35-38 therefore increased into the micromolar range.

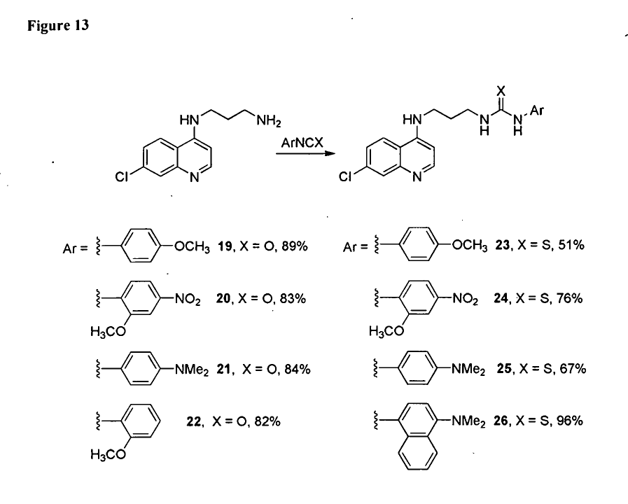

4-Amino-7-chloroquinolyl-derived ureas and thioureas 39-46 were prepared in good to high yields from N-(7-chloro-4-quinolyl)-1,3-diamine and the corresponding isocyanate and isothiocyanate, respectively, as shown in FIG. 13. Almost all compounds studied showed submicromolar antiplasmodial activity (FIG. 14). These results compare favorably with the majority of previously reported chloroquine-derived ureas and thioureas. Leon, C.; Rodriguez, J.; de Dominguez, N. G.; Charris, J.; Gut, J.; Rosenthal, P. J.; Dominguez, J. N. Eur. J. Med. Chem. 2007, 42, 735-742; and Mahajan, A.; Yeh, S.; Nell, M.; van Rensburg, C. E. J.; Chibale, K. Bioorg. Med. Chem. Lett. 2007, 17, 5683-5685. However, Chibale et al. found that urea analogues of ferrochloroquine afford superior antiplasmodial activity against a sensitive (D10) and a resistant (K1) strain compared to CQ. Chibale, K.; Moss, J. R.; Blackie, M.; van Schalkwyk, D.; Smith, P. J. Tetrahedron Lett. 2000, 41, 6231-6235. In analogy to the sulfonamides discussed above, the low RI values of 39-46 are impressive and suggest that incorporation of a rigid urea or thiourea group into the side chain provides new leads that overcome drug resistance to heme-targeted antimalarials.

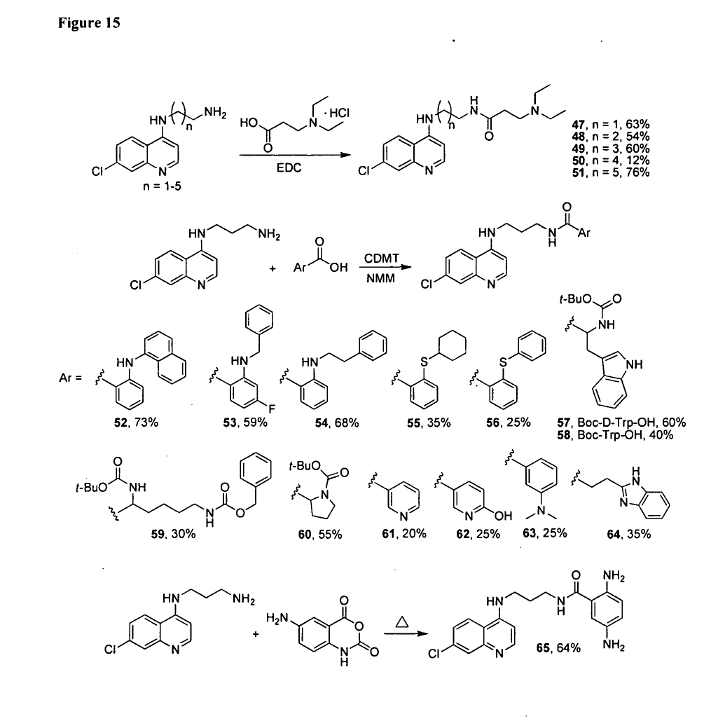

The incorporation of amide functionalities into the side chain of primaquine, amodiaquine and chloroquine has led to a remarkable range of promising antimalarial agents. For comparison with the sulfonamides and ureas discussed above, chloroquine-derived amides 57-65 (FIG. 15) were prepared. Kaur, K.; Patel, S. R.; Patil, P.; Jain, M.; Khan, S. I.; Jacob, M. R.; Ganesan, S.; Tekwani, B. L.; Jain, R. Bioorg. Med. Chem. 2007, 15, 915-930; Go, M.-L.; Ngiam, T.-L.; Wan, A. S.C. J. Med. Chem. 1981, 24, 1471-1475; Delarue, S.; Girault, S.; Maes, L.; Debreu-Fontaine, M.-A.; Labaeid, M.; Grellier, P.; Sergheraert, C. J. Med. Chem. 2001, 44, 2827-2833; Ryckebusch, A.; Deprez-Poulain, R.; Maes, L.; Debreu-Fontaine, M.-A.; Mouray, E.; Grellier, P.; Sergheraert, C. J. Med. Chem. 2003, 46, 542-557; Musonda, C. C.; Taylor, D.; Lehman, J.; Gut, J.; Rosenthal, P. J.; Chibale, K. Bioorg. Med. Chem. 2004, 14, 3901-3905; Ryckebusch, A.; Fruchart, J.-S.; Cattiaux, L.; Rousselot-Paillet, P.; Leroux, V.; Melnyk, O.; Grellier, P.; Mouray, E.; Sergheraert, C.; Melnyk, P. Bioorg. Med. Chem. Lett. 2004, 14, 4439-4443; Musonda, C. C.; Gut, J.; Rosenthal, P. J.; Yardley, V. de Souza, R. C. C.; Chibale, K. Bioorg. Med. Chem. 2006, 14, 5605-5615; Freitag, M.; Kaiser, M.; Larsen, T.; Zohrabi-Kalantari, V.; Heidler, P.; Link, A. Bioorg. Med. Chem. 2007, 15, 2782-2788. Coupling of N-(7-chloro-4-quinolyl)-1,n-diaminoalkanes of varying chain length and N,N-diethylamino-3-propionic acid in the presence of 1-[3-(dimethylaminopropyl]-3-ethylcarbodiimide (EDC) gave 57-61. By contrast, it was found that superior results in the syntheses of 52-64 are obtained when 2-chloro-4,6-dimethoxy-1,3,5-triazine (CDMT) is used as coupling agent. The anthranilic acids and 2-alkylthio- and 2-arylthiobenzoic acids used in the final coupling step towards 52-66 were prepared as reported previously. Mei, X.; August, A. T.; Wolf, C. J. Org. Chem. 2006, 71, 142-149; Wolf, C.; Liu, S.; Mei, X.; August, A. T.; Casimir, M. D. J. Org. Chem. 2006, 71, 3270-3273; and Liu, S.; Pestano, J. P. C.; Wolf, C. Synthesis 2007, 3519-3527. Chloroquine-derived amide 65 was directly prepared from N-(7-chloro-4-quinolyl)-1,3-diamine and 5-aminoisatoic anhydride in 64% yield.

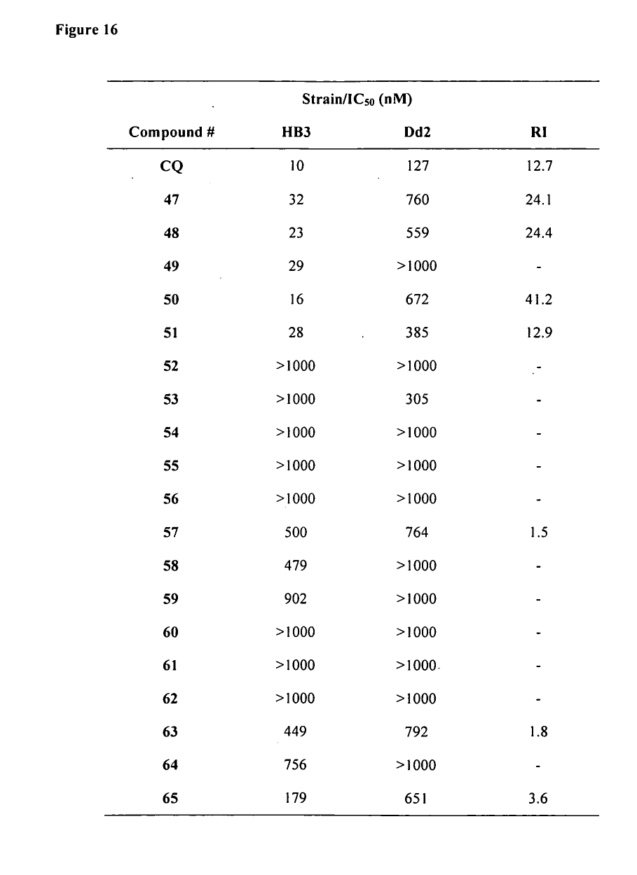

The amide series 57-61 shows high activity against HB3 (IC₅₀'s range from 16.3 to 31.5) but generally less potency against the chloroquine resistant strain Dd2 (FIG. 16). The IC₅₀'s against HB3 do not vary substantially with the chain length. However, comparison of the IC₅₀'s obtained with Dd2 reveals a maximum for 59 which has 4 methylene groups between the 4-aminoquinolyl unit and the amido nitrogen. Apparently, alteration of the chain length again provides an effective tool in the search of new drug candidates that retain their antiplasmodial potency against CQR strains. All other amides prepared proved less effective against both HB3 and Dd2 but it was noticed that the 2-benzylamino-4-fluorobenzoyl derivative 53 was significantly more active against Dd2 than HB3. The higher activity against the CQR strain was even more surprising because this was not the case for its defluorinated analogue 54.

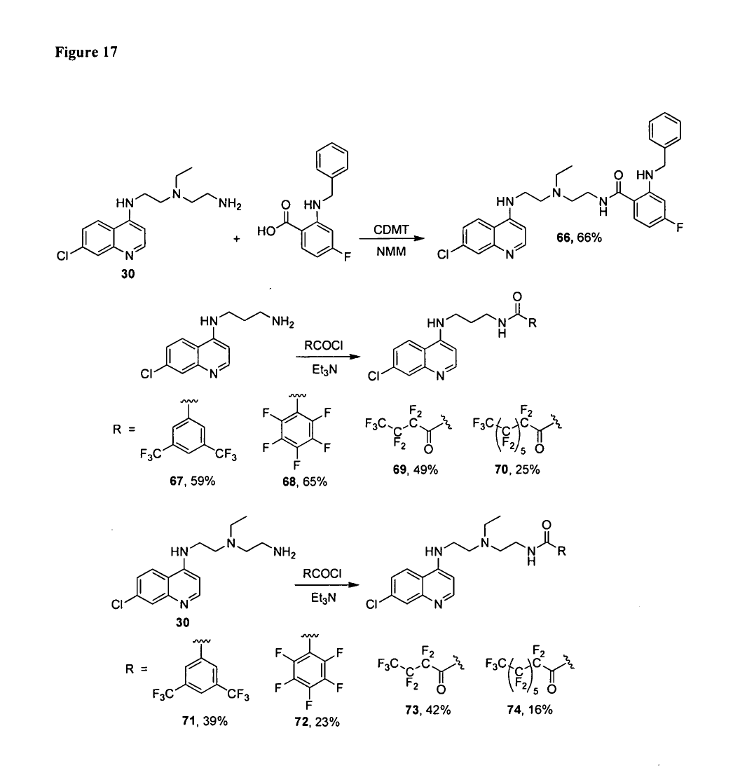

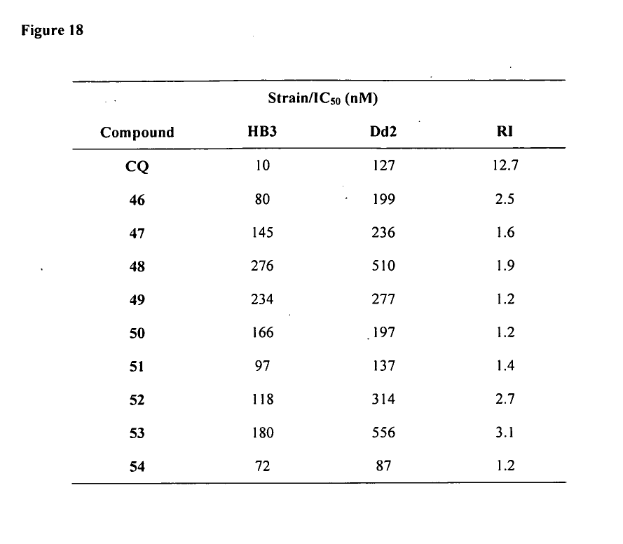

Based on the relatively high activity of 33 against Dd2, several additional fluorinated CQ amides were synthesized (FIG. 17). While amide 46 was prepared via CDMT mediated coupling of 30 with the corresponding benzoic acid derivative, all other amides were obtained using acyl chlorides. We were pleased to find that these fluorinated CQ amides show improved activity compared to 32-45 (FIG. 18). More importantly, fluoro amides 46-54 have excellent RI values ranging from 1.2 to 3.1. This compares favorably with the high RI's determined for amides 47-51, and it underscores that incorporation of fluorinated terminal groups into the CQ side chain can possibly provide a means to circumvent the CQR mechanism.

In sum, more than fifty antiplasmodial 7-chloro-4-aminoquinolyl-derived sulfonamides, ureas, thioureas and amides have been synthesized and tested against CQR and CQS P. falciparum. Many of the CQ analogues prepared showed submicromolar antimalarial activity versus HB3 and Dd2 and low resistance indices. The effects of side chain length, the presence of urea, thiourea, amide and sulfonamide functionalities, and the introduction of fluorinated aliphatic and aromatic termini on the potency against CQS and CQR strains of P. falciparum was investigated. Although none of the quinolyl antimalarials tested was as active as CQ against HB3, more importantly, sulfonamide 23 showed improved activity against the CQR strain Dd2. The results revealed interesting SAR principles leading to promising new directions for the design of antimalarials that address CQ resistance. In particular, sulfonamide 23 exhibiting a short side chain with a terminal dansyl moiety proved significantly more potent against the resistant strain Dd2 than CQ, and incorporation of fluorinated termini into the CQ side chain gave desirable RI indices.

### Structure-Function Principles for Antimalarial Drug Design

Taken together, the results described herein suggest important new structure-function principles for quinoline antimalarial drug design based on chloroquine, including 1) replacement of the terminal tertiary amino function by a secondary moiety reduces the potency vs. CQR strains which suggests the side chain amino group is recognized by the CQ resistance mechanism; 2) substitution of S or O for N at position 4 significantly alters the quinolyl N basicity and lowers the antimalarial potency while improving the selectivity index (defined as the ratio of CQR strain IC₅₀/CQS strain IC₅₀); 3) introduction of an additional basic amino group to the side chain of 4O CQ derivatives can improve the potency while retaining an improved selectivity index; 4) surprisingly, no straightforward relationships between the ability to bind FPIX μ-oxo dimer vs. inhibition of Hz formation and antimalarial potency exists for this series of CQ derivatives.

Results. Recently atomic level resolution structures for CQ, QN, quinidine (QD) and AQ vs. μ-oxo dimer FPIX, as well as the existence of a coordinate CQ-monomeric FPIX complex under acidic aqueous conditions were reported. Leed, A.; DuBay, K.; Ursos, L. M.; Sears, D.; de Dios, A. C.; Roepe, P. D. Solution structures of antimalarial drug-heme complexes. Biochemistry 2002, 41, 10245-10255; de Dios, A. C.; Casabianca, L. B.; Kosar, A.; Roepe, P. D. Structure of the amodiaquine-FPIX μ-oxo dimer solution complex at atomic resolution. Inorg Chem. 2004, 43, 8078-8084; and de Dios, A. C.; Tycko, R.; Ursos, L. M. B.; Roepe, P. D. NMR Studies of Chloroquine-Ferriprotoporphyrin IX Complex J. Phys. Chem. A 2003, 107, 5821-5825. Other data suggest that DV pH may differ for CQS vs. CQR parasites. Bennett, T. N.; Kosar, A. D.; Ursos, L. M.; Dzekunov, S.; Singh Sidhu, A. B.; Fidock, D. A.; Roepe, P. D. Drug resistance-associated PfCRT mutations confer decreased Plasmodium falciparum digestive vacuolar pH. Mol. Biochem. Parasitol. 2004, 133, 99-114. As previously suggested, these data led to several structure-function predictions for CQ analogues that can now be systematically tested via strategic modifications of the CQ structure. These include that simultaneously fine tuning both basicity of the quinolyl N and the length of the CQ side chain may be important for optimizing interactions with FPIX, and that basicity of the tertiary aliphatic N for CQ is important for accumulation within the parasite DV, but not for previously predicted ionic stabilization of CQ-FPIX structures. Along with these principles, previous studies have demonstrated that desethyl CQ has similar activity relative to CQ for CQS strains, but lower activity vs. some CQR strains of P. falciparum. Vippagunta, S. R.; Dorn, A.; Matile, H.; Bhattacharjee, A. K.; Karle, J. M.; Ellis, W. Y.; Ridley, R. G.; Vennerstrom, J. L. Structural specificity of chloroquine-hematin binding related to inhibition of hematin polymerization and parasite growth. J. Med. Chem. 1999, 42, 4630-4639; and Ridley, R. G.; Hofheinz, W.; Matile, H.; Jaquet, C.; Dorn, A.; Masciadri, R.; Jolidon, S.; Richter, W. F.; Guenzi, A.; Girometta, M-A.; Urwyler, H.; Huber, W.; Thaithong, S.; Peters, W.; 4-Aminoquinoline analogs of chloroquine with shortened side chains retain activity against chloroquine-resistant Plasmodium falciparum. Antimicrob. Agents Chemother. 1996, 40, 1846-1854. In addition, shortening or lengthening the aliphatic side chain of CQ has in general been shown to have little effect on the activity vs. CQS strains, but to increase activity vs. CQR strains. De, D.; Krogstad, F. M.; Byers, L. D.; Krogstad, D. J. Structure-activity relationships for antiplasmodial activity among 7-substituted 4-aminoquinolines. J. Med. Chem. 1998, 41, 4918-4926; Stocks, P. A.; Raynes, K. J.; Bray, P. G.; Park, B. K.; O'Neill, P. M.; Ward, S. A. Novel short chain chloroquine analogues retain activity against chloroquine resistant K1 Plasmodium falciparum. J. Med. Chem. 2002, 45, 4975-4983; and Ridley, R. G.; Hofheinz, W.; Matile, H.; Jaquet, C.; Dorn, A.; Masciadri, R.; Jolidon, S.; Richter, W. F.; Guenzi, A.; Girometta, M-A.; Urwyler, H.; Huber, W.; Thaithong, S.; Peters, W.; 4-Aminoquinoline analogs of chloroquine with shortened side chains retain activity against chloroquine-resistant Plasmodium falciparum. Antimicrob. Agents Chemother. 1996, 40, 1846-1854. However, these two modifications have not previously been systematically varied in tandem, which might result in additive or opposing effects. Herein is disclosed data that test the above structure-function predictions for CQ analogues.

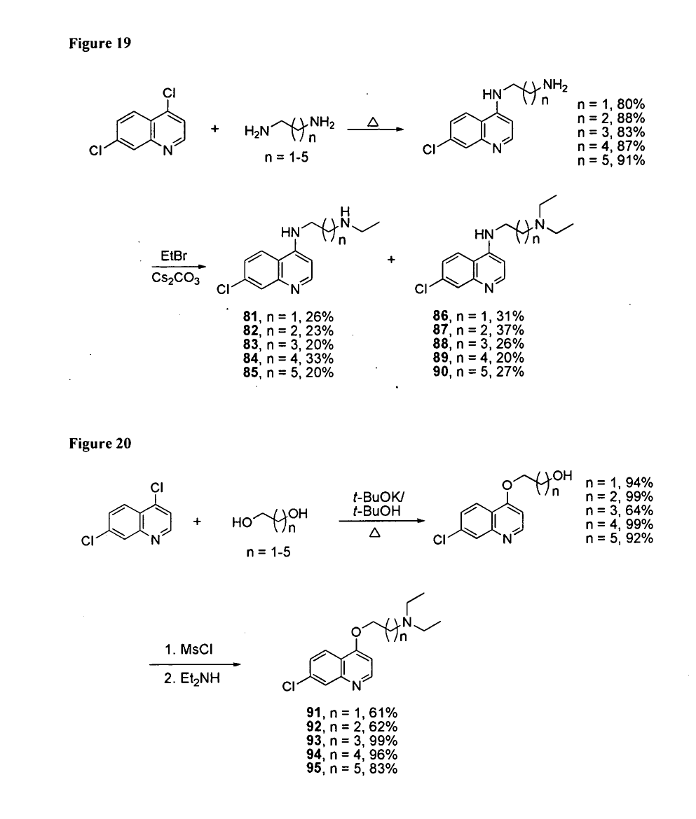

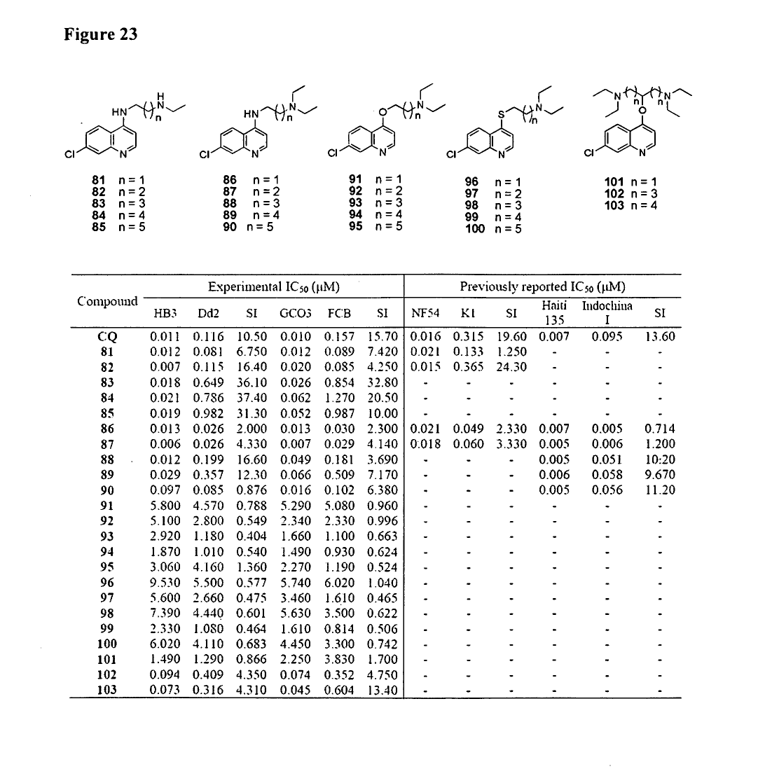

Compounds 81-90 were designed to systematically explore the relationship between mono- vs. diethyl substitution at the terminal aliphatic N and the length of the aliphatic side chain vs. activity against CQS and CQR parasites (FIG. 23). These compounds were prepared in two steps from 4,7-dichloroquinoline and a series of α,ω-diamines. The amination reaction proceeded at elevated temperatures with high yields and the subsequent alkylation with ethyl bromide gave a mixture of approximately 50% of the desired secondary and tertiary amine leaving about 50% of remaining starting materials were recovered in all cases (see FIG. 19). Aminoquinolines 81-90 were then analyzed for activity vs. two CQS and two CQR laboratory strains of P. falciparum using a new semi high-throughput SYBR Green I based assay. This assay was developed independently in two laboratories, is easily standardized, and was recently validated vs. a large collection of antimalarial compounds by the Walter Reed Army Institute. Bennett T. N.; Paguio, M.; Gligorijevic, B.; Seudieu, C.; Kosar, A. D.; Davidson, E.; Roepe, P. D. Novel, rapid, and inexpensive cell-based quantification of antimalarial drug efficacy. Antimicrob. Agents Chemother. 2004, 48, 1807-1810; Smilkstein, M.; Sriwilaijaroen, N.; Kelly, J. X.; Wilairat, P.; Riscoe, M. Simple and inexpensive fluorescence-based technique for high-throughput antimalarial drug screening. Antimicrob. Agents Chemother. 2004, 48, 1803-1806; and Johnson, J. D.; Dennull, R. A.; Gerena, L.; Lopez-Sanchez, M.; Roncal, N. E.; Waters, N.C. Assessment and continued validation of the malaria SYBR green I-based fluorescence assay for use in malaria drug screening. Antimicrob. Agents Chemother. 2007, 51, 1926-1933. As described below, standardization of the activity of candidate antimalarials against different strains and species of Plasmodium is essential for future progress, and the SYBR Green I assay offers one inexpensive route that should be accessible to most laboratories engaged in malaria research.

Aminoquinolines 83, 84 and 85 are novel and have not previously been analyzed vs. malarial parasites, whereas 81, 82 and 86-90 have been synthesized previously using similar but not identical methods (FIG. 19) and tested vs. less commonly used laboratory strains of P. falciparum. Hofheinz, W.; Jaquet, C.; Jolidon, S. Aminochinolin-Derivate mit einer Wirksamkeit gegen Malariaerreger. European patent application 94116281.0, June 1995; Tarbell, D. S.; Shakespeare, N.; Claus, C. J.; Bunnett, J. F. The synthesis of some 7-chloro-4-(3-alkylaminopropylamino)-quinolines. J. Am. Chem. Soc. 1946, 68, 1217-1219; De, D.; Byers, L. D.; Krogstad, D. J. Antimalarials: synthesis of 4-aminoquinolines that circumvent drug resistance in malaria parasites. J. Heterocycl. Chem. 1997, 34, 315-320; Surrey, A. R.; Lesher, G. Y.; Mayer, J. R.; Webb, W. G. Hypotensive agents. 11. The preparation of quaternary salts of some 4-dialkylaminoalkylaminoquinolines J. Am. Chem. Soc. 1959, 81, 2894-2897. Assessment of the activities of all of these related CQ analogues has not previously been standardized using the same strains, culture conditions, and malarial growth inhibition assays. HB3 (CQS, Honduras) and Dd2 (CQR, Indochina) are parents of a genetic cross that produced a collection of progeny (GCO03 [CQS] being one) for which a very large amount of data has been collected regarding the biochemistry and genetics of chloroquine drug resistance. Fidock, D. A.; Nomura, T.; Talley, A. K.; Cooper, R. A.; Dzekunov, S. M.; Ferdig, M. T.; Ursos, L. M.; Sidhu, A. B.; Naude, B.; Deitsch, K. W.; Su, X. Z.; Wootton, J. C.; Roepe, P. D.; Wellems, T. E. Mutations in the P. falciparum digestive vacuole transmembrane protein PfCRT and evidence for their role in chloroquine resistance. Mol. Cell. 2000, 6, 861-871.; Wellems, T. E.; Walker-Jonah, A.; Panton, L. J. Genetic mapping of the chloroquine-resistance locus on Plasmodium falciparum chromosome 7. Proc. Natl. Acad. Sci. U.S.A. 1991, 88, 3382-3386; and Bennett, T. N.; Patel, J.; Ferdig, M. T.; Roepe, P. D. Plasmodium falciparum Na⁺/H⁺ exchanger activity and quinine resistance. Mol. Biochem. Parasitol. 2007, 153, 48-58. Strain FCB (CQR, SE Asia) expresses similar CQR-causing PfCRT mutations relative to Dd2 yet in most laboratories shows 50 to 100% higher levels of CQR relative to Dd2. As such, these strains are valuable reference points for future quinoline based antimalarial drug design guided by ongoing elucidation of the CQR mechanism(s).

Similar, but not identical, IC₅₀ for 92 vs. CQS (HB3, GCO3) and CQR (Dd2, FCB) parasites was measured, consistent with earlier work that assayed CQS strains NF54 and Haiti 135 or CQR strains K1 and Indochina I. Ridley, R. G.; Hofheinz, W.; Matile, H.; Jaquet, C.; Dorn, A.; Masciadri, R.; Jolidon, S.; Richter, W. F.; Guenzi, A.; Girometta, M-A.; Urwyler, H.; Huber, W.; Thaithong, S.; Peters, W.; 4-Aminoquinoline analogs of chloroquine with shortened side chains retain activity against chloroquine-resistant Plasmodium falciparum. Antimicrob. Agents Chemother. 1996, 40, 1846-1854. Differences in precise IC₅₀ are likely due to strain variation, our use of synchronized culture vs. asynchronous culture by others, the use of ³H hypoxanthine incorporation assays vs. the present SYBR Green I approach, or some combination. Regardless, the analysis was expanded to include compounds bearing 4 to 6 methylene groups between the two amino moieties (compounds 83-85) to explore the role of deethylation (as occurs as a consequence of human metabolism) vs. side chain length could be analyzed in more depth (compare the structure of compounds 81-85 vs. 86-90).

Previously, Krogstad and colleagues as well as Ridley et al. observed that the desethyl CQ derivatives 81 or 82 still exhibit high IC₅₀ vs. CQR strains, whereas the diethyl analogues 86 or 87 show substantially lower IC₅₀. de Dios, A. C.; Tycko, R.; Ursos, L. M. B.; Roepe, P. D. NMR Studies of Chloroquine-Ferriprotoporphyrin IX Complex J. Phys. Chem. A 2003, 107, 5821-5825; and Ridley, R. G.; Hofheinz, W.; Matile, H.; Jaquet, C.; Dorn, A.; Masciadri, R.; Jolidon, S.; Richter, W. F.; Guenzi, A.; Girometta, M-A.; Urwyler, H.; Huber, W.; Thaithong, S.; Peters, W.; 4-Aminoquinoline analogs of chloroquine with shortened side chains retain activity against chloroquine-resistant Plasmodium falciparum. Antimicrob. Agents Chemother. 1996, 40, 1846-1854. One hypothesis for the trend in the diethyl side chain series that has been offered previously is that both longer and shorter side chain analogues are less well recognized by the resistance mechanism (for example, drug binding to the mutated PfCRT protein responsible for CQR could be weaker for long and short chain CQ analogues). Zhang, H.; Paguio, M.; Roepe, P. D. The antimalarial drug resistance protein Plasmodium falciparum chloroquine resistance transporter binds chloroquine. Biochemistry, 2004, 43, 8290-8296. If this is indeed the case, then these data suggest that removal of one alkyl group reverses this effect quite dramatically for longer chain analogues. For example, the selectivity index (SI, defined in FIG. 23 caption) obtained for 83-85 is 3 fold higher than for CQ, while IC₅₀ of 83-85 remains near that seen for CQ in CQS strains. That is, the basic tertiary side chain amino group likely contributes to recognition by the CQ resistance mechanism (further discussion below).

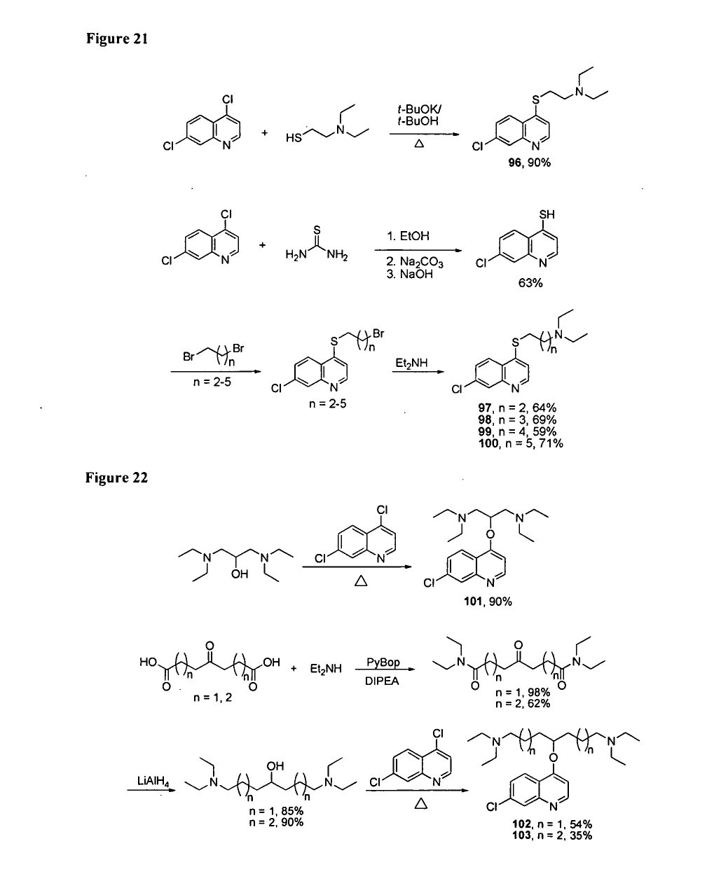

Compounds 91-95 were synthesized from 4,7-dichloroquinoline and α,ω-alkanediols via consecutive nucleophilic displacements (FIG. 20). Compound 96 was synthesized in a single step from 4,7-dichloroquinoline and 2-diethylaminoethanethiol (Scheme 3), and compounds 97-100 were prepared from 7-chloroquinolyl-4-thiol and α,ω-dibromoalkanes via two consecutive S_{N}2 displacements (FIG. 21). These compounds were synthesized in order to inspect the combined effects of heteroatom substitution at the 4 position and the side chain modifications described above. This strategy was pursued (in part) because recent solid state NMR studies have shown that CQ may form a covalent coordinate complex with monomeric FPIX (via a heme Fe-quinoline N bond) under acidic conditions that mimic those of the parasite digestive vacuole (DV). de Dios, A. C.; Tycko, R.; Ursos, L. M. B.; Roepe, P. D. NMR Studies of Chloroquine-Ferriprotoporphyrin IX Complex J. Phys. Chem. A 2003, 107, 5821-5825. Thus, assuming other structural features remain constant, altering the nucleophilicity of the quinolyl N (as predicted for 4O and 4S substitutions) would influence reactivity of the drug vs. monomeric heme without necessarily altering the structure required for non-covalent association to μ-oxo dimer heme, and hence preference for drug-monomer vs. drug heme dimer complexes. Leed, A.; DuBay, K.; Ursos, L. M.; Sears, D.; de Dios, A. C.; Roepe, P. D. Solution structures of antimalarial drug-heme complexes. Biochemistry 2002, 41, 10245-102551; and de Dios, A. C.; Casabianca, L. B.; Kosar, A.; Roepe, P. D. Structure of the amodiaquine-FPIX μ-oxo dimer solution complex at atomic resolution. Inorg Chem. 2004, 43, 8078-8084.

Yet, none of these compounds showed heightened activity relative to CQ, and in fact exhibited only modest antimalarial activity, with IC₅₀ values in the μM range (FIG. 23). However, the selectivity index (SI; c.f. FIG. 23) is substantially improved for several of these compounds. Thus, the 4S and 4O CQ analogue structures are valid starting points for quinoline based antimalarial drug design wherein the goal is improved activity vs. CQR strains (e.g., lower SI, see results for compounds 101-103, below).

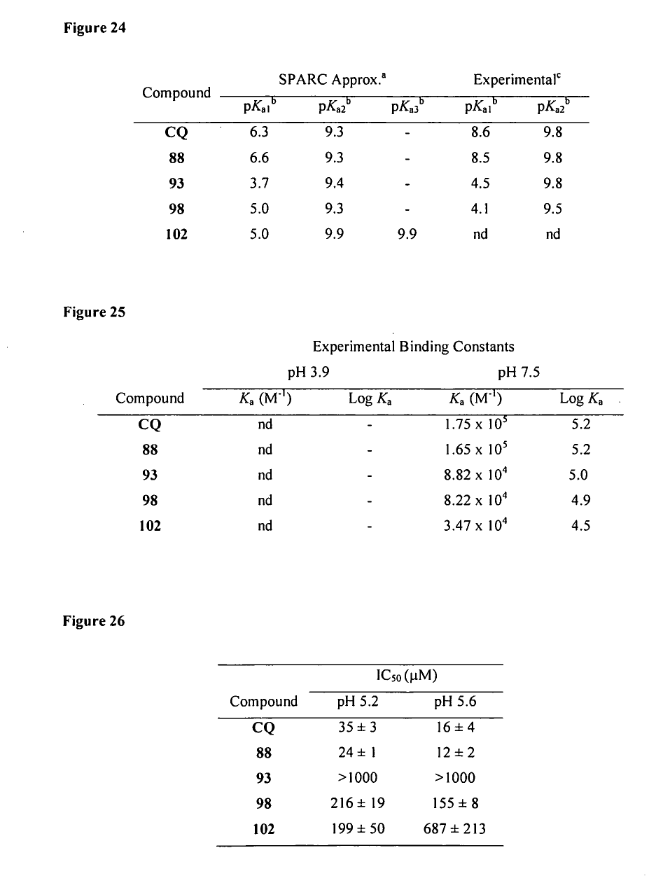

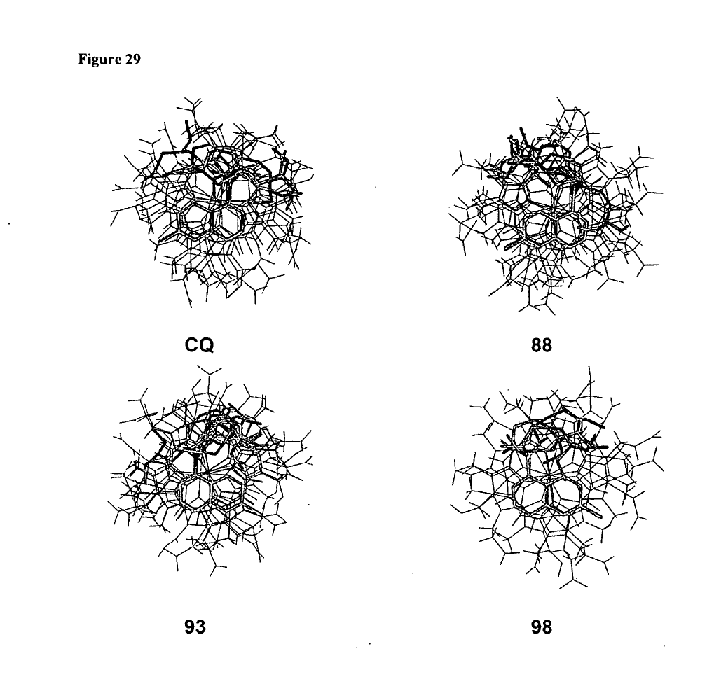

To further probe the molecular basis of these trends in relative activity and selectivity index, other features of quinoline based drugs that are believed to be critical with regard to their antimalarial potency were analyzed. A structurally related set that best mimics the overall structure of CQ (namely, the members of this set include those compounds that contain side chains of similar length relative to CQ; 88, 93, 98) was examined in detail. The pKa of titratable N were calculated and measured (FIG. 24), binding constants for μ-oxo dimeric and monomeric heme were measured in aqueous and 40% DMSO solutions, respectively (FIG. 25), and the ability to inhibit Hz formation in vitro at DV pH measured for CQS (5.6) and CQR (5.2) parasites are tabulated (FIG. 26). In addition, inversion recovery experiments at varied drug:dimer heme ratios were performed and solved the atomic level structures of complexes formed between these drugs and μ-oxo dimer heme (FIGS. 28 and 29).

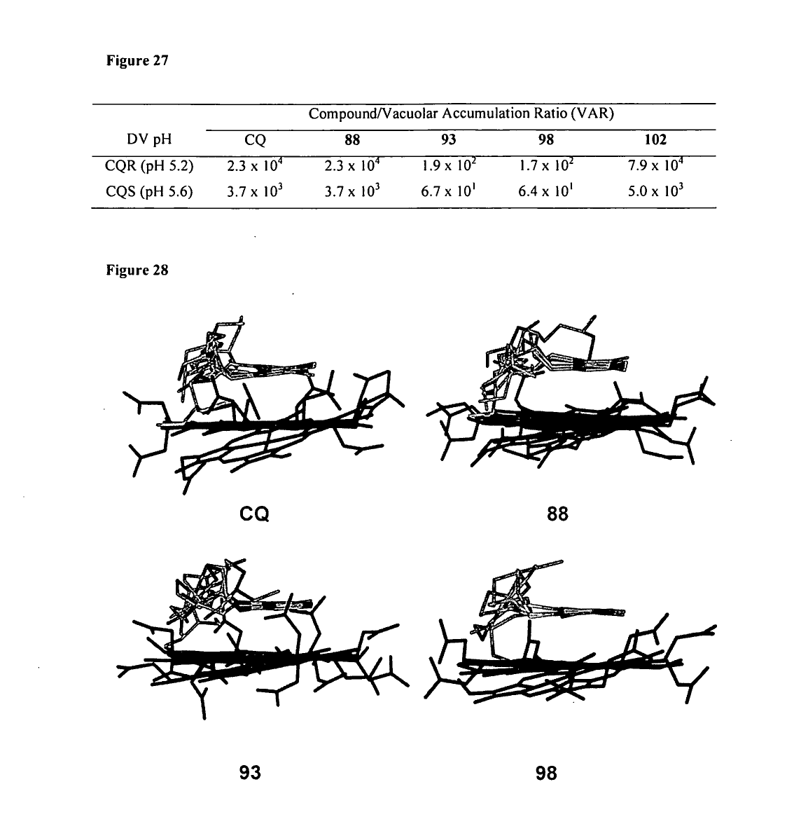

The pKa data show that incorporation of alkoxy and alkylthio substituents into position 4 affords CQ analogues that are effectively monoprotic weak bases at physiologic pH. CQ and the 4N CQ derivatives have pKa's of approximately 10 and 8.5 (FIG. 24) and are effectively diprotic weak bases and thus concentrate within the acidic parasite DV proportional to the square of the net pH gradient (DV interior to outside). However, concentration of the effectively monoprotic 4S and 4O analogues will be linearly related to the net pH gradient as shown in FIG. 27, which summarizes our calculations for DV accumulation for each compound. Ursos, L. M.; Roepe, P. D. Chloroquine resistance in the malarial parasite, Plasmodium falciparum. Med. Res. Rev. 2002, 22, 465-491. Thus one possible explanation for the reduced activity of these compounds is a lowered ability to concentrate within the DV (site of hemoglobin digestion and release of free heme).

To test whether binding to Hz precursors is also altered for the 4S and 4O derivatives, binding constants were measured as previously described for both μ-oxo dimeric and monomeric heme (FIG. 25). Constantinidis, I.; Satterlee, J. D. UV-visible and carbon NMR studies of chloroquine binding to urohemin I chloride and uroporphyrin I in aqueous solutions. J. Am. Chem. Soc. 1988, 110, 4391-4395; Egan, T. J.; Mavuso, W. W.; Ross, D. C.; Marques, H. M. Thermodynamic factors controlling the interaction of quinoline antimalarial drugs with ferriprotoporphyrin IX. J. Inorg. Biochem. 1997, 68, 137-145; and Egan, T. J.; Ncokazi, K. K. Effects of solvent composition and ionic strength on the interaction of quinoline antimalarials with ferriprotoporphyrin IX. J. Inorg. Biochem. 2004, 98, 144-152. Due to the instability of monomeric heme in aqueous solution, the affinity to monomer measured by conventional absorbance experiments can only be estimated using 40% DMSO in water as solvent. Egan, T. J.; Ncokazi, K. K. Effects of solvent composition and ionic strength on the interaction of quinoline antimalarials with ferriprotoporphyrin IX. J. Inorg. Biochem. 2004, 98, 144-152. At appropriately acidic solution pH (≦5.0) FPIX heme is primarily monomeric, whereas at pH 7, appreciable dimer is formed. As shown (FIG. 25), CQ and compounds 88, 93, and 98 all have poor affinity for monomeric heme in acidic 40% DMSO. To further test if CQ interacts with monomeric heme, T₁ measurements of the CQ protons were made with samples containing 10 mM CQ and 2 mM hemin chloride in 40% DMSO at pH 5.0. Although the lines are broadened in this solution due to paramagnetic susceptibility, the measured T₁'s indicate only weak paramagnetic relaxation. For example, the measured T₁ for CQ proton 1 in this sample is 0.70 s, whereas at pH 7.0 the T₁ for the same proton is 0.039 s. The longer T₁'s in the lower pH sample indicate that CQ does not interact appreciably with monomeric heme.

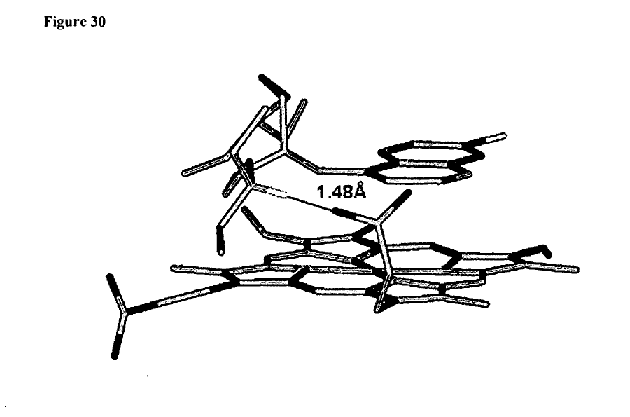

FIG. 25 also tabulates similar measured affinities for compounds 88, 93, and 98 vs. μ-oxo dimer in aqueous solution. Inspection of the side and top-down views of the noncovalent solution structures formed between these drugs and μ-oxo dimeric heme solved via T₁ measurements (FIGS. 29 and 30) shows that the overall geometries (and hence calculated binding energies) are quite similar. Thus, to a first approximation, interactions between either CQ, 88, 93, or 98 and monomeric or dimeric heme are all similar.

Surprisingly then, the ability of 93, 98 to inhibit Hz formation was found to be significantly lower vs. that measured for CQ and 8 (FIG. 26). These results, viewed alongside data in FIGS. 25, 28 and 29, suggest that noncovalent complexation with μ-oxo dimer heme is unlikely to be the primary mode of inhibition of Hz formation. Interactions between these compounds and other heme aggregates or the growing faces of Hz must play an important role, since the relative μ-oxo dimer binding constants and complex geometries (energies) do not correlate with the relative ability of these compounds to perturb Hz growth in vitro (FIG. 26 vs. FIGS. 25, 28 and 29).

Since the SI was improved for several of the 4O CQ derivatives, but since accumulation of these effectively monobasic drugs into the DV of the parasite is predicted to be lower than CQ and the 4N derivatives (FIG. 27), “symmetrically branched”, dibasic 4O CQ analogues 101-103 (Scheme 4) were designed and synthesized. Starting from either 5-oxoazelaic acid or 4-ketopimelic acid, two α,ω-bis(diethylamido)alkanones were synthesized by coupling with diethylamine in the presence of PyBop (benzotriazol-1-yl-oxytripyrrolidinophosphonium hexafluorophosphate). As expected, reduction with lithium aluminum hydride gave the corresponding α,ω-bis(diethylamino)alkanols. Deprotonation with potassium tert-butoxide and subsequent nucleophilic aromatic substitution at position 4 of 4,7-dichloroquinoline then yielded 7-chloro-4-(1′,7′-bis(diethylamino)-4′-heptoxy) quinoline, 102, and 7-chloro-4-(1′,9′-bis(diethylamino)-5′-nonoxy) quinoline, 103, respectively. Since 1,3-bis(diethylamino)-2-propanol is readily available, we were able to prepare 101 in a single step. Unfortunately, attempts to extend this synthetic strategy to symmetrically branched 4S CQ derivatives via thiation of α,ω-bis(diethylamido)alkanones with Lawesson's reagent and subsequent reduction towards secondary thiols were not successful. As a result, our optimization efforts were restricted to 4O CQ analogues carrying two basic terminal amino groups. One candidate was designed based on the monobasic 4O compound (93) that showed good activity (μM IC₅₀) vs. both CQR strains. Compound 102 harbors one extended aliphatic chain of similar length relative to 93 (4 methylenes between the 4N-quinolyl unit and the tertiary aliphatic amino group) such that it is predicted to wrap around the periphery of the protoporphyrin ring when forming a non-covalent complex with dimeric FPIX as previously observed for 93 (FIG. 29), and a second aliphatic chain of appropriate length for possible ion pairing with a free FPIX propionate (FIG. 30). Interestingly, this compound and its homologue 103 showed improved activity in vivo vs. both CQR and CQS strains relative to 93 (FIG. 23), whereas the shorter chain analogue (101) that cannot ion pair with free propionate, remained significantly less active. Also, importantly, although it was initially expected that improved activity would be due merely to increased accumulation (addition of the diamino branched side chain converts the monoprotic derivative 93 to a diprotic weak base at physiologic pH; (c.f. FIGS. 24 and 27), FIG. 28 shows that 102 is also a more potent inhibitor of Hz formation relative to monobasic 4O derivative 13. Interestingly, this is in spite of similar affinity for heme (FIG. 25), further emphasizing the lack of a simple relationship between heme monomer or heme μ-oxo dimer binding affinity and the ability of a drug to inhibit the formation of Hz. It is noted however that we measure Hz formation using an in vitro assay that may not completely mimic Hz formation in vivo.

Discussion. The collection of compounds discussed above introduce systematic modifications of the CQ side chain structure and cover a range of antiplasmodial activities. These modifications were suggested by detailed analysis of CQ-heme target structures that have recently been solved, as described elsewhere and, taken in their entirety, are more subtle and systematic than most previous quinoline antimalarial drug design studies. Such structure-function based analysis of candidate antimalarials is relatively rare but required since inexpensive antimalarial drugs active against CQR malaria are desperately needed. From these experiments several new and important conclusions relevant to inexpensive quinoline antimalarial drug design were drawn:

1) Substitution of the amino function by a secondary derivative at the terminus of the side chain of 4-amino-7 chloroquinolines generally reduces the potency against CQR strains, but shows little effect on the antimalarial activity vs. CQS strains.

2) Replacement of the 4-position nitrogen atom of the 7-chloroquinoline by either sulfur or oxygen substantially decreases the basicity of the quinolyl nitrogen which correlates with a general decrease in antimalarial activity. Thus, without further modification (see point 3 below) the basicity of the quinolyl N is crucial to antiplasmodial activity.

3) However, introduction of an additional basic amino group to the side chain of 4O CQ derivatives improves potency vs. both CQS and CQR strains while preserving an improved selectivity index, and also substantially increases the ability of 4O CQ analogues to inhibit formation of Hz while not altering the affinity to either monomeric or μ-oxo dimeric FPIX.

4) Surprisingly, and in contrast to many assumptions in the literature, we find no straightforward relationship between the ability to bind FPIX μ-oxo dimer and inhibition of Hz formation, nor any simple relationship between either of these drug characteristics and antimalarial potency.

With regard to conclusion 1, the well known observation that either shortening or lengthening the aliphatic side chain of CQ specifically improves activity vs. CQR parasites only holds for diethyl derivatives. Surprisingly, longer chain monoethyl analogues with otherwise identical side chains (e.g., 85 vs. 90) show relatively high IC₅₀ vs. CQR parasites, whereas either mono or diethyl short chain analogues are improved (e.g., 81, 86). The observed trends for 81-90 are important for two reasons; first, the data suggest that substituents at the terminal aliphatic N may interact with the CQ resistance mechanism, Fidock, D. A.; Nomura, T.; Talley, A. K.; Cooper, R. A.; Dzekunov, S. M.; Ferdig, M. T.; Ursos, L. M.; Sidhu, A. B.; Naude, B.; Deitsch, K. W.; Su, X. Z.; Wootton, J. C.; Roepe, P. D.; Wellems, T. E. Mutations in the P. falciparum digestive vacuole transmembrane protein PfCRT and evidence for their role in chloroquine resistance. Mol. Cell. 2000, 6, 861-871; and De, D.; Byers, L. D.; Krogstad, D. J. Antimalarials: synthesis of 4-aminoquinolines that circumvent drug resistance in malaria parasites. J. Heterocycl. Chem. 1997, 34, 315-320. Current models for the CQ resistance mechanism propose direct interaction of CQ with mutant PfCRT protein. Fidock, D. A.; Nomura, T.; Talley, A. K.; Cooper, R. A.; Dzekunov, S. M.; Ferdig, M. T.; Ursos, L. M.; Sidhu, A. B.; Naude, B.; Deitsch, K. W.; Su, X. Z.; Wootton, J. C.; Roepe, P. D.; Wellems, T. E. Mutations in the P. falciparum digestive vacuole transmembrane protein PfCRT and evidence for their role in chloroquine resistance. Mol. Cell. 2000, 6, 861-871; and Zhang, H.; Paguio, M.; Roepe, P. D. The antimalarial drug resistance protein Plasmodium falciparum chloroquine resistance transporter binds chloroquine. Biochemistry, 2004, 43, 8290-8296. If correct, then results with 81-85 vs. 86-90 suggest a secondary amino group at the terminus of the side chain allows for better binding to PfCRT. Along with guiding additional modifications of quinolines, this concept should be useful for determining the nature of PfCRT CQ binding sites via the design of azido-drug analogues or other probes. Zhang, H.; Paguio, M.; Roepe, P. D. The antimalarial drug resistance protein Plasmodium falciparum chloroquine resistance transporter binds chloroquine. Biochemistry, 2004, 43, 8290-8296. Second, these results suggest that metabolism to desethyl derivatives will impair the activity of longer chain CQ analogues vs. CQR parasites much more so than is the case for short chain analogues.

With regard to conclusion 2, it is noted that another recent report on one compound in which carbon is substituted for nitrogen at position 4. Cheruku S R, Maiti S, Dorn A, Scorneaux B, Bhattacharjee A K, Ellis W Y, Vennerstrom J L. Carbon isosteres of the 4-aminopyridine substructure of chloroquine: effects on pK(a), hematin binding, inhibition of Hz formation, and parasite growth. J Med. Chem. 2003, 46(14), 3166-9. This CQ isotere has the same length side chain as does CQ (as is the case for 93, 98) and showed a similarly reduced quinolyl N pKa (measured to be 4.8 vs. 4.5 and 4.1 for 93 and 98, respectively). However, the 4C CQ isotere showed even more significantly reduced potency, with no affect on parasite growth measured up to 3 μM drug. Thus, substitution with sulfur or oxygen at position 4 is not analogous to substitution with carbon even though all three affect quinolyl N pKa to a similar extent. Specifically, the improved SI of the analogues with oxygen at position 4, the about 1 μM IC₅₀ vs. CQR parasites for some compounds (e.g., 93, 94) and the ability to further titrate potency without fully reversing improved SI via addition of additional basic N to the aliphatic side chain (e.g., compounds 102, 103) suggests the 4O CQ pharmacophore is an attractive scaffold for drug design schemes to circumvent CQR.

Conclusions 3 and 4 have several important implications for antimalarial drug design, and force a rethinking of recent proposals for the action of CQ and related quinoline antimalarials. Importantly, it was found that binding to heme (either the μ-oxo or monomeric forms) is not necessarily correlated with the ability of a CQ analogue to inhibit Hz formation. Association constants, Kₐ, (μ-oxo dimer) are quite similar for a representative set of compounds with side chain length similar to CQ (88, 93, and 98), whereas IC₅₀ for Hz inhibition among the same group of compounds varies by 100 fold. This is particularly impressive since 88, 93, and 98 differ only at position 4 but are otherwise identical. Association to monomer (in 40% DMSO) is similarly very weak for all compounds, and lowest energy geometries for the 88, 93, or 98μ-oxo dimer complex structures deduced by inversion recovery experiments are very similar. It is noted that although the biologically relevant dimer for Hz crystallization is the tethered head-to-tail dimer and not the μ-oxo, noncovalent association with this dimer is likely quite similar and governed by similar π-π and van der Waals interactions as described. Thus these data suggest that quinoline compounds inhibit Hz formation via some other mechanism. Possibilities include binding to one or more growing crystal faces, or by association with monomeric heme that cannot be measured in 40% DMSO solution. It is also suggested that the lipophilicity of the noncovalent complex, which depends on the protonation state of the quinolyl N, needs to be accounted for, since recent work suggests Hz formation at a rate commensurate with what is observed in vivo is catalyzed by a lipid environment. Chong, C. R.; Sullivan, D. J. Jr. Inhibition of heme crystal growth by antimalarials and other compounds: implications for drug discovery. Biochem Pharmacol. 2003, 66, 2201-2212; Egan, T. J.; Mavuso, W. W.; Ross, D.C.; Marques, H. M. Thermodynamic factors controlling the interaction of quinoline antimalarial drugs with ferriprotoporphyrin IX. J. Inorg. Biochem. 1997, 68, 137-145; and Egan, T. J.; Ncokazi, K. K. Effects of solvent composition and ionic strength on the interaction of quinoline antimalarials with ferriprotoporphyrin IX. J. Inorg. Biochem. 2004, 98, 144-152.

Observed trends in this rationally designed series of compounds point out that even subtle variation in the quinoline structure can very significantly influence the ability to inhibit Hz formation, and that complex relationships between heme affinity and Hz inhibition exist for even very closely related quinoline antimalarials. It is noted that the improved activity of 102 relative to 93 is due to both an unanticipated improved ability to inhibit Hz as well as increased accumulation within the DV due to an improved VAR (vacuolar accumulation ratio). In addition, it is noted that the relative ability of these compounds to inhibit Hz formation at either pH 5.6 (approximate DV pH measured for CQS parasites) or pH 5.2 (approximate DV pH measured for CQR parasites) is not well correlated with their antimalarial activity vs. CQS or CQR strains. Bennett, T. N.; Kosar, A. D.; Ursos, L. M.; Dzekunov, S.; Singh Sidhu, A. B.; Fidock, D. A.; Roepe, P. D. Drug resistance-associated PfCRT mutations confer decreased Plasmodium falciparum digestive vacuolar pH. Mol. Biochem. Parasitol. 2004, 133, 99-114; and Gligorijevic, B.; Bennett, T.; McAllister, R.; Urbach, J. S.; Roepe, P. D. Spinning disk confocal microscopy of live, intraerythrocytic malarial parasites. 2. Altered vacuolar volume regulation in drug resistant malaria. Biochemistry 2006, 45, 12411-12423. This conclusion is in contrast to previous work with other quinoline-based antimalarials. Dorn, A.; Vippagunta, S. R.; Matile, H.; Jaquet, C.; Vennerstrom, J. L.; Ridley, R. G. An assessment of drug-haematin binding as a mechanism for inhibition of haematin polymerisation by quinoline antimalarials. Biochem. Pharmacol. 1998, 55, 727-736. For example, 102 has a 10 fold lower IC₅₀ vs. strains Dd2 and FCB relative to 98, but a nearly identical IC₅₀ for Hz inhibition at pH 5.2. More dramatically, 93 shows a 4 fold lower IC₅₀ vs. strain Dd2 relative to 98, but roughly 5-fold higher IC₅₀ for Hz inhibition. In the previously reported trend, only one CQS strain (NF54) was tested and the drugs examined were not as structurally similar as those in this study. Importantly then, either the chemistry of drug inhibition of Hz formation differs in some interesting way for CQR vs. CQS parasites, or DV accumulation for many of these compounds is also influenced by substitution at the 4 position and differs significantly for CQS vs. CQR parasites. Perhaps both concepts are relevant, since we also now find differences in Hz inhibition IC₅₀ at pH 5.2 vs. 5.6 for CQ and other members of this series. Although the concept remains controversial, several reports have noted that mutant PfCRT found in the DV membrane of CQR parasites confers lower endosomal pH, and that the pH for CQR DV is about 5.2 whereas for CQS it is closer to 5.6. Bennett, T. N.; Kosar, A. D.; Ursos, L. M.; Dzekunov, S.; Singh Sidhu, A. B.; Fidock, D. A.; Roepe, P. D. Drug resistance-associated PfCRT mutations confer decreased Plasmodium falciparum digestive vacuolar pH. Mol. Biochem. Parasitol. 2004, 133, 99-114; Gligorijevic, B.; Bennett, T.; McAllister, R.; Urbach, J. S.; Roepe, P. D. Spinning disk confocal microscopy of live, intraerythrocytic malarial parasites. 2. Altered vacuolar volume regulation in drug resistant malaria. Biochemistry 2006, 45, 12411-12423; 39) Reeves, D.C.; Liebelt, D. A.; Lakshmanan, V.; Roepe, P. D.; Fidock, D. A.; Akabas, M. H. Chloroquine-resistant isoforms of the Plasmodium falciparum chloroquine resistance transporter acidify lysosomal pH in HEK293 cells more than chloroquine-sensitive isoforms. Mol. Biochem. Parasitol. 2006, 150, 288-299; and Naude, B.; Brzostowski, J. A.; Kimmel, A. R.; Wellems, T. E. Dictyostelium discoideum expresses a malaria chloroquine resistance mechanism upon transfection with mutant, but not wild-type, Plasmodium falciparum transporter PfCRT. J. Biol. Chem. 2005, 280, 25596-25603. Also, the volume of the DV, and apparent Cl⁻-dependent volume regulatory processes differ for CQR vs. CQS parasites, with DV volume for CQR parasites recently measured to be significantly larger. Gligorijevic, B.; Bennett, T.; McAllister, R.; Urbach, J. S.; Roepe, P. D. Spinning disk confocal microscopy of live, intraerythrocytic malarial parasites. 2. Altered vacuolar volume regulation in drug resistant malaria. Biochemistry 2006, 45, 12411-12423. Assuming a similar rate of hemoglobin metabolism (and hence liberation of free heme) within the DV as suggested, then these simple changes in the chemical environment for heme within the DV (i.e., bulk pH and heme concentration) likely affect the ability of a given quinoline compound to exert toxic effects via the production of heme-drug complexes. Leed, A.; DuBay, K.; Ursos, L. M.; Sears, D.; de Dios, A. C.; Roepe, P. D. Solution structures of antimalarial drug-heme complexes. Biochemistry 2002, 41, 10245-10255; de Dios, A. C.; Casabianca, L. B.; Kosar, A.; Roepe, P. D. Structure of the amodiaquine-FPIX μ-oxo dimer solution complex at atomic resolution. Inorg Chem. 2004, 43, 8078-8084; de Dios, A. C.; Tycko, R.; Ursos, L. M. B.; Roepe, P. D. NMR Studies of Chloroquine-Ferriprotoporphyrin IX Complex J. Phys. Chem. A 2003, 107, 5821-5825; and Gligorijevic, B.; Bennett, T.; McAllister, R.; Urbach, J. S.; Roepe, P. D. Spinning disk confocal microscopy of live, intraerythrocytic malarial parasites. 2. Altered vacuolar volume regulation in drug resistant malaria. Biochemistry 2006, 45, 12411-12423.

In summary, a set of CQ structural modifications has been prepared based upon predictions from recent atomic-level elucidation of drug-heme complexes. Overall, the results suggest additional modifications to CQ that can promote improved selectivity vs. CQR parasites and illustrate that relationships between heme binding, Hz inhibition, and antimalarial activity are more complex than previously thought. The data also show that additional modification of compounds with an improved SI, that work to promote improved bioavailability, can provide valuable new leads for further development of inexpensive quinoline antimalarials with good activity vs. CQR parasites (e.g., compounds 102 and 103).

### Selected Compounds of the Invention

One aspect of the invention relates to the preparation a series of new heme-targeted antimalarials obtained by systematically varying both the structure and basicity of the side chain of quinoline antimalarial compounds.

For example, one aspect of the invention relates to a compound of formula I-V:

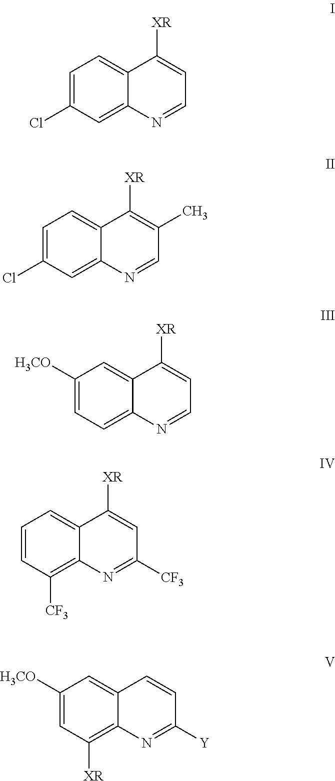

wherein, independently for each occurrence,

X is —N(H)—, —O— or —S—;

Y is hydrogen, alkyl, aryl or heteroaryl;

R is

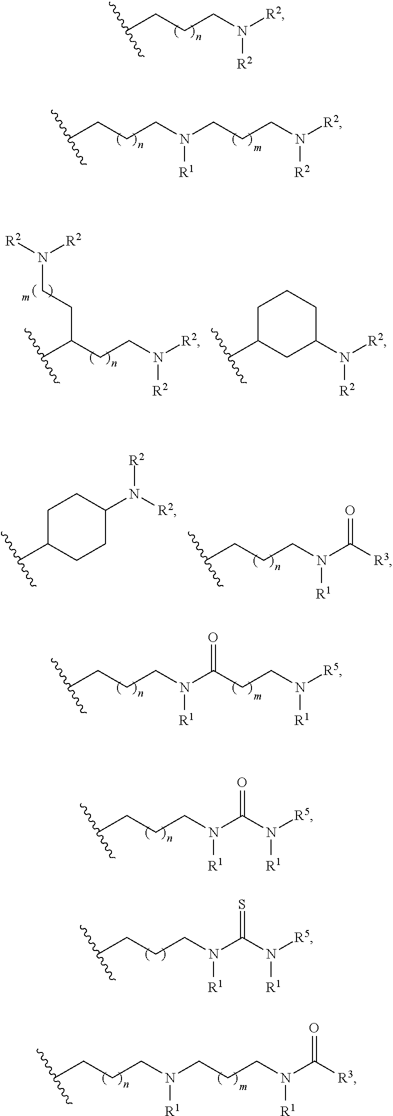

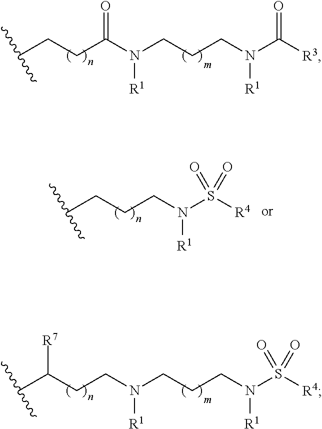

R¹ is hydrogen or alkyl;

R² is hydrogen or alkyl;

R³ is haloalkyl, aryl, aralkyl, heteroaryl, heteroaralkyl,

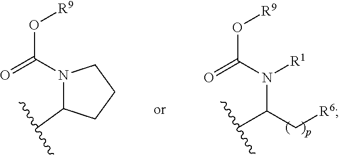

R⁴ is aryl or heteroaryl;

R⁵ is aryl or heteroaryl;

R⁶ is aryl, heteroaryl or

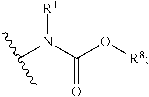

R⁷ is hydrogen or alkyl;

R⁸ is aryl, heteroaryl, aralkyl or heteroaralkyl;

R⁹ is hydrogen or alkyl;

n is 0-5 inclusive;

m is 0-5 inclusive;

p is 0-5 inclusive; and

each aryl and heteroaryl moiety, including those which are a part of an aralkyl or heteroaralkyl moiety, is optional substituted with 1-3 substitutents selected from the group consisting of alkyl, cycloalkyl, halo, perhaloalkyl, aralkyl, heteroaralkyl, alkenyl, alkynyl, carbonyl, ester, carboxyl, carboxylic acid, formyl, thiocarbonyl, thioester, thiocarboxylic acid, thioformyl, ketone, aldehyde, cyano, isocyano, amino, acylamino, amido, nitro, hydroxyl, alkoxy, aryloxy, heteroaryloxy, aralkyloxy, sulfhydryl, alkylthio, arylthio, heteroarylthio, aralkylthio, heteroaralkylthio, sulfoxido, sulfonyl, oxysulfonyl, sulfonylamino, sulfamoyl, carbocyclyl, polycyclyl, aryl, heteroaryl, and heterocyclyl.

In certain embodiments, the present invention relates to any one of the aforementioned compounds and any attendant definitions, wherein X is —N(H)—. In certain embodiments, the present invention relates to any one of the aforementioned compounds and any attendant definitions, wherein X is —O—. In certain embodiments, the present invention relates to any one of the aforementioned compounds and any attendant definitions, wherein X is —S—.

In certain embodiments, the present invention relates to any one of the aforementioned compounds and any attendant definitions, wherein Y is hydrogen.

In certain embodiments, the present invention relates to any one of the aforementioned compounds and any attendant definitions, wherein the compound is represented by

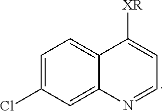

In certain embodiments, the present invention relates to any one of the aforementioned compounds and any attendant definitions, wherein the compound is represented by

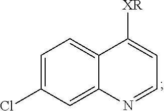

and X is —N(H)—. In certain embodiments, the present invention relates to any one of the aforementioned compounds and any attendant definitions, wherein the compound is represented by

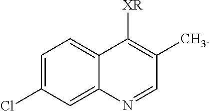

In certain embodiments, the present invention relates to any one of the aforementioned compounds and any attendant definitions, wherein the compound is represented by

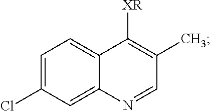

and X is —N(H)—. In certain embodiments, the present invention relates to any one of the aforementioned compounds and any attendant definitions, wherein the compound is represented by

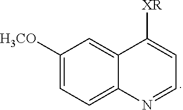

In certain embodiments, the present invention relates to any one of the aforementioned compounds and any attendant definitions, wherein the compound is represented by

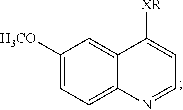

and X is —N(H)—. In certain embodiments, the present invention relates to any one of the aforementioned compounds and any attendant definitions, wherein the compound is represented by

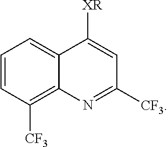

In certain embodiments, the present invention relates to any one of the aforementioned compounds and any attendant definitions, wherein the compound is represented by

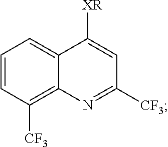

and X is —N(H)—. In certain embodiments, the present invention relates to any one of the aforementioned compounds and any attendant definitions, wherein the compound is represented by

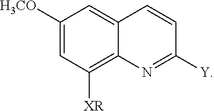

In certain embodiments, the present invention relates to any one of the aforementioned compounds and any attendant definitions, wherein the compound is represented by

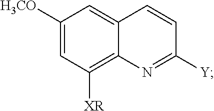

and Y is hydrogen. In certain embodiments, the present invention relates to any one of the aforementioned compounds and any attendant definitions, wherein the compound is represented by

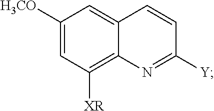

Y is hydrogen; and X is —N(H)—.

In certain embodiments, the present invention relates to any one of the aforementioned compounds and any attendant definitions, wherein R is

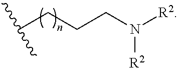

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein R² is hydrogen, methyl, ethyl, propyl or isopropyl. In certain embodiments, the present invention relates to any one of the aforementioned compounds and any attendant definitions, wherein one R² is hydrogen; and one R² is methyl, ethyl, propyl or isopropyl. In certain embodiments, the present invention relates to any one of the aforementioned compounds and any attendant definitions, wherein one R² is hydrogen; and one R² is ethyl. In certain embodiments, the present invention relates to any one of the aforementioned compounds and any attendant definitions, wherein R² is C₁₋₄ alkyl. In certain embodiments, the present invention relates to any one of the aforementioned compounds and any attendant definitions, wherein R² is methyl, ethyl, propyl or isopropyl. In certain embodiments, the present invention relates to any one of the aforementioned compounds and any attendant definitions, wherein R² is ethyl.

In certain embodiments, the present invention relates to any one of the aforementioned compounds and any attendant definitions, wherein the compound is selected from the group consisting of

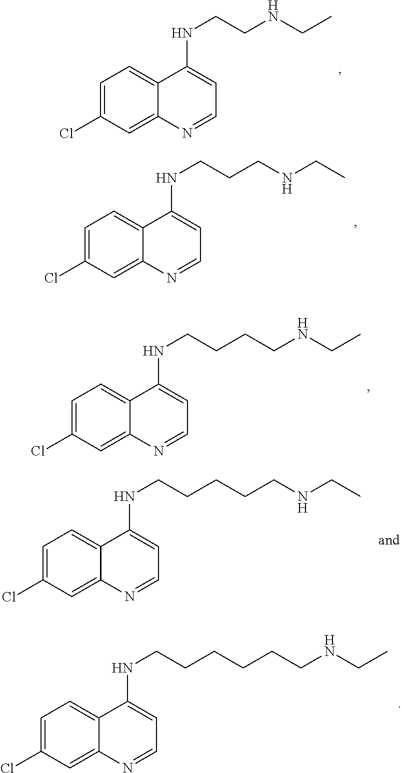

In certain embodiments, the present invention relates to any one of the aforementioned compounds and any attendant definitions, wherein the compound is selected from the group consisting of

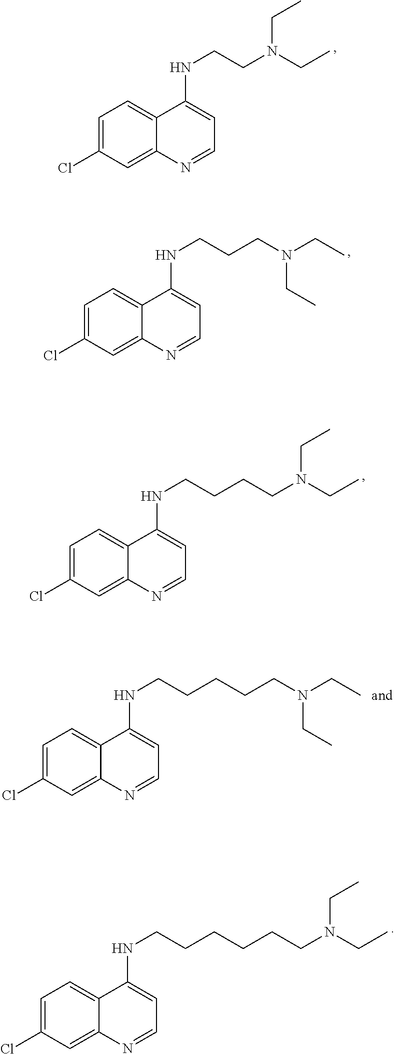

In certain embodiments, the present invention relates to any one of the aforementioned compounds and any attendant definitions, wherein the compound is selected from the group consisting of

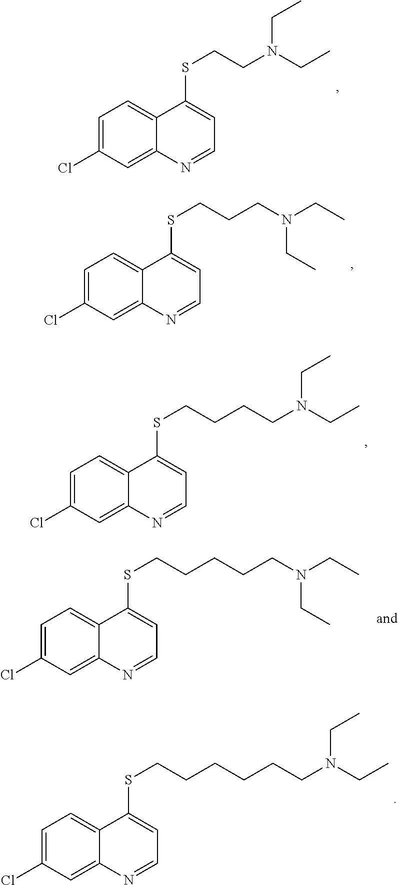

In certain embodiments, the present invention relates to any one of the aforementioned compounds and any attendant definitions, wherein the compound is selected from the group consisting of

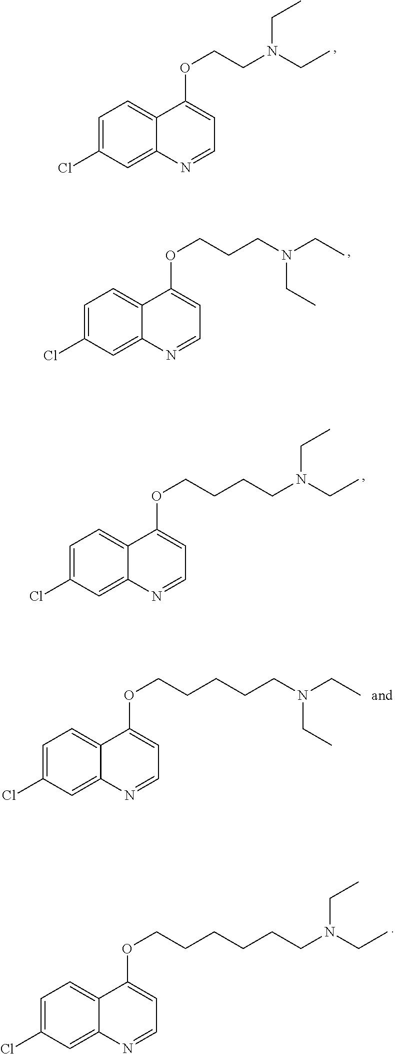

In certain embodiments, the present invention relates to any one of the aforementioned compounds and any attendant definitions, wherein R is

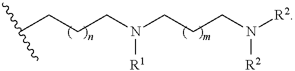

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein m is 0. In certain embodiments, the present invention relates to any one of the aforementioned compounds and any attendant definitions, wherein m is 1. In certain embodiments, the present invention relates to any one of the aforementioned compounds and any attendant definitions, wherein m is 2. In certain embodiments, the present invention relates to any one of the aforementioned compounds and any attendant definitions, wherein m is 3. In certain embodiments, the present invention relates to any one of the aforementioned compounds and any attendant definitions, wherein m is 4. In certain embodiments, the present invention relates to any one of the aforementioned compounds and any attendant definitions, wherein m is 5.

In certain embodiments, the present invention relates to any one of the aforementioned compounds and any attendant definitions, wherein R¹ is hydrogen, methyl, ethyl, propyl or isopropyl. In certain embodiments, the present invention relates to any one of the aforementioned compounds and any attendant definitions, wherein R¹ is hydrogen. In certain embodiments, the present invention relates to any one of the aforementioned compounds and any attendant definitions, wherein R¹ is ethyl.

In certain embodiments, the present invention relates to any one of the aforementioned compounds and any attendant definitions, wherein R² is C₁₋₄ alkyl. In certain embodiments, the present invention relates to any one of the aforementioned compounds and any attendant definitions, wherein R² is methyl, ethyl, propyl or isopropyl. In certain embodiments, the present invention relates to any one of the aforementioned compounds and any attendant definitions, wherein R² is ethyl.

In certain embodiments, the present invention relates to any one of the aforementioned compounds and any attendant definitions, wherein the compound is selected from the group consisting of

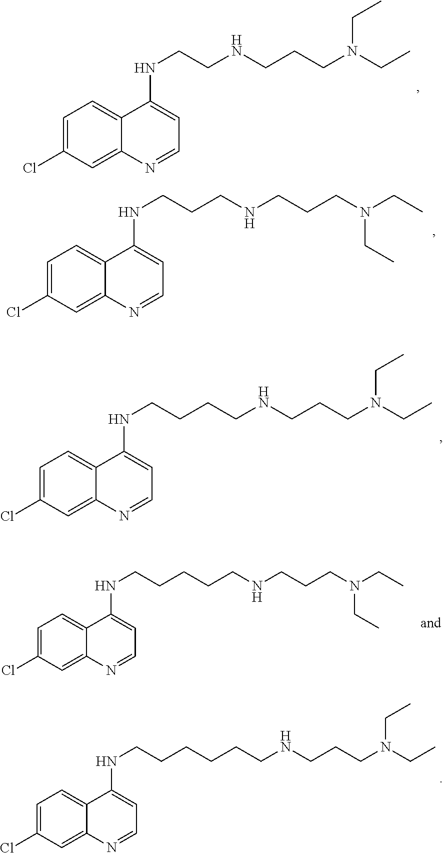

In certain embodiments, the present invention relates to any one of the aforementioned compounds and any attendant definitions, wherein the compound is selected from the group consisting of

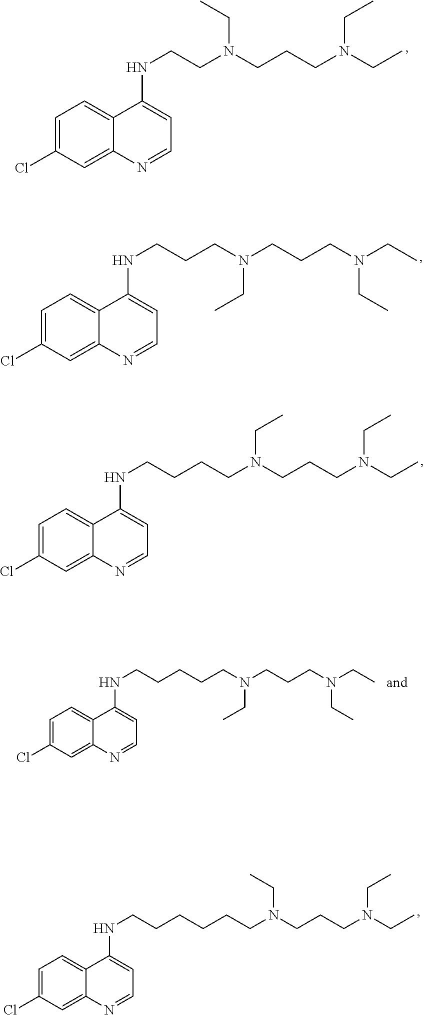

In certain embodiments, the present invention relates to any one of the aforementioned compounds and any attendant definitions, wherein R is

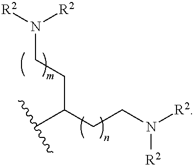

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein m is 0. In certain embodiments, the present invention relates to any one of the aforementioned compounds and any attendant definitions, wherein m is 1. In certain embodiments, the present invention relates to any one of the aforementioned compounds and any attendant definitions, wherein m is 2. In certain embodiments, the present invention relates to any one of the aforementioned compounds and any attendant definitions, wherein m is 3. In certain embodiments, the present invention relates to any one of the aforementioned compounds and any attendant definitions, wherein m is 4. In certain embodiments, the present invention relates to any one of the aforementioned compounds and any attendant definitions, wherein m is 5.

In certain embodiments, the present invention relates to any one of the aforementioned compounds and any attendant definitions, wherein R² is C₁₋₄ alkyl. In certain embodiments, the present invention relates to any one of the aforementioned compounds and any attendant definitions, wherein R² is methyl, ethyl, propyl or isopropyl. In certain embodiments, the present invention relates to any one of the aforementioned compounds and any attendant definitions, wherein R² is ethyl. In certain embodiments, the present invention relates to any one of the aforementioned compounds and any attendant definitions, wherein R² is isopropyl.

In certain embodiments, the present invention relates to any one of the aforementioned compounds and any attendant definitions, wherein the compound is selected from the group consisting of

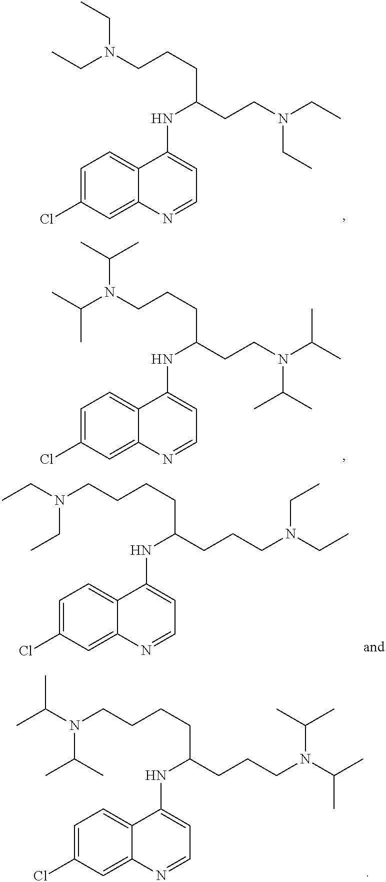

In certain embodiments, the present invention relates to any one of the aforementioned compounds and any attendant definitions, wherein the compound is selected from the group consisting of

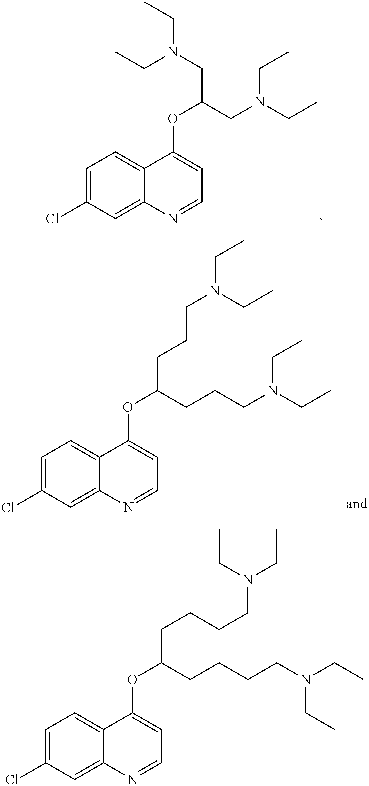

In certain embodiments, the present invention relates to any one of the aforementioned compounds and any attendant definitions, wherein R is

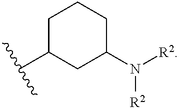

In certain embodiments, the present invention relates to any one of the aforementioned compounds and any attendant definitions, wherein R² is hydrogen or C₁₋₄ alkyl. In certain embodiments, the present invention relates to any one of the aforementioned compounds and any attendant definitions, wherein R² is hydrogen. In certain embodiments, the present invention relates to any one of the aforementioned compounds and any attendant definitions, wherein R² is methyl, ethyl, propyl or isopropyl. In certain embodiments, the present invention relates to any one of the aforementioned compounds and any attendant definitions, wherein R² is ethyl. In certain embodiments, the present invention relates to any one of the aforementioned compounds and any attendant definitions, wherein R² is isopropyl.

In certain embodiments, the present invention relates to any one of the aforementioned compounds and any attendant definitions, wherein the compound is selected from the group consisting of

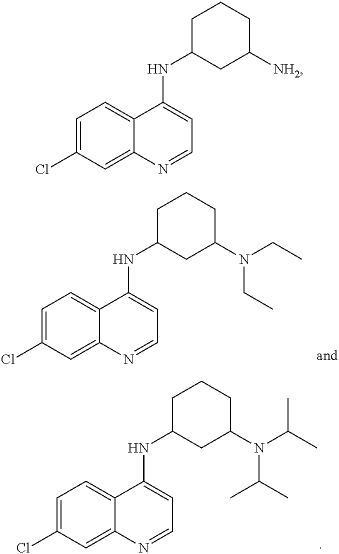

In certain embodiments, the present invention relates to any one of the aforementioned compounds and any attendant definitions, wherein R is

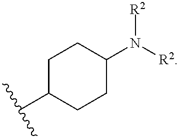

In certain embodiments, the present invention relates to any one of the aforementioned compounds and any attendant definitions, wherein R² is hydrogen or C₁₋₄ alkyl. In certain embodiments, the present invention relates to any one of the aforementioned compounds and any attendant definitions, wherein R² is hydrogen. In certain embodiments, the present invention relates to any one of the aforementioned compounds and any attendant definitions, wherein R² is methyl, ethyl, propyl or isopropyl. In certain embodiments, the present invention relates to any one of the aforementioned compounds and any attendant definitions, wherein R² is ethyl. In certain embodiments, the present invention relates to any one of the aforementioned compounds and any attendant definitions, wherein R² is isopropyl.

In certain embodiments, the present invention relates to any one of the aforementioned compounds and any attendant definitions, wherein the compound is selected from the group consisting of

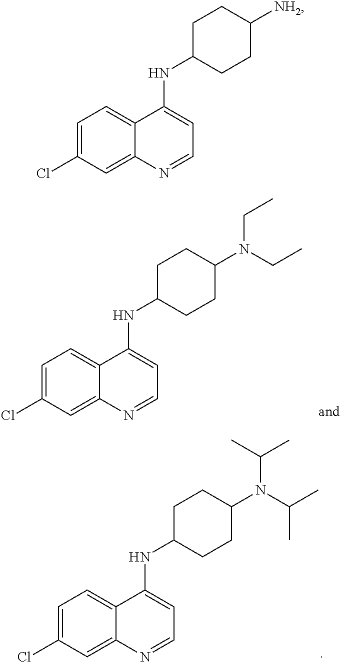

In certain embodiments, the present invention relates to any one of the aforementioned compounds and any attendant definitions, wherein R is

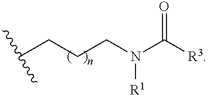

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein R¹ is hydrogen.

In certain embodiments, the present invention relates to any one of the aforementioned compounds and any attendant definitions, wherein R³ is haloalkyl. In certain embodiments, the present invention relates to any one of the aforementioned compounds and any attendant definitions, wherein R³ is perfluoroalkyl. In certain embodiments, the present invention relates to any one of the aforementioned compounds and any attendant definitions, wherein R³ is —(CF₂)_{w}CF₃; and w is 1-7 inclusive. In certain embodiments, the present invention relates to any one of the aforementioned compounds and any attendant definitions, wherein R³ is —(CF₂)₂CF₃. In certain embodiments, the present invention relates to any one of the aforementioned compounds and any attendant definitions, wherein R³ is —(CF₂)₅CF₃

In certain embodiments, the present invention relates to any one of the aforementioned compounds and any attendant definitions, wherein the compound is selected from the group consisting of

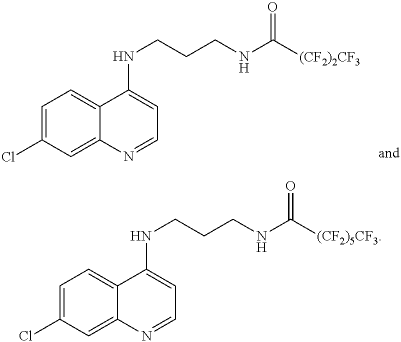

In certain embodiments, the present invention relates to any one of the aforementioned compounds and any attendant definitions, wherein R³ is aryl. In certain embodiments, the present invention relates to any one of the aforementioned compounds and any attendant definitions, wherein R³ is an phenyl substituted with at least one amino, haloalkyl, halo, arylthio, alkylthio, or hydroxyl substituent. In certain embodiments, the present invention relates to any one of the aforementioned compounds and any attendant definitions, wherein R³ is selected from the group consisting of

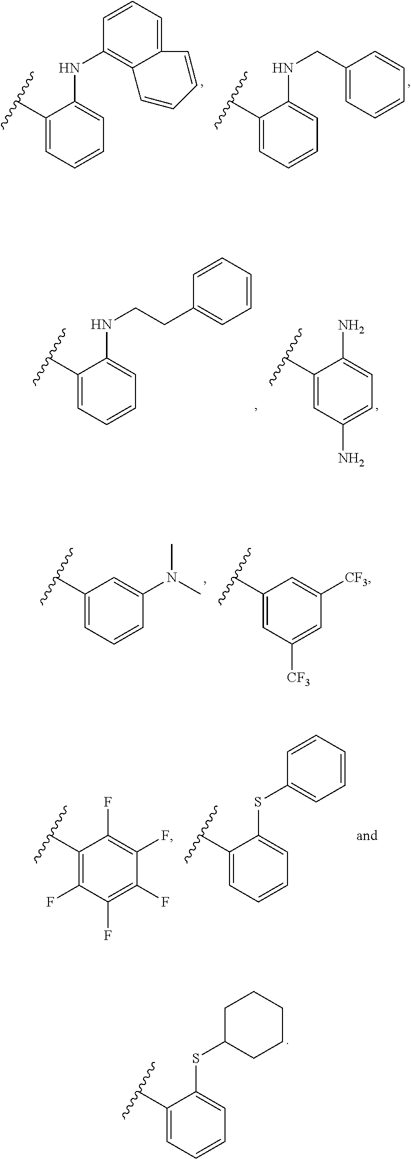

In certain embodiments, the present invention relates to any one of the aforementioned compounds and any attendant definitions, wherein the compound is selected from the group consisting of

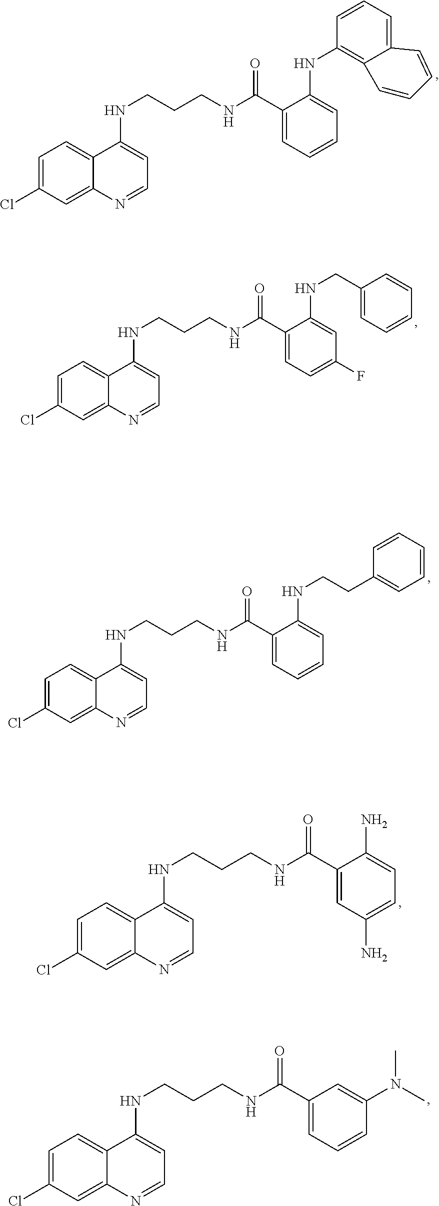

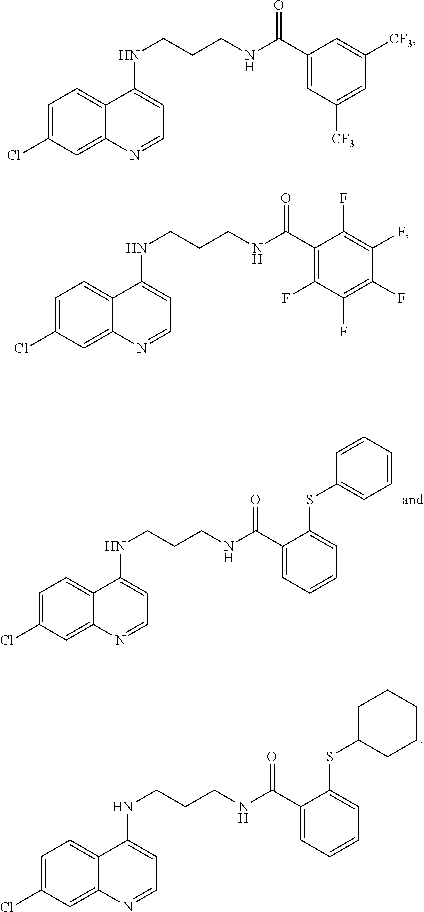

In certain embodiments, the present invention relates to any one of the aforementioned compounds and any attendant definitions, wherein R³ is heteroaryl. In certain embodiments, the present invention relates to any one of the aforementioned compounds and any attendant definitions, wherein R³ is a pyridinyl substituted with at least one amino, haloalkyl, halo, arylthio, alkylthio, or hydroxyl substituent. In certain embodiments, the present invention relates to any one of the aforementioned compounds and any attendant definitions, wherein R³ is selected from the group consisting of

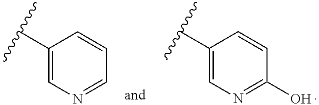

In certain embodiments, the present invention relates to any one of the aforementioned compounds and any attendant definitions, wherein the compound is selected from the group consisting of

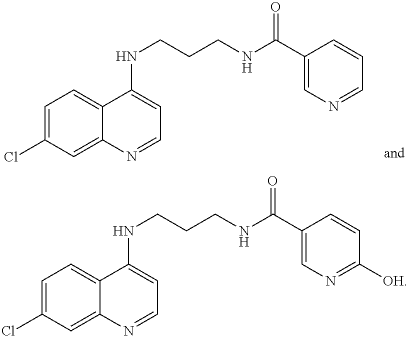

In certain embodiments, the present invention relates to any one of the aforementioned compounds and any attendant definitions, wherein R³ is heteroaralkyl. In certain embodiments, the present invention relates to any one of the aforementioned compounds and any attendant definitions, wherein R³ is an alkyl substituted with a 1H-benzo[d]imidazole substituent. In certain embodiments, the present invention relates to any one of the aforementioned compounds and any attendant definitions, wherein R³ is

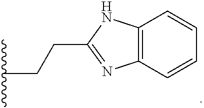

In certain embodiments, the present invention relates to any one of the aforementioned compounds and any attendant definitions, wherein the compound is

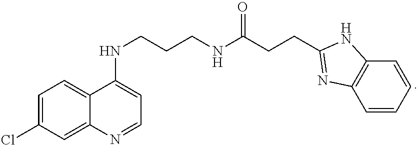

In certain embodiments, the present invention relates to any one of the aforementioned compounds and any attendant definitions, wherein R³ is

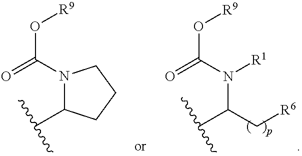

In certain embodiments, the present invention relates to any one of the aforementioned compounds and any attendant definitions, wherein R⁹ is alkyl. In certain embodiments, the present invention relates to any one of the aforementioned compounds and any attendant definitions, wherein R⁹ is t-butyl.

In certain embodiments, the present invention relates to any one of the aforementioned compounds and any attendant definitions, wherein R¹ is hydrogen.

In certain embodiments, the present invention relates to any one of the aforementioned compounds and any attendant definitions, wherein R⁶ is heteroaryl. In certain embodiments, the present invention relates to any one of the aforementioned compounds and any attendant definitions, wherein R⁶ is

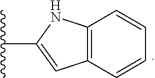

In certain embodiments, the present invention relates to any one of the aforementioned compounds and any attendant definitions, wherein R⁶ is

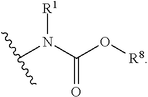

In certain embodiments, the present invention relates to any one of the aforementioned compounds and any attendant definitions, wherein R⁸ is aralkyl. In certain embodiments, the present invention relates to any one of the aforementioned compounds and any attendant definitions, wherein R⁸ is benzyl.

In certain embodiments, the present invention relates to any one of the aforementioned compounds and any attendant definitions, wherein p is 0. In certain embodiments, the present invention relates to any one of the aforementioned compounds and any attendant definitions, wherein p is 1. In certain embodiments, the present invention relates to any one of the aforementioned compounds and any attendant definitions, wherein p is 2. In certain embodiments, the present invention relates to any one of the aforementioned compounds and any attendant definitions, wherein p is 3. In certain embodiments, the present invention relates to any one of the aforementioned compounds and any attendant definitions, wherein p is 4. In certain embodiments, the present invention relates to any one of the aforementioned compounds and any attendant definitions, wherein p is 5.

In certain embodiments, the present invention relates to any one of the aforementioned compounds and any attendant definitions, wherein the compound is selected from the group consisting of

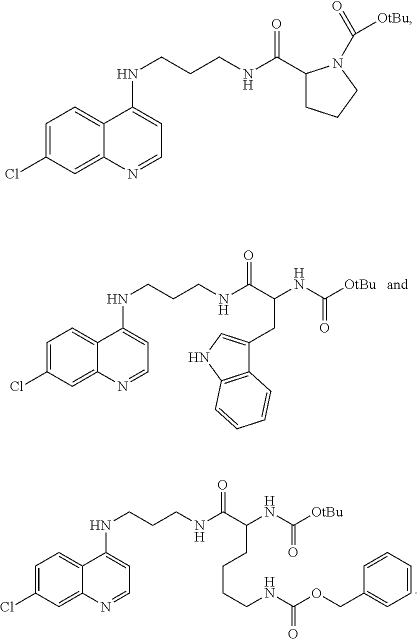

In certain embodiments, the present invention relates to any one of the aforementioned compounds and any attendant definitions, wherein R is

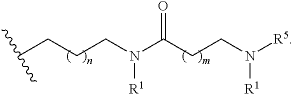

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein m is 0. In certain embodiments, the present invention relates to any one of the aforementioned compounds and any attendant definitions, wherein m is 1. In certain embodiments, the present invention relates to any one of the aforementioned compounds and any attendant definitions, wherein m is 2. In certain embodiments, the present invention relates to any one of the aforementioned compounds and any attendant definitions, wherein m is 3. In certain embodiments, the present invention relates to any one of the aforementioned compounds and any attendant definitions, wherein m is 4. In certain embodiments, the present invention relates to any one of the aforementioned compounds and any attendant definitions, wherein m is 5.

In certain embodiments, the present invention relates to any one of the aforementioned compounds and any attendant definitions, wherein R¹ is hydrogen.

In certain embodiments, the present invention relates to any one of the aforementioned compounds and any attendant definitions, wherein R⁵ is C₁₋₄ alkyl. In certain embodiments, the present invention relates to any one of the aforementioned compounds and any attendant definitions, wherein R⁵ is methyl, ethyl, propyl or isopropyl. In certain embodiments, the present invention relates to any one of the aforementioned compounds and any attendant definitions, wherein R⁵ is ethyl.

In certain embodiments, the present invention relates to any one of the aforementioned compounds and any attendant definitions, wherein the compound is selected from the group consisting of

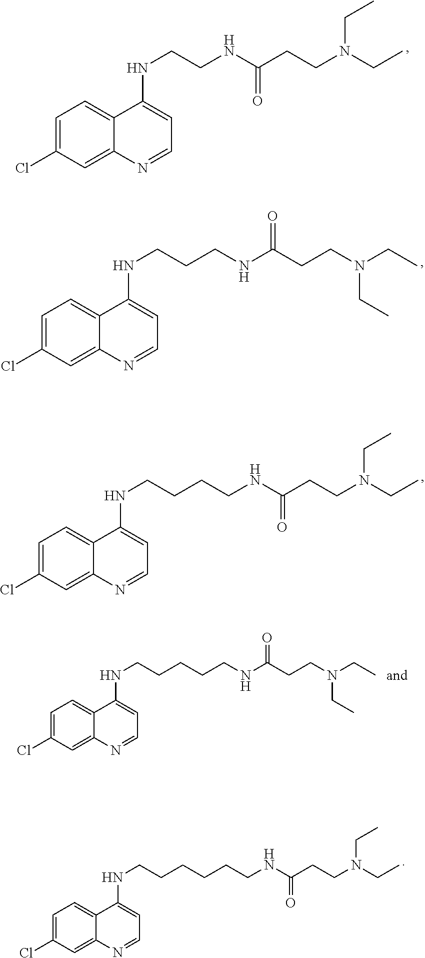

In certain embodiments, the present invention relates to any one of the aforementioned compounds and any attendant definitions, wherein R is

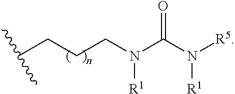

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein R¹ is hydrogen.

In certain embodiments, the present invention relates to any one of the aforementioned compounds and any attendant definitions, wherein R⁵ is aryl. In certain embodiments, the present invention relates to any one of the aforementioned compounds and any attendant definitions, wherein R⁵ is an phenyl substituted with at least one amino, alkoxy, or nitro substituent. In certain embodiments, the present invention relates to any one of the aforementioned compounds and any attendant definitions, wherein R⁵ is selected from the group consisting of

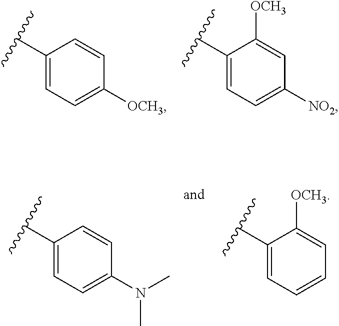

In certain embodiments, the present invention relates to any one of the aforementioned compounds and any attendant definitions, wherein the compound is selected from the group consisting of

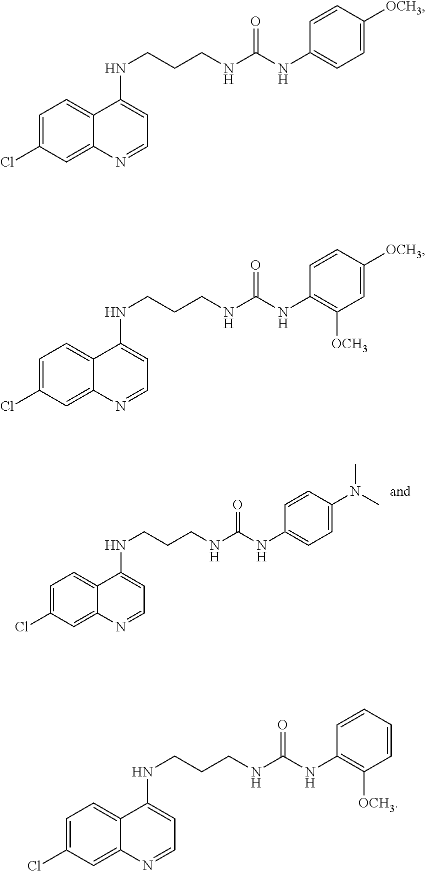

In certain embodiments, the present invention relates to any one of the aforementioned compounds and any attendant definitions, wherein R is

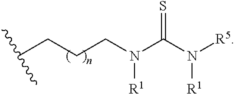

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein R¹ is hydrogen.

In certain embodiments, the present invention relates to any one of the aforementioned compounds and any attendant definitions, wherein R⁵ is aryl. In certain embodiments, the present invention relates to any one of the aforementioned compounds and any attendant definitions, wherein R⁵ is an phenyl or naphthyl substituted with at least one amino, alkoxy, or nitro substitutent. In certain embodiments, the present invention relates to any one of the aforementioned compounds and any attendant definitions, wherein R⁵ is selected from the group consisting of

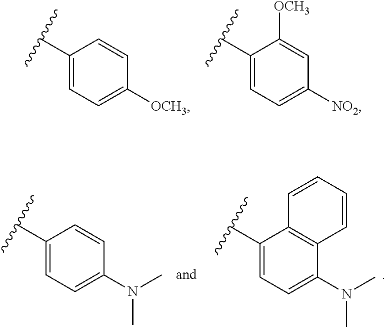

In certain embodiments, the present invention relates to any one of the aforementioned compounds and any attendant definitions, wherein the compound is selected from the group consisting of

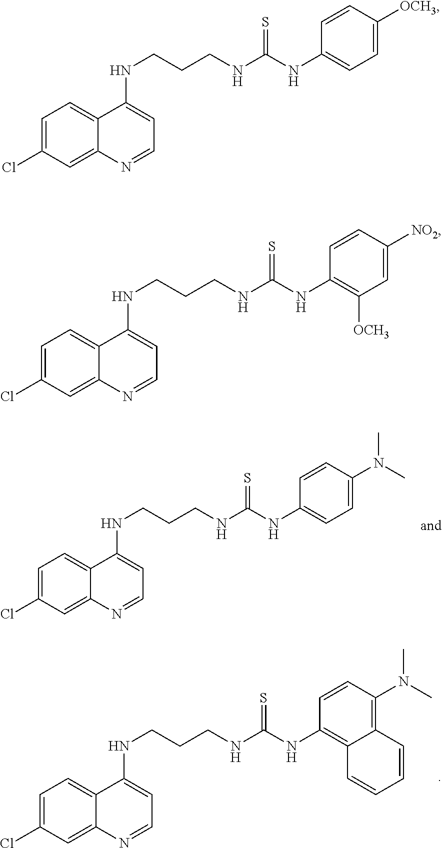

In certain embodiments, the present invention relates to any one of the aforementioned compounds and any attendant definitions, wherein R is

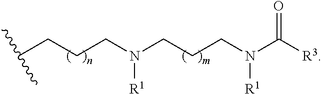

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein m is 0. In certain embodiments, the present invention relates to any one of the aforementioned compounds and any attendant definitions, wherein m is 1. In certain embodiments, the present invention relates to any one of the aforementioned compounds and any attendant definitions, wherein m is 2. In certain embodiments, the present invention relates to any one of the aforementioned compounds and any attendant definitions, wherein m is 3. In certain embodiments, the present invention relates to any one of the aforementioned compounds and any attendant definitions, wherein m is 4. In certain embodiments, the present invention relates to any one of the aforementioned compounds and any attendant definitions, wherein m is 5.

In certain embodiments, the present invention relates to any one of the aforementioned compounds and any attendant definitions, wherein R¹ is hydrogen.

In certain embodiments, the present invention relates to any one of the aforementioned compounds and any attendant definitions, wherein R³ is haloalkyl. In certain embodiments, the present invention relates to any one of the aforementioned compounds and any attendant definitions, wherein R³ is perfluoroalkyl. In certain embodiments, the present invention relates to any one of the aforementioned compounds and any attendant definitions, wherein R³ is —(CF₂)_{w}CF₃; and w is 1-7 inclusive. In certain embodiments, the present invention relates to any one of the aforementioned compounds and any attendant definitions, wherein R³ is —(CF₂)₂CF₃. In certain embodiments, the present invention relates to any one of the aforementioned compounds and any attendant definitions, wherein R³ is —(CF₂)₅CF₃

In certain embodiments, the present invention relates to any one of the aforementioned compounds and any attendant definitions, wherein the compound is selected from the group consisting of

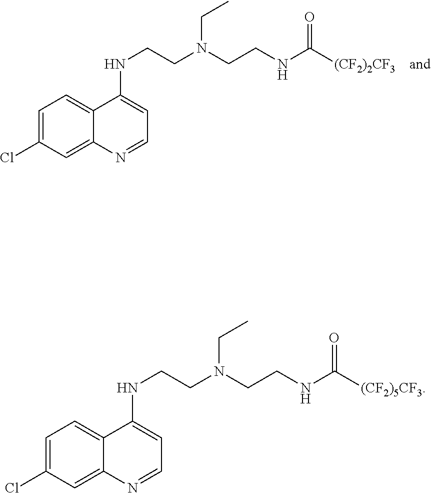

In certain embodiments, the present invention relates to any one of the aforementioned compounds and any attendant definitions, wherein R³ is aryl. In certain embodiments, the present invention relates to any one of the aforementioned compounds and any attendant definitions, wherein R³ is an phenyl substituted with at least one amino, haloalkyl, or halo substituent. In certain embodiments, the present invention relates to any one of the aforementioned compounds and any attendant definitions, wherein R³ is selected from the group consisting of

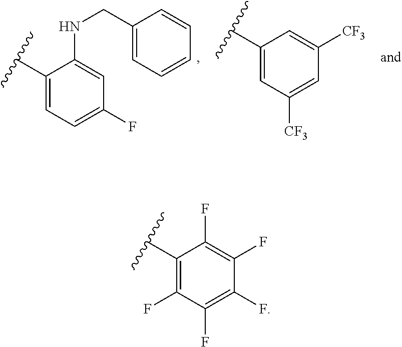

In certain embodiments, the present invention relates to any one of the aforementioned compounds and any attendant definitions, wherein the compound is selected from the group consisting of

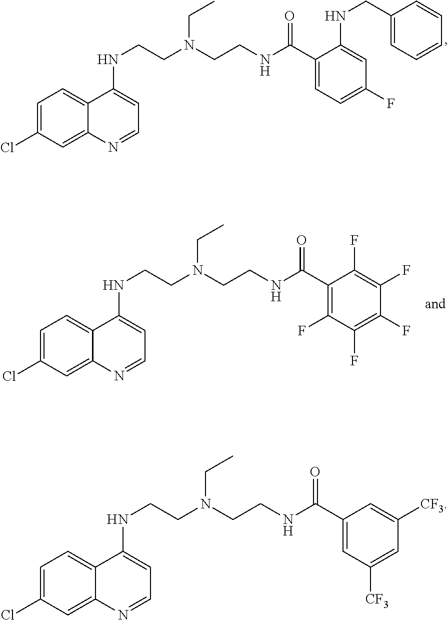

In certain embodiments, the present invention relates to any one of the aforementioned compounds and any attendant definitions, wherein R is

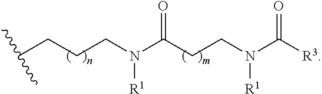

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein m is 0. In certain embodiments, the present invention relates to any one of the aforementioned compounds and any attendant definitions, wherein m is 1. In certain embodiments, the present invention relates to any one of the aforementioned compounds and any attendant definitions, wherein m is 2. In certain embodiments, the present invention relates to any one of the aforementioned compounds and any attendant definitions, wherein m is 3. In certain embodiments, the present invention relates to any one of the aforementioned compounds and any attendant definitions, wherein m is 4. In certain embodiments, the present invention relates to any one of the aforementioned compounds and any attendant definitions, wherein m is 5.

In certain embodiments, the present invention relates to any one of the aforementioned compounds and any attendant definitions, wherein R¹ is hydrogen.

In certain embodiments, the present invention relates to any one of the aforementioned compounds and any attendant definitions, wherein R³ is haloalkyl. In certain embodiments, the present invention relates to any one of the aforementioned compounds and any attendant definitions, wherein R³ is perfluoralkyl. In certain embodiments, the present invention relates to any one of the aforementioned compounds and any attendant definitions, wherein R³ is aryl. In certain embodiments, the present invention relates to any one of the aforementioned compounds and any attendant definitions, wherein R³ is phenyl. In certain embodiments, the present invention relates to any one of the aforementioned compounds and any attendant definitions, wherein R³ is heteroaryl. In certain embodiments, the present invention relates to any one of the aforementioned compounds and any attendant definitions, wherein R³ is pyridinyl.

In certain embodiments, the present invention relates to any one of the aforementioned compounds and any attendant definitions, wherein R is

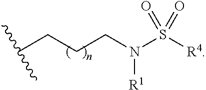

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein R¹ is hydrogen.

In certain embodiments, the present invention relates to any one of the aforementioned compounds and any attendant definitions, wherein R⁴ is aryl. In certain embodiments, the present invention relates to any one of the aforementioned compounds and any attendant definitions, wherein R⁴ is an naphthyl substituted with at least one amino substituent, a quinolinyl, an N-alkyl 3,4-dihydro-2H-1,4-benzoxazine, or a pyridinyl substituted with at least one aryloxy substituent. In certain embodiments, the present invention relates to any one of the aforementioned compounds and any attendant definitions, wherein R⁴ is selected from the group consisting of

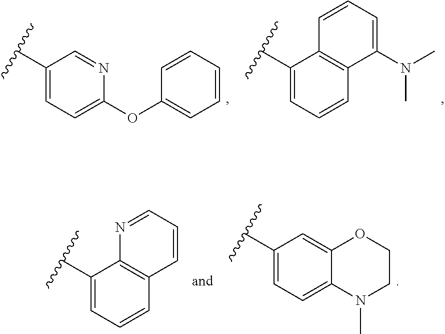

In certain embodiments, the present invention relates to any one of the aforementioned compounds and any attendant definitions, wherein the compound is selected from the group consisting of

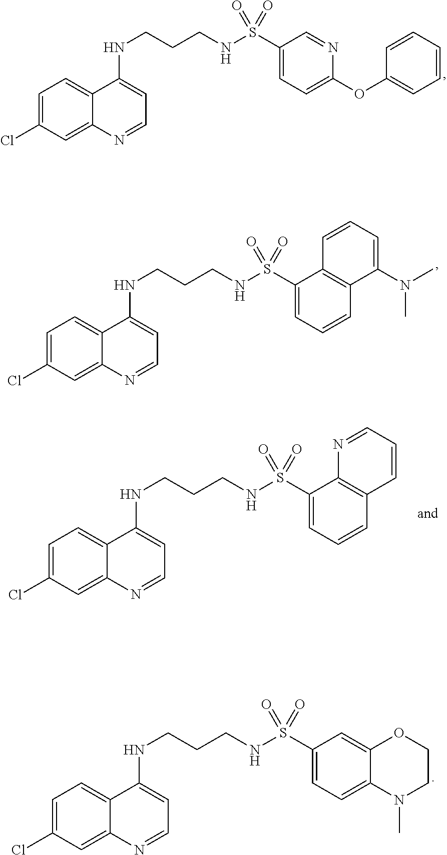

In certain embodiments, the present invention relates to any one of the aforementioned compounds and any attendant definitions, wherein R is

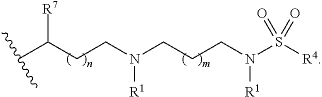

In certain embodiments, the present invention relates to any one of the aforementioned compounds and any attendant definitions, wherein n is 0. In certain embodiments, the present invention relates to any one of the aforementioned compounds and any attendant definitions, wherein n is 1. In certain embodiments, the present invention relates to any one of the aforementioned compounds and any attendant definitions, wherein n is 2. In certain embodiments, the present invention relates to any one of the aforementioned compounds and any attendant definitions, wherein n is 3. In certain embodiments, the present invention relates to any one of the aforementioned compounds and any attendant definitions, wherein n is 4. In certain embodiments, the present invention relates to any one of the aforementioned compounds and any attendant definitions, wherein n is 5.

In certain embodiments, the present invention relates to any one of the aforementioned compounds and any attendant definitions, wherein m is 0. In certain embodiments, the present invention relates to any one of the aforementioned compounds and any attendant definitions, wherein m is 1. In certain embodiments, the present invention relates to any one of the aforementioned compounds and any attendant definitions, wherein m is 2. In certain embodiments, the present invention relates to any one of the aforementioned compounds and any attendant definitions, wherein m is 3. In certain embodiments, the present invention relates to any one of the aforementioned compounds and any attendant definitions, wherein m is 4. In certain embodiments, the present invention relates to any one of the aforementioned compounds and any attendant definitions, wherein m is 5.

In certain embodiments, the present invention relates to any one of the aforementioned compounds and any attendant definitions, wherein R¹ is hydrogen.

In certain embodiments, the present invention relates to any one of the aforementioned compounds and any attendant definitions, wherein R⁷ is hydrogen. In certain embodiments, the present invention relates to any one of the aforementioned compounds and any attendant definitions, wherein R⁷ is alkyl. In certain embodiments, the present invention relates to any one of the aforementioned compounds and any attendant definitions, wherein R⁷ is methyl.

In certain embodiments, the present invention relates to any one of the aforementioned compounds and any attendant definitions, wherein R⁴ is aryl. In certain embodiments, the present invention relates to any one of the aforementioned compounds and any attendant definitions, wherein R⁴ is an naphthyl substituted with at least one amino substituent, a quinolinyl, an N-alkyl 3,4-dihydro-2H-1,4-benzoxazine, a pyridinyl, or a pyridinyl substituted with at least one aryloxy substituent. In certain embodiments, the present invention relates to any one of the aforementioned compounds and any attendant definitions, wherein R⁴ is selected

from the group consisting of

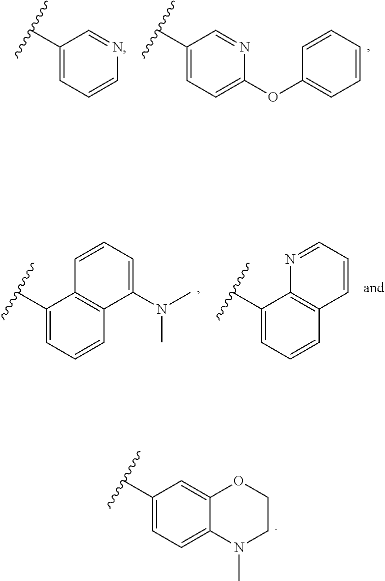

In certain embodiments, the present invention relates to any one of the aforementioned compounds and any attendant definitions, wherein the compound is selected from the group consisting of

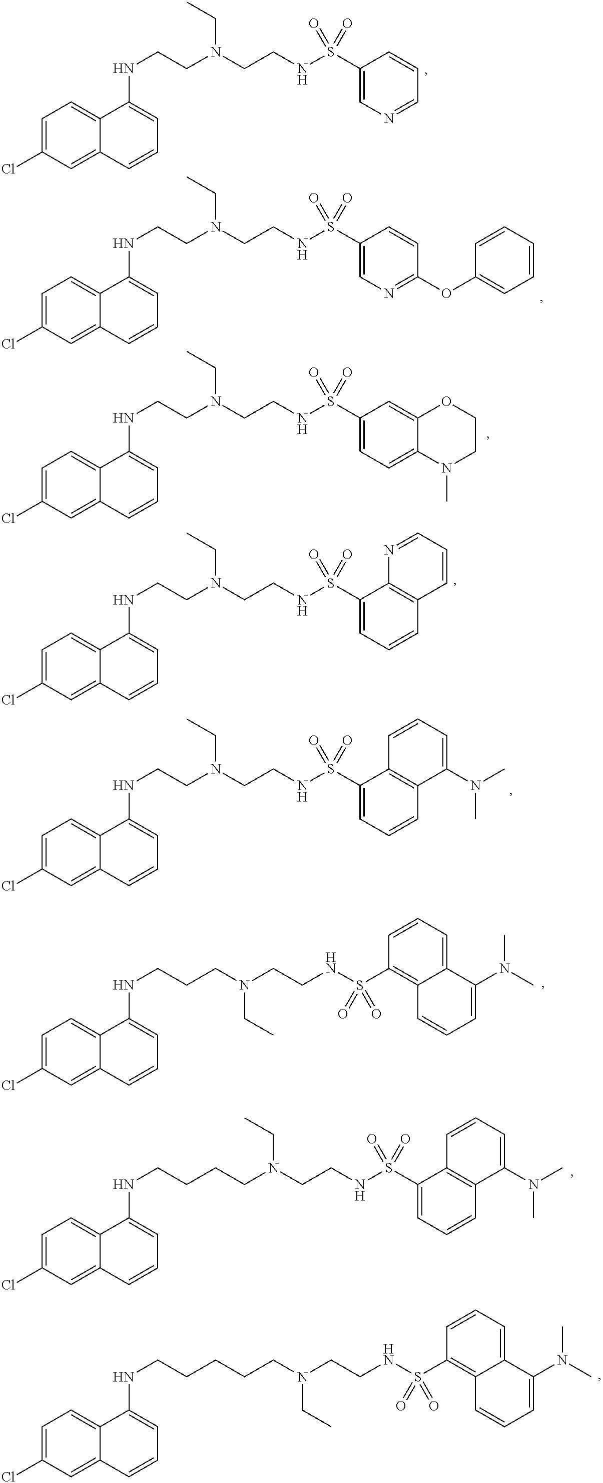

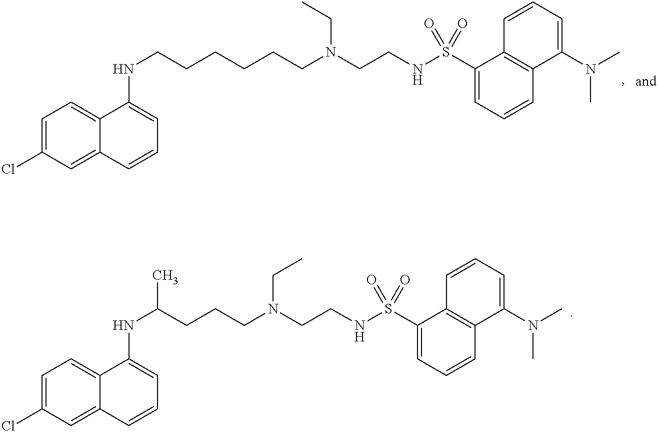

The compounds of the present invention may be prepared according to the procedures described herein, especially as described in the experimental part. In general, all chemical transformations can be performed according to well-known standard methodologies as described in the literature or as described in the procedures below.

It will be appreciated by those skilled in the art that it may be desirable to use protected derivatives of intermediates used in the preparation of the compounds described above. Protection and deprotection of functional groups may be performed by methods known in the art. Hydroxyl or amino groups may be protected with any hydroxyl or amino protecting group (for example, as described in Green and Wuts. Protective Groups in Organic Synthesis. John Wiley and Sons, New York, 1999). The protecting groups may be removed by conventional techniques. For example, acyl groups (such as alkanoyl, alkoxycarbonyl and aryloyl groups) may be removed by solvolysis (e.g., by hydrolysis under acidic or basic conditions). Arylmethoxycarbonyl groups (e.g., benzyloxycarbonyl) may be cleaved by hydrogenolysis in the presence of a catalyst such as palladium-on-carbon.

The synthesis of the target compound is completed by removing any protecting groups, which are present in the penultimate intermediate using standard techniques, which are well-known to those skilled in the art. The deprotected final product is then purified, as necessary, using standard techniques such as silica gel chromatography, HPLC on silica gel and the like, or by recrystallization.

### Selected Methods of the Invention

One aspect of the invention relates to a method of treating or preventing malaria comprises administration of a compound of the invention (e.g., a compound of formula I-V, as described above). Further object of the present invention is the use of the compounds described below for all the indications that have been already described and/or suggested for chloroquine, including in a non-limitative way: prevention and/or treatment of inflammatory articular and non-articular diseases, cancer, prevention and/or treatment of other major infective diseases, including as non-limitative examples: viral infections such as avian, seasonal and pandemic influenzae, severe acute respiratory syndrome (SARS) or acquired immunodeficiency syndrome (AIDS) and bacterial infections such as tuberculosis, etc, alone or in combination with at least a proper therapeutic agents/tools.

In certain embodiments, the invention relates to a method for the therapeutic and/or prophylactic treatment of malaria in a subject in need of such treatment comprising administering to the subject a therapeutically effective amount of a compound of formula I-V:

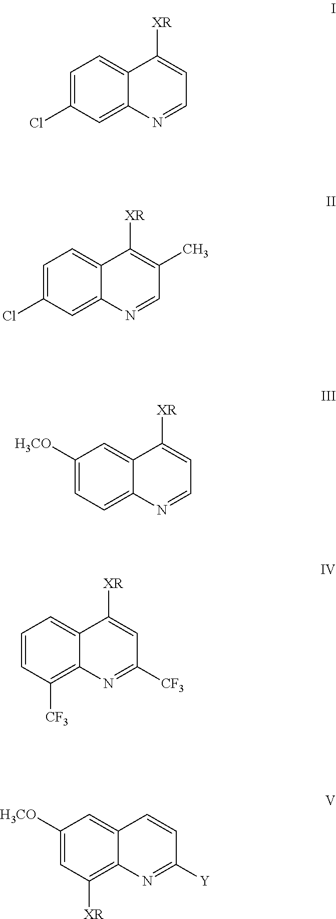

wherein, independently for each occurrence,

X is —N(H)—, —O— or —S—;

Y is hydrogen, alkyl, aryl or heteroaryl;

R is

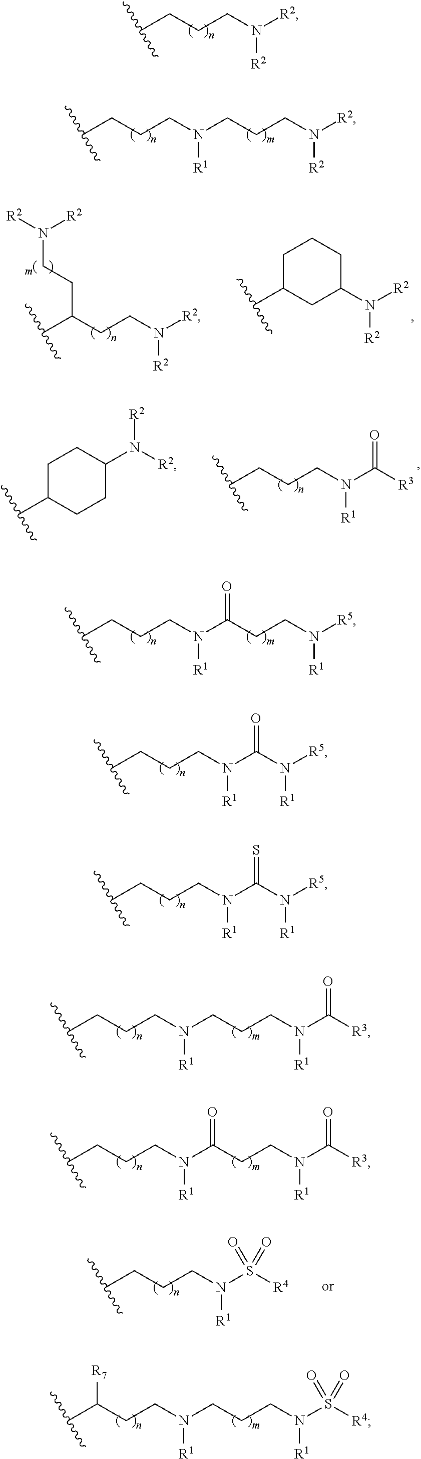

R¹ is hydrogen or alkyl;

R² is hydrogen or alkyl;

R³ is haloalkyl, aryl, aralkyl, heteroaryl, heteroaralkyl,

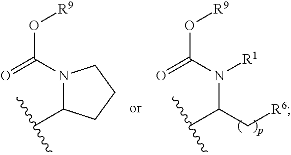

R⁴ is aryl or heteroaryl;

R⁵ is aryl or heteroaryl;

R⁶ is aryl, heteroaryl or

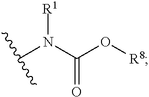

R⁷ is hydrogen or alkyl;

R⁸ is aryl, heteroaryl, aralkyl or heteroaralkyl;

R⁹ is hydrogen or alkyl;

n is 0-5 inclusive;

m is 0-5 inclusive;

p is 0-5 inclusive; and

each aryl and heteroaryl moiety, including those which are a part of an aralkyl or heteroaralkyl moiety, is optional substituted with 1-3 substitutents selected from the group consisting of alkyl, cycloalkyl, halo, perhaloalkyl, aralkyl, heteroaralkyl, alkenyl, alkynyl, carbonyl, ester, carboxyl, carboxylic acid, formyl, thiocarbonyl, thioester, thiocarboxylic acid, thioformyl, ketone, aldehyde, cyano, isocyano, amino, acylamino, amido, nitro, hydroxyl, alkoxy, aryloxy, heteroaryloxy, aralkyloxy, sulfhydryl, alkylthio, arylthio, heteroarylthio, aralkylthio, heteroaralkylthio, sulfoxido, sulfonyl, oxysulfonyl, sulfonylamino, sulfamoyl, carbocyclyl, polycyclyl, aryl, heteroaryl, and heterocyclyl.

In certain embodiments, the present invention relates to any one of the aforementioned methods and any attendant definitions, wherein X is —N(H)—. In certain embodiments, the present invention relates to any one of the aforementioned methods and any attendant definitions, wherein X is —O—. In certain embodiments, the present invention relates to any one of the aforementioned methods and any attendant definitions, wherein X is —S—.

In certain embodiments, the present invention relates to any one of the aforementioned methods and any attendant definitions, wherein Y is hydrogen.

In certain embodiments, the present invention relates to any one of the aforementioned methods and any attendant definitions, wherein the compound is represented by

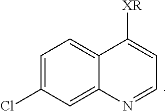

In certain embodiments, the present invention relates to any one of the aforementioned methods and any attendant definitions, wherein the compound is represented by

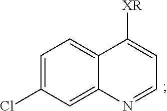

and X is —N(H)—. In certain embodiments, the present invention relates to any one of the aforementioned methods and any attendant definitions, wherein the compound is represented by

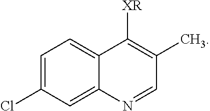

In certain embodiments, the present invention relates to any one of the aforementioned methods and any attendant definitions, wherein the compound is represented by

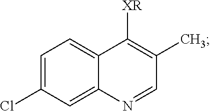

and X is —N(H)—. In certain embodiments, the present invention relates to any one of the aforementioned methods and any attendant definitions, wherein the compound is represented by

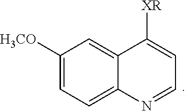

In certain embodiments, the present invention relates to any one of the aforementioned methods and any attendant definitions, wherein the compound is represented by

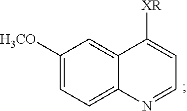

and X is —N(H)—. In certain embodiments, the present invention relates to any one of the aforementioned methods and any attendant definitions, wherein the compound is represented by

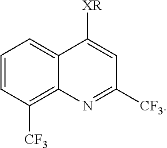

In certain embodiments, the present invention relates to any one of the aforementioned methods and any attendant definitions, wherein the compound is represented by

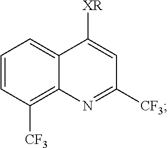

### and X is —N(H)—.

In certain embodiments, the present invention relates to any one of the aforementioned methods and any attendant definitions, wherein the compound is represented by

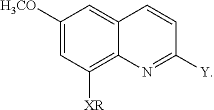

In certain embodiments, the present invention relates to any one of the aforementioned methods and any attendant definitions, wherein the compound is represented by

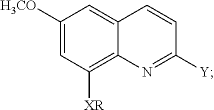

and Y is hydrogen. In certain embodiments, the present invention relates to any one of the aforementioned methods and any attendant definitions, wherein the compound is represented by

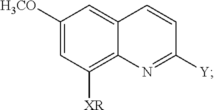

Y is hydrogen; and X is —N(H)—.

In certain embodiments, the present invention relates to any one of the aforementioned methods and any attendant definitions, wherein R is

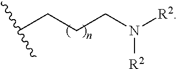

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein R² is hydrogen, methyl, ethyl, propyl or isopropyl. In certain embodiments, the present invention relates to any one of the aforementioned methods and any attendant definitions, wherein one R² is hydrogen; and one R² is methyl, ethyl, propyl or isopropyl. In certain embodiments, the present invention relates to any one of the aforementioned methods and any attendant definitions, wherein one R² is hydrogen; and one R² is ethyl. In certain embodiments, the present invention relates to any one of the aforementioned methods and any attendant definitions, wherein R² is C₁₋₄ alkyl. In certain embodiments, the present invention relates to any one of the aforementioned methods and any attendant definitions, wherein R² is methyl, ethyl, propyl or isopropyl. In certain embodiments, the present invention relates to any one of the aforementioned methods and any attendant definitions, wherein R² is ethyl.

In certain embodiments, the present invention relates to any one of the aforementioned methods and any attendant definitions, wherein the compound is selected from the group consisting of

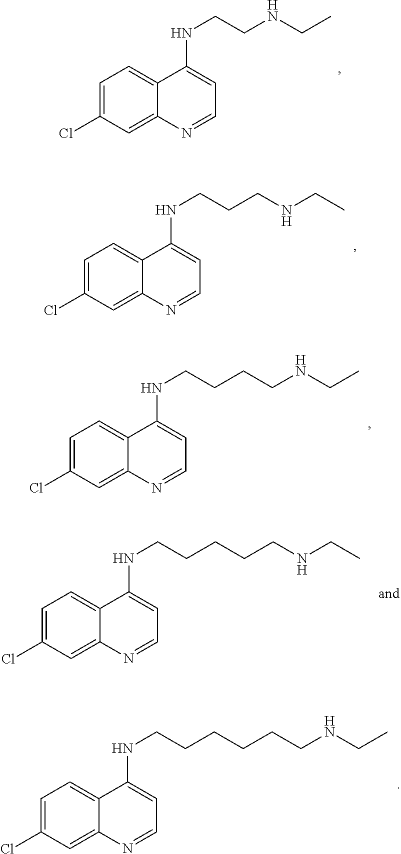

In certain embodiments, the present invention relates to any one of the aforementioned methods and any attendant definitions, wherein the compound is selected from the group consisting of

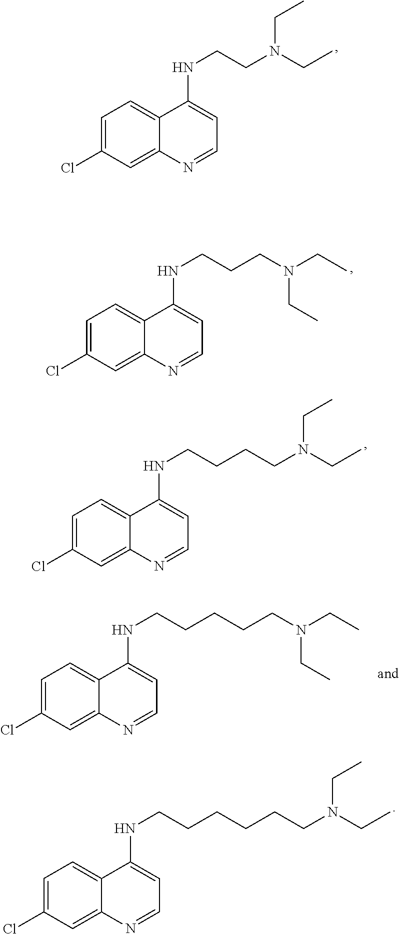

In certain embodiments, the present invention relates to any one of the aforementioned methods and any attendant definitions, wherein the compound is selected from the group consisting of

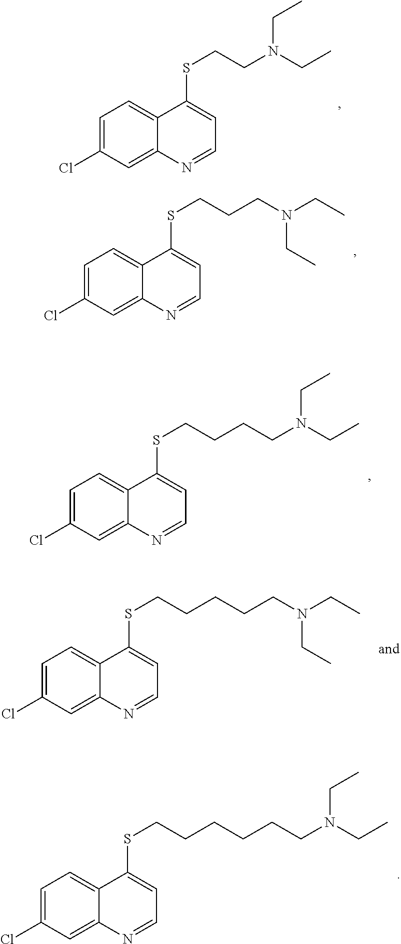

In certain embodiments, the present invention relates to any one of the aforementioned methods and any attendant definitions, wherein the compound is selected from the group consisting of

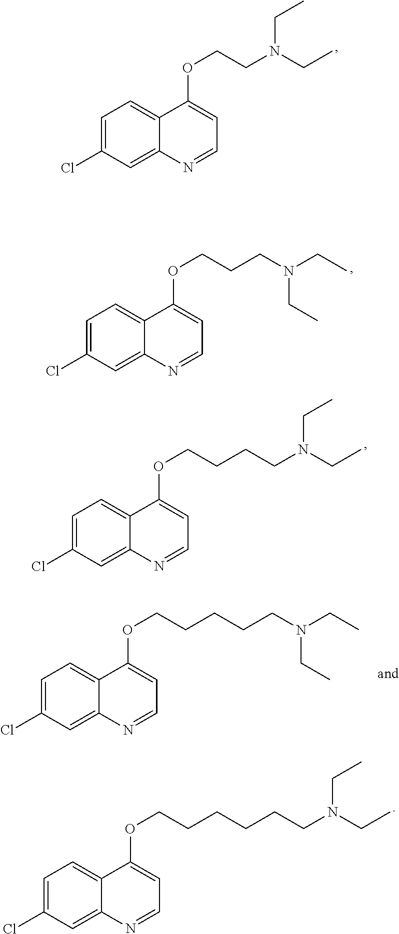

In certain embodiments, the present invention relates to any one of the aforementioned methods and any attendant definitions, wherein R is

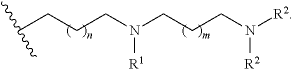

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein m is 0. In certain embodiments, the present invention relates to any one of the aforementioned methods and any attendant definitions, wherein m is 1. In certain embodiments, the present invention relates to any one of the aforementioned methods and any attendant definitions, wherein m is 2. In certain embodiments, the present invention relates to any one of the aforementioned methods and any attendant definitions, wherein m is 3. In certain embodiments, the present invention relates to any one of the aforementioned methods and any attendant definitions, wherein m is 4. In certain embodiments, the present invention relates to any one of the aforementioned methods and any attendant definitions, wherein m is 5.

In certain embodiments, the present invention relates to any one of the aforementioned methods and any attendant definitions, wherein R¹ is hydrogen, methyl, ethyl, propyl or isopropyl. In certain embodiments, the present invention relates to any one of the aforementioned methods and any attendant definitions, wherein R¹ is hydrogen. In certain embodiments, the present invention relates to any one of the aforementioned methods and any attendant definitions, wherein R¹ is ethyl.

In certain embodiments, the present invention relates to any one of the aforementioned methods and any attendant definitions, wherein R² is C₁₋₄ alkyl. In certain embodiments, the present invention relates to any one of the aforementioned methods and any attendant definitions, wherein R² is methyl, ethyl, propyl or isopropyl. In certain embodiments, the present invention relates to any one of the aforementioned methods and any attendant definitions, wherein R² is ethyl.

In certain embodiments, the present invention relates to any one of the aforementioned methods and any attendant definitions, wherein the compound is selected from the group consisting of

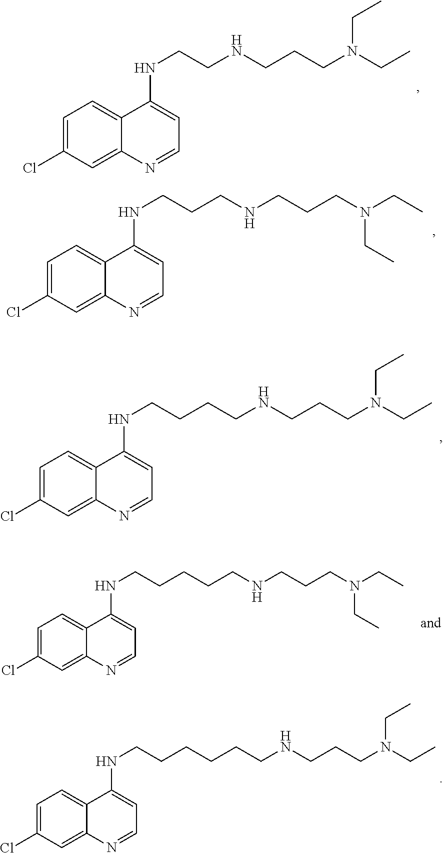

In certain embodiments, the present invention relates to any one of the aforementioned methods and any attendant definitions, wherein the compound is selected from the group consisting of

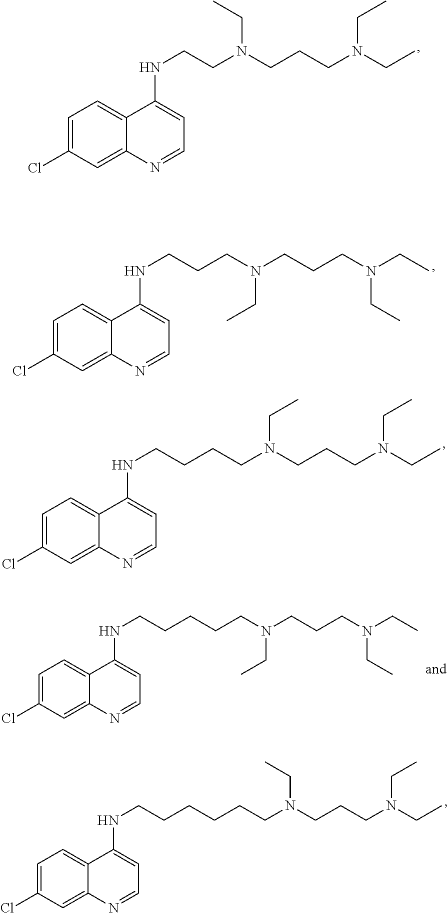

In certain embodiments, the present invention relates to any one of the aforementioned methods and any attendant definitions, wherein R is

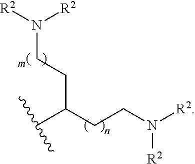

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein m is 0. In certain embodiments, the present invention relates to any one of the aforementioned methods and any attendant definitions, wherein m is 1. In certain embodiments, the present invention relates to any one of the aforementioned methods and any attendant definitions, wherein m is 2. In certain embodiments, the present invention relates to any one of the aforementioned methods and any attendant definitions, wherein m is 3. In certain embodiments, the present invention relates to any one of the aforementioned methods and any attendant definitions, wherein m is 4. In certain embodiments, the present invention relates to any one of the aforementioned methods and any attendant definitions, wherein m is 5.

In certain embodiments, the present invention relates to any one of the aforementioned methods and any attendant definitions, wherein R² is C₁₋₄ alkyl. In certain embodiments, the present invention relates to any one of the aforementioned methods and any attendant definitions, wherein R² is methyl, ethyl, propyl or isopropyl. In certain embodiments, the present invention relates to any one of the aforementioned methods and any attendant definitions, wherein R² is ethyl. In certain embodiments, the present invention relates to any one of the aforementioned methods and any attendant definitions, wherein R² is isopropyl.

In certain embodiments, the present invention relates to any one of the aforementioned methods and any attendant definitions, wherein the compound is selected from the group consisting of

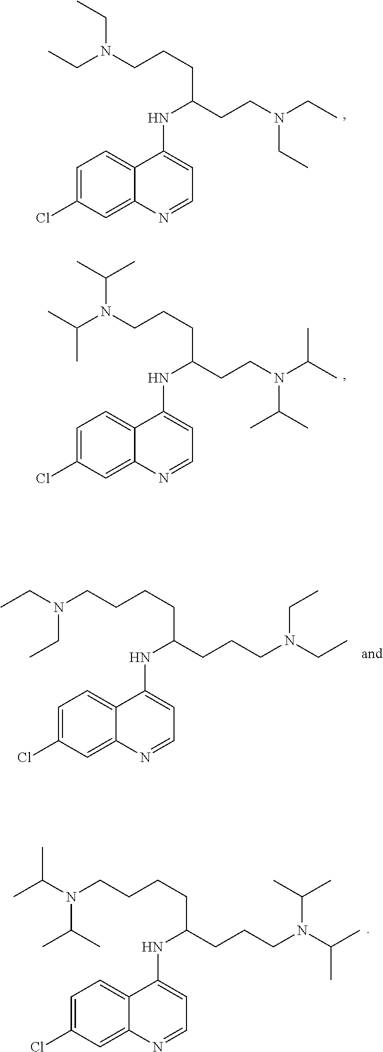

In certain embodiments, the present invention relates to any one of the aforementioned methods and any attendant definitions, wherein the compound is selected from the group consisting of

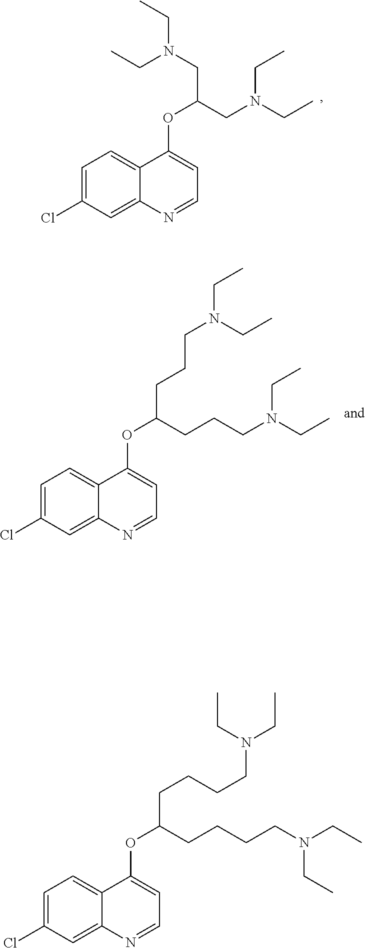

In certain embodiments, the present invention relates to any one of the aforementioned methods and any attendant definitions, wherein R is

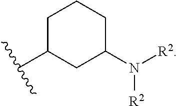

In certain embodiments, the present invention relates to any one of the aforementioned methods and any attendant definitions, wherein R² is hydrogen or C₁₋₄ alkyl. In certain embodiments, the present invention relates to any one of the aforementioned methods and any attendant definitions, wherein R² is hydrogen. In certain embodiments, the present invention relates to any one of the aforementioned methods and any attendant definitions, wherein R² is methyl, ethyl, propyl or isopropyl. In certain embodiments, the present invention relates to any one of the aforementioned methods and any attendant definitions, wherein R² is ethyl. In certain embodiments, the present invention relates to any one of the aforementioned methods and any attendant definitions, wherein R² is isopropyl.

In certain embodiments, the present invention relates to any one of the aforementioned methods and any attendant definitions, wherein the compound is selected from the group consisting of

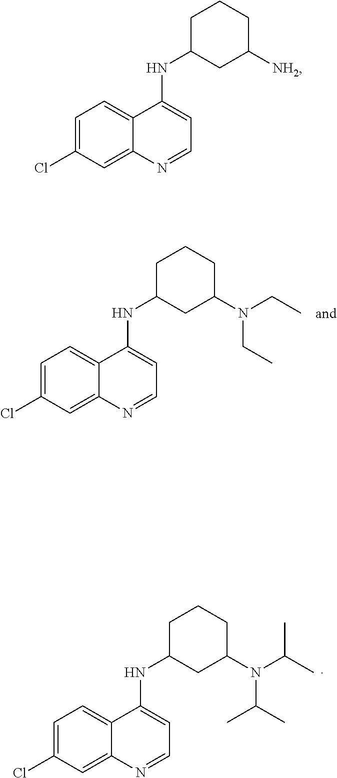

In certain embodiments, the present invention relates to any one of the aforementioned methods and any attendant definitions, wherein R is

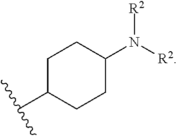

In certain embodiments, the present invention relates to any one of the aforementioned methods and any attendant definitions, wherein R² is hydrogen or C₁₋₄ alkyl. In certain embodiments, the present invention relates to any one of the aforementioned methods and any attendant definitions, wherein R² is hydrogen. In certain embodiments, the present invention relates to any one of the aforementioned methods and any attendant definitions, wherein R² is methyl, ethyl, propyl or isopropyl. In certain embodiments, the present invention relates to any one of the aforementioned methods and any attendant definitions, wherein R² is ethyl. In certain embodiments, the present invention relates to any one of the aforementioned methods and any attendant definitions, wherein R² is isopropyl.

In certain embodiments, the present invention relates to any one of the aforementioned methods and any attendant definitions, wherein the compound is selected from the group consisting of

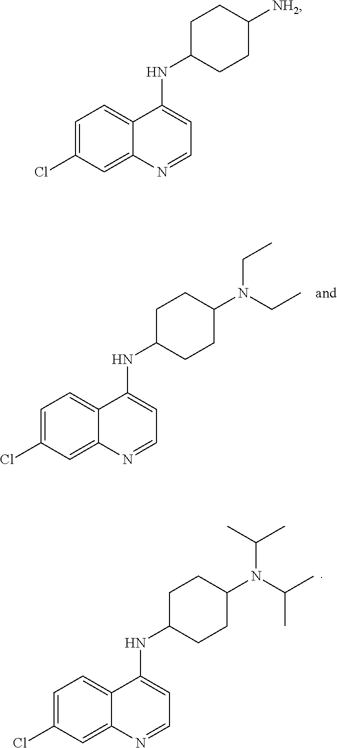

In certain embodiments, the present invention relates to any one of the aforementioned methods and any attendant definitions, wherein R is

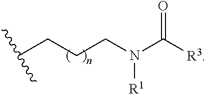

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein R¹ is hydrogen.

In certain embodiments, the present invention relates to any one of the aforementioned methods and any attendant definitions, wherein R³ is haloalkyl. In certain embodiments, the present invention relates to any one of the aforementioned methods and any attendant definitions, wherein R³ is perfluoroalkyl. In certain embodiments, the present invention relates to any one of the aforementioned methods and any attendant definitions, wherein R³ is —(CF₂)_{w}CF₃; and w is 1-7 inclusive. In certain embodiments, the present invention relates to any one of the aforementioned methods and any attendant definitions, wherein R³ is —(CF₂)₂CF₃. In certain embodiments, the present invention relates to any one of the aforementioned methods and any attendant definitions, wherein R³ is —(CF₂)₅CF₃

In certain embodiments, the present invention relates to any one of the aforementioned methods and any attendant definitions, wherein the compound is selected from the group consisting of

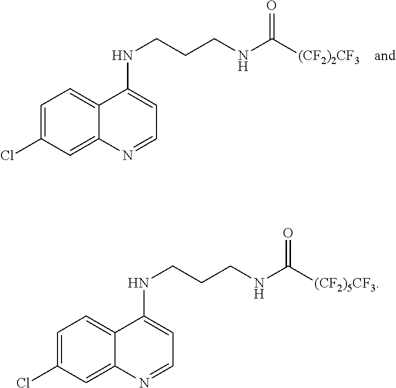

In certain embodiments, the present invention relates to any one of the aforementioned methods and any attendant definitions, wherein R³ is aryl. In certain embodiments, the present invention relates to any one of the aforementioned methods and any attendant definitions, wherein R³ is an phenyl substituted with at least one amino, haloalkyl, halo, arylthio, alkylthio, or hydroxyl substituent. In certain embodiments, the present invention relates to any one of the aforementioned methods and any attendant definitions, wherein R³ is selected from the group consisting of

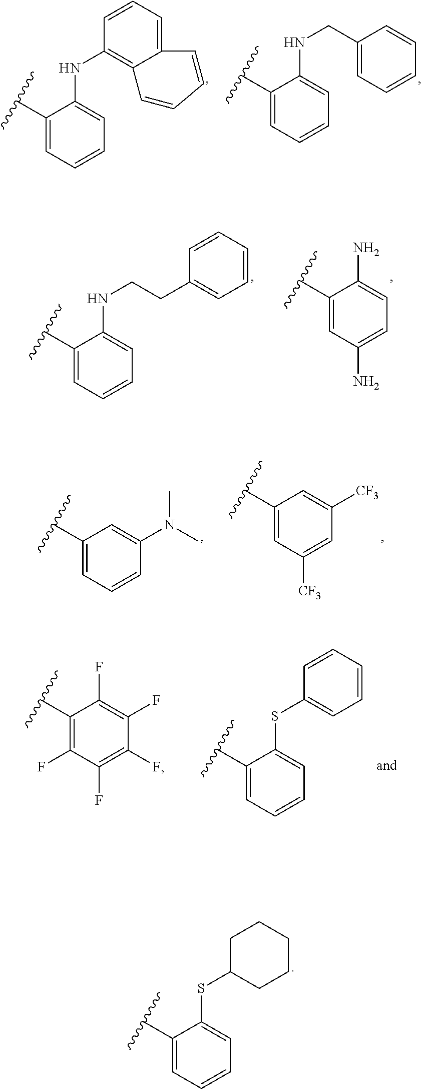

In certain embodiments, the present invention relates to any one of the aforementioned methods and any attendant definitions, wherein the compound is selected from the group consisting of

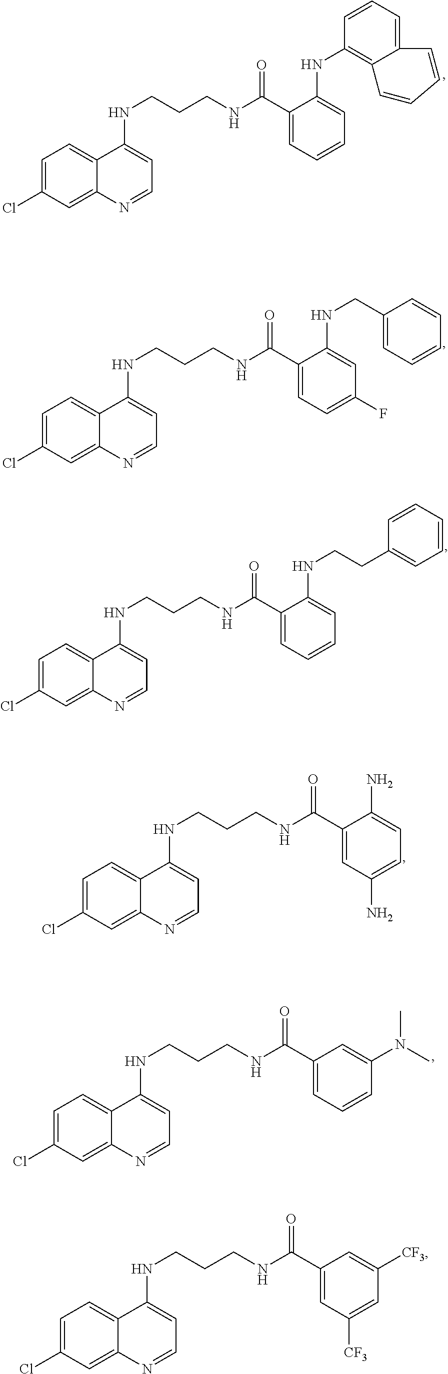

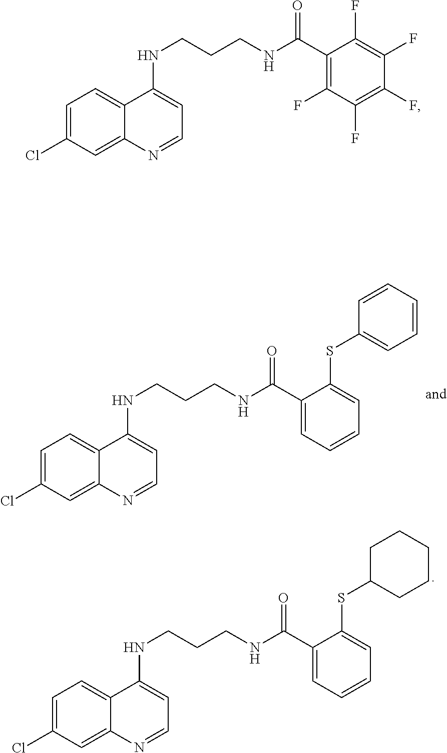

In certain embodiments, the present invention relates to any one of the aforementioned methods and any attendant definitions, wherein R³ is heteroaryl. In certain embodiments, the present invention relates to any one of the aforementioned methods and any attendant definitions, wherein R³ is a pyridinyl substituted with at least one amino, haloalkyl, halo, arylthio, alkylthio, or hydroxyl substituent. In certain embodiments, the present invention relates to any one of the aforementioned methods and any attendant definitions, wherein R³ is selected from the group consisting of

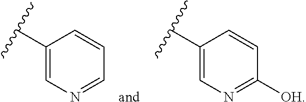

In certain embodiments, the present invention relates to any one of the aforementioned methods and any attendant definitions, wherein the compound is selected from the group consisting of

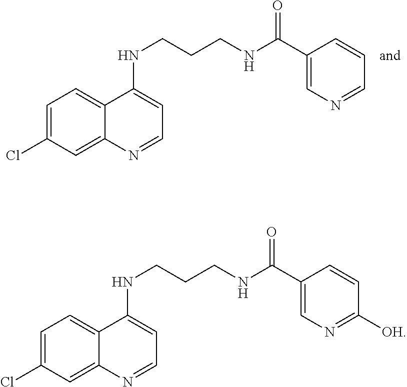

In certain embodiments, the present invention relates to any one of the aforementioned methods and any attendant definitions, wherein R³ is heteroaralkyl. In certain embodiments, the present invention relates to any one of the aforementioned methods and any attendant definitions, wherein R³ is an alkyl substituted with a 1H-benzo[d]imidazole substituent. In certain embodiments, the present invention relates to any one of the aforementioned methods and any attendant definitions, wherein R³ is

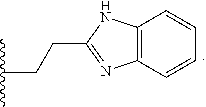

In certain embodiments, the present invention relates to any one of the aforementioned methods and any attendant definitions, wherein the compound is

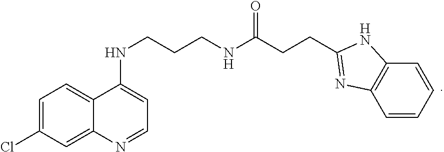

In certain embodiments, the present invention relates to any one of the aforementioned methods and any attendant definitions, wherein R³ is

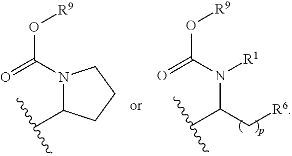

In certain embodiments, the present invention relates to any one of the aforementioned methods and any attendant definitions, wherein R⁹ is alkyl. In certain embodiments, the present invention relates to any one of the aforementioned methods and any attendant definitions, wherein R⁹ is t-butyl.

In certain embodiments, the present invention relates to any one of the aforementioned methods and any attendant definitions, wherein R¹ is hydrogen.

In certain embodiments, the present invention relates to any one of the aforementioned methods and any attendant definitions, wherein R⁶ is heteroaryl. In certain embodiments, the present invention relates to any one of the aforementioned methods and any attendant definitions, wherein R⁶ is

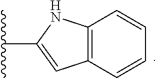

In certain embodiments, the present invention relates to any one of the aforementioned methods and any attendant definitions, wherein R⁶ is

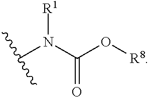

In certain embodiments, the present invention relates to any one of the aforementioned methods and any attendant definitions, wherein R⁸ is aralkyl. In certain embodiments, the present invention relates to any one of the aforementioned methods and any attendant definitions, wherein R⁸ is benzyl.

In certain embodiments, the present invention relates to any one of the aforementioned methods and any attendant definitions, wherein p is 0. In certain embodiments, the present invention relates to any one of the aforementioned methods and any attendant definitions, wherein p is 1. In certain embodiments, the present invention relates to any one of the aforementioned methods and any attendant definitions, wherein p is 2. In certain embodiments, the present invention relates to any one of the aforementioned methods and any attendant definitions, wherein p is 3. In certain embodiments, the present invention relates to any one of the aforementioned methods and any attendant definitions, wherein p is 4. In certain embodiments, the present invention relates to any one of the aforementioned methods and any attendant definitions, wherein p is 5.

In certain embodiments, the present invention relates to any one of the aforementioned methods and any attendant definitions, wherein the compound is selected from the group consisting of

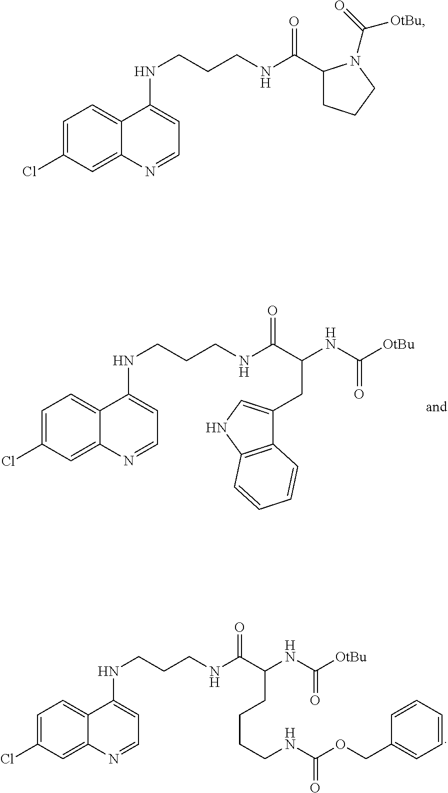

In certain embodiments, the present invention relates to any one of the aforementioned methods and any attendant definitions, wherein R is

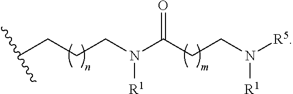

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein m is 0. In certain embodiments, the present invention relates to any one of the aforementioned methods and any attendant definitions, wherein m is 1. In certain embodiments, the present invention relates to any one of the aforementioned methods and any attendant definitions, wherein m is 2. In certain embodiments, the present invention relates to any one of the aforementioned methods and any attendant definitions, wherein m is 3. In certain embodiments, the present invention relates to any one of the aforementioned methods and any attendant definitions, wherein m is 4. In certain embodiments, the present invention relates to any one of the aforementioned methods and any attendant definitions, wherein m is 5.

In certain embodiments, the present invention relates to any one of the aforementioned methods and any attendant definitions, wherein R¹ is hydrogen.

In certain embodiments, the present invention relates to any one of the aforementioned methods and any attendant definitions, wherein R⁵ is C₁₋₄ alkyl. In certain embodiments, the present invention relates to any one of the aforementioned methods and any attendant definitions, wherein R⁵ is methyl, ethyl, propyl or isopropyl. In certain embodiments, the present invention relates to any one of the aforementioned methods and any attendant definitions, wherein R⁵ is ethyl.

In certain embodiments, the present invention relates to any one of the aforementioned methods and any attendant definitions, wherein the compound is selected from the group consisting of

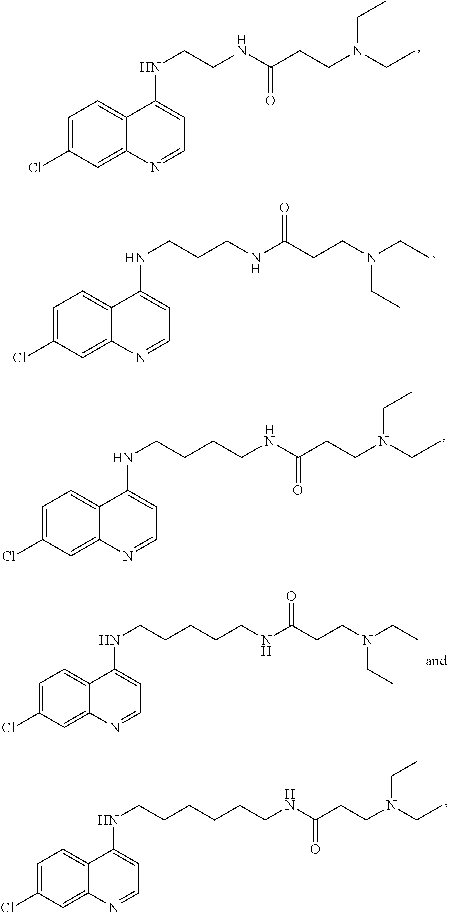

In certain embodiments, the present invention relates to any one of the aforementioned methods and any attendant definitions, wherein R is

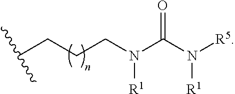

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein R¹ is hydrogen.

In certain embodiments, the present invention relates to any one of the aforementioned methods and any attendant definitions, wherein R⁵ is aryl. In certain embodiments, the present invention relates to any one of the aforementioned methods and any attendant definitions, wherein R⁵ is an phenyl substituted with at least one amino, alkoxy, or nitro substituent. In certain embodiments, the present invention relates to any one of the aforementioned methods and any attendant definitions, wherein R⁵ is selected from the group consisting of

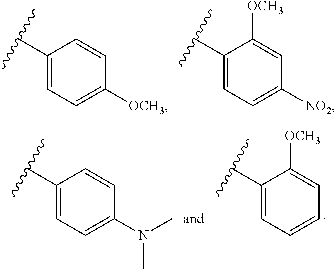

In certain embodiments, the present invention relates to any one of the aforementioned methods and any attendant definitions, wherein the compound is selected from the group consisting of

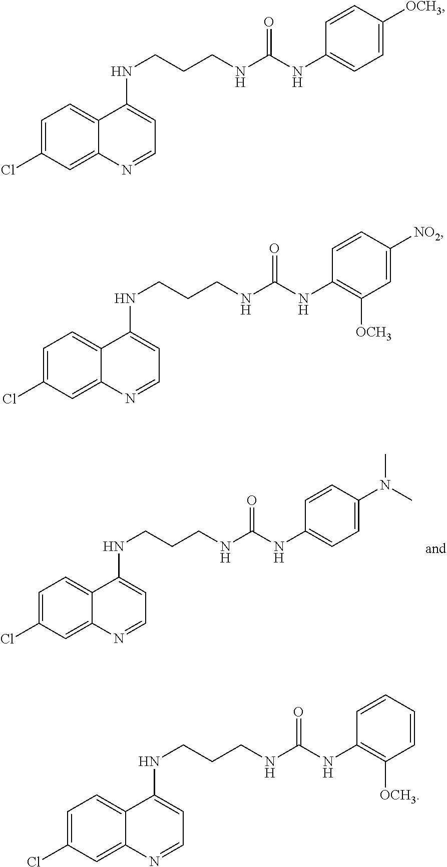

In certain embodiments, the present invention relates to any one of the aforementioned methods and any attendant definitions, wherein R is

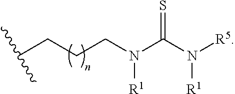

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein R¹ is hydrogen.

In certain embodiments, the present invention relates to any one of the aforementioned methods and any attendant definitions, wherein R⁵ is aryl. In certain embodiments, the present invention relates to any one of the aforementioned methods and any attendant definitions, wherein R⁵ is an phenyl or naphthyl substituted with at least one amino, alkoxy, or nitro substitutent. In certain embodiments, the present invention relates to any one of the aforementioned methods and any attendant definitions, wherein R⁵ is selected from the group consisting of

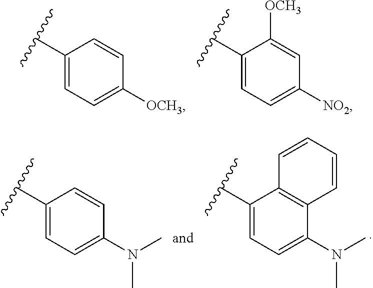

In certain embodiments, the present invention relates to any one of the aforementioned methods and any attendant definitions, wherein the compound is selected from the group consisting of

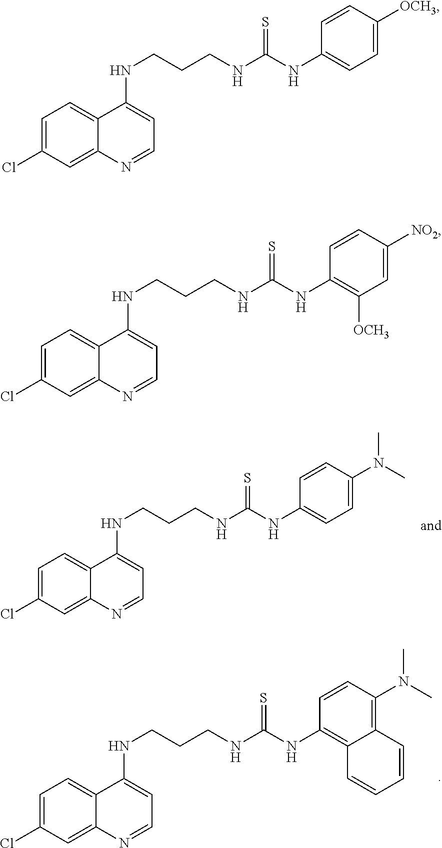

In certain embodiments, the present invention relates to any one of the aforementioned methods and any attendant definitions, wherein R is

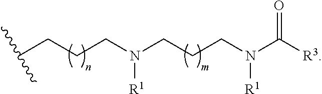

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein m is 0. In certain embodiments, the present invention relates to any one of the aforementioned methods and any attendant definitions, wherein m is 1. In certain embodiments, the present invention relates to any one of the aforementioned methods and any attendant definitions, wherein m is 2. In certain embodiments, the present invention relates to any one of the aforementioned methods and any attendant definitions, wherein m is 3. In certain embodiments, the present invention relates to any one of the aforementioned methods and any attendant definitions, wherein m is 4. In certain embodiments, the present invention relates to any one of the aforementioned methods and any attendant definitions, wherein m is 5.

In certain embodiments, the present invention relates to any one of the aforementioned methods and any attendant definitions, wherein R¹ is hydrogen.

In certain embodiments, the present invention relates to any one of the aforementioned methods and any attendant definitions, wherein R³ is haloalkyl. In certain embodiments, the present invention relates to any one of the aforementioned methods and any attendant definitions, wherein R³ is perfluoroalkyl. In certain embodiments, the present invention relates to any one of the aforementioned methods and any attendant definitions, wherein R³ is —(CF₂)_{w}CF₃; and w is 1-7 inclusive. In certain embodiments, the present invention relates to any one of the aforementioned methods and any attendant definitions, wherein R³ is —(CF₂)₂CF₃. In certain embodiments, the present invention relates to any one of the aforementioned methods and any attendant definitions, wherein R³ is —(CF₂)₅CF₃

In certain embodiments, the present invention relates to any one of the aforementioned methods and any attendant definitions, wherein the compound is selected from the group consisting of

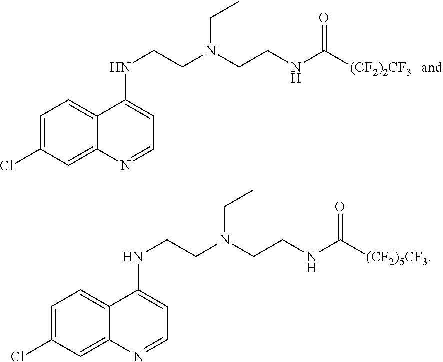

In certain embodiments, the present invention relates to any one of the aforementioned methods and any attendant definitions, wherein R³ is aryl. In certain embodiments, the present invention relates to any one of the aforementioned methods and any attendant definitions, wherein R³ is an phenyl substituted with at least one amino, haloalkyl, or halo substituent. In certain embodiments, the present invention relates to any one of the aforementioned methods and any attendant definitions, wherein R³ is selected from the group consisting of

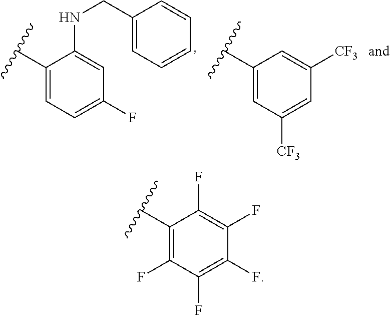

In certain embodiments, the present invention relates to any one of the aforementioned methods and any attendant definitions, wherein the compound is selected from the group consisting of

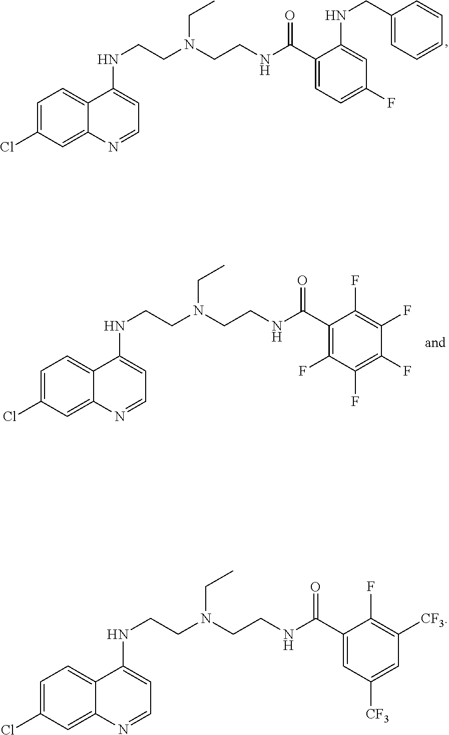

In certain embodiments, the present invention relates to any one of the aforementioned methods and any attendant definitions, wherein R is

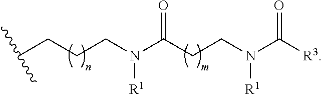

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein m is 0. In certain embodiments, the present invention relates to any one of the aforementioned methods and any attendant definitions, wherein m is 1. In certain embodiments, the present invention relates to any one of the aforementioned methods and any attendant definitions, wherein m is 2. In certain embodiments, the present invention relates to any one of the aforementioned methods and any attendant definitions, wherein m is 3. In certain embodiments, the present invention relates to any one of the aforementioned methods and any attendant definitions, wherein m is 4. In certain embodiments, the present invention relates to any one of the aforementioned methods and any attendant definitions, wherein m is 5.

In certain embodiments, the present invention relates to any one of the aforementioned methods and any attendant definitions, wherein R¹ is hydrogen.

In certain embodiments, the present invention relates to any one of the aforementioned methods and any attendant definitions, wherein R³ is haloalkyl. In certain embodiments, the present invention relates to any one of the aforementioned methods and any attendant definitions, wherein R³ is perfluoralkyl. In certain embodiments, the present invention relates to any one of the aforementioned methods and any attendant definitions, wherein R³ is aryl. In certain embodiments, the present invention relates to any one of the aforementioned methods and any attendant definitions, wherein R³ is phenyl. In certain embodiments, the present invention relates to any one of the aforementioned methods and any attendant definitions, wherein R³ is heteroaryl. In certain embodiments, the present invention relates to any one of the aforementioned methods and any attendant definitions, wherein R³ is pyridinyl.

In certain embodiments, the present invention relates to any one of the aforementioned methods and any attendant definitions, wherein R is

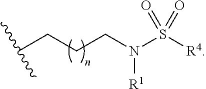

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein R¹ is hydrogen.

In certain embodiments, the present invention relates to any one of the aforementioned methods and any attendant definitions, wherein R⁴ is aryl. In certain embodiments, the present invention relates to any one of the aforementioned methods and any attendant definitions, wherein R⁴ is an naphthyl substituted with at least one amino substituent, a quinolinyl, an N-alkyl 3,4-dihydro-2H-1,4-benzoxazine, or a pyridinyl substituted with at least one aryloxy substituent. In certain embodiments, the present invention relates to any one of the aforementioned methods and any attendant definitions, wherein R⁴ is selected from the group consisting of

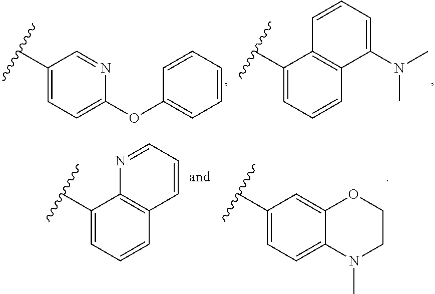

In certain embodiments, the present invention relates to any one of the aforementioned compounds and any attendant definitions, wherein the compound is selected from the group consisting of

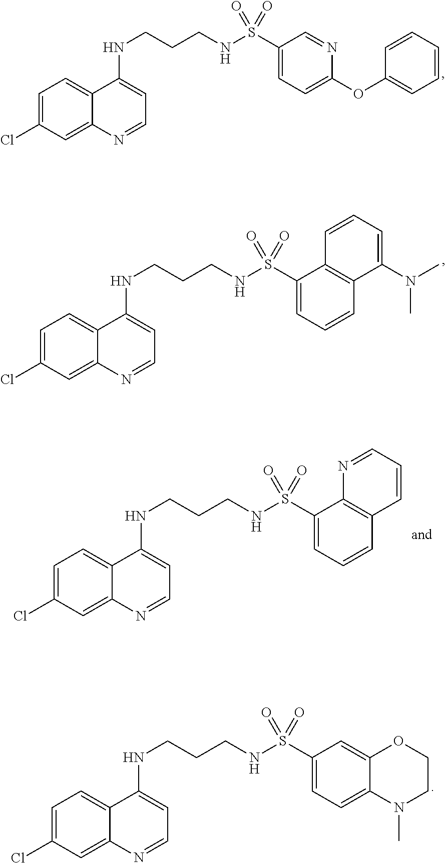

In certain embodiments, the present invention relates to any one of the aforementioned methods and any attendant definitions, wherein R is

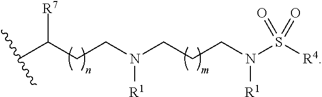

In certain embodiments, the present invention relates to any one of the aforementioned methods and any attendant definitions, wherein n is 0. In certain embodiments, the present invention relates to any one of the aforementioned methods and any attendant definitions, wherein n is 1. In certain embodiments, the present invention relates to any one of the aforementioned methods and any attendant definitions, wherein n is 2. In certain embodiments, the present invention relates to any one of the aforementioned methods and any attendant definitions, wherein n is 3. In certain embodiments, the present invention relates to any one of the aforementioned methods and any attendant definitions, wherein n is 4. In certain embodiments, the present invention relates to any one of the aforementioned methods and any attendant definitions, wherein n is 5.

In certain embodiments, the present invention relates to any one of the aforementioned methods and any attendant definitions, wherein m is 0. In certain embodiments, the present invention relates to any one of the aforementioned methods and any attendant definitions, wherein m is 1. In certain embodiments, the present invention relates to any one of the aforementioned methods and any attendant definitions, wherein m is 2. In certain embodiments, the present invention relates to any one of the aforementioned methods and any attendant definitions, wherein m is 3. In certain embodiments, the present invention relates to any one of the aforementioned methods and any attendant definitions, wherein m is 4. In certain embodiments, the present invention relates to any one of the aforementioned methods and any attendant definitions, wherein m is 5.

In certain embodiments, the present invention relates to any one of the aforementioned methods and any attendant definitions, wherein R¹ is hydrogen.

In certain embodiments, the present invention relates to any one of the aforementioned methods and any attendant definitions, wherein R⁷ is hydrogen. In certain embodiments, the present invention relates to any one of the aforementioned methods and any attendant definitions, wherein R⁷ is alkyl. In certain embodiments, the present invention relates to any one of the aforementioned methods and any attendant definitions, wherein R⁷ is methyl.

In certain embodiments, the present invention relates to any one of the aforementioned methods and any attendant definitions, wherein R⁴ is aryl. In certain embodiments, the present invention relates to any one of the aforementioned methods and any attendant definitions, wherein R⁴ is an naphthyl substituted with at least one amino substituent, a quinolinyl, an N-alkyl 3,4-dihydro-2H-1,4-benzoxazine, a pyridinyl, or a pyridinyl substituted with at least one aryloxy substituent. In certain embodiments, the present invention relates to any one of the aforementioned methods and any attendant definitions, wherein R⁴ is selected from the group consisting of

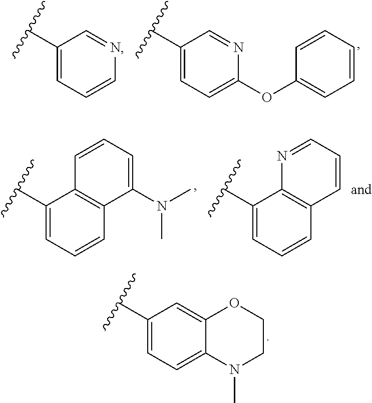

In certain embodiments, the present invention relates to any one of the aforementioned compounds and any attendant definitions, wherein the compound is selected from the group consisting of

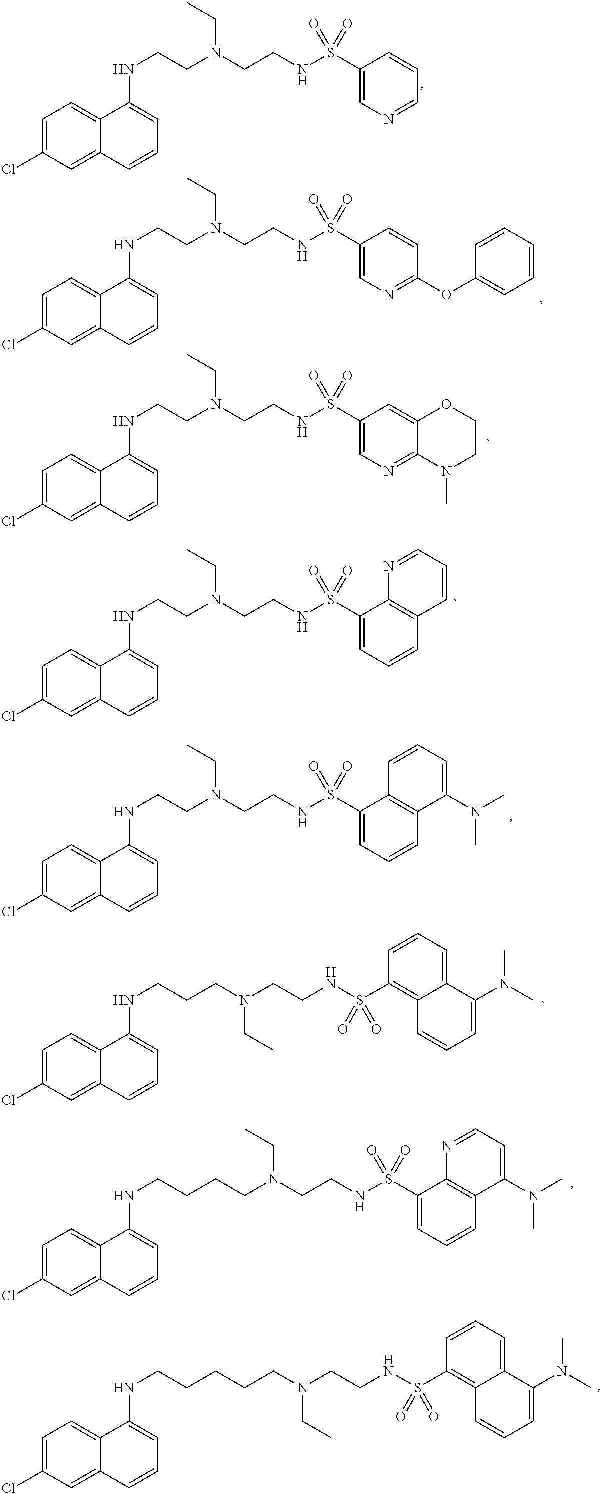

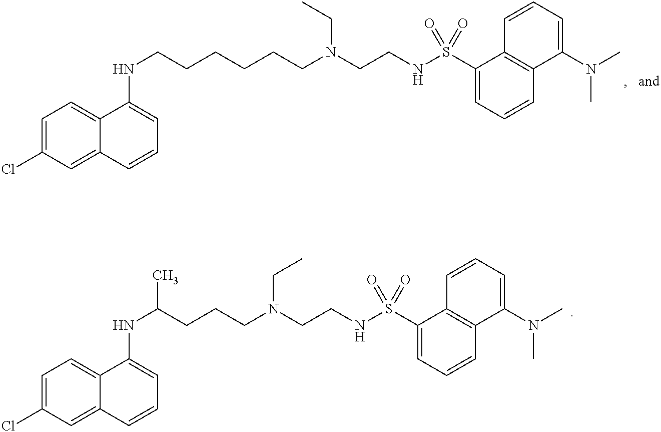

In certain embodiments, the present invention relates to any one of the aforementioned methods and any attendant definitions, wherein the subject has been infected with Plasmodium falciparum.

In certain embodiments, the present invention relates to any one of the aforementioned methods and any attendant definitions, wherein the subject has been infected with P. vivax.

In certain embodiments, the present invention relates to any one of the aforementioned methods and any attendant definitions, wherein the subject has been infected with P. ovale.

In certain embodiments, the present invention relates to any one of the aforementioned methods and any attendant definitions, wherein the subject has been infected with P. malariae.

In certain embodiments, the present invention relates to any one of the aforementioned methods and any attendant definitions, wherein the compound is administered after the subject has been exposed to the malaria parasite.

In certain embodiments, the present invention relates to any one of the aforementioned methods and any attendant definitions, wherein the malaria parasite is a drug-resistant malarial strain.

In certain embodiments, the present invention relates to any one of the aforementioned methods and any attendant definitions, wherein the drug-resistant malarial strain is resistant to one or more of chloroquine, mefloquine, halofantrine, artemisinin, atovaquone/proguanil, doxycycline or primaquine.

In certain embodiments, the present invention relates to any one of the aforementioned methods and any attendant definitions, wherein the compound is administered before the subject travels to a country where malaria is endemic.

The compounds described above, or the below-mentioned pharmaceutical compositions, may also be used in combination with one or more other therapeutically useful substances e.g., with other antimalarials like quinolines (quinine, chloroquine, amodiaquine, mefloquine, primaquine, tafenoquine), peroxide antimalarials (artemisinin, artemether, artesunate), pyrimethamine-sulfadoxine antimalarials (e.g., Fansidar), hydroxynaphtoquinones (e.g., atovaquone), acroline-type antimalarials (e.g., pyronaridine) and other antiprotozoal agents like ethylstibamine, hydroxystilbamidine, pentamidine, stilbamidine, quinapyramine, puromycine, propamidine, nifurtimox, melarsoprol, nimorazole, nifuroxime, aminitrozole and the like.

### DEFINITIONS

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

For purposes of this invention, the chemical elements are identified in accordance with the Periodic Table of the Elements, CAS version, Handbook of Chemistry and Physics, 67th Ed., 1986-87, inside cover.

The term “alkyl” refers to the radical of saturated aliphatic groups, including straight-chain alkyl groups (e.g., methyl, ethyl, propyl, butyl, pentyl, hexyl, heptyl, octyl), branched-chain alkyl groups (e.g., i-propyl, i-butyl, t-butyl), cycloalkyl (alicyclic) groups (e.g., cyclopropyl, cyclobutyl, cyclopentyl, cyclohexyl), alkyl substituted cycloalkyl groups, and cycloalkyl substituted alkyl groups. For example, C₁₋₆ alkyl means a straight or branched alkyl chain containing from 1 to 6 carbon atoms; examples of such group include methyl, ethyl, propyl, isopropyl, n-butyl, isobutyl, tert-butyl, pentyl, 3-methyl-butyl, hexyl and 2,3-dimethylbutyl and like. Similarly, the term C₁₋₄ alkyl means a straight or branched alkyl chain containing from 1 to 4 carbon atoms. Likewise, C₄₋₁₀ cycloalkyls have from 4-10 carbon atoms in their ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

The term “halo” designates —F, —Cl, —Br or —I. The term “haloalkyl” refers to “alkyl” as defined above substituted with one are more halogen, where the halogen is a fluorine, chlorine, bromine or iodine atom. The term “perhaloalkyl” as used herein as a group or a part of a group refers to a straight or branched fluorocarbon chain containing the specified number of carbon atoms. For example, C₁₋₆ perhaloalkyl means a straight or branched alkyl chain containing from 1 to 6 carbon atoms; examples of such group include trifluoromethyl, pentafluoroethyl, heptafluoropropyl, heptafluoroisopropyl and like. Similarly, the term C₁₋₄ perhaloalkyl means a straight or branched alkyl chain containing from 1 to 4 carbon atoms and 3 to 9 fluorine atoms.

The term “aralkyl” is art-recognized and refers to an alkyl group substituted with an aryl group (as defined below). The term “heteroaralkyl” is art-recognized and refers to an alkyl group substituted with an heteroaryl group (as defined below).

The terms “alkenyl” and “alkynyl” refer to radicals of unsaturated aliphatic groups analogous in length and possible substitution to the alkyls described herein, but which contain at least one double or triple carbon-carbon bond, respectively.

The term “carbonyl” is art-recognized and includes such moieties as can be represented by the general formula:

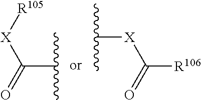

wherein X is a bond or represents an —O—, —S— or —N(R¹⁰⁵), and R¹⁰⁵ represents a pharmaceutically acceptable salt, hydrogen, alkyl, alkenyl, alkynyl, aryl, heteroaryl or —(CH₂)_{b}R²⁰⁰, wherein b is 1-10 and R²⁰⁰ represents a group permitted by the rules of valence, such as hydrogen, alkyl, alkenyl, alkynyl, aryl, and heteroaryl, and R¹⁰⁶ represents hydrogen, alkyl, alkenyl, alkynyl, aryl, heteroaryl or —(CH₂)_{b}R²⁰⁰, wherein b and R²⁰⁰ are defined above. Where X is an oxygen and R¹⁰⁵ or R¹⁰⁶ is not hydrogen, the formula represents an “ester”. Where X is an oxygen, and R¹⁰⁵ is as defined above, the moiety is referred to herein as a “carboxyl”, and particularly when R¹⁰⁵ is a hydrogen, the formula represents a “carboxylic acid”. Where X is an oxygen, and R¹⁰⁶ is hydrogen, the formula represents a “formyl”. In general, where the oxygen atom of the above formula is replaced by sulfur, the formula represents a “thiocarbonyl” group. Where X is a sulfur and R¹⁰⁵ or R¹⁰⁶ is not hydrogen, the formula represents a “thioester.” Where X is a sulfur and R¹⁰⁵ is hydrogen, the formula represents a “thiocarboxylic acid.” Where X is a sulfur and R¹⁰⁶ is hydrogen, the formula represents a “thioformyl.” On the other hand, where X is a bond, and R¹⁰⁵ is not hydrogen, the above formula represents a “ketone” radical. Where X is a bond, and R¹⁰⁶ is hydrogen, the above formula represents an “aldehyde” radical.

The term “amino” is art-recognized and as used herein refers to radicals of both unsubstituted and substituted amines, e.g., a moiety that can be represented by the general formula:

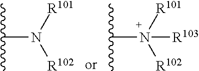

wherein R¹⁰¹, R¹⁰² and R¹⁰³ each independently represent hydrogen, alkyl, alkenyl, alkynyl, aryl, heteroaryl, —(CH₂)_{b}R²⁰⁰, wherein b and R²⁰⁰ are defined above.

The term “amino” also includes “acylamino,” which is art-recognized and refers to a radical that can be represented by the general formula:

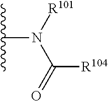

wherein R¹⁰¹ is as defined above, and R¹⁰⁴ represents hydrogen, alkyl, alkenyl, alkynyl, aryl, heteroaryl or —(CH₂)_{b}R²⁰⁰, wherein b and R²⁰⁰ are defined above.

The term “amido” is art-recognized as an amino-substituted carbonyl and includes a radical that can be represented by the general formula:

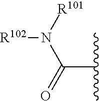

wherein R¹⁰¹ and R¹⁰² are as defined above. Preferred embodiments of the amide will not include those which are unstable.

The term “hydroxyl” means —OH. The term “alkoxy”, as used herein, refers to an alkyl group, as previously defined, attached to the parent molecular moiety through an oxygen atom. Examples of “alkoxy” radicals as used herein include, but are not limited to, methoxy, ethoxy, propoxy, prop-2-oxy, butoxy, but-2-oxy, 2-methylprop-1-oxy and 2-methylprop-2-oxy. The terms “aryloxy”, “heteroaryloxy”, “aralkyloxy” and “heteroaralkyloxy” are likewise defined.

The term “oxo” means ═O. The term “nitro” means —NO₂. The term “cyano” means —C≡N. The term “isocyano” means “—N≡C”.

The term “sulfhydryl” means —SH. The term “alkylthio”, as used herein, refers to an alkyl group, as previously defined, attached to the parent molecular moiety through an sulfur atom (i.e., an alkyl sulfenyl group). The terms “arylthio”, “heteroarylthio”, “aralkylthio” and “heteroaralkylthio” are likewise defined.

The term “sulfoxido” as used herein, refers to a radical that can be represented by the general formula:

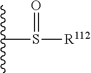

wherein R¹¹² represents hydrogen, alkyl, alkenyl, alkynyl, aryl, heteroaryl or —(CH₂)_{b}R²⁰⁰, wherein b and R²⁰⁰ are defined above.

The term “sulfonyl”, as used herein, refers to a radical that can be represented by the general formula:

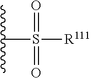

wherein R¹¹¹ represents hydrogen, alkyl, alkenyl, alkynyl, aryl, heteroaryl or —(CH₂)_{b}R²⁰⁰, wherein b and R²⁰⁰ are defined above.

The term “oxysulfonyl” is art-recognized and includes a radical that can be represented by the general formula:

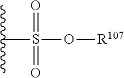

in which R¹⁰⁷ is an electron pair, hydrogen, alkyl, alkenyl, alkynyl, aryl, heteroaryl or —(CH₂)_{b}R²⁰⁰, wherein b and R²⁰⁰ are defined above.

The term “sulfonylamino” is art-recognized and includes a radical that can be represented by the general formula:

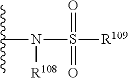

in which R¹⁰⁸ and R¹⁰⁹ independently represents hydrogen, alkyl, alkenyl, alkynyl, aryl, heteroaryl or —(CH₂)_{b}R²⁰⁰, wherein b and R²⁰⁰ are defined above.

The term “sulfamoyl” is art-recognized and includes a radical that can be represented by the general formula:

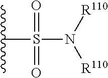

wherein R¹¹⁰ independently for each occurrence represents hydrogen, alkyl, alkenyl, alkynyl, aryl, heteroaryl or —(CH₂)_{b}R²⁰⁰, wherein b and R²⁰⁰ are defined above.

The term “carbocyclyl” is art-recognized and refers to univalent radical formed by removing a hydrogen atom from an benzene, napthalene, antracene or cycloalkane. Each of the rings of the carbocyclyl may be substituted with any of the radicals described herein.

The term “polycyclyl” is art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with any of the radicals described herein.

The term “aryl” refer to 5 to 10-membered mono-, bi- or tri-cyclic radicals (i.e., a univalent radical formed by removing a hydrogen atom from a benzene, napthalene or antracene). The aryl radical can be substituted at one or more ring positions with any of the radicals described herein.

The term “heteroaryl” refer to 5 to 10-membered mono-, bi- or tri-cyclic radicals which contain one to four heteroatoms (i.e., a univalent radical formed by removing a hydrogen atom from a heteroaromatic compound). The heteroaryl radical can be substituted at one or more ring positions with any of the radicals described herein.

The term “heterocyclyl” refers to 3 to 10-membered radical ring structures which contain one to four heteroatoms (i.e., univalent radicals formed by removing a hydrogen atom from a ring of a heterocyclic compound). Heterocyclic compounds include thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with any of the radicals described herein.

The term “heteroatom” as used herein means an atom of any element other than carbon or hydrogen. Preferred heteroatoms are nitrogen, oxygen, sulfur, phosphorus and selenium.

As used herein, the term “substituted” is contemplated to include all permissible number and types of substituents of organic compounds (e.g., monsubstituted, disubstituted, trisubstituted, tetrasubstituted, and the like). In a broad aspect, the permissible substituents include acyclic and cyclic, branched and unbranched, carbocyclic and heterocyclic, aromatic and nonaromatic substituents of organic compounds. Illustrative substituents include, for example, those described hereinabove (such as alkyl, cycloalkyl, halo, perhaloalkyl, aralkyl, heteroaralkyl, alkenyl, alkynyl, carbonyl, ester, carboxyl, carboxylic acid, formyl, thiocarbonyl, thioester, thiocarboxylic acid, thioformyl, ketone, aldehyde, cyano, isocyano, amino, acylamino, amido, nitro, hydroxyl, alkoxy, aryloxy, heteroaryloxy, aralkyloxy, sulfhydryl, alkylthio, arylthio, heteroarylthio, aralkylthio, heteroaralkylthio, sulfoxido, sulfonyl, oxysulfonyl, sulfonylamino, sulfamoyl, carbocyclyl, polycyclyl, aryl, heteroaryl, and heterocyclyl). The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The following abbreviations are used herein: CQ, chloroquine; CQR, chloroquine resistant; CQS, chloroquine sensitive; Dd2, CQR strain; FCB, CQR strain; HB3 CQS strain; GCO3, CQS strain; FPIX, ferriprotoporphyrin IX; Hb, hemoglobin; P. falciparum, Plasmodium falciparum; IC₅₀, 50% inhibitory drug concentration; EDC, 1-ethyl-3-(3-dimethylaminopropyl)carbodiimide hydrochloride; Pybop, benzotriazol-1-yl-oxytripyrrolidinophosphonium hexafluorophosphate; CDMT, 2-chloro-4,6-dimethoxy-1,3,5-triazine. In addition, the abbreviations Me, Et, iPr, tBu, Ph, Tf, Nf, Ts, and Ms represent methyl, ethyl, isopropyl, tertbutyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

The term “salts” can include acid addition salts or addition salts of free bases. Examples of acids which may be employed to form pharmaceutically acceptable acid addition salts include but are not limited to salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al. “Pharmaceutical Salts,” J. of Pharma. Sci., 1977; 66:1).

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopoeia or other generally recognized pharmacopeias for use in mammals, and more particularly in humans.

The term “pharmaceutically acceptable derivative” as used herein means any pharmaceutically acceptable salt, solvate or prodrug, e.g., ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Preferred pharmaceutically acceptable derivatives are salts, solvates, esters, carbamates and phosphate esters. Particularly preferred pharmaceutically acceptable derivatives are salts, solvates and esters. Most preferred pharmaceutically acceptable derivatives are salts and esters. Any reference to a compound is therefore to be understood as referring also to the corresponding pharmaceutically acceptable derivative of the compound, as appropriate and expedient.

The present invention also encompasses prodrugs, i.e., compounds which release an active parent drug in vivo when administered to a mammalian subject. Any reference to a compound is therefore to be understood as referring also to the corresponding pro-drugs of the compound, as appropriate and expedient. Prodrugs of a compound of the invention are prepared by modifying functional groups present in the compound described herein in such a way that the modifications may be cleaved in vivo to release the parent compound. Prodrugs include compounds wherein a hydroxy, amino, or carboxy group of a compound described herein is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds of Formula I or any other derivative which upon being brought to the physiological pH or through enzyme action is converted to the active parent drug.

The compounds of the invention may be administered with one or more carriers. The term “carrier” applied to pharmaceutical compositions of the invention refers to a diluent, excipient, or vehicle with which an active compound is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by E. W. Martin, 18th Edition. Particularly preferred for the present invention are carriers suitable for immediate-release, i.e., release of most or all of the active ingredient over a short period of time, such as 60 minutes or less, and make rapid absorption of the drug possible.

The present invention also encompasses solvates of the compounds described herein or their salts. Preferred solvates are hydrates.

The compounds of the invention may have one or more chirality centers and, depending on the nature of individual substituents, they can also have geometrical isomers. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has a chiral center, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomer respectively). A chiral compound can exist as either an individual enantiomer or as a mixture of enantiomers. A mixture containing equal proportions of the enantiomers is called a “racemic mixture”. The present invention encompasses all individual isomers of compounds of Formula I. The description or naming of a particular compound in the specification and claims is intended to include both individual enantiomers and mixtures, racemic or otherwise (i.e., enriched in one or more isomers) thereof. Methods for the determination of stereochemistry and the resolution of stereoisomers are well-known in the art.

The present invention also encompasses stereoisomers of the syn-anti type, and mixtures thereof encountered when an oxime or similar group is present. The group of highest Cahn Ingold Prelog priority attached to one of the terminal doubly bonded atoms of the oxime, is compared with hydroxyl group of the oxime. The stereoisomer is designated as Z (zusammen=together) or Syn if the oxime hydroxyl lies on the same side of a reference plane passing through the C═N double bond as the group of highest priority; the other stereoisomer is designated as E (entgegen=opposite) or Anti.

Depending on the type of formulation, in addition to a therapeutically effective quantity of one or more compounds, they will contain solid or liquid excipients or diluents for pharmaceutical use and possibly other additives normally used in the preparation of pharmaceutical formulations, such as thickeners, aggregating agents, lubricants, disintegrating agents, flavorings and colorants.

“Treating” or “treatment” of malaria includes (1) preventing or delaying the appearance of clinical symptoms of malaria developing in a mammal that has been in contact with the parasite; (2) inhibiting the malaria, i.e., arresting, reducing or delaying the development of malaria or a relapse thereof or at least one clinical or subclinical symptom thereof; or (3) relieving or attenuating one or more of the clinical or subclinical symptoms of malaria.

The benefit to a subject to be treated is either statistically significant or at least perceptible to the patient or to the physician.

Prophylactic treatment” of malaria includes treating subjects who are at risk of developing malaria. This includes the treatment of subjects who have been exposed to malaria-bearing mosquitoes, the treatment of subjects who intend to travels to a country where malaria is endemic and the treatment of subjects who otherwise risk exposure to malaria-bearing mosquitoes.

An example of “relieving” a subclinical symptom is the observation in a treated individual of abatement in the number of immune cells that secrete pro inflammatory cytokines or lymphokines or a decrease in the mRNA encoding such lymphokines or cytokines.

“Maintenance therapy” is therapy during a phase of malaria following the acute phase, where the parasite achievement of remission (total or partial) of one or more symptoms of the disease until the next flare-up of the disease. The Plasmodium vivax and P. ovale parasites have dormant liver stages that can remain silent for years. Maintenance therapy for these strains is particularly important. The hallmarks of the acute phase include symptoms like chills, and fever.

“Responder” refers to a patient that has previously responded to a treatment for a non-infective inflammatory disease involving administration of a particular active agents (or combination of active agents) in particular amount or amounts.

“Subject” refers to an animal, which is preferably a mammal and more preferably human or a domestic animal. Most preferably, the subject is a human. As used herein, the term patient is used synonymously with subject.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

While it is possible that, for use in the methods of the invention, a compound of the invention may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice.

The phrase “pharmaceutically acceptable” refers to molecular entities and compositions that are generally regarded as safe. In particular, pharmaceutically acceptable carriers used in the pharmaceutical compositions of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

### Pharmaceutical Compositions

The compounds of the invention (e.g., compounds of formula I-V) may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

It will be appreciated that pharmaceutical compositions for use in accordance with the present invention may be in the form of oral, parenternal, transdermal, inhalation, sublingual, topical, implant, nasal, or enterally administered (or other mucosally administered) suspensions, capsules or tablets, which may be formulated in conventional manner using one or more pharmaceutically acceptable carriers or excipients.

There may be different composition/formulation requirements depending on the different delivery systems. It is to be understood that not all of the compounds need to be administered by the same route. Likewise, if the composition comprises more than one active component, then those components may be administered by different routes. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

The present invention further relates to pharmaceutical formulations containing a therapeutically effective quantity of a compound described herein or one of its salts mixed with a pharmaceutically acceptable vehicle. The pharmaceutical formulations of the present invention can be liquids that are suitable for oral and/or parenteral administration, for example, drops, syrups, solutions, injectable solutions that are ready for use or are prepared by the dilution of a freeze-dried product but are preferably solid or semisolid as tablets, capsules, granules, powders, pellets, pessaries, suppositories, creams, salves, gels, ointments; or solutions, suspensions, emulsions, or other forms suitable for administration by the transdermal route or by inhalation.

The compounds of the invention can be administered for immediate-, delayed-, modified-, sustained-, pulsed- or controlled-release applications.

The most preferred oral compositions are slow, delayed or positioned release (e.g., enteric especially colonic release) tablets or capsules. This release profile can be achieved without limitation by use of a coating resistant to conditions within the stomach but releasing the contents in the colon or other portion of the GI tract wherein a lesion or inflammation site has been identified. Or a delayed release can be achieved by a coating that is simply slow to disintegrate. Or the two (delayed and positioned release) profiles can be combined in a single formulation by choice of one or more appropriate coatings and other excipients. Such formulations constitute a further feature of the present invention.

Suitable compositions for delayed or positioned release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof. Plasticizers such as, but not limited to polyethylene glycol, dibutylphthalate, triacetin and castor oil may be used. A pigment may also be used to color the film. Suppositories are be prepared by using carriers like cocoa butter, suppository bases such as Suppocire C, and Suppocire NA50 (supplied by Gattefosse Deutschland GmbH, D-Weil am Rhein, Germany) and other Suppocire type excipients obtained by interesterification of hydrogenated palm oil and palm kernel oil (C₈₋₁₈ triglycerides), esterification of glycerol and specific fatty acids, or polyglycosylated glycerides, and whitepsol (hydrogenated plant oils derivatives with additives). Enemas are formulated by using the appropriate active compound according to the present invention and solvents or excipients for suspensions. Suspensions are produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropylmethylcellulose, hydroxyethylcellulose, ethylcellulose, methylcellulose, polyvinyl alcohol, polyvinyl pyrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated caster oil, polyoxyethylene alkyl ethers, and pluronic and appropriate buffer system in pH range of 6.5 to S. The use of preservatives, masking agents is suitable. Compounds can also be incorporated in the formulation by using their water-soluble salt forms.

Alternatively, materials may be incorporated into the matrix of the tablet e.g., hydroxypropyl methylcellulose, ethyl cellulose or polymers of acrylic and metacrylic acid esters. These latter materials may also be applied to tablets by compression coating.

Pharmaceutical compositions can be prepared by mixing a therapeutically effective amount of the active substance with a pharmaceutically acceptable carrier that can have different forms, depending on the way of administration. Pharmaceutical compositions can be prepared by using conventional pharmaceutical excipients and methods of preparation. The forms for oral administration can be capsules, powders or tablets where usual solid vehicles including lactose, starch, glucose, methylcellulose, magnesium stearate, di-calcium phosphate, mannitol may be added, as well as usual liquid oral excipients including, but not limited to, ethanol, glycerol, and water. All excipients may be mixed with disintegrating agents, solvents, granulating agents, moisturizers and binders. When a solid carrier is used for preparation of oral compositions (e.g., starch, sugar, kaolin, binders disintegrating agents) preparation can be in the form of powder, capsules containing granules or coated particles, tablets, hard gelatin capsules, or granules without limitation, and the amount of the solid carrier can vary (between 1 mg to 1 g). Tablets and capsules are the preferred oral composition forms.

Pharmaceutical compositions containing compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and odorants.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT″″) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10%, more preferably 0.01-1% of the active material.

A therapeutically effective amount of the compound of the present invention can be determined by methods known in the art. The therapeutically effective quantities will depend on the age and on the general physiological condition of the patient, the route of administration and the pharmaceutical formulation used. It will also be determine by the strain of malaria parasite that has infected the subject. The therapeutic doses will generally be between about 10 and 2000 mg/day and preferably between about 30 and 1500 mg/day. Other ranges may be used, including, for example, 50-500 mg/day, 50-300 mg/day, 100-200 mg/day. The amount of the compound required for prophylactic treatment, referred to as a prophylactically-effective dosage, is generally the same as described for therapeutic treatment.

Administration may be once a day, twice a day, or more often, and may be decreased during a maintenance phase of the disease or disorder, e.g., once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

### Exemplification

The invention now being generally described, it will be more readily understood by reference to the following examples, which are included merely for purposes of illustration of certain aspects and embodiments of the present invention, and are not intended to limit the invention.

### I. Cell Culture and Antimalarial Activity Measurements

Drug activities were assessed and IC₅₀ were quantified essentially as described previously. Delarue, S.; Girault, S.; Maes, L.; Debreu-Fontaine, M. A.; Labaeid, M.; Grellier, P.; Sergheraert, C. Synthesis and in vitro and in vivo antimalarial activity of new 4-anilinoquinolines. J. Med. Chem. 2001, 44, 2827-2833; and O'Neill, P. M.; Willock, D. J.; Hawley, S. R.; Bray, P. G.; Storr, R. C.; Ward, S. A.; Park, B. K. Synthesis, antimalarial activity, and molecular modeling of tebuquine analogues. J. Med. Chem. 1997, 40, 437-448. The aminoquinolines were diluted using complete media under sterile conditions and plated in a 96 well plate format. Sorbitol synchronized cultures were utilized with >95% of the parasites at the ring stage. Cultures were diluted to give a working stock of 0.5% parasitemia and 2% hematocrit (final hematocrit 1% & 0.5% Parasitemia). The plates were incubated for 72 h at 37° C. After 72 h, 50 μL of 10×SYBR green I dye was added to each well, and the plate was incubated for 1 h at 37° C. Fluorescence was measured at 530 nm (490 nm excitation) using a spectra geminiEM plate reader. Data analysis was performed using sigma plot 9.0 software after downloading data in Excel format. For each assay, each drug dilution was analyzed in triplicate, and the results from at least two separate assays were averaged in each case (S.D. <10% in each case). All drugs were all tested against at least one chloroquine sensitive, and at least one chloroquine resistant strains of P. falciparum (e.g., GCO3, HB3 and FCB, Dd2, respectively). Based on NMR spectroscopic and HPLC chromatographic analyses, all compounds were at least of 98% purity.

### II. Synthetic Procedures and Product Characterization

All reagents and solvents commercially available were used without further purification. Flash chromatography was performed on Kieselgel 60, particle size 0.032-0.063 mm. NMR spectra were obtained on a 300 MHz (¹H-NMR) and 75 MHz (¹³C-NMR) Varian FT-NMR spectrometer using CDCl₃ as solvent unless indicated otherwise. Electrospray mass spectra (ESI-MS) were collected on a Thermo Finnigan LCQ instrument. Samples were dissolved in acetonitrile/water (1:1 v/v) containing 1% acetic acid (1 mg/mL) for MS analysis.

Representative Procedure for the Synthesis of N-(7-Chloro-4-quinolyl)-1,n-diaminoalkanes. A mixture of 4,7-dichloroquinoline (1.0 g, 5.1 mmol) and ethylenediamine (1.7 mL, 25.3 mmol) was heated to 110° C. for 6 h under inert atmosphere and then cooled to room temperature. Aqueous NaOH (1N, 10 mL) was then added and the mixture was extracted with CH₂Cl₂. The organic layers were washed with water, brine, dried over anhydrous Na₂SO₄ and evaporated under reduced pressure. N-(7-Chloro-4-quinolyl)-1,2-diaminoethane (1.04 g, 4.4 mmol, 87% yield) was obtained as pale yellow crystals and used without further purification.

N-(7-Chloro-4-quinolyl)-1,2-diaminoethane. ¹H-NMR (300 MHz, CDCl₃) δ=1.26 (bs, 2H), 3.07-3.16 (m, 2H), 3.25-3.36 (m, 2H), 5.60-5.80 (m, 1H), 6.42 (d, J=5.7 Hz, 1H), 7.37 (dd, J=2.4 Hz, 8.7 Hz, 1H), 7.72 (d, J=8.7 Hz, 1H), 7.96 (d, J=2.4 Hz, 1H), 8.54 (d, J=5.7 Hz, 1H); ¹³C-NMR (75 MHz, CDCl₃) δ=39.2, 44.9, 97.7, 116.6, 122.1, 123.3, 126.8, 133.1, 148.1, 149.5, 150.8.

N-(7-Chloro-4-quinolyl)-1,3-diaminopropane. Employing 1.0 g (5.1 mmol) of 4,7-dichloroquinoline in the procedure described above gave 1.05 g (4.5 mmol, 88% yield) of pale yellow crystals. ¹H-NMR (300 MHz, CDCl₃) δ=1.48 (bs, 2H), 1.84-1.96 (m, 2H), 3.00-3.10 (m, 2H), 3.38-3.48 (m, 2H), 6.33 (d, J=5.4 Hz, 1H), 7.30 (dd, J=2.1 Hz, 9.0 Hz, 1H), 7.72 (d, J=9.0 Hz, 1H), 7.92 (d, J=2.1 Hz, 1H), 8.50 (d, J=5.4 Hz, 1H); ¹³C-NMR (75 MHz, CDCl₃) δ=29.5, 40.8, 42.8, 97.8, 117.1, 122.0, 124.2, 127.6, 133.9, 148.6, 150.0, 151.5.

N-(7-Chloro-4-quinolyl)-1,4-diaminobutane. Employing 2.0 g (10.1 mmol) of 4,7-dichloroquinoline in the procedure described above gave 2.09 g (8.4 mmol, 83% yield) of pale yellow crystals. ¹H-NMR (300 MHz, CDCl₃) δ=1.28 (bs, 2H), 1.56-1.68 (m, 2H), 1.76-1.90 (m, 2H), 2.79 (t, J=6.6 Hz, 2H), 3.22-3.32 (m, 2H), 6.09 (bs, 1H), 6.34 (d, J=5.4 Hz, 1H), 7.29 (dd, J=2.7 Hz, 9.0 Hz, 1H), 7.72 (d, J=9.0 Hz, 1H), 7.91 (d, J=2.7 Hz, 1H), 8.49 (d, J=5.4 Hz, 1H); ¹³C-NMR (75 MHz, CDCl₃) δ=25.9, 30.6, 41.4, 43.0, 98.6, 117.3, 121.6, 124.8, 128.3, 134.5, 149.0, 150.0, 151.9.

N-(7-Chloro-4-quinolyl)-1,5-diaminopentane. Employing 1.0 g (5.1 mmol) of 4,7-dichloroquinoline in the procedure described above gave 1.16 g (4.4 mmol, 87% yield) of pale yellow crystals. ¹H-NMR (300 MHz, CDCl₃) δ=1.15 (bs, 2H), 1.40-1.60 (m, 4H), 1.66-1.86 (m, 2H), 2.71 (t, J=6.6 Hz, 2H), 3.20-3.38 (m, 2H), 5.49 (t, J=4.8 Hz, 1H), 6.36 (d, J=5.4 Hz, 1H), 7.28 (dd, J=2.1, 9.0 Hz, 1H), 7.72 (d, J=9.0 Hz, 1H), 7.92 (d, J=2.1 Hz, 1H), 8.50 (d, J=5.4 Hz, 1H); ¹³C-NMR (75 MHz, CDCl₃) δ=24.3, 28.5, 33.1, 41.9, 43.0, 98.9, 117.0, 120.9, 125.0, 128.6, 134.6, 149.0, 149.6, 151.9.

N-(7-Chloro-4-quinolyl)-1,6-diaminohexane. Employing 1.0 g (5.1 mmol) of 4,7-dichloroquinoline in the procedure described above gave 1.28 g (4.6 mmol, 91% yield) of pale yellow crystals. ¹H-NMR (300 MHz, DMSO-d₆) δ=1.30-1.42 (m, 6H), 1.60-1.72 (m, 2H), 2.48-2.56 (m, 2H, overlapping with DMSO signal), 3.20-3.34 (m, 2H, overlapping with water signal), 6.46 (d, J=5.4 Hz, 1H), 7.29 (t, J=4.8 Hz, 1H), 7.44 (dd, J=2.1 Hz, 9.0 Hz, 1H), 7.77 (d, J=2.1 Hz, 1H), 8.27 (d, J=9.0 Hz, 1H), 8.39 (d, J=5.4 Hz, 1H); ¹³C-NMR (75 MHz, CD₃OD) δ=27.7, 28.0, 29.3, 33.5, 42.3, 43.9, 99.5, 118.7, 120.2, 124.3, 125.8, 127.5, 136.2, 149.6, 152.3.

N-(7-Chloro-4-quinolyl)-N-(3-diethylaminopropanoyl)-1,2-diaminoethane. A mixture of N-(7-chloro-4-quinolyl)-1,2-diaminoethane (0.1 g, 0.45 mmol), N,N-diethylamino-3-propionic acid (0.11 g, 0.6 mmol), EDC (0.11 g, 0.6 mmol) and Et₃N (0.19 mL, 1.35 mmol) in 4 mL of anhydrous DMF and CHCl₃ (1:1 v/v) was stirred at room temperature for 2 days. Saturated NaHCO₃ solution was added to the cooled reaction mixture, which was then extracted with CH₂Cl₂, dried over anhydrous MgSO₄, and concentrated in vacuo. Flash chromatography using EtOH:Et₃N (1:0.05 v/v) as the mobile phase afforded 0.10 g (0.44 mmol, 63% yield) of yellow crystals. ¹H-NMR (300 MHz, CDCl₃) δ=1.05 (t, J=7.1 Hz, 6H), 2.48 (t, J=6.1 Hz, 2H), 2.58 (q, J=7.1 Hz, 4H), 2.69 (t, J=6.1 Hz, 2H), 3.30-3.45 (m, 2H), 3.64-3.78 (m, 2H), 6.28 (d, J=5.4 Hz, 1H), 7.11 (bs, 1H), 7.40 (dd, J=2.1 Hz, J=9.0 Hz, 1H), 7.87 (d, J=9.0 Hz, 1H), 7.94 (d, J=2.1 Hz, 1H), 8.50 (d, J=5.4 Hz, 1H), 9.51 (bs, 1H); ¹³C-NMR (75 MHz, CDCl₃) δ=11.6, 32.4, 38.4, 46.3, 46.5, 48.9, 98.2, 117.5, 122.7, 125.7, 128.2, 135.2, 148.9, 150.7, 151.8, 176.2; MS (ESI) m/z calcd for C₁₈H₂₅ClN₄O 348.2. Found (M+H)⁺: 349.1.

N-(7-Chloro-4-quinolyl)-N-(3-diethylaminopropyl)-1,2-diaminoethane. A solution of N-(7-chloro-4-quinolyl)-N′-(3-diethylaminopropanoyl)-1,2-diaminoethane (0.08 g, 0.23 mmol) in 2 mL of THF was heated to reflux and borane-dimethyl sulfide complex (0.13 mL, 1.4 mmol) was added. After 14 h, 6M HCl (1 mL) was added and the mixture was heated to 100° C. for 30 minutes. The clear solution was cooled to room temperature, basified with saturated NaOH and extracted with CH₂Cl₂. The combined organic layers were dried over anhydrous MgSO₄ and concentrated in vacuo. Purification by flash chromatography using EtOH:Et₃N (1:0.03 v/v) as the mobile phase gave a brown oil (0.07 g, 0.21 mmol, 89% yield). ¹H-NMR (300 MHz, CDCl₃) δ=1.03 (t, J=7.1 Hz, 6H), 1.62-1.78 (m, 2H), 2.46-2.60 (m, 6H), 2.74 (t, J=6.6 Hz, 2H), 3.01-3.19 (m, 2H), 3.30-3.41 (m, 2H), 5.98 (bs, 1H), 6.41 (d, J=5.4 Hz, 1H), 7.38 (dd, J=2.2 Hz, J=9.0 Hz, 1H), 7.76 (d, J=9.0 Hz, 1H), 7.97 (d, J=2.2 Hz, 1H), 8.54 (d, J=5.4 Hz); ¹³C-NMR (75 MHz, CDCl₃) δ=11.6, 26.9, 42.1, 47.1, 47.6, 48.5, 51.5, 99.2, 117.7, 122.0, 125.4, 128.7, 135.1, 149.3, 150.1, 152.2; MS (ESI) m/z calcd for C₁₈H₂₇ClN₄ 334.2. Found (M+H)⁺: 335.2.

N-(7-Chloro-4-quinolyl)-N-ethyl-N-(3-diethylaminopropyl)-1,2-diaminoethane. To a solution of N-(7-chloro-4-quinolyl)-N′-(3-diethylaminopropyl)-1,2-diaminoethane (0.06 g, 0.18 mmol) in 1 mL of glacial acetic acid, NaBH₄ (0.16 g, 4.3 mmol) was added at 5° C. and the reaction temperature was increased to 50° C. After 18 h, the reaction mixture was cooled, basified with saturated NaOH and extracted with CH₂Cl₂. The combined organic layers were dried over anhydrous MgSO₄, and concentrated in vacuo. Purification by flash chromatography using EtOH:Et₃N (1:0.03 v/v) as the mobile phase gave a yellow oil (0.03 g, 0.08 mmol, 46% yield). ¹H-NMR (300 MHz, CDCl₃) δ=1.03 (t, J=7.1 Hz, 6H), 1.10 (t, J=7.1 Hz, 3H), 1.63-1.82 (m, 2H), 2.43-2.74 (m, 10H), 2.85 (t, J=5.8 Hz, 2H), 3.26-3.39 (m, 2H), 6.11 (bs, 1H), 6.40 (d, J=5.3 Hz, 1H), 7.40 (dd, J=2.1 Hz, J=8.8 Hz, 1H), 7.73 (d, J=8.8 Hz, 1H), 7.97 (d, J=2.1 Hz, 1H), 8.55 (d, J=5.3 Hz, 1H); ¹³C-NMR (75 MHz, CDCl₃) δ=11.3, 12.1, 24.8, 30.0, 40.0. 46.9, 47.2, 51.3, 51.4, 99.5, 117.7, 121.6, 125.4, 128.9, 135.0, 149.4, 150.2, 152.4; MS (ESI) m/z calcd for C₂₀H₃₁ClN₄ 362.2. Found (M+H)⁺: 363.1.

N-(7-Chloro-4-quinolyl)-N-(3-diethylaminopropanoyl)-1,3-diaminopropane. A mixture of N-(7-chloro-4-quinolyl)-1,3-diaminopropane (1.0 g, 4.24 mmol), N,N-diethylamino-3-propionic acid (0.78 g, 4.3 mmol), EDC (0.98 g, 5.1 mmol) and triethylamine (1.8 mL, 12.9 mmol) in 30 mL of anhydrous DMF and chloroform (1:1 v/v) was stirred at room temperature for 2.5 days. The reaction mixture was concentrated in vacuo, then dissolved in dichloromethane and extracted with aqueous NaOH. The combined organic layers were dried over anhydrous MgSO₄ and concentrated in vacuo. The crude product was purified by flash chromatography (1:1:0.05 ethanol:hexanes:triethylamine v/v) to give 0.83 g of (2.3 mmol, 54% yield) pale yellow crystals. ¹H-NMR (300 MHz, CDCl₃) δ=1.02 (t, J=7.1 Hz, 6H), 1.74-1.83 (m, 2H), 2.41 (t, J=5.7 Hz, 2H), 2.53 (q, J=7.1 Hz, 4H), 2.67 (t, J=5.9 Hz, 2H), 3.32-3.43 (m, 4H), 6.37 (d, J=5.6 Hz, 1H), 6.76 (t, J=5.7 Hz, 1H), 7.36 (dd, J=2.1 Hz, J=9.0 Hz, 1H), 7.90 (d, J=2.1 Hz, 1H), 8.02 (d, J=9.0 Hz, 1H), 8.45 (d, J=5.6 Hz, 1H), 9.04 (t, J=5.7 Hz, 1H); ¹³C-NMR (75 MHz, CDCl₃) δ=11.8, 28.6, 32.7, 35.7, 39.2, 46.5, 49.2, 98.6, 117.9, 122.5, 125.7, 128.5, 135.4, 149.4, 150.5, 151.9, 174.8; MS (ESI) m/z calcd for C₁₉H₂₇ClN₄O 362.2. Found (M+H)⁺: 362.9.

N-(7-Chloro-4-quinolyl)-N-(3-diethylaminopropyl)-1,3-diaminopropane. To N-(7-chloro-4-quinolyl)-N′-(3-diethylaminopropanoyl)-1,3-diaminopropane (0.2 g, 0.55 mmol) in 9 mL of anhydrous DMF, borane-dimethyl sulfide complex (0.35 mL, 3.69 mmol) was added dropwise at 0° C. The reaction mixture was heated to reflux for 2.5 h and then quenched with 1.6 mL of water. Concentrated HCl (1.0 mL) was added and the reaction was refluxed for another 1.5 h. The reaction mixture was cooled to room temperature, basified (pH>10) with NaOH and extracted with chloroform. The combined organic layers were dried over anhydrous MgSO₄ and concentrated in vacuo to give a yellow oil (0.16 g, 0.46 mmol, 82% yield). ¹H-NMR (300 MHz, CDCl₃) δ=1.01 (t, J=7.1 Hz, 6H), 1.71-1.80 (m, 2H), 1.90-1.97 (m, 2H), 2.48-2.55 (m, 6H), 2.74 (t, J=6.9 Hz, 2H), 2.89-2.93 (m, 2H), 3.37-3.42 (m, 2H), 6.30 (d, J=5.6 Hz, 1H), 7.29 (dd, J=2.2 Hz, J=9.0 Hz, 1H), 7.76 (d, J=9.0 Hz, 1H), 7.92 (d, J=2.2 Hz, 1H), 8.50 (d, J=5.4 Hz, 1H); ¹³C-NMR (75 MHz, CDCl₃) δ=11.6, 25.1, 26.4, 42.2, 46.9, 49.6, 52.2, 53.7, 98.4, 117.9, 123.2, 125.2, 128.4, 135.0, 149.3, 150.7, 152.1; MS (ESI) m/z calcd for C₁₉H₂₉ClN₄ 348.2. Found (M+H)⁺: 349.1.

N-(7-Chloro-4-quinolyl)-N-ethyl-N-(3-diethylaminopropyl)-1,3-diaminopropane. To a solution of N-(7-chloro-4-quinolyl)-N′-(3-diethylaminopropyl)-1,3-diaminopropane (0.09 g, 0.24 mmol) in 4 mL of glacial acetic acid, sodium borohydride (0.24 g, 6.3 mmol) was added at 5° C. The reaction was warmed to room temperature for 1 h and then heated to 60° C. for 30 h. After cooling to room temperature, the mixture was basified (pH>10) with NaOH and extracted with dichloromethane. The combined organic layers were dried over anhydrous MgSO₄ and concentrated in vacuo. The crude product was purified by flash chromatography (1.0:0.05 ethanol:triethylamine v/v) to give 0.075 g (0.12 mmol, 81% yield) of a yellow oil. ¹H-NMR (300 MHz, CDCl₃) δ=0.94 (t, J=7.2 Hz, 6H), 1.11 (t, J=7.2 Hz, 3H), 1.62-1.73 (m, 2H), 1.88-1.98 (m, 2H), 2.38-2.48 (m, 6H), 2.56 (t, J=7.7 Hz, 2H), 2.62-2.71 (m, 4H), 3.32-3.42 (m, 2H), 6.31 (d, J=5.4 Hz, 1H), 7.32 (dd, J=2.1 Hz, 8.8 Hz, 1H), 7.71 (d, J=8.8 Hz, 1H), 7.86 (bs, 1H), 7.93 (d, J=2.1 Hz, 1H), 8.50 (d, J=5.4 Hz, 1H); ¹³C-NMR (75 MHz, CDCl₃) δ=11.8, 24.7, 24.9, 44.9, 47.1, 48.1, 51.4, 52.3, 54.2, 98.6, 117.9, 122.3, 124.9, 128.9, 134.8, 149.5, 150.8, 152.5; MS (ESI) m/z calcd for C₂₁H₃₃ClN₄ 376.2. Found (M+H)⁺: 376.9.

N-(7-Chloro-4-quinolyl)-N-(3-diethylaminopropanoyl)-1,4-diaminobutane. A mixture of N-(7-chloro-4-quinolyl)-1,4-diaminobutane (2.0 g, 8.0 mmol), N,N-diethylamino-3-propionic acid (1.45 g, 8.0 mmol), EDC (1.84 g, 9.6 mmol), and triethylamine (3.35 mL, 24.0 mmol) in 80 mL of anhydrous DMF and chloroform (1:1 v/v) was stirred at room temperature for 2.5 days. The reaction mixture was concentrated in vacuo and partitioned between dichloromethane and 1N NaOH solution. The combined organic layers were dried over anhydrous Na₂SO₄ and concentrated in vacuo. The crude product was purified by flash chromatography (0.5% ammonium hydroxide in MeOH) to give 1.8 g (4.8 mmol, 60% yield) of colorless crystals. ¹H-NMR (300 MHz, CDCl₃) δ=1.02 (t, J=7.2 Hz, 6H), 1.60-1.88 (m, 4H), 2.36 (t, J=6.0 Hz, 2H), 2.54 (q, J=7.2 Hz, 4H), 2.65 (t, J=6.0 Hz, 2H), 3.28-3.42 (m, 4H), 5.71 (bt, 1H), 6.38 (d, J=5.7 Hz, 1H), 7.35 (dd, J=2.4 Hz, J=9.0 Hz, 1H), 7.86 (d, J=9.0 Hz, 1H), 7.93 (d, J=2.4 Hz, 1H), 8.51 (d, J=5.7 Hz, 1H), 8.85 (bt, 1H); ¹³C-NMR (75 MHz, CDCl₃) δ=11.3, 25.2, 27.8, 32.3, 38.1, 42.9, 45.8, 48.6, 98.6, 117.3, 121.9, 124.7, 128.0, 134.4, 148.9, 150.0, 151.6, 173.1; MS (ESI) m/z calcd for C₂₀H₂₉ClN₄O 376.2. Found (M+H)⁺: 376.9.

N-(7-Chloro-4-quinolyl)-N-(3-diethylaminopropyl)-1,4-diaminobutane. To a solution of N-(7-chloro-4-quinolyl)-N′-(3-diethylaminopropanoyl)-1,4-diaminobutane (0.11 g, 0.29 mmol) in anhydrous THF, borane-dimethyl sulfide complex (0.15 mL, 1.59 mmol) was added dropwise at 0° C. After stirring at 0° C. for 15 minutes, the reaction mixture was heated to reflux for 3 h, cooled to room temperature and carefully quenched with 1.0 mL of water. Concentrated HCl (0.4 mL) and 1.0 mL of water were then added and the mixture was heated to reflux for 1.5 h. After cooling to room temperature, the mixture was basified (pH>10) with 5N NaOH and extracted with chloroform. The combined organic layers were dried over anhydrous Na₂SO₄ and concentrated in vacuo. The crude product was purified by flash chromatography on basic alumina (2% MeOH in CH₂Cl₂) to afford 0.084 g (0.23 mmol, 79% yield) of a light yellow oil. ¹H-NMR (300 MHz, CDCl₃) δ=1.01 (t, J=7.2 Hz, 6H), 1.66-1.79 (m, 4H), 1.81-1.94 (m, 2H), 2.44-2.59 (m, 6H), 2.68-2.79 (m, 4H), 3.10-3.40 (m, 3H), 6.22 (bs, 1H), 6.36 (d, J=5.4 Hz, 1H), 7.33 (dd, J=2.1 Hz, J=9.0 Hz, 1H), 7.83 (d, J=9.0 Hz, 1H), 7.92 (d, J=2.1 Hz, 1H), 8.50 (d, J=5.4 Hz, 1H); ¹³C-NMR (75 MHz, CDCl₃) δ=11.6, 26.1, 27.0, 27.7, 43.0, 46.7, 48.9, 49.0, 51.3, 98.6, 98.7, 117.3, 121.5, 124.7, 128.3, 134.5, 149.0, 150.0, 151.8; MS (ESI) m/z calcd for C₂₀H₃₁ClN₄ 362.2. Found (M+H)⁺: 362.9.

N-(7-Chloro-4-quinolyl)-N-ethyl-N-(3-diethylaminopropyl)-1,4-diaminobutane. To N-(7-chloro-4-quinolyl)-N′-(3-diethylaminopropyl)-1,4-diaminobutane (0.14 g, 0.39 mmol) in 5 mL of glacial acetic acid, sodium borohydride (0.3 g, 7.8 mmol) was added portionwise at 0° C. The reaction was stirred at room temperature for 1 h and then heated at 60° C. for 18 h. The reaction mixture was cooled to room temperature, basified (pH>10) with 12N NaOH and extracted with dichloromethane. The combined organic layers were dried over anhydrous Na₂SO₄ and concentrated in vacuo. The crude product was purified by flash chromatography on basic alumina (1% MeOH in CH₂Cl₂) to afford 0.12 g (0.31 mmol, 79% yield) of a yellow oil. ¹H-NMR (300 MHz, CDCl₃) δ=0.95-1.08 (m, 9H), 1.55-1.72 (m, 4H), 1.77-1.89 (m, 2H), 2.36-2.63 (m, 12H), 3.25-3.36 (m, 2H), 5.78 (bs, 1H), 6.39 (d, J=5.4 Hz, 1H), 7.34 (dd, J=2.1 Hz, J=8.7 Hz, 1H), 7.71 (d, J=8.7 Hz, 1H), 7.94 (d, J=2.1 Hz, 1H), 8.52 (d, J=5.4 Hz, 1H); ¹³C-NMR (75 MHz, CDCl₃) δ=11.1, 11.4, 24.1, 25.1, 26.6, 43.1, 46.7, 47.3, 50.9, 51.4, 52.7, 98.7, 98.8, 117.2, 121.4, 124.7, 128.4, 134.5, 149.0, 149.9, 151.9; MS (ESI) m/z calcd for C₂₂H₃₅ClN₄ 390.3. Found (M+H)⁺: 391.0.

N-(7-Chloro-4-quinolyl)-N-(3-diethylaminopropanoyl)-1,5-diaminopentane. A mixture of N-(7-chloro-4-quinolyl)-1,5-diaminopentane (0.25 g, 0.95 mmol), N,N-diethylamino-3-propionic acid (0.17 g, 0.93 mmol), EDC (0.22 g, 1.14 mmol), and triethylamine (0.4 mL, 2.9 mmol) in 12 mL of anhydrous DMF and chloroform (1:1 v/v) was stirred at room temperature for 2.5 days. The reaction mixture was concentrated in vacuo, then dissolved in dichloromethane and extracted with aqueous NaOH. The combined organic layers were dried over anhydrous MgSO₄ and concentrated in vacuo. The crude product was purified by flash chromatography (1.0:0.05 methanol:ammonium hydroxide v/v) to afford 0.045 g (0.11 mmol, 12%) of colorless crystals. ¹H-NMR (300 MHz, CDCl₃) δ=1.01 (t, J=7.2 Hz, 6H), 1.49-1.59 (m, 4H), 1.82-1.87 (m, 2H), 2.53 (t, J=6.0 Hz, 2H), 2.53 (q, J=7.2 Hz, 4H), 2.64 (t, J=6.0 Hz, 2H), 3.26-3.33 (m, 4H), 5.46 (bs, 1H), 6.37 (d, J=5.4 Hz, 1H), 7.35 (dd, J=2.2 Hz, 8.8 Hz, 1H), 7.94 (d, J=2.2 Hz, 1H), 7.96 (d, J=8.8 Hz, 1H), 8.51 (d, J=5.4 Hz, 1H), 8.80 (bs, 1H); ¹³C-NMR (75 MHz, CDCl₃) δ=11.8, 24.3, 28.0, 30.0, 32.8, 37.9, 43.4, 46.3, 49.2, 100.6, 117.6, 122.0, 128.9, 134.9, 149.5, 150.3, 152.3, 173.7; MS (ESI) m/z calcd for C₂₁H₃₁ClN₄O 390.2. Found (M+H)⁺: 391.0.

N-(7-Chloro-4-quinolyl)-N-(3-diethylaminopropyl)-1,5-diaminopentane. To N-(7-chloro-4-quinolyl)-N′-(3-diethylaminopropanoyl)-1,5-diaminopentane (0.14 g, 0.35 mmol) in 9 mL of anhydrous DMF, borane-dimethyl sulfide complex (0.23 mL, 2.42 mmol) was added dropwise at 0° C. The reaction mixture was heated to reflux for 2.5 h and then quenched with 1.8 mL of water. Concentrated HCl (0.75 mL) was added and the mixture was refluxed for another 1.5 h. The product mixture was cooled to room temperature, basified (pH>10) with NaOH and extracted with dichloromethane. The combined organic layers were dried over anhydrous MgSO₄ and concentrated in vacuo. The residue was purified by flash chromatography (1.0:1.0:0.10 ethanol:dichloromethane:triethylamine v/v) to afford 0.09 g (0.24 mmol, 68% yield) as a light yellow oil. ¹H-NMR (300 MHz, CDCl₃) δ=0.99 (t, J=7.1 Hz, 6H), 1.53-1.61 (m, 2H), 1.67-1.83 (m, 6H), 2.50-2.58 (m, 6H), 2.74 (t, J=6.7 Hz, 2H), 2.84 (t, J=6.4 Hz, 2H), 3.36 (q, J=6.1 Hz, 2H), 5.72 (bs, 1H), 6.28 (d, J=5.4 Hz, 1H), 7.37 (dd, J=2.2 Hz, 9.0 Hz, 1H), 7.93 (d, J=2.2 Hz, 1H), 8.00 (d, J=9.0 Hz, 1H), 8.51 (d, J=5.4 Hz, 1H). ¹³C-NMR (75 MHz, CDCl₃) δ=11.8, 24.8, 26.3, 28.5, 29.2, 43.1, 46.9, 49.5, 51.9 99.2, 117.5, 121.8, 125.4, 128.9, 135.0, 149.4, 150.1, 152.2; MS (ESI) m/z calcd for C₂₁H₃₃ClN₄ 376.2. Found (M+H)⁺: 377.1.

N-(7-Chloro-4-quinolyl)-N-ethyl-N-(3-diethylaminopropyl)-1,5-diaminopentane. To N-(7-chloro-4-quinolyl)-N′-(3-diethylaminopropyl)-1,5-diaminopentane (0.064 g, 0.17 mmol) in 4 mL of glacial acetic acid, sodium borohydride (0.16 g, 4.3 mmol) was added at 5° C. The reaction was stirred at room temperature for 40 minutes and then heated to 55° C. for 36 h. After cooling to room temperature, the mixture was basified (pH>10) with NaOH and extracted with dichloromethane. The combined organic layers were dried over anhydrous MgSO₄ and concentrated in vacuo. The crude product was purified by flash chromatography (1.0:0.05 ethanol:triethylamine v/v) to afford 0.021 g (0.05 mmol, 31% yield) as colorless crystals. ¹H-NMR (300 MHz, CDCl₃) δ=1.02 (t, J=7.2 Hz, 6H), 1.04 (t, J=7.2 Hz, 3H), 1.46-1.55 (m, 6H), 1.74-1.83 (m, 2H), 2.43-2.59 (m, 12H), 3.29-3.36 (m, 2H), 5.11 (bs, 1H), 6.41 (d, J=5.5 Hz, 1H), 7.37 (dd, J=2.2 Hz, 9.0 Hz, 1H), 7.72 (d, J=9.0 Hz, 1H), 7.95 (d, J=2.2 Hz, 1H), 8.54 (d, J=5.5 Hz, 1H). ¹³C-NMR (75 MHz, CDCl₃) δ=11.5, 11.8, 24.4, 25.3, 27.0, 28.9, 43.4, 46.3, 47.1, 47.8, 51.2, 51.8, 99.3, 117.4, 121.4, 125.4, 128.5, 135.0, 149.4, 150.0, 152.3; MS (ESI) m/z calcd for C₂₃H₃₇ClN₄ 404.3. Found (M+H)⁺: 405.1.

N-(7-Chloro-4-quinolyl)-N-(3-diethylaminopropanoyl)-1,6-diaminohexane. A mixture of N-(7-chloro-4-quinolyl)-1,6-diaminohexane (0.1 g, 0.36 mmol), N,N-diethylamino-3-propionic acid (0.08 g, 0.43 mmol), EDC (0.08 g, 0.43 mmol) and Et₃N (0.19 mL, 1.35 mmol) was stirred at room temperature in 4 mL of DMF/CHCl₃ (1:1 v/v) for 2 days. Saturated NaHCO₃ was added to the cooled reaction mixture, which was then extracted with CH₂Cl₂ and dried over anhydrous MgSO₄, and concentrated in vacuo. Purification by flash chromatography using EtOH:Et₃N (1:0.05 v/v) as the mobile phase gave yellow crystals (0.12 g, 0.27 mmol, 76% yield). ¹H-NMR (300 MHz, CDCl₃) δ=1.06 (t, J=7.1 Hz, 6H), 1.25-1.62 (m, 6H), 1.63-1.82 (m, 2H), 2.40 (t, J=6.1 Hz, 2H), 2.58 (q, J=7.1 Hz, 4H), 2.69 (t, J=6.1 Hz, 2H), 3.20-3.41 (m, 4H), 5.37 (bs, 1H), 6.41 (d, J=5.4 Hz, 1H), 7.38 (dd, J=2.1 Hz, J=9.0 Hz, 1H), 7.80 (d, J=9.0 Hz, 1H), 7.97 (d, J=2.1 Hz, 1H), 8.53 (d, J=5.4 Hz, 1H), 8.67 (bs, 1H); ¹³C-NMR (75 MHz, CDCl₃) δ=11.7, 26.7, 28.7, 29.8, 32.7, 38.7, 43.1, 46.3, 49.2, 99.1, 117.5, 121.9, 125.3, 128.6, 135.0, 149.2, 150.3, 152.0, 173.2; MS (ESI) m/z calcd for C₂₂H₃₃ClN₄O 404.2. Found (M+H)⁺: 405.1.

N-(7-Chloro-4-quinolyl)-N-(3-diethylaminopropyl)-1,6-diaminohexane. A solution of N-(7-chloro-4-quinolyl)-N′-(3-diethylaminopropanoyl)-1,6-diaminohexane (0.08 g, 0.18 mmol) in 2 mL of THF was heated to reflux and borane-dimethyl sulfide complex (0.1 mL, 1.1 mmol.) was added. After 14 h, 1 mL of 6M HCl was added and the mixture was heated to 100° C. for 30 minutes. The clear solution was cooled, basified with saturated NaOH and extracted with CH₂Cl₂. The combined organic layers were dried over anhydrous MgSO₄, and concentrated in vacuo. Purification by flash chromatography using EtOH:Et₃N (1:0.03 v/v) as the mobile phase gave a brown oil (0.065 g, 0.14 mmol, 85% yield). ¹H-NMR (300 MHz, CDCl₃) δ=1.05 (t, J=7.1 Hz, 6H), 1.40-1.61 (m, 4H), 1.62-1.73 (m, 2H), 1.74-1.88 (m, 4H), 2.54-2.68 (m, 6H), 2.72 (t, J=7.1 Hz, 2H), 2.84 (t, J=6.4 Hz, 2H), 3.28-3.42 (m, 2H), 5.50 (bs, 1H), 6.41 (d, J=5.4 Hz, 1H), 7.36 (dd, J=2.1 Hz, J=9.0 Hz, 1H), 7.87 (d, J=9.0 Hz, 1H), 7.95 (d, J=2.1 Hz, 1H), 8.53 (d, J=5.4 Hz, 1H); ¹³C-NMR (75 MHz, CDCl₃) δ=11.7, 24.7, 26.7, 26.8, 28.5, 28.6, 43.0, 46.8, 48.7, 49.8, 52.5, 99.1, 117.6, 122.0, 125.4, 128.8, 135.0, 149.4, 150.2, 152.2; MS (ESI) m/z calcd for C₂₂H₃₅ClN₄ 390.3. Found (M+H)⁺: 391.1.

N-(7-Chloro-4-quinolyl)-N-ethyl-N-(3-diethylaminopropyl)-1,6-diaminohexane. To N-(7-chloro-4-quinolyl)-N′-(3-diethylaminopropyl)-1,6-diaminohexane (0.05 g, 0.13 mmol) in 1 mL of glacial acetic acid, NaBH₄ (0.11 g, 2.8 mmol) was added at 5° C. and the mixture was stirred at 50° C. for 18 h. After cooling to room temperature, the mixture was basified with saturated NaOH and extracted with CH₂Cl₂. The combined organic layers were dried over anhydrous MgSO₄ and concentrated in vacuo. Purification by flash chromatography using EtOH:Et₃N (1:0.03 v/v) as the mobile phase gave a yellow oil (0.03 g, 0.07 mmol, 55% yield). ¹H-NMR (300 MHz, CDCl₃) δ=1.04 (t, J=7.1 Hz, 9H), 1.29-1.58 (m, 6H), 1.58-1.70 (m, 2H), 1.71-1.84 (m, 2H), 2.42 (t, J=7.1 Hz, 6H), 2.45-2.62 (m, 6H), 3.25-3.39 (m, 2H), 5.10 (bs, 1H), 6.41 (d, J=5.4 Hz, 1H), 7.37 (dd, J=2.1 Hz, J=9.0 Hz, 1H), 7.69 (d, J=9.0 Hz, 1H), 7.95 (d, J=2.1 Hz, 1H), 8.54 (d, J=5.4 Hz, 1H); ¹³C-NMR (75 MHz, CDCl₃) δ=11.7, 11.8, 24.5, 27.2, 27.3, 27.5, 29.1, 43.4, 47.1, 47.8, 51.3, 51.9, 53.6, 99.3, 117.4, 121.3, 125.4, 129.1, 135.0, 149.4, 150.0, 152.3; MS (ESI) m/z calcd for C₂₄H₃₉ClN₄ 418.3. Found (M+H)⁺: 419.3.

1,7-Bis(diethylamido)heptan-4-one. To a solution of 4-ketopimelic acid (0.2 g, 1.2 mmol) in CH₃CN was added diisopropylamine (0.5 mL, 2.9 mmol), Pybop (1.19 g, 2.3 mmol) and N,N-diisopropylethylamine (0.5 mL, 3.2 mmol). The reaction was refluxed at 80° C. for 48 h. The solvents were removed in vacuo and the residue was dissolved in CH₂Cl₂ and extracted with 2M HCl and water. The organic layer was dried over anhydrous MgSO₄ and evaporated under reduced pressure to give 0.31 g (1.1 mmol, 98% yield) of a brown oil. ¹H-NMR (300 MHz, CDCl₃) δ=1.07 (t, J=7.2 Hz, 6H), 1.15 (t, J=7.2 Hz, 6H), 2.56 (t, J=6.6 Hz, 4H), 2.82 (t, J=6.6 Hz, 4H), 3.25-3.44 (m, 8H); ¹³C-NMR (75 MHz, CDCl₃) δ=13.2, 14.3, 27.1, 37.7, 40.4, 42.0, 171.0, 211.5.

1,7-Bis(diethylamido)-4-aminoheptane. 1,7-Bis(diethylamido)heptan-4-one (0.5 g, 0.7 mmol), ammonium acetate (0.45 g, 4.2 mmol) and sodium cyanoborohydride (0.11 g, 1.8 mmol) were dissolved in 4 mL of MeOH and the solution was stirred at room temperature for 36 h. After removing the solvents in vacuo, the residue was dissolved in CH₂Cl₂ and extracted with 4M NaOH. The combined organic layers were concentrated and extracted with 6M HCl. The aqueous layer was basified with concentrated NaOH and extracted with CH₂Cl₂. The combined organic layers were dried over anhydrous MgSO₄ and evaporated under reduced pressure to give 0.16 g (0.56 mmol, 80% yield) of a yellow oil. ¹H-NMR (300 MHz, CDCl₃) δ=1.10 (t, J=6.9 Hz, 6H), 1.16 (t, J=6.9 Hz, 6H), 1.52-1.70 (m, 2H), 1.72-1.88 (m, 2H), 2.29-2.50 (m, 4H), 2.72-2.86 (m, 3H), 3.18-3.38 (m, 8H); ¹³C-NMR (75 MHz, CDCl₃) δ=13.3, 14.6, 29.9, 33.7, 40.3, 42.2, 51.1, 172.3.

1,7-Bis(diethylamino)-4-aminoheptane. 1,7-Bis(diethylamido)-4-aminoheptane (0.1 g, 0.35 mmol) and lithium aluminum hydride in 1M THF (2.1 mL, 2.1 mmol) were mixed in 3 mL of anhydrous toluene and refluxed at 110° C. for 48 h. The reaction mixture was quenched with 4M NaOH and extracted with CH₂Cl₂. The organic layer was dried over anhydrous MgSO₄ and evaporated under reduced pressure to give 0.07 g (0.24 mmol, 70% yield) of a brown oil. ¹H-NMR (300 MHz, CDCl₃) δ=1.03 (t, J=7.2 Hz, 12H), 1.35-1.64 (m, 8H), 2.42 (t, J=7.2 Hz, 4H), 2.54 (q, J=7.2 Hz, 8H), 3.49-3.36 (s, 2H); ¹³C-NMR (75 MHz, CDCl₃) δ=11.6, 23.5, 35.6, 46.8, 50.1 53.1.

N-(7-Chloro-4-quinolyl)-1,7-bis(diethylamino)-4-aminoheptane. 4,7-Dichloroquinoline (0.6 g, 3.0 mmol) and 1,7-bis(diethylamino)-4-aminoheptane (0.06 g, 0.23 mmol) were mixed in a closed vessel and heated to 120° C. for 3 days. The crude product was treated with 4M NaOH and extracted with CHCl₃. The combined organic layers were extracted with brine, dried over anhydrous MgSO₄ and evaporated under reduced pressure. The crude product was purified by flash column chromatography using EtOH:CH₂Cl₂:Et₃N (100:50:5 v/v) as the mobile phase to give 0.04 g (0.09 mmol, 40% yield) of a yellow oil. ¹H-NMR (300 MHz, CDCl₃) δ=1.03 (t, J=7.2 Hz, 12H), 1.52-1.80 (m, 8H), 2.47 (t, J=7.2 Hz, 4H), 2.55 (q, J=7.2 Hz, 8H), 3.58-3.72 (m, 1H), 5.62 (bs, 1H), 6.44 (d, J=3.1 Hz, 1H), 7.36 (dd, J=1.8 Hz, 9.0 Hz, 1H), 7.76 (d, J=9.0 Hz, 1H), 7.96 (d, J=1.8 Hz, 1H), 8.51 (d, J=3.1 Hz, 1H); ¹³C-NMR (75 MHz, CDCl₃) δ=11.5, 23.8, 32.3, 47.0, 52.8, 52.9, 99.4, 117.6, 121.8, 125.1, 128.9, 135.0, 149.6, 149.8, 152.2; MS (ESI) m/z calcd for C₂₄H₃₉ClN₄ 418.3. Found: 419.2.

1,7-Bis(diisopropylamido)heptan-4-one. 4-Ketopimelic acid (4.0 g, 23.0 mmol), diethylamine (20.0 mL, 50.0 mmol), and 2-chloro-4,6-dimethoxy-1,3,5-triazine (8.0 g, 46.0 mmol) were dissolved in 70 mL of CH₃CN and N-methyl morpholine (12.0 g, 117.0 mmol) was added at once. After 24 h, 2M HCl was added and the solution was extracted with CH₂Cl₂. The combined organic layers were dried over anhydrous MgSO₄ and evaporated under reduced pressure to give 6.2 g (18.4 mmol, 80% yield) of a yellow oil. ¹H-NMR (300 MHz, CDCl₃) δ=1.20 (d, J=6.6 Hz, 12H), 1.34 (d, J=6.6 Hz, 12H), 2.59 (t, J=6.6 Hz, 4H), 2.82 (t, J=6.6 Hz, 4H), 3.21-3.42 (m, 4H); ¹³C-NMR (75 MHz, CDCl₃) δ=20.9, 21.0, 28.9, 37.7, 45.7, 48.3, 56.1, 170.4, 211.5.

1,7-Bis(diisopropylamido)-4-aminoheptane. 1,7-Bis(diisopropylamido)heptan-4-one (4.5 g, 13.3 mmol), ammonium acetate (13.0 g, 165.0 mmol) and sodium cyanoborohydride (5.0 g, 79.5 mmol) were dissolved in 100 mL of anhydrous MeOH and stirred at room temperature for 48 h. The solvents were removed in vacuo. The residue was dissolved in CH₂Cl₂ and extracted with 4M NaOH solution. The combined organic layers were concentrated and extracted with 6M HCl. The aqueous layer was basified and then extracted with CH₂Cl₂. The combined organic layers were dried over anhydrous MgSO₄ and evaporated under reduced pressure providing 3.1 g (9.3 mmol, 70% yield) of a yellow oil. ¹H-NMR (300 MHz, CDCl₃) δ=1.20 (d, J=6.9 Hz, 12H), 1.34 (d, J=6.6 Hz, 12H), 1.59-1.85 (m, 4H), 2.27-2.58 (m, 4H), 2.66-3.11 (m, 3H), 3.46 (bs, 2H), 3.82-4.12 (m, 2H); ¹³C-NMR (75 MHz, CDCl₃) δ=20.9, 21.1, 32.1, 33.0, 45.8, 49.1, 51.3, 171.9.

1,7-Bis(diisopropylamino)-4-aminoheptane. A solution of 1,7-Bis(diisopropylamido)-4-aminoheptane (0.15 g, 0.57 mmol) and lithium aluminum hydride in 2M THF (2.1 mL, 4.2 mmol) in 3 mL of anhydrous toluene was refluxed at 110° C. for 24 h. The reaction mixture was quenched with 4M NaOH and extracted with CH₂Cl₂. The combined organic layers were dried over anhydrous MgSO₄ and evaporated under reduced pressure to give 0.14 g (0.46 mmol, 80% yield) of a yellow oil. ¹H-NMR (300 MHz, CDCl₃) δ=1.01 (d, J=6.6 Hz, 24H), 1.15-1.52 (m, 8H), 2.32-2.48 (m, 4H), 2.68-2.82 (m, 1H), 2.92-3.11 (m, 4H); ¹³C-NMR (75 MHz, CDCl₃) δ=14.4, 20.8, 28.1, 45.6, 48.6, 51.5.

N-(7-Chloro-4-quinolyl)-1,7-bis(diisopropylamino)-4-aminoheptane. 4,7-Dichloroquinoline (0.6 g, 3.0 mmol) and 1,7-bis(diisopropylamino)-4-aminoheptane (0.06 g, 0.23 mmol) were mixed in a closed vessel and the mixture was heated to 120° C. for 3 days. The mixture was treated with 4M NaOH and extracted with CHCl₃. The combined organic layers were washed with brine, dried over anhydrous MgSO₄ and evaporated under reduced pressure. The crude product was purified by flash column chromatography using EtOH:CH₂Cl₂:Et₃N (100:75:5 v/v) as the mobile phase to give 0.03 g (0.07 mmol, 30%) of a yellow oil. ¹H-NMR (300 MHz, CDCl₃) δ=1.00 (d, J=6.6 Hz, 24H), 1.28-1.75 (m, 8H), 2.43 (t, J=6.6 Hz, 4H), 2.85-3.12 (m, 4H), 3.58-3.74 (m, 1H), 4.77 (d, J=8.2 Hz, 1H), 6.44 (d, J=6.1 Hz, 1H), 7.38 (dd, J=1.8 Hz, 9.0 Hz, 1H), 7.65 (d, J=9.0 Hz, 1H), 7.97 (d, J=1.8 Hz, 1H), 8.53 (d, J=6.1 Hz, 1H); ¹³C-NMR (75 MHz, CDCl₃) δ=13.9, 19.0, 20.2, 31.8, 36.7, 52.3, 98.8, 117.0, 124.9, 128.6, 134.6, 149.2, 151.8; MS (ESI) m/z calcd for C₂₈H₄₇ClN₄ 474.4. Found (M+H)⁺: 475.3.

1,9-Bis(diethylamido)nonan-5-one. To a mixture of 5-oxoazelaic acid (2.5 g, 12.4 mmol) and PyBop (15.4 g, 29.7 mmol) in anhydrous CH₃CN (18.0 mL) under inert atmosphere was added diethylamine (5.11 mL, 49.9 mmol) and N,N-diisopropylethylamine (6.0 mL, 34.2 mmol). The reaction proceeded with good stirring at 35° C. for 64 h and then solvents were removed in vacuo. The residue was dissolved in CH₂Cl₂, washed with a 2M HCl, dried over anhydrous MgSO₄, and concentrated in vacuo to produce a yellow oil (2.39 g, 7.7 mmol, 62% yield). ¹H-NMR (300 MHz, CDCl₃) δ=1.08 (t, J=7.1 Hz, 6H), 1.16 (t, J=7.2 Hz, 6H), 1.65-1.90 (m, 4H), 2.33 (t, J=7.5 Hz, 4H), 2.50 (t, J=7.1 Hz, 4H), 3.10-3.30 (m, 8H); ¹³C-NMR (75 MHz, CDCl₃) δ=14.9, 14.1, 19.3, 31.8, 40.0, 41.6, 41.9, 171.6, 210.5.

1,9-Bis(diethylamido)-5-aminononane. To a mixture of 1,9-bis(diethylamido)nonan-5-one (2.39 g, 7.7 mmol) in 24 mL of anhydrous MeOH under inert atmosphere was added ammonium acetate (15.4 g, 46.0 mmol) and sodium cyanoborohydride (1.2 g, 19.2 mmol). The reaction mixture was stirred at room temperature for 4 days. The solvents were removed under reduced pressure and the residue was dissolved in CH₂Cl₂ and extracted with 6M HCl. The aqueous layer was basified using a concentrated NaOH solution, extracted with CH₂Cl₂, dried over anhydrous MgSO₄, and concentrated in vacuo to give 1.39 g of a yellow oil (4.4 mmol, 58% yield). ¹H-NMR (300 MHz, CDCl₃) δ=0.97 (t, J=7.2 Hz, 6H), 1.04 (t, J=7.2 Hz, 6H), 1.27-1.64 (m, 8H), 2.21 (t, J=7.2 Hz, 4H), 2.70-2.81 (m, 1H), 3.20-3.51 (m, 8H), 3.83 (bs, 2H); ¹³C-NMR (75 MHz, CDCl₃) δ=13.1, 14.3, 21.8, 32.9, 37.4, 40.0, 41.9, 50.8, 171.8.

1,9-Bis(diethylamino)-5-aminononane. To a mixture of 1,9-bis(diethylamido)-5-aminononane (0.2 g, 0.64 mmol) in 1.5 mL of anhydrous toluene under inert atmosphere was added dropwise lithium aluminum hydride as a 2M THF solution (1.4 mL, 3.8 mmol). The reaction mixture was stirred for 24 h at 110° C. Then, 10 mL of a 4M NaOH was added and the mixture was extracted with CH₂Cl₂, dried over anhydrous MgSO₄, and concentrated in vacuo to give 0.15 g of a yellow oil (0.51 mmol, 80% yield). ¹H-NMR (300 MHz, CDCl₃) δ=0.95 (t, J=7.1 Hz, 12H), 1.17-1.48 (m, 12H), 2.31-2.33 (m, 2H), 2.35 (t, J=7.7 Hz, 4H), 2.45 (q, J=7.1 Hz, 8H), 2.59-2.62 (m, 1H); ¹³C-NMR (75 MHz, CDCl₃) δ=11.9, 24.5, 27.4, 38.36, 47.1, 51.3, 53.1.

N-(7-Chloro-4-quinolyl)-1,9-bis(diethylamino)-5-aminononane. A mixture of 4,7-dichloroquinoline (0.75 g, 3.8 mmol) and 1,9-bis(diethylamino)-5-aminononane (0.07 g, 0.25 mmol) was heated to 120° C. for 72 h in a closed vessel. Saturated NaHCO₃ solution was added to the cooled reaction mixture, which was then extracted with CH₂Cl₂, dried over anhydrous MgSO₄, and concentrated in vacuo. The crude product was purified by flash chromatography using EtOAc:EtOH:Et₃N (1:1:0.01 v/v) as the mobile phase to give a yellow oil (0.05 g, 0.11 mmol, 47% yield). ¹H-NMR (300 MHz, CDCl₃) δ=0.99 (t, J=7.2 Hz, 12H), 1.29-1.74 (m, 12H), 2.39 (t, J=7.4 Hz, 4H), 2.48 (q, J=7.2 Hz, 8H), 3.6 (m, 1H), 4.83 (d, J=8.1 Hz, 1H), 6.40 (d, J=5.4 Hz, 1H), 7.34 (dd, J=9.0 Hz, 2.4 Hz, 1H), 7.37 (d, J=9.0 Hz, 1H), 7.94 (d, J=2.4 Hz, 1H), 8.49 (d, J=5.4 Hz, 1H); ¹³C-NMR (75 MHz, CDCl₃) δ=11.8, 24.2, 27.3, 34.8, 47.1, 52.9, 53.0, 99.3, 121.1, 125.3, 129.2, 135.1, 149.6, 149.7, 152.3; MS (ESI) m/z calcd for C₂₆H₄₃ClN₄ 446.3. Found (M+H)⁺: 447.3.

1,9-Bis(diisopropylamido)nonan-5-one. To a mixture of 5-oxoazelaic acid (1.0 g, 5.0 mmol) and 2-chloro-4,6-dimethoxy-1,3,5-triazine (2.0 g, 11.4 mmol) in anhydrous CH₃CN (18.0 mL) under inert atmosphere was added N-methyl morpholine (2.5 g, 24.7 mmol) and diisopropylamine (1.0 g, 9.9 mmol). The mixture was stirred at room temperature for 48 h and then the solvents were removed in vacuo. The residue was dissolved in CH₂Cl₂, extracted with 2M HCl, dried over anhydrous MgSO₄, and concentrated in vacuo to give a yellow oil (1.5 g, 4.1 mmol, 82% yield). ¹H-NMR (300 MHz, CDCl₃) δ=1.17 (d, J=6.6 Hz, 12H), 1.34 (d, J=6.9 Hz, 12H), 1.85 (m, 4H), 2.30 (t, J=7.2 Hz, 4H), 2.48 (t, J=6.9 Hz, 4H), 3.37-3.59 (m, 2H), 3.86-3.95 (m, 2H); ¹³C-NMR (75 MHz, CDCl₃) δ=19.7, 20.9, 21.2, 34.5, 42.1, 45.9, 48.6, 171.7, 210.8.

1,9-Bis(diisopropylamido)-5-aminononane. To a mixture of 1,9-bis(diisopropylamido)nonan-5-one (0.73 g, 2.0 mmol) in 4 mL of anhydrous MeOH under inert atmosphere was added ammonium acetate (1.25 g, 16.2 mmol) and sodium cyanoborohydride (0.43 g, 6.8 mmol). The reaction mixture was stirred at room temperature for 3 days. Solvents were removed under reduced pressure and the residue was dissolved in CH₂Cl₂ and extracted with 6M HCl. The aqueous layer was basified using a concentrated NaOH solution, extracted with CH₂Cl₂, dried over anhydrous MgSO₄, and concentrated in vacuo to a yellow oil (0.37 g, 1.0 mmol, 50% yield). ¹H-NMR (300 MHz, CDCl₃) δ=1.17 (d, J=6.6 Hz, 12H), 1.34 (d, J=6.9 Hz, 12H), 1.40-1.77 (m, 6H), 2.30 (t, J=7.2 Hz, 4H), 2.35-2.50 (m, 2H), 2.75-2.82 (m, 1H), 3.40-3.59 (m, 2H), 3.90-4.00 (m, 2H); ¹³C-NMR (75 MHz, CDCl₃) δ=20.9, 21.4, 21.6, 34.9, 35.6, 42.6, 46.3, 51.8, 172.3.

1,9-Bis(diisopropylamino)-5-aminononane. To a mixture of 1,9-bis(diisopropylamido)-5-aminononane (0.18 g, 0.5 mmol) in 1.5 mL of anhydrous toluene under inert atmosphere was added dropwise lithium aluminum hydride as a 2M THF solution (1.4 mL, 3.8 mmol). The reaction mixture was stirred for 24 h at 110° C. Then, 10 mL of 4M NaOH was added and the mixture was extracted with CH₂Cl₂, dried over anhydrous MgSO₄, and concentrated in vacuo to a yellow oil (0.15 g, 0.42 mmol, 85% yield). ¹H-NMR (300 MHz, CDCl₃) δ=1.02 (d, J=6.6 Hz, 24H), 1.24-1.46 (m, 12H), 2.40 (t, J=7.4 Hz, 4H), 2.62-2.69 (m, 1H), 3.02 (sept, J=6.6 Hz, 4H); ¹³C-NMR (75 MHz, CDCl₃) δ=20.9, 24.2, 31.9, 38.3, 45.5, 48.7, 51.5.

N-(7-Chloro-4-quinolyl)-1,9-bis(diisopropylamino)-5-aminononane. A mixture of 4,7-dichloroquinoline (0.75 g, 3.8 mmol) and 1,9-bis(diisopropylamino)nonan-5-amine (0.07 g, 0.21 mmol) was heated to 120° C. for 72 h under nitrogen in a closed vessel. After cooling to room temperature, aqueous NaHCO₃ was added and the mixture was extracted with CH₂Cl₂, dried over anhydrous MgSO₄, and concentrated in vacuo. The crude product was purified by flash column chromatography using CH₂Cl₂:EtOH:Et₃N (2:1:0.04 v/v) as the mobile phase and a yellow oil (0.03 g, 0.06 mmol, 30% yield) was obtained. ¹H-NMR (300 MHz, CDCl₃) δ=0.96 (d, J=6.6 Hz, 24H), 1.25-1.70 (m, 12H), 2.35 (t, J=7.1 Hz, 4H), 2.97 (sep, J=6.6 Hz, 4H), 3.50-3.70 (m, 1H), 4.73 (d, J=8.6 Hz, 1H), 6.41 (d, J=5.5 Hz, 1H), 7.35 (dd, J=9.0, 2.0 Hz, 1H), 7.65 (d, J=9.0 Hz, 1H), 7.95 (d, J=2.0 Hz, 1H), 8.50 (d, J=5.5 Hz, 1H); MS (ESI) m/z calcd for C₃₀H₅₁ClN₄ 502.4. Found (M+H)⁺: 503.3.

N-(7-Chloro-4-quinolyl)-1,3-diaminocyclohexane. A mixture of 4,7-dichloroquinoline (0.28 g, 1.4 mmol) and 1,3-diaminocyclohexane (cis- and trans-mixture) (0.5 mL, 4.2 mmol) was heated to 110° C. for 18 h under inert atmosphere and then cooled to room temperature. Aqueous NaOH (1N, 10 mL) was then added and the mixture was extracted with CH₂Cl₂. The combined organic layers were washed with water, brine, dried over anhydrous Na₂SO₄, and evaporated under reduced pressure. Purification by flash chromatography using 0.5% Et₃N in MeOH as the mobile phase gave a brown oil (0.31 g, 1.1 mmol, 78% yield). ¹H-NMR (300 MHz, CDCl₃) δ=1.24-1.96 (m, 10H, cis+trans), 2.13 (d, J=7.8 Hz, 1H, cis+trans), 3.16 (m, 1H, cis+trans), 3.70 (s, 1H, cis or trans), 4.01 (s, 1H, cis or trans), 6.35 (d, J=3.3 Hz, 1H, cis or trans), 6.44 (d, J=3.0 Hz, 1H, cis or trans), 7.31 (m, 1H, cis+trans), 7.63 (d, J=5.1 Hz, 1H, cis or trans), 7.68 (d, J=5.4 Hz, 1H, cis or trans), 7.91 (s, 1H, cis+trans), 7.94 (s, 1H, cis+trans), 8.47 (d, J=3.0 Hz, 1H, cis or trans), 8.50 (d, J=3.3 Hz, 1H, cis or trans); ¹³C-NMR (75 MHz, CDCl₃) δ=19.1, 21.9, 29.2, 30.5, 33.0, 33.7, 37.5, 39.8, 45.5, 46.9, 47.5, 49.9, 98.3, 98.8, 116.9, 121.7, 122.0, 124.6, 124.8, 126.5, 126.7, 134.8, 148.0, 149.2, 149.4, 150.6, 150.7.

N-(7-Chloro-4-quinolyl)-1,4-diaminocyclohexane. A mixture of 4,7-dichloroquinoline (0.28 g, 1.4 mmol) and 1,4-diaminocyclohexane (cis- and trans-mixture) (0.5 mL, 4.2 mmol) was heated to 110° C. for 18 h under inert atmosphere and then cooled to room temperature. Aqueous NaOH (1N, 10 mL) was then added and the mixture was extracted with CH₂Cl₂. The combined organic layers were washed with water, brine, dried over anhydrous Na₂SO₄ and evaporated under reduced pressure. Purification by flash chromatography using 0.5% Et₃N in MeOH as the mobile phase gave a brown oil (0.32 g, 1.16 mmol, 82% yield). ¹H-NMR (300 MHz, CDCl₃) δ=1.22-1.62 (m, 2H, cis+trans), 1.72-2.12 (m, 8H, cis+trans), 2.78 (m, 1H, cis or trans), 3.02 (m, 1H, cis or trans), 3.71 (m, 1H, cis+trans), 4.95 (d, J=7.2 Hz, 1H, cis or trans), 5.14 (d, J=6.3 Hz, 1H, cis or trans), 6.41 (d, J=5.4 Hz, 1H, cis+trans), 7.34 (dd, J=2.1 Hz, J=9.0 Hz, 1H, cis+trans), 7.67 (d, J=9.0 Hz, 1H, cis+trans), 7.94 (d, J=2.1 Hz, 1H, cis+trans), 8.49 (d, J=5.4 Hz, 1H, cis+trans); ¹³C-NMR (75 MHz, CDCl₃) δ=27.4, 30.3, 31.2, 33.6, 49.6, 50.5, 51.8, 99.6, 100.0, 118.4, 118.5, 124.0, 124.1, 125.6, 125.7, 127.3, 127.4, 136.0, 136.1, 149.4, 151.1, 151.4, 151.9.

N-(7-Chloro-4-quinolyl)-N,N′-diethyl-1,3-diaminocyclohexane. To a solution of N-(7-chloro-4-quinolyl)-1,3-diaminocyclohexane (0.1 g, 0.36 mmol) in 4 mL of glacial acetic acid, sodium borohydride (0.53 g, 14.0 mmol) was added portionwise at 0° C. The reaction was heated at 60° C. for 24 h and then cooled to room temperature, basified (pH>10) with 12N NaOH and extracted with dichloromethane. The combined organic layers were dried over anhydrous Na₂SO₄ and concentrated in vacuo. Purification by flash chromatography using 0.5% Et₃N in MeOH as the mobile phase gave yellow crystals (0.09 g, 0.27 mmol, 73% yield). ¹H-NMR (300 MHz, CDCl₃) δ=1.03 (m, 6H, cis+trans), 1.30-2.20 (m, 8H, cis+trans), 2.61 (m, 4H, cis+trans), 2.75 (m, 1H, cis or trans), 2.88 (m, 1H, cis or trans), 3.61 (m, 1H, cis or trans), 4.03 (m, 1H, cis or trans), 5.13 (d, J=6.6 Hz, 1H, cis+trans), 6.38 (m, 1H, cis+trans), 7.29 (m, 1H, cis+trans), 7.68 (m, 1H, cis+trans), 7.91 (m, 1H, cis+trans), 8.47 (m, 1H, cis+trans); ¹³C-NMR (75 MHz, CDCl₃) δ=12.8, 12.9, 20.4, 21.3, 28.0, 28.2, 30.0, 31.4, 33.1, 34.3, 43.1, 47.9, 50.4, 54.4, 56.9, 98.8, 99.3, 117.1, 117.2, 120.6, 121.3, 124.7, 125.0, 128.4, 128.6, 134.5, 134.6, 148.4, 148.7, 149.0, 149.1, 151.7, 151.8; MS (ESI) m/z calcd for C₁₉H₂₆ClN₃ 331.2. Found (M+H)⁺: 332.2.

N-(7-Chloro-4-quinolyl)-N,N′-diethyl-1,4-diaminocyclohexane. To a solution of N-(7-chloro-4-quinolyl)-1,4-diaminocyclohexane (0.11 g, 0.38 mmol) in 4 mL of glacial acetic acid, sodium borohydride (0.43 g, 11.4 mmol) was added portionwise at 0° C. The reaction was heated at 60° C. for 24 h, cooled to room temperature, basified (pH>10) with 12N NaOH and extracted with dichloromethane. The combined organic layers were dried over anhydrous Na₂SO₄ and concentrated in vacuo. Purification by flash chromatography using 0.5% Et₃N in MeOH as the mobile phase gave yellow crystals (0.07 g, 0.2 mmol, 53% yield). ¹H-NMR (300 MHz, CDCl₃) δ=1.02 (t, J=7.2 Hz, 6H, cis+trans), 1.20-2.12 (m, 8H, cis+trans), 2.50-2.71 (m, 5H, cis+trans), 3.40 (m, 1H, cis or trans), 3.78 (m, 1H, cis or trans), 4.98 (d, J=7.2 Hz, 1H, cis or trans), 5.11 (d, J=6.6 Hz, 1H, cis or trans), 6.40 (d, J=5.4 Hz, 1H, cis+trans), 7.28 (m, 1H, cis or trans), 7.33 (dd, J=2.1 Hz, J=9.0 Hz, 1H, cis or trans), 7.64 (m, 1H, cis+trans), 7.93 (m, 1H, cis+trans), 8.49 (d, J=5.4 Hz, 1H, cis+trans); ¹³C-NMR (75 MHz, CDCl₃) δ=12.4, 13.7, 24.6, 27.3, 28.2, 31.9, 42.8, 43.5, 47.3, 51.5, 57.3, 58.7, 99.2, 117.0, 117.1, 120.7, 124.9, 128.5, 134.5, 148.3, 148.6, 149.0, 151.7; MS (ESI) m/z calcd for C₁₉H₂₆ClN₃ 331.2. Found (M+H)⁺: 331.9.

N-(7-Chloro-4-quinolyl)-N′-isopropyl-1,3-diaminocyclohexane. To a solution of N-(7-chloro-4-quinolyl)-1,3-diaminocyclohexane (0.1 g, 0.36 mmol), acetone (0.13 mL, 1.8 mmol) and glacial acetic acid (0.04 mL, 0.72 mmol) in anhydrous CH₂Cl₂, sodium triacetoxyborohydride (0.23 g, 1.1 mmol) was added at room temperature and stirred for 2 h. The reaction mixture was quenched with water, basified with 1N NaOH and extracted with dichloromethane. The combined organic layers were dried over anhydrous Na₂SO₄ and concentrated in vacuo. Purification by flash chromatography using 0.5% Et₃N in MeOH as the mobile phase gave pale yellow crystals (0.09 g, 0.29 mmol, 81% yield). ¹H-NMR (300 MHz, CDCl₃) δ=1.04 (m, 2H, cis+trans), 1.13 (m, 4H, cis+trans), 1.31-2.12 (m, 10H, cis+trans), 2.86-3.10 (m, 2H, cis+trans), 3.75 (m, 1H, cis or trans), 3.96 (m, 1H, cis or trans), 6.32 (d, J=5.4 Hz, 1H, cis or trans), 6.44 (d, J=5.4 Hz, 1H, cis or trans), 7.29 (m, 1H, cis+trans), 7.65 (d, J=9.0 Hz, 1H, cis or trans), 7.74 (d, J=9.0 Hz, 1H, cis or trans), 7.91 (d, J=2.4 Hz, 1H, cis or trans), 7.93 (d, J=2.4 Hz, 1H, cis or trans), 8.45 (d, J=5.4 Hz, 1H, cis or trans), 8.49 (d, J=5.4 Hz, 1H, cis or trans); ¹³C-NMR (75 MHz, CDCl₃) δ=19.0, 19.6, 23.1, 23.2, 23.3, 29.8, 30.8, 31.4, 31.9, 36.5, 36.9, 45.0, 45.6, 47.2, 48.8, 49.0, 50.7, 98.3, 99.1, 117.0, 117.3, 120.8, 121.8, 124.4, 124.8, 128.1, 128.4, 134.4, 134.5, 148.4, 148.9, 149.0, 149.1, 151.6; MS (ESI) m/z calcd for C₁₈H₂₄ClN₃ 317.2. Found (M+H)⁺: 318.2.

N-(7-Chloro-4-quinolyl)-N′-isopropyl-1,4-diaminocyclohexane. To a solution of N-(7-chloro-4-quinolyl)-1,4-diaminocyclohexane (0.1 g, 0.36 mmol), acetone (0.13 mL, 1.8 mmol) and glacial acetic acid (0.04 mL, 0.72 mmol) in anhydrous CH₂Cl₂, sodium triacetoxyborohydride (0.23 g, 1.1 mmol) was added at room temperature and stirred for 2 h. The reaction mixture was quenched with water, basified with 1N NaOH and extracted with dichloromethane. The combined organic layers were dried over anhydrous Na₂SO₄ and concentrated in vacuo. Purification by flash chromatography using 100% MeOH as the mobile phase gave colorless crystals (0.07 g, 0.22 mmol, 64% yield). ¹H-NMR (300 MHz, CDCl₃) δ=1.05 (m, 6H, cis+trans), 1.43-1.60 (m, 2H, cis+trans), 1.67-1.96 (m, 6H, cis+trans), 2.80 (m, 1H, cis+trans), 2.90 (m, 1H, cis+trans), 3.70 (s, 1H, cis+trans), 5.14 (d, J=6.6 Hz, 1H, cis+trans), 6.38 (m, 1H, cis+trans), 7.30 (m, 1H, cis+trans), 7.64 (dd, J=2.4 Hz, J=9.0 Hz, 1H, cis+trans), 7.92 (m, 1H, cis+trans), 8.48 (m, 1H, cis+trans); ¹³C-NMR (75 MHz, CDCl₃) δ=23.3, 27.5, 28.7, 44.9, 48.1, 50.8, 99.1, 117.0, 120.7, 124.9, 128.6, 134.6, 148.3, 149.1, 151.7; MS (ESI) m/z calcd for C₁₈H₂₄ClN₃ 317.2. Found (M+H)⁺: 318.1.

2-(5-Dimethylaminonaphthalene-1-sulfonamido)ethyl methanesulfonate, 22. To a solution of 5-dimethylamino-N-(2-hydroxyethyl)naphthalene-1-sulfonamide, 21 (1.5 g, 5.1 mmol) and Et₃N (1.07 mL, 7.64 mmol) in anhydrous CH₂Cl₂, methanesulfonyl chloride (0.44 mL, 5.61 mmol) was added at room temperature and stirred for 1 hour. After addition of water, the reaction mixture was extracted with CH₂Cl₂, dried over anhydrous Na₂SO₄ and concentrated in vacuo. Flash chromatography using EtOAc:hexanes (1:4 v/v) as mobile phase and gradually changing the ratio of EtOAc:hexanes to 1:1.7 (v/v) afforded 1.71 g (4.6 mmol, 90% yield) of a light yellow oil. ¹H-NMR (300 MHz, CDCl₃) δ=2.86 (s, 3H), 2.89 (s, 6H), 3.27 (m, 2H), 4.17 (t, J=6.0 Hz, 2H), 5.11 (t, J=6.0 Hz, 1H), 7.21 (d, J=7.5 Hz, 1H), 7.50-7.64 (m, 2H), 8.22-8.28 (m, 2H), 8.57 (d, J=5.7 Hz, 1H); ¹³C-NMR (75 MHz, CDCl₃) δ=37.0, 42.1, 45.2, 68.1, 115.2, 118.5, 123.1, 128.5, 129.2, 129.4, 129.7, 130.6, 134.3, 151.9.

Representative procedure for the synthesis of sulfonamide analogs 23-28. A solution of N-(7-chloro-4-quinolyl)-N′-ethyl-1,4-diaminobutane (0.076 g, 0.27 mmol) and 22 (0.05 g, 0.14 mmol) in anhydrous DMF was heated at 90° C. for 3 hours. After cooling to room temperature, DMF was removed in vacuo. Saturated NaHCO₃ solution was added to the residue, which was then extracted with CH₂Cl₂, dried over anhydrous Na₂SO₄ and concentrated in vacuo. Flash chromatography using MeOH:CH₂Cl₂ (1:19 v/v) as the mobile phase gave 0.056 g (0.1 mmol, 75% yield) of 25 as a yellow oil. For the syntheses of 23, 24 and 27, the reactions were carried out in anhydrous THF and refluxed for 60 hours.

N-[2-{(N′-4-(7-chloro-4-quinolyl)aminobutyl-N″-ethyl}aminoethyl]-5-dimethylaminonaphthalene-1-sulfonamide, 25. ¹H-NMR (300 MHz, CDCl₃) δ=0.79 (t, J=7.2 Hz, 3H), 1.44 (m, 2H), 1.64 (m, 2H), 2.22-2.34 (m, 4H), 2.43 (t, J=6.0 Hz, 2H), 2.85 (s, 6H), 2.91 (t, J=6.0 Hz, 2H), 3.24 (q, J=6.0 Hz, 2H), 5.32 (bs, 1H), 6.37 (d, J=5.4 Hz, 1H), 7.13 (dd, J=0.9 Hz, J=7.5 Hz, 1H), 7.32 (dd, J=2.1 Hz, J=8.7 Hz, 1H), 7.46-7.56 (m, 2H), 7.76 (d, J=9.3 Hz, 1H), 7.94 (d, J=2.4 Hz, 1H), 8.25 (dd, J=1.2 Hz, J=7.5 Hz, 1H), 8.32 (d, J=8.4 Hz, 1H), 8.50-8.57 (m, 2H); ¹³C-NMR (75 MHz, CDCl₃) δ=11.1, 24.6, 26.4, 40.4, 42.9, 45.3, 46.7, 51.5, 52.1, 98.8, 115.0, 117.1, 118.7, 121.5, 123.0, 125.1, 128.2, 129.5, 129.6, 129.7, 130.3, 134.3, 134.8, 148.7, 149.9, 151.6, 151.9.

N-[2-{(N′-2-(7-chloro-4-quinolyl)aminoethyl-N″-ethyl}aminoethyl]-5-dimethylaminonaphthalene-1-sulfonamide, 23. Employing 0.16 g (0.44 mmol) of 22, 0.15 g (0.6 mmol) of N-(7-chloro-4-quinolyl)-N′-ethyl-1,2-diaminoethane and 0.3 mL (1.7 mmol) of N,N-diisopropylethylamine in the procedure described above and purification by flash chromatography using MeOH:CH₂Cl₂ (1:19 v/v) as the mobile phase gave 0.18 g (0.33 mmol, 79% yield) of a yellow oil. ¹H-NMR (300 MHz, CDCl₃) δ=0.87 (t, J=7.2 Hz, 3H), 2.42 (q, J=7.2 Hz, 2H), 2.55 (t, J=6.0 Hz, 2H), 2.68 (t, J=6.0 Hz, 2H), 2.84 (s, 6H), 2.99 (t, J=5.7 Hz, 2H), 3.19 (q, J=5.7 Hz, 2H), 5.61 (bs, 2H), 6.25 (d, J=5.4 Hz, 1H), 7.10 (d, J=7.5 Hz, 1H), 7.28-7.50 (m, 3H), 7.73 (d, J=9.0 Hz, 1H), 7.92 (d, J=2.1 Hz, 1H), 8.20 (dd, J=1.2 Hz, J=7.2 Hz, 1H), 8.25 (d, J=8.4 Hz, 1H), 8.45-8.56 (m, 2H); ¹³C-NMR (75 MHz, CDCl₃) δ=10.9, 39.9, 40.8, 45.1, 46.6, 50.8, 52.0, 98.7, 114.9, 117.0, 118.4, 121.6, 122.9, 125.0, 127.7, 128.1, 129.1, 129.3, 129.6, 130.2, 134.5, 134.6, 148.4, 149.6, 151.5, 151.7.

N-[2-{(N′-3-(7-chloro-4-quinolyl)aminopropyl-N″-ethyl}aminoethyl]-5-dimethylaminonaphthalene-1-sulfonamide, 24. Employing 0.16 g (0.44 mmol) of 22, 0.15 g (0.6 mmol) of N-(7-chloro-4-quinolyl)-N′-ethyl-1,3-diaminopropane and 0.3 mL (1.7 mmol) of N,N-diisopropylethylamine in the procedure described above and purification by flash chromatography using MeOH:CH₂Cl₂ (1:19 v/v) as the mobile phase furnished 0.133 g (0.25 mmol, 57% yield) of a yellow oil. ¹H-NMR (300 MHz, CDCl₃) δ=0.88 (t, J=7.2 Hz, 3H), 1.74 (m, 2H), 2.35-2.49 (m, 4H), 2.52 (t, J=6.0 Hz, 2H), 2.84 (s, 6H), 2.97 (t, J=6.0 Hz, 2H), 3.24 (q, J=6.0 Hz, 2H), 5.48 (bs, 1H), 6.03 (bs, 1H), 6.30 (d, J=5.4 Hz, 1H), 7.10 (d, J=7.5 Hz, 1H), 7.28 (m, 1H), 7.40-7.52 (m, 2H), 7.61 (d, J=8.7 Hz, 1H), 7.93 (t, J=1.8 Hz, 1H), 8.21 (dd, J=1.2 Hz, J=7.5 Hz, 1H), 8.25 (d, J=8.7 Hz, 1H), 8.47-8.55 (m, 2H); ¹³C-NMR (75 MHz, CDCl₃) δ=11.0, 25.1, 40.6, 42.2, 45.2, 47.1, 51.5, 52.2, 98.5, 115.0, 177.1, 118.5, 121.6, 122.9, 124.9, 128.0, 128.2, 129.3, 129.4, 129.6, 130.3, 134.3, 134.5, 148.7, 150.0, 151.6, 151.8.

N-[2-{(N′-4-(7-chloro-4-quinolyl)aminopentyl-N″-ethyl}aminoethyl]-5-dimethylaminonaphthalene-1-sulfonamide, 26. Employing 0.063 g (0.17 mmol) of 22 and 0.1 g (0.34 mmol) of monodesethylchloroquine (Ansari, A. M.; Craig, J. C. Synthesis 1995, 147-149) in the procedure described above and purification by flash chromatography using MeOH:CH₂Cl₂ (1:19 v/v) as the mobile phase afforded 0.058 g (0.10 mmol, 60% yield) of a yellow oil. ¹H-NMR (300 MHz, CDCl₃) δ=0.76 (t, J=7.2 Hz, 3H), 1.31 (d, J=6.3 Hz, 3H), 1.36-1.72 (m, 4H), 2.16-2.33 (m, 4H), 2.40 (m, 2H), 2.81-2.96 (m, 8H), 3.66 (m, 1H), 4.96 (d, J=7.8 Hz, 1H), 5.51 (bs, 1H), 6.38 (d, J=5.4 Hz, 1H), 7.13 (d, J=7.5 Hz, 1H), 7.34 (m, 1H), 7.46-7.57 (m, 2H), 7.74 (d, J=9.3 Hz, 1H), 7.95 (d, J=1.8 Hz, 1H), 8.25 (m, 1H), 8.31 (d, J=9.0 Hz, 1H), 8.48-8.58 (m, 2H); ¹³C-NMR (75 MHz, CDCl₃) δ=11.2, 20.2, 23.6, 34.3, 40.3, 45.3, 46.6, 48.2, 51.5, 52.5, 99.0, 115.0, 117.1, 118.7, 121.3, 123.0, 125.0, 128.2, 128.4, 129.5, 129.6, 129.8, 130.5, 134.3, 134.7, 149.0, 149.1, 151.7, 151.9.

N-[2-{(N′-5-(7-chloro-4-quinolyl)aminopentyl-N″-ethyl}aminoethyl]-5-dimethylaminonaphthalene-1-sulfonamide, 27. Employing 0.064 g (0.17 mmol) of 22 and 0.1 g (0.34 mmol) of N-(7-chloro-4-quinolyl)-N′-ethyl-1,5-diaminopentane in the procedure described above and purification by flash chromatography using MeOH:CH₂Cl₂ (1:19 v/v) as the mobile phase afforded 0.073 g (0.13 mmol, 75% yield) of a yellow oil. ¹H-NMR (300 MHz, CDCl₃) δ=0.76 (t, J=7.2 Hz, 3H), 1.29-1.39 (m, 4H), 1.67 (m, 2H), 2.16-2.28 (m, 4H), 2.39 (t, J=6.0 Hz, 2H), 2.82-2.93 (m, 8H), 3.28 (q, J=6.0 Hz, 2H), 5.36 (t, J=4.8 Hz, 1H), 6.37 (d, J=5.7 Hz, 1H), 7.14 (d, J=7.2 Hz, 1H), 7.26 (dd, J=2.4 Hz, J=9.0 Hz, 1H), 7.47-7.57 (m, 2H), 7.77 (d, J=9.0 Hz, 1H), 7.93 (d, J=2.4 Hz, 1H), 8.25 (dd, J=1.5 Hz, J=7.2 Hz, 1H), 8.32 (d, J=8.4 Hz, 1H), 8.48-8.58 (m, 2H); ¹³C-NMR (75 MHz, CDCl₃) δ=11.1, 24.9, 26.6, 28.4, 40.3, 43.1, 45.3, 46.6, 51.4, 52.3, 98.8, 115.0, 117.1, 118.7, 121.5, 123.0, 125.0, 128.1, 128.2, 129.5, 129.5, 129.7, 130.2, 134.2, 134.7, 148.8, 149.9, 151.6, 151.8.

N-[2-{(N′-6-(7-chloro-4-quinolyl)aminohexyl-N″-ethyl}aminoethyl]-5-dimethylaminonaphthalene-1-sulfonamide, 28. Employing 0.091 g (0.25 mmol) of 22 and 0.15 g (0.49 mmol) of N-(7-chloro-4-quinolyl)-N′-ethyl-1,6-diaminohexane in the procedure described above and purification by flash chromatography using MeOH:CH₂Cl₂ (1:19 v/v) as the mobile phase provided 0.108 g (0.19 mmol, 76% yield) of a yellow oil. ¹H-NMR (300 MHz, CDCl₃) δ=0.74 (t, J=7.2 Hz, 3H), 1.20-1.50 (m, 6H), 1.76 (m, 2H), 2.14-2.28 (m, 4H), 2.39 (t, J=6.0 Hz, 2H), 2.81-2.94 (m, 8H), 3.31 (q, J=7.2 Hz, 2H), 5.24 (bs, 1H), 6.40 (d, J=5.4 Hz, 1H), 7.14 (d, J=7.5 Hz, 1H), 7.28 (dd, J=2.1 Hz, J=9.0 Hz, 1H), 7.48-7.58 (m, 2H), 7.74 (d, J=9.0 Hz, 1H), 7.93 (d, J=2.1 Hz, 1H), 8.25 (dd, J=1.2 Hz, J=7.2 Hz, 1H), 8.32 (d, J=8.7 Hz, 1H), 8.49-8.58 (m, 2H); ¹³C-NMR (75 MHz, CDCl₃) δ=11.1, 26.6, 26.8, 26.9, 28.5, 40.3, 43.0, 45.2, 46.5, 51.4, 52.3, 98.7, 115.0, 117.1, 118.6, 121.5, 123.0, 124.9, 128.1, 129.5, 129.7, 130.2, 134.2, 134.6, 148.7, 150.0, 151.6, 151.8.

N-t-Boc N′-ethyl-N′-[2-(7-chloro-4-quinolyl)aminoethyl]-1,2-diaminoethane, 29. To a solution of N-(7-chloro-4-quinolyl)-N′-ethyl-1,2-diaminoethane (0.95 g, 3.8 mmol) and N-t-Boc-glycinal (1.1 g, 6.9 mmol; Myers, M. C.; Pokorski, J. K.; Appella D. H. Org. Lett. 2004, 6, 4699-4702) in anhydrous CH₂Cl₂, sodium triacetoxyborohydride (1.46 g, 6.9 mmol) was added at room temperature and stirred for 24 hours. The reaction mixture was quenched with water, basified with 10N NaOH and extracted with dichloromethane. The combined organic layers were dried over anhydrous Na₂SO₄ and concentrated in vacuo. Flash chromatography using MeOH:CH₂Cl₂ (1:24 v/v) as the mobile phase afforded 0.8 g (2.0 mmol, 54% yield) of a yellow oil. ¹H-NMR (300 MHz, CDCl₃) δ=1.05 (t, J=7.2 Hz, 3H), 1.34 (s, 9H), 2.56-2.70 (m, 4H), 2.81 (t, J=6.0 Hz, 2H), 3.15-3.36 (m, 4H), 5.25 (bs, 1H), 6.08 (s, 1H), 6.31 (d, J=5.4 Hz, 1H), 7.30 (dd, J=2.4 Hz, J=9.0 Hz, 1H), 7.73 (d, J=9.0 Hz, 1H), 7.92 (d, J=2.4 Hz, 1H), 8.47 (d, J=5.4 Hz, 1H); ¹³C-NMR (75 MHz, CDCl₃) δ=11.6, 28.1, 38.5, 39.8, 47.2, 51.2, 52.6, 78.9, 98.9, 117.1, 121.3, 125.0, 128.0, 134.5, 148.6, 149.7, 151.6, 155.9.

N-Ethyl-N-[2-(7-chloro-4-quinolyl)aminoethyl]-1,2-diaminoethane, 30. To a solution of 29 (0.25 g, 0.62 mmol) in anhydrous methanol, 2M HCl (3.1 mL, 6.2 mmol) was added at room temperature and stirred overnight. The solvents were removed in vacuo. The reaction mixture was basified with 10N NaOH, extracted with dichloromethane, dried over anhydrous Na₂SO₄ and concentrated in vacuo to 0.17 g (0.57 mmol, 88% yield) of a brown oil. ¹H-NMR (300 MHz, CD₃OD) δ=1.43 (t, J=7.2 Hz, 3H), 3.40-3.58 (m, 4H), 3.60-3.78 (m, 4H), 4.16 (m, 2H), 7.13 (d, J=6.9 Hz, 1H), 7.73 (dd, J=1.8 Hz, J=9.0 Hz, 1H), 7.92 (d, J=1.8 Hz, 1H), 8.53 (d, J=6.9 Hz, 1H), 8.64 (d, J=9.0 Hz, 1H); ¹³C-NMR (75 MHz, CDCl₃) δ=11.5, 39.6, 40.1, 47.2, 50.9, 55.5, 98.8, 117.2, 121.5, 124.7, 128.1, 134.3, 148.8, 149.8, 151.7.

Representative Procedure for the Synthesis of Sulfonamide Analogs 31-34. To a solution of N-(7-chloro-4-quinolyl)-N′-ethyl-N′-(2-aminoethyl)-1,2-diaminoethane 30 (0.055 g, 0.21 mmol) and Et₃N (0.06 mL, 0.42 mmol) in anhydrous CH₂Cl₂, 6-phenoxypyridine-3-sulfonyl chloride (0.06 g, 0.21 mmol) was added and the mixture was stirred at room temperature for 1 hour. Saturated NaHCO₃ solution was added to the reaction mixture, which was then extracted with CH₂Cl₂, dried over anhydrous Na₂SO₄, and concentrated in vacuo. Flash chromatography using MeOH:CH₂Cl₂ (1:49 v/v) and gradually changing the ratio of MeOH:CH₂Cl₂ to 1:19 (v/v) gave 0.065 g (0.12 mmol, 61% yield) of a yellow oil.

N-[2-{(N′-2-(7-chloro-4-quinolyl)aminoethyl-N″-ethyl}aminoethyl]-2-phenoxy-5-pyridinesulfonamide, 31. ¹H-NMR (300 MHz, CDCl₃) δ=1.00 (t, J=7.2 Hz, 3H), 2.60 (q, J=7.2 Hz, 2H), 2.69 (t, J=6.0 Hz, 2H), 2.79 (t, J=6.0 Hz, 2H), 3.10 (t, J=6.0 Hz, 2H), 3.18 (q, J=6.0 Hz, 2H), 5.92 (bt, 1H), 6.09 (d, J=5.4 Hz, 1H), 6.89 (d, J=8.7 Hz, 1H), 7.09-7.16 (m, 1H), 7.21-7.30 (m, 2H), 7.37-7.46 (m, 2H), 7.64 (d, J=9.0 Hz, 1H), 7.74 (d, J=2.1 Hz, 1H), 8.02 (dd, J=2.7 Hz, J=8.7 Hz, 1H), 8.34 (d, J=5.4 Hz, 1H), 8.63 (d, J=2.4 Hz, 1H); ¹³C-NMR (75 MHz, CDCl₃) δ=11.1, 39.8, 40.9, 46.8, 50.9, 52.0, 98.6, 111.3, 116.8, 121.3, 121.7, 125.3, 125.5, 127.3, 129.7, 131.1, 134.8, 138.1, 147.3, 147.9, 149.6, 151.3, 152.8, 165.8.

N-[2-{(N′-2-(7-chloro-4-quinolyl)aminoethyl-N″-ethyl}aminoethyl]-3-pyridinesulfonamide, 32. Employing 0.045 g (0.26 mmol) of 3-pyridinesulfonyl chloride and 0.075 g (0.26 mmol) of 30 in the procedure described above followed by flash chromatography using MeOH:CH₂Cl₂ (1:19 v/v) and gradually changing the ratio of MeOH:CH₂Cl₂ to 1:9 (v/v) afforded 0.06 g (0.13 mmol, 53% yield) of a yellow oil. ¹H-NMR (300 MHz, CDCl₃) δ=1.02 (t, J=7.2 Hz, 3H), 2.61 (q, J=7.2 Hz, 2H), 2.70 (t, J=6.0 Hz, 2H), 2.80 (t, J=6.0 Hz, 2H), 3.13 (t, J=6.0 Hz, 2H), 3.19 (q, J=6.0 Hz, 2H), 5.86 (bt, 1H), 6.11 (d, J=5.4 Hz, 1H), 7.28 (dd, J=2.1 Hz, J=9.0 Hz, 1H), 7.35 (m, 1H), 7.64 (d, J=9.0 Hz, 1H), 7.75 (d, J=2.1 Hz, 1H), 8.05 (m, 1H), 8.36 (d, J=5.4 Hz, 1H), 8.74 (dd, J=1.8 Hz, J=5.1 Hz, 1H), 9.06 (dd, J=0.9 Hz, J=2.4 Hz, 1H); ¹³C-NMR (75 MHz, CDCl₃) δ=11.1, 39.8, 40.9, 46.9, 50.9, 52.0, 98.8, 116.9, 121.6, 123.6, 125.3, 127.5, 134.4, 134.8, 136.8, 147.7, 148.1, 149.5, 151.5, 152.9.

N-[2-{(N′-2-(7-chloro-4-quinolyl)aminoethyl-N″-ethyl}aminoethyl]-8-quinolinesulfonamide, 33. Employing 0.04 g (0.17 mmol) of quinoline-8-sulfonyl chloride and 0.05 g (0.17 mmol) of 30 in the procedure described above followed by flash chromatography using MeOH:CH₂Cl₂ (1:49 v/v) and gradually changing the ratio of MeOH:CH₂Cl₂ to 1:24 (v/v) generated 0.056 g (0.12 mmol, 67% yield) of yellow crystals. ¹H-NMR (300 MHz, CDCl₃) δ=0.88 (t, J=7.2 Hz, 3H), 2.42 (q, J=7.2 Hz, 2H), 2.62 (t, J=6.0 Hz, 2H), 2.75 (t, J=6.0 Hz, 2H), 2.98 (q, J=6.0 Hz, 2H), 3.22 (q, J=6.0 Hz, 2H), 5.98 (t, J=4.2 Hz, 1H), 6.28 (d, J=5.4 Hz, 1H), 6.72 (t, J=5.4 Hz, 1H), 7.29 (dd, J=2.1 Hz, J=8.7 Hz, 1H), 7.41 (dd, J=5.2 Hz, J=8.1 Hz, 1H), 7.59 (dd, J=7.2 Hz, J=8.1 Hz, 1H), 7.87 (d, J=8.7 Hz, 1H), 7.90 (d, J=2.1 Hz, 1H), 7.99 (dd, J=1.5 Hz, J=8.1 Hz, 1H), 8.18 (dd, J=1.5 Hz, J=8.1 Hz, 1H), 8.39 (dd, J=1.5 Hz, J=7.2 Hz, 1H), 8.48 (d, J=5.4 Hz, 1H), 8.77 (dd, J=1.5 Hz, J=4.5 Hz, 1H); ¹³C-NMR (75 MHz, CDCl₃) δ=10.9, 39.9, 41.2, 46.3, 50.9, 52.1, 61.5, 98.8, 98.9117.1, 121.8, 122.0, 122.1, 125.4, 125.4, 125.5, 128.0, 128.5, 131.1, 133.3, 134.8, 135.2, 136.9, 142.9, 148.6, 149.7, 150.9, 151.5, 151.6.

N-[2-{(N′-2-(7-chloro-4-quinolyl)aminoethyl-N″-ethyl}aminoethyl]-4-methyl-3,4-dihydro-2H-benzo[b][1,4]oxazine-7-sulfonamide, 34. Employing 0.045 g (0.18 mmol) of 4-methyl-3,4-dihydro-2H-benzo[b][1,4]oxazine-7-sulfonyl chloride and 0.053 g (0.18 mmol) of 30 in the procedure described above followed by flash chromatography using MeOH:CH₂Cl₂ (1:16 v/v) as the mobile phase afforded 0.062 g (0.12 mmol, 68% yield) of a yellow oil. ¹H-NMR (300 MHz, CDCl₃) δ=0.97 (t, J=7.2 Hz, 3H), 2.54 (q, J=7.2 Hz, 2H), 2.64 (t, J=6.0 Hz, 2H), 2.72-2.81 (m, 5H), 3.04 (t, J=6.0 Hz, 2H), 3.16-3.30 (m, 4H), 4.27 (t, J=4.5 Hz, 2H), 5.80 (bs, 1H), 5.91 (bt, 1H), 6.22 (d, J=5.4 Hz, 1H), 6.73 (d, J=8.4 Hz, 1H), 7.06 (d, J=2.1 Hz, 1H), 7.10 (dd, J=2.1 Hz, J=8.4 Hz, 1H), 7.33 (dd, J=2.1 Hz, J=9.0 Hz, 1H), 7.76 (d, J=9.0 Hz, 1H), 7.86 (d, J=2.1 Hz, 1H), 8.44 (d, J=5.4 Hz, 1H); ¹³C-NMR (75 MHz, CDCl₃) δ=11.2, 38.4, 40.1, 40.9, 46.9, 48.1, 51.0, 52.1, 64.8, 98.7, 110.3, 115.7, 117.0, 117.2, 121.8, 125.5, 127.6, 131.6, 135.0, 136.7, 147.5, 148.1, 149.8, 151.2.

Representative Procedure for the Synthesis of Sulfonamide Analogs 35-38. To a mixture of N-(7-chloro-4-quinolyl)-1,3-diaminopropane (0.15 g, 0.64 mmol) in 4.5 mL of anhydrous THF under nitrogen at room temperature was added triethylamine (0.084 g, 0.83 mmol) and dansyl chloride (0.21 g, 0.76 mmol). After stirring for 36 hours at room temperature, the mixture was quenched with water and extracted with dichloromethane. The combined organic layers were dried over anhydrous MgSO₄, concentrated in vacuo, and purified by recrystallization from chloroform to give 35 as a white solid (0.06 g, 0.13 mmol, 20% yield).

N—(N′-3-(7-chloro-4-quinolyl)aminopropyl)-5-dimethylaminonaphthalene-1-sulfonamide, 35. ¹H-NMR (300 MHz, DMSO-d₆) δ=1.69 (tt, J=6.6 Hz, J=6.6 Hz, 2H), 2.80 (s, 6H), 2.94 (dt, J=6.6 Hz, J=6.1 Hz, 2H), 3.10 (dt, J=6.6 Hz, J=5.8 Hz, 2H), 6.16 (d, J=7.3 Hz, 1H), 7.15 (t, J=5.8 Hz, 1H), 7.23 (d, J=7.9 Hz, 1H), 7.41 (dd, J=2.2 Hz, J=8.5 Hz, 1H), 7.54 (d, J=7.9 Hz, 1H), 7.59 (d, J=8.5 Hz, 1H), 7.76 (d, J=2.2 Hz, 1H), 7.99 (t, J=6.1 Hz, 1H), 8.09 (dd, J=1.0 Hz, J=7.3 Hz, 1H), 8.15 (d, J=7.3 Hz, 1H), 8.30 (m, 2H), 8.41 (d, J=7.3 Hz, 1H); ¹³C-NMR (75 MHz, DMSO-d₆) δ=27.8, 45.0, 98.4, 115.0, 117.3, 118.9, 123.43, 123.9, 127.3, 127.8, 128.3, 129.0, 129.3, 133.3, 135.8, 148.8, 149.80, 151.3, 151.6

N—(N′-3-(7-chloro-4-quinolyl)aminopropyl)-8-quinolinesulfonamide, 36. Employing 0.19 g (0.82 mmol) of N-(7-chloro-4-quinolyl)-1,3-diaminopropane and 8-quinolinesulfonyl chloride (0.22 g, 0.98 mmol) in the procedure described above gave 0.14 g (0.33 mmol, 40% yield) of white crystals. ¹H-NMR (300 MHz, DMSO-d₆) δ=1.71 (tt, J=6.5 Hz, J=6.5 Hz, 2H), 2.95 (dt, J=6.5 Hz, J=5.8 Hz, 2H), 3.15 (dt, J=6.5 Hz, J=6.2 Hz, 2H), 6.23 (d, J=5.5 Hz, 1H), 7.17 (t, J=5.8 Hz, 1H), 7.35 (t, J=6.2 Hz, 1H), 7.42 (dd, J=2.2 Hz, J=9.0 Hz, 1H), 7.65-7.74 (m, 2H), 7.76 (d, J=2.2 Hz, 1H), 8.12 (d, J=9.1 Hz, 1H), 8.24 (dd, J=1.3 Hz, J=8.3 Hz, 1H), 8.30-8.34 (m, 2H), 8.49 (dd, J=1.8 Hz, J=8.4 Hz, 1H), 9.03 (dd, J=1.8 Hz, J=4.2 Hz, 1H); ¹³C-NMR (75 MHz, DMSO-d₆) δ=27.6, 39.6, 40.7, 98.4, 117.2, 122.4, 124.0, 125.6, 127.1, 128.4, 130.6, 133.4, 133.5, 136.2, 136.9, 142.6, 148.6, 149.9, 151.2, 151.5.

N—(N′-3-(7-chloro-4-quinolyl)aminopropyl)-2-phenoxy-5-pyridinesulfonamide, 37. Employing 0.15 g (0.64 mmol) of N-(7-chloro-4-quinolyl)-1,3-diaminopropane and 6-phenoxy-3-pyridinesulfonyl chloride (0.2 g, 0.76 mmol) in the procedure described above gave 0.038 g (0.081 mmol, 13% yield) of white crystals. ¹H-NMR (300 MHz, CDCl₃) δ=1.94 (tt, J=6.2 Hz, J=6.2 Hz, 2H), 3.16 (t, J=6.2 Hz, 2H), 3.54 (dt, J=6.2 Hz, 2H), 5.57 (bs, 1H), 6.32 (d, J=5.7 Hz, 1H), 7.00 (d, J=8.4 Hz, 1H), 7.13 (d, J=8.4 Hz, 2H), 7.28 (d, J=8.4 Hz, 1H), 7.36 (dd, J=2.0 Hz, J=8.9 Hz, 1H), 7.41-7.46 (m, 2H), 7.70 (d, J=8.9 Hz, 1H), 7.90 (d, J=1.9 Hz, 1H), 8.08 (dd, J=2.7 Hz, J=8.6 Hz, 1H), 8.46 (d, J=5.7 Hz, 1H), 8.64 (d, J=2.7 Hz, 1H); ¹³C-NMR (75 MHz, DMSO-d₆) δ=28.5, 41.2, 99.4, 112.4, 118.1, 122.3, 124.8, 126.1, 128.1, 130.6, 132.4, 134.1, 139.4, 147.1, 149.6, 150.7, 152.5, 153.6, 165.8.

N—(N′-3-(7-chloro-4-quinolyl)aminopropyl)-4-methyl-3,4-dihydro-2H-benzo[b][1,4]oxazine-7-sulfonamide, 38. Employing 0.102 g (0.43 mmol) of N-(7-chloro-4-quinolyl)-1,3-diaminopropane and 1,4-benzoxazinesulfonyl chloride (0.123 g, 0.49 mmol) in the procedure described above provided 0.02 g (0.04 mmol, 10% yield) of off-white crystals. ¹H-NMR (400 MHz, CDCl₃) δ=1.79-1.86 (m, 2H), 2.76 (s, 3H), 3.02 (t, J=4.5 Hz, 2H), 3.19 (t, J=3.4 Hz, 2H), 3.44 (t, J=4.5 Hz, 2H), 4.23 (t, J=3.8 Hz, 2H), 5.79 (bs, 1H), 6.25 (d, J=4.1 Hz, 1H), 6.72 (d, J=6.0 Hz, 1H), 6.99 (d, J=1.5 Hz, 1H), 7.06 (dd, J=1.8 Hz, J=6.6 Hz, 1H), 7.28 (dd, J=1.8 Hz, J=6.6 Hz, 1H), 7.72 (d, J=6.6 Hz, 1H), 7.83 (d, J=1.2 Hz, 1H), 8.37 (d, J=4.1 Hz, 1H); ¹³C-NMR (75 MHz, CDCl₃) δ=26.8, 32.0, 37.5, 38.6, 39.5, 47.3, 63.9, 97.7, 109.3, 115.0, 116.3, 120.5, 124.5, 127.2, 134.2, 135.9, 146.8, 148.7, 150.4, 164.2.

Representative Procedure for the Synthesis of Urea and Thiourea Analogs. A mixture of N-(7-chloro-4-quinolyl)-1,3-diaminopropane (0.15 g, 0.64 mmol) and the appropriate isothiocyanate or isocyanate (0.53 mmol) in anhydrous THF was stirred at room temperature until the reaction was complete. In all cases, the desired urea or thiourea product precipitated from solution. The precipitate was collected via vacuum filtration and dried in vacuo.

N-(3-(7-chloro-4-quinolyl)aminopropyl)-N′-(4-methoxyphenyl)urea, 39. Employing 0.195 g (0.83 mmol) of N-(7-chloro-4-quinolyl)-1,3-diaminopropane and 4-methoxyphenyl isocyanate (0.09 mL, 0.69 mmol) in the procedure described above gave 0.244 g (0.64 mmol, 89% yield) of white crystals. ¹H-NMR (300 MHz, DMSO-d₆) δ=1.81-1.89 (m, 2H), 3.19-3.38 (m, 4H), 3.73 (s, 3H), 6.18 (t, J=5.6 Hz, 1H), 6.52 (d, J=5.6 Hz, 2H), 6.85 (dd, J=2.1 Hz, J=6.7 Hz, 2H), 7.31-7.37 (m, 3H), 7.49 (dd, J=3.2 Hz, J=10.0 Hz, 1H), 7.83 (d, J=2.2 Hz, 1H), 8.30 (d, J=2.7 Hz, 1H), 8.32 (s, 1H), 8.44 (d, J=5.4 Hz, 1H); ¹³C-NMR (75 MHz, DMSO-d₆) δ=25.8, 29.3, 37.8, 55.8, 114.9, 118.2, 120.2, 124.8, 128.2, 134.1, 134.3, 149.8, 150.8, 152.7, 154.7, 156.4.

N-(3-(7-chloro-4-quinolyl)aminopropyl)-N′-(2-methoxy-4-nitrophenyl)urea, 40. Employing 0.146 g (0.65 mmol) of N-(7-chloro-4-quinolyl)-1,3-diaminopropane and 2-methoxy-4-nitrophenyl isocyanate (0.1 g, 0.52 mmol) in the procedure described above gave 0.186 g (0.43 mmol, 83% yield) of yellow crystals. ¹H-NMR (300 MHz, DMSO-d₆) δ=1.85-1.90 (m, 2H), 3.25-3.31 (m, 3H), 3.61 (t, J=6.4 Hz, 1H), 4.00 (s, 3H), 6.51 (d, J=5.4 Hz, 1H), 7.32 (t, J=5.3 Hz, 2H), 7.46 (dd, J=2.2 Hz, J=8.8 Hz, 1H), 7.78 (dd, J=2.2 Hz, J=10.7 Hz, 2H), 7.88 (dd, J=2.4 Hz, J=9.0 Hz, 1H), 8.29 (d, J=9.3 Hz, 1H), 8.40 (d, J=3.4 Hz, 1H), 8.43 (s, 1H), 8.57 (s, 1H); ¹³C-NMR (75 MHz, DMSO-d₆) δ=28.9, 57.1, 79.9, 106.1, 116.4, 118.2, 118.4, 124.8, 128.2, 134.1, 137.4, 140.9, 147.2, 149.8, 150.7, 152.6, 155.2.

N-(3-(7-chloro-4-quinolyl)aminopropyl)-N′-(4-dimethylaminophenyl)urea, 41. Employing 0.178 g (0.75 mmol) of N-(7-chloro-4-quinolyl)-1,3-diaminopropane and 4-dimethylaminophenyl isocyanate (0.1 g, 0.62 mmol) in the procedure described above furnished 0.208 g (0.52 mmol, 84% yield) of white crystals. ¹H-NMR (300 MHz, DMSO-d₆) δ=1.81-1.88 (m, 2H), 2.84 (s, 6H), 3.24 (q, J=6.4 Hz, 2H), 3.31-3.39 (m, 2H), 6.11 (t, J=5.7 Hz, 1H), 6.52 (d, J=5.4 Hz, 1H), 6.69 (d, J=9.0 Hz, 2H), 7.23 (d, J=9.0 Hz, 2H), 7.37 (t, J=5.0 Hz, 1H), 7.49 (dd, J=2.2 Hz, J=9.0 Hz, 1H), 7.83 (d, J=2.2 Hz, 1H), 8.12 (s, 1H), 8.30 (d, J=9.0 Hz, 1H), 8.44 (d, J=5.7 Hz, 1H); ¹³C-NMR (75 MHz, DMSO-d₆) δ=2.8, 29.4, 37.8, 67.7, 95.2, 99.3, 113.9, 116.2, 118.2, 120.6, 124.8, 128.2, 131.2, 134.1, 135.5, 138.2, 143.7, 146.8, 149.3, 150.7, 152.6, 156.6.

N-(3-(7-chloro-4-quinolyl)aminopropyl)-N′-(2-methoxyphenyl)urea, 42. Employing 0.191 g (0.81 mmol) of N-(7-chloro-4-quinolyl)-1,3-diaminopropane and 2-methoxyphenyl isocyanate (0.10 mL, 0.75 mmol) in the procedure described above gave 0.254 g (0.66 mmol, 82% yield) of white crystals. ¹H-NMR (300 MHz, DMSO-d₆) δ=1.85-1.89 (m, 2H), 3.23-3.39 (m, 4H), 3.86 (s, 3H), 6.53 (d, J=5.9 Hz, 1H), 6.84-7.02 (m, 4H), 7.36 (t, J=5.1 Hz, 1H), 7.49 (dd, J=2.2 Hz, J=8.8 Hz, 1H), 7.83 (d, J=2.2 Hz, 1H), 7.94 (s, 1H), 8.14 (dd, J=2.0 Hz, J=7.1 Hz, 1H), 8.32 (d, J=9.0 Hz, 1H), 8.44 (d, J=5.6 Hz, 1H); ¹³C-NMR (75 MHz, DMSO-d₆) δ=25.8, 29.2, 37.7, 67.7, 99.4, 118.2, 118.7, 121.7, 124.7, 128.2, 130.2, 134.1, 148.0, 149.8, 150.8, 152.6, 156.0.

N-(3-(7-chloro-4-quinolyl)aminopropyl)-N′-(4-methoxyphenyl)thiourea, 43. Employing 0.162 g (0.69 mmol) of N-(7-chloro-4-quinolyl)-1,3-diaminopropane and 4-methoxyphenyl isothiocyanate (0.08 mL, 0.58 mmol) in the procedure described above gave 0.116 g (0.29 mmol, 51% yield) of white crystals. ¹H-NMR (300 MHz, DMSO-d₆) δ=1.95-1.99 (m, 2H), 3.35 (q, J=6.3 Hz, 2H), 3.38 (bs, 2H), 3.77 (s, 3H), 6.52 (d, J=5.4 Hz, 1H), 6.93 (dd, J=2.2 Hz, J=6.8 Hz, 2H), 7.25 (d, J=9.0 Hz, 2H), 7.40 (t, J=5.1 Hz, 1H), 7.50 (dd, J=2.2 Hz, J=9.0 Hz, 1H), 7.64 (bs, 1H), 7.83 (d, J=2.2 Hz, 1H), 8.29 (d, J=9.0 Hz, 1H), 8.44 (d, J=5.4 Hz, 1H), 9.38 (bs, 1H); ¹³C-NMR (75 MHz, DMSO-d₆) δ=25.8, 28.2, 55.9, 67.7, 114.7, 118.2, 124.8, 126.8, 128.0, 132.3, 134.2, 149.6, 150.8, 152.4, 157.3, 181.4.

N-(3-(7-chloro-4-quinolyl)aminopropyl)-N′-(2-methoxy-4-nitrophenyl)thiourea, 44. Employing 0.136 g (0.58 mmol) of N-(7-chloro-4-quinolyl)-1,3-diaminopropane and 2-methoxy-4-nitrophenyl isothiocyanate (0.101 g, 0.48 mmol) in the procedure described above afforded 0.144 g (0.33 mmol, 76% yield) of yellow crystals. ¹H-NMR (300 MHz, DMSO-d₆) δ=1.98-2.03 (m, 2H), 3.36-3.42 (m, 3H), 3.63-3.69 (m, 2H), 4.02 (s, 3H), 6.54 (d, J=5.4 Hz, 1H), 7.37 (t, J=5.3 Hz, 1H), 7.49 (dd, J=2.4 Hz, J=9.0 Hz, 1H), 7.83 (t, J=2.4 Hz, 2H), 7.90 (dd, J=2.7 Hz, J=9.0 Hz, 3H), 8.31 (d, J=9.3 Hz, 1H), 8.45 (d, J=5.4 Hz, 1H), 8.79 (d, J=9.0 Hz, 1H), 9.13 (bs, 1H); ¹³C-NMR (75 MHz, DMSO-d₆) δ=27.7, 42.5, 57.2, 79.9, 106.5, 116.8, 118.2, 124.8, 128.2, 134.1, 136.1, 142.9, 149.8, 150.7, 152.7, 180.5.

N-(3-(7-chloro-4-quinolyl)aminopropyl)-N′-(4-dimethylaminophenyl)thiourea, 45. Employing 0.159 g (0.67 mmol) of N-(7-chloro-4-quinolyl)-1,3-diaminopropane and 4-dimethylaminophenyl isothiocyanate (0.101 g, 0.57 mmol) in the procedure described above gave 0.157 g (0.38 mmol, 67% yield) of white crystals. ¹H-NMR (300 MHz, DMSO-d₆) δ=1.90-1.99 (m, 2H), 2.90 (s, 6H), 3.30-3.38 (m, 2H), 3.61-3.64 (m, 2H), 6.50 (d, J=5.4 Hz, 1H), 6.71 (d, J=8.8 Hz, 2H), 7.10 (d, J=8.8 Hz, 2H), 7.36 (t, J=5.3 Hz, 1H), 7.49 (dd, J=2.2 Hz, J=9.0 Hz, 2H), 7.82 (d, J=2.2 Hz, 1H), 8.27 (d, J=9.0 Hz, 1H), 8.44 (d, J=5.4, 1H), 9.28 (s, 1H); ¹³C-NMR (75 MHz, DMSO-d₆) δ=28.2, 79.9, 113.3, 118.2, 124.7, 126.7, 128.2, 134.0, 149.0, 149.8, 150.7, 152.6, 181.3.

N-(3-(7-chloro-4-quinolyl)aminopropyl)-N′-(4-dimethylaminonaphthyl)thiourea, 46. N-(7-chloro-4-quinolyl)-1,3-diaminopropane (0.123 g, 0.52 mmol) and 4-dimethylamino-1-naphthyl isothiocyanate (0.10 g, 0.44 mmol) were employed in the procedure described above. The solution was then cooled to −45° C. and 0.175 g (0.38 mmol, 96% yield) of white crystals were obtained. ¹H-NMR (300 MHz, DMSO-d₆) δ=1.92 (bs, 2H), 2.87 (s, 6H), 3.28 (bs, 2H), 3.59-3.66 (m, 2H), 6.43 (s, 1H), 7.12 (d, J=8.1 Hz, 1H), 7.34 (d, J=8.1 Hz, 2H), 7.48 (dd, J=2.2 Hz, J=9.0 Hz, 1H), 7.54-7.57 (m, 2H), 7.81-7.87 (m, 2H), 8.20-8.27 (m, 2H), 8.40 (d, J=5.6 Hz, 1H), 9.57 (bs, 1H); ¹³C-NMR (75 MHz, DMSO-d₆) δ=28.1, 45.6, 79.9, 99.3, 118.2, 124.7, 126.1, 128.2, 129.5, 130.4, 132.1, 134.1, 149.8, 150.6, 151.6, 152.6, 167.4, 182.5.

N-(7-Chloro-4-quinolyl)-N-(3-diethylaminopropanoyl)-1,2-diaminoethane, 47. A mixture of N-(7-chloro-4-quinolyl)-1,2-diaminoethane (0.1 g, 0.45 mmol), N,N-diethylamino-3-propionic acid (0.11 g, 0.6 mmol), EDC (0.11 g, 0.6 mmol) and Et₃N (0.19 mL, 1.35 mmol) in 4 mL of anhydrous DMF and CHCl₃ (1:1 v/v) was stirred at room temperature for 2 days. Saturated NaHCO₃ solution was added to the cooled reaction mixture, which was then extracted with CH₂Cl₂, dried over anhydrous MgSO₄, and concentrated in vacuo. Flash chromatography using EtOH:Et₃N (1:0.05 v/v) as the mobile phase afforded 0.10 g (0.44 mmol, 63% yield) of yellow crystals. ¹H-NMR (300 MHz, CDCl₃) δ=1.05 (t, J=7.1 Hz, 6H), 2.48 (t, J=6.1 Hz, 2H), 2.58 (q, J=7.1 Hz, 4H), 2.69 (t, J=6.1 Hz, 2H), 3.30-3.45 (m, 2H), 3.64-3.78 (m, 2H), 6.28 (d, J=5.4 Hz, 1H), 7.11 (bs, 1H), 7.40 (dd, J=2.1 Hz, J=9.0 Hz, 1H), 7.87 (d, J=9.0 Hz, 1H), 7.94 (d, J=2.1 Hz, 1H), 8.50 (d, J=5.4 Hz, 1H), 9.51 (bs, 1H); ¹³C-NMR (75 MHz, CDCl₃) δ=11.6, 32.4, 38.4, 46.3, 46.5, 48.9, 98.2, 117.5, 122.7, 125.7, 128.2, 135.2, 148.9, 150.7, 151.8, 176.2.

N-(7-Chloro-4-quinolyl)-N-(3-diethylaminopropanoyl)-1,3-diaminopropane, 48. A mixture of N-(7-chloro-4-quinolyl)-1,3-diaminopropane (1.0 g, 4.24 mmol), N,N-diethylamino-3-propionic acid (0.78 g, 4.3 mmol), EDC (0.98 g, 5.1 mmol) and triethylamine (1.8 mL, 12.9 mmol) in 30 mL of anhydrous DMF and chloroform (1:1 v/v) was stirred at room temperature for 2.5 days. The reaction mixture was concentrated in vacuo, then dissolved in dichloromethane and extracted with aqueous NaOH. The combined organic layers were dried over anhydrous MgSO₄ and concentrated in vacuo. The crude product was purified by flash chromatography (ethanol:hexanes:triethylamine 1:1:0.05 v/v) to give 0.83 g of (2.3 mmol, 54% yield) pale yellow crystals. ¹H-NMR (300 MHz, CDCl₃) δ=1.02 (t, J=7.1 Hz, 6H), 1.74-1.83 (m, 2H), 2.41 (t, J=5.7 Hz, 2H), 2.53 (q, J=7.1 Hz, 4H), 2.67 (t, J=5.9 Hz, 2H), 3.32-3.43 (m, 4H), 6.37 (d, J=5.6 Hz, 1H), 6.76 (t, J=5.7 Hz, 1H), 7.36 (dd, J=2.1 Hz, J=9.0 Hz, 1H), 7.90 (d, J=2.1 Hz, 1H), 8.02 (d, J=9.0 Hz, 1H), 8.45 (d, J=5.6 Hz, 1H), 9.04 (t, J=5.7 Hz, 1H); ¹³C-NMR (75 MHz, CDCl₃) δ=11.8, 28.6, 32.7, 35.7, 39.2, 46.5, 49.2, 98.6, 117.9, 122.5, 125.7, 128.5, 135.4, 149.4, 150.5, 151.9, 174.8.

N-(7-Chloro-4-quinolyl)-N-(3-diethylaminopropanoyl)-1,4-diaminobutane, 49. A mixture of N-(7-chloro-4-quinolyl)-1,4-diaminobutane (2.0 g, 8.0 mmol), N,N-diethylamino-3-propionic acid (1.45 g, 8.0 mmol), EDC (1.84 g, 9.6 mmol), and triethylamine (3.35 mL, 24.0 mmol) in 80 mL of anhydrous DMF and chloroform (1:1 v/v) was stirred at room temperature for 2.5 days. The reaction mixture was concentrated in vacuo and partitioned between dichloromethane and 1N NaOH solution. The combined organic layers were dried over anhydrous Na₂SO₄ and concentrated in vacuo. The crude product was purified by flash chromatography (methanol:ammonium hydroxide 1.0:0.005 v/v) to give 1.8 g (4.8 mmol, 60% yield) of colorless crystals. ¹H-NMR (300 MHz, CDCl₃) δ=1.02 (t, J=7.2 Hz, 6H), 1.60-1.88 (m, 4H), 2.36 (t, J=6.0 Hz, 2H), 2.54 (q, J=7.2 Hz, 4H), 2.65 (t, J=6.0 Hz, 2H), 3.28-3.42 (m, 4H), 5.71 (bt, 1H), 6.38 (d, J=5.7 Hz, 1H), 7.35 (dd, J=2.4 Hz, J=9.0 Hz, 1H), 7.86 (d, J=9.0 Hz, 1H), 7.93 (d, J=2.4 Hz, 1H), 8.51 (d, J=5.7 Hz, 1H), 8.85 (bt, 1H); ¹³C-NMR (75 MHz, CDCl₃) δ=11.3, 25.2, 27.8, 32.3, 38.1, 42.9, 45.8, 48.6, 98.6, 117.3, 121.9, 124.7, 128.0, 134.4, 148.9, 150.0, 151.6, 173.1.

N-(7-Chloro-4-quinolyl)-N-(3-diethylaminopropanoyl)-1,5-diaminopentane, 50. A mixture of N-(7-chloro-4-quinolyl)-1,5-diaminopentane (0.25 g, 0.95 mmol), N,N-diethylamino-3-propionic acid (0.17 g, 0.93 mmol), EDC (0.22 g, 1.14 mmol), and triethylamine (0.4 mL, 2.9 mmol) in 12 mL of anhydrous DMF and chloroform (1:1 v/v) was stirred at room temperature for 2.5 days. The reaction mixture was concentrated in vacuo, then dissolved in dichloromethane and extracted with aqueous NaOH. The combined organic layers were dried over anhydrous MgSO₄ and concentrated in vacuo. The crude product was purified by flash chromatography (methanol:ammonium hydroxide 1.0:0.05 v/v) to afford 0.045 g (0.11 mmol, 12% yield) of colorless crystals. ¹H-NMR (300 MHz, CDCl₃) δ=1.01 (t, J=7.2 Hz, 6H), 1.49-1.59 (m, 4H), 1.82-1.87 (m, 2H), 2.53 (t, J=6.0 Hz, 2H), 2.53 (q, J=7.2 Hz, 4H), 2.64 (t, J=6.0 Hz, 2H), 3.26-3.33 (m, 4H), 5.46 (bs, 1H), 6.37 (d, J=5.4 Hz, 1H), 7.35 (dd, J=2.2 Hz, 8.8 Hz, 1H), 7.94 (d, J=2.2 Hz, 1H), 7.96 (d, J=8.8 Hz, 1H), 8.51 (d, J=5.4 Hz, 1H), 8.80 (bs, 1H); ¹³C-NMR (75 MHz, CDCl₃) δ=11.8, 24.3, 28.0, 30.0, 32.8, 37.9, 43.4, 46.3, 49.2, 100.6, 117.6, 122.0, 128.9, 134.9, 149.5, 150.3, 152.3, 173.7.

N-(7-Chloro-4-quinolyl)-N-(3-diethylaminopropanoyl)-1,6-diaminohexane, 51. A mixture of N-(7-chloro-4-quinolyl)-1,6-diaminohexane (0.1 g, 0.36 mmol), N,N-diethylamino-3-propionic acid (0.08 g, 0.43 mmol), EDC (0.08 g, 0.43 mmol) and Et₃N (0.19 mL, 1.35 mmol) was stirred at room temperature in 4 mL of DMF:CHCl₃ (1:1 v/v) for 2 days. Saturated NaHCO₃ was added to the cooled reaction mixture, which was then extracted with CH₂Cl₂ and dried over anhydrous MgSO₄, and concentrated in vacuo. Purification by flash chromatography using EtOH:Et₃N (1:0.05 v/v) as the mobile phase gave yellow crystals (0.12 g, 0.27 mmol, 76% yield). ¹H-NMR (300 MHz, CDCl₃) δ=1.06 (t, J=7.1 Hz, 6H), 1.25-1.62 (m, 6H), 1.63-1.82 (m, 2H), 2.40 (t, J=6.1 Hz, 2H), 2.58 (q, J=7.1 Hz, 4H), 2.69 (t, J=6.1 Hz, 2H), 3.20-3.41 (m, 4H), 5.37 (bs, 1H), 6.41 (d, J=5.4 Hz, 1H), 7.38 (dd, J=2.1 Hz, J=9.0 Hz, 1H), 7.80 (d, J=9.0 Hz, 1H), 7.97 (d, J=2.1 Hz, 1H), 8.53 (d, J=5.4 Hz, 1H), 8.67 (bs, 1H); ¹³C-NMR (75 MHz, CDCl₃) δ=11.7, 26.7, 28.7, 29.8, 32.7, 38.7, 43.1, 46.3, 49.2, 99.1, 117.5, 121.9, 125.3, 128.6, 135.0, 149.2, 150.3, 152.0, 173.2.

Representative Procedure for the Synthesis of Amide Analogs 52-56. To a solution of N-(1-naphthyl)anthranilic acid (0.15 g, 0.57 mmol) and 2-chloro-4,6-dimethoxy-1,3,5-triazine (CDMT) (0.1 g, 0.57 mmol) in anhydrous CHCl₃, 0.1 mL (0.63 mmol) of N-methylmorpholine (NMM) was added dropwise at 0° C. and stirred at room temperature for 2 hours. N-(7-Chloro-4-quinolyl)-1,3-diaminopropane (0.41 g, 1.7 mmol) in anhydrous DMF was then added. The reaction mixture was stirred for another 2 hours and concentrated under reduced pressure. The residue was dissolved in CH₂Cl₂, extracted with water, dried over anhydrous Na₂SO₄ and concentrated in vacuo. Flash chromatography (MeOH:EtOAc 1:32 v/v) allowed the isolation of 0.2 g of 52 (0.41 mmol, 73% yield) as light brown crystals.

N-(7-Chloro-4-quinolyl)-N-(2-naphthylaminobenzoyl)-1,3-diaminopropane, 52. ¹H-NMR (300 MHz, CDCl₃) δ=1.91 (m, 2H), 3.37 (q, J=5.7 Hz, 2H), 3.58 (q, J=6.3 Hz, 2H), 6.32 (d, J=5.7 Hz, 1H), 6.46 (t, J=5.7 Hz, 1H), 6.71 (m, 1H), 6.97 (bs, 1H), 7.16-7.28 (m, 3H), 7.38-7.56 (m, 5H), 7.62 (d, J=8.1 Hz, 1H), 7.84-7.95 (m, 3H), 8.17 (m, 1H), 8.42 (d, J=5.7 Hz, 1H), 9.90 (s, 1H); ¹³C-NMR (75 MHz, DMSO-d₆) δ=27.6, 37.1, 40.2, 98.5, 98.7, 114.8, 115.7, 117.4, 117.8, 118.1, 121.4, 122.6, 124.0, 126.0, 126.2, 127.2, 127.3, 128.4, 128.7, 131.9, 133.4, 134.3, 137.1, 145.4, 148.8, 150.1, 151.6, 169.3.

N-(7-Chloro-4-quinolyl)-N-(2-benzylamino-4-fluorobenzoyl)-1,3-diaminopropane, 53. Employing 0.15 g (0.6 mmol) of 4-fluoro-N-benzylanthranilic acid and N-(7-chloro-4-quinolyl)-1,3-diaminopropane (0.43 g, 1.83 mmol) in the procedure described above and purification by flash chromatography (MeOH:EtOAc 1:49 v/v) gave 0.16 g (0.35 mmol, 59% yield) of colorless crystals. ¹H-NMR (300 MHz, CD₃OD) δ=2.00 (m, 2H), 3.40-3.54 (m, 4H), 4.31 (s, 2H), 6.23-6.38 (m, 2H), 6.52 (d, J=5.7 Hz, 1H), 7.18-7.38 (m, 6H), 7.50 (dd, J=2.1 Hz, 9.0 Hz, 1H), 7.75 (d, J=2.1 Hz, 1H), 8.05 (d, J=9.0 Hz, 1H), 8.31 (d, J=5.7 Hz, 1H); ¹³C-NMR (75 MHz, DMSO-d₆) δ=27.7, 36.8, 36.9, 45.9, 46.0, 97.5, 97.9, 98.6, 100.8, 101.1, 111.8, 117.4, 117.5, 124.0, 126.9, 127.1, 127.4, 128.5, 130.6, 130.8, 133.3, 138.9, 149.0, 149.9, 150.0, 151.1, 151.2, 151.3, 151.4, 151.8, 163.3, 166.5, 168.4, 168.4, 168.5.

N-(7-Chloro-4-quinolyl)-N-(2-phenylethylaminobenzoyl)-1,3-diaminopropane, 54. Employing 0.15 g (0.6 mmol) of N-phenethylanthranilic acid and N-(7-chloro-4-quinolyl)-1,3-diaminopropane (0.44 g, 1.86 mmol) in the procedure described above and purification by flash chromatography (MeOH:EtOAc 1:24 v/v) gave 0.19 g (0.42 mmol, 68% yield) of colorless crystals. ¹H-NMR (300 MHz, CD₃OD) δ=1.98 (m, 2H), 2.90 (t, J=7.2 Hz, 2H), 3.36-3.52 (m, 6H), 6.50 (d, J=5.7 Hz, 1H), 6.59 (m, 1H), 6.74 (d, J=8.1 Hz, 1H), 7.13 (m, 1H), 7.20-7.48 (m, 7H), 7.78 (d, J=2.4 Hz, 1H), 8.06 (d, J=9.0 Hz, 1H), 8.33 (d, J=5.7 Hz, 1H); ¹³C-NMR (75 MHz, DMSO-d₆) δ=22.7, 34.8, 36.8, 43.8, 43.9, 79.1, 98.6, 111.0, 114.1, 115.0, 115.1, 115.2, 117.4, 117.5, 124.0, 126.0, 127.4, 128.2, 128.8, 132.3, 133.4, 139.4, 148.7, 148.8, 148.9, 149.9, 150.0, 151.7, 169.1, 169.1, 169.2, 169.2.

N-(7-Chloro-4-quinolyl)-N-(2-cyclohexylthiobenzoyl)-1,3-diaminopropane, 55. Employing 0.12 g (0.51 mmol) of 2-(cyclohexylthio)benzoic acid and N-(7-chloro-4-quinolyl)-1,3-diaminopropane (0.1 g, 0.43 mmol) in the procedure described above (this reaction was conducted at 70° C.) and purification by flash chromatography (MeOH:CH₂Cl₂ 1:49 v/v and gradually changing the ratio of MeOH:CH₂Cl₂ to 1:11.5 v/v) gave 0.07 g (0.15 mmol, 35% yield) of light yellow crystals. ¹H-NMR (300 MHz, CDCl₃) δ=1.12-1.44 (m, 5H), 1.59 (s, 1H), 1.72 (m, 2H), 1.85-2.04 (m, 4H), 3.11 (m, 1H), 3.50-3.70 (m, 4H), 6.41 (d, J=5.4 Hz, 1H), 6.77 (bt, 1H), 7.29-7.53 (m, 5H), 7.75 (dd, J=7.5 Hz, J=2.1 Hz, 1H), 7.90 (d, J=2.1 Hz, 1H), 8.02 (d, J=9.0 Hz, 1H), 8.46 (d, J=5.4 Hz, 1H); ¹³C-NMR (75 MHz, CDCl₃) δ=25.2, 25.8, 28.1, 33.1, 36.5, 39.0, 48.0, 98.2, 98.3, 117.5, 122.2, 125.3, 127.4, 127.8, 129.2, 130.5, 132.0, 134.0, 135.0, 138.0, 148.7, 150.2, 151.2, 151.3, 169.6.

N-(7-Chloro-4-quinolyl)-N-(2-phenylthiobenzoyl)-1,3-diaminopropane, 56. Employing 0.12 g (0.51 mmol) of 2-(phenylthio)benzoic acid and N-(7-chloro-4-quinolyl)-1,3-diaminopropane (0.1 g, 0.43 mmol) in the procedure described above (this reaction was conducted at 70° C.) and purification by flash chromatography (MeOH:CH₂Cl₂ 1:24 v/v) gave 0.05 g (0.1 mmol, 25% yield) of light yellow crystals. ¹H-NMR (300 MHz, CDCl₃) δ=1.84 (m, 2H), 3.42 (q, J=6.0 Hz, 2H), 3.54 (q, J=6.0 Hz, 1H), 6.36 (d, J=5.4 Hz, 1H), 6.60 (t, J=6.0 Hz, 1H), 6.89 (t, J=6.0 Hz, 1H), 7.22-7.39 (m, 9H), 7.66 (m, 1H), 7.89 (d, J=1.8 Hz, 1H), 7.96 (d, J=9.0 Hz, 1H), 8.44 (d, J=5.4 Hz, 1H); ¹³C-NMR (75 MHz, CDCl₃) δ=28.0, 36.6, 38.8, 98.2, 98.3, 117.5, 122.1, 125.2, 125.3, 127.3, 127.7, 128.0, 128.1, 128.8, 129.5, 131.0, 131.2, 132.3, 133.7, 134.3, 134.9, 136.5, 148.9, 150.0, 151.4, 151.5, 169.3.

Representative Procedure for the Synthesis of Amide Analogs (57-64). N-(7-Chloro-4-quinolyl)-1,3-diaminopropane (0.1 g, 0.43 mmol), Boc-Trp-OH (0.16 g, 0.52 mmol) and 2-chloro-4,6-dimethoxy-1,3,5-triazine (CDMT) (0.09 g, 0.52 mmol) were dissolved in 3 mL of acetonitrile and 1 mL of DMF. N-Methylmorpholine (NMM) (0.165 g, 0.65 mmol) was added and the reaction was stirred at 40° C. for 24 hours. The solvents were removed under reduced pressure and dissolved in 25 mL of CH₂Cl₂ and washed twice with 1 mL of water and brine, respectively. The combined organic layers were dried over anhydrous MgSO₄ and concentrated in vacuo. Purification by flash chromatography using EtOAc:EtOH:Et₃N (4:1:0.02 v/v) as the mobile phase gave 0.134 g of 57 as a colorless oil (0.26 mmol, 60% yield) from Boc-D-Trp-OH. The same procedure gave 0.09 g of 58 as a colorless oil (0.17 mmol, 40% yield) from Boc-Trp-OH.

N-(7-Chloro-4-quinolyl)-N-1,3-diaminopropan-N″-t-Boc-tryptophan amide, 57 and 58. ¹H-NMR (300 MHz, CDCl₃) δ=1.40 (s, 9H), 2.74-2.92 (m, 4H), 1.65-1.84 (m, 2H), 3.18-3.36 (m, 6H), 4.34 (t, J=6.0 Hz, 1H), 6.36 (d, J=5.6 Hz, 1H), 6.89-7.11 (m, 3H), 7.12 (s, 1H), 7.31 (d, J=7.5 Hz, 1H), 7.40 (dd, J=2.0 Hz, J=7.2 Hz, 1H), 7.77 (d, J=2.0 Hz, 1H), 8.06 (d, J=9.0 Hz, 1H), 8.31 (d, J=6.0 Hz, 1H); ¹³C-NMR (75 MHz, CDCl₃) δ=27.5, 28.2, 36.6, 39.7, 56.2, 78.3, 79.5, 98.5, 109.8, 111.2, 117.6, 118.3, 118.7, 121.3, 123.1, 123.4, 124.8, 126.4, 127.7, 135.1, 136.9, 148.4, 151.3, 156.4, 174.0.

N-(7-Chloro-4-quinolyl)-N-1,3-diaminopropan-N″-Z-lysine amide, 59. Employing 0.1 g (0.43 mmol) of N-(7-chloro-4-quinolyl)-1,3-diaminopropane and Z-Lys(Boc)-OH (0.198 g, 0.52 mmol) in the procedure described above and purification by flash chromatography using EtOAc:EtOH:Et₃N (1:1:0.02 v/v) as the mobile phase gave a colorless oil (0.077 g, 0.13 mmol, 30% yield). ¹H-NMR (300 MHz, CDCl₃) δ=1.42 (s, 9H), 1.61-1.98 (m, 6H), 2.95 (m, 1H), 3.01-3.18 (m, 2H), 3.22-3.45 (m, 4H), 4.11-4.25 (m, 1H), 4.83 (t, J=6.0 Hz, 1H), 5.10 (s, 2H), 6.12 (d, J=6.3 Hz, 1H), 6.48 (d, J=5.6 Hz, 1H), 7.25-7.40 (m, 5H), 7.92 (d, J=2.2 Hz, 1H), 7.93 (d, J=9.0 Hz, 1H), 8.42 (d, J=5.6 Hz, 1H); ¹³C-NMR (75 MHz, CDCl₃) δ=22.8, 28.2, 28.7, 29.9, 31.9, 36.5, 39.3, 39.8, 55.6, 67.5, 79.6, 98.7, 117.8, 122.4, 125.5, 128.3, 128.5, 128.8, 135.1, 136.4, 149.4, 150.3, 152.2, 156.7, 156.8, 162.9, 173.7.

N-(7-Chloro-4-quinolyl)-N-1,3-diaminopropan-N″-t-Boc-proline amide, 60. Employing 0.1 g (0.43 mmol) of N-(7-chloro-4-quinolyl)-1,3-diaminopropane and Boc-Pro-OH (0.112 g, 0.52 mmol) in the procedure described above and purification by flash chromatography using EtOH:Et₃N (1:0.02 v/v) as the mobile phase gave a colorless oil (0.092 g, 0.24 mmol, 55% yield). ¹H-NMR (300 MHz, CDCl₃) δ=1.45 (s, 9H), 1.82-2.10 (m, 6H), 3.36-3.62 (m, 6H), 4.18-4.22 (m, 1H), 6.62 (d, J=9.0 Hz, 1H), 7.44 (dd, J=2.2 Hz, J=6.8 Hz, 1H), 7.81 (d, J=2.2 Hz, 1H), 8.17 (d, J=9.0 Hz, 1H), 8.38 (d, J=6.8 Hz, 1H); ¹³C-NMR (75 MHz, CDCl₃) δ=24.6, 28.1, 28.4, 36.0, 38.9, 47.2, 60.3, 80.6, 98.2, 117.4, 122.2, 125.5, 127.6, 135.3, 148.3, 150.4, 150.9.

N-(7-Chloro-4-quinolyl)-N-(3-pyridoyl)-1,3-diaminopropane, 61. Employing 0.1 g (0.43 mmol) of N-(7-chloro-4-quinolyl)-1,3-diaminopropane and 3-nicotinic acid (0.064 g, 0.52 mmol) in the procedure described above and purification by flash chromatography using EtOH:Et₃N (1:0.02 v/v) as mobile phase afforded a colorless oil (0.032 g, 0.084 mmol, 20% yield). ¹H-NMR (300 MHz, CDCl₃) δ=2.01-2.72 (m, 2H), 3.51 (t, J=6.9 Hz, 2H), 3.75 (t, J=6.9 Hz, 2H), 6.66 (d, J=8.4 Hz, 1H), 7.45-7.60 (m, 2H), 7.82 (dd, J=2.1 Hz, J=5.8 Hz, 1H), 8.24-8.31 (m, 2H), 8.39 (d, J=2.1 Hz, 1H), 8.71 (dd, J=2.1 Hz, J=5.8 Hz, 1H), 9.10 (d, J=8.4 Hz, 1H); ¹³C-NMR (75 MHz, CDCl₃) δ=28.2, 38.7, 39.5, 98.2, 116.3, 119.4, 123.6, 124.7, 127.3, 129.5, 133.9, 134.1, 134.7, 147.4, 147.8, 151.0, 151.7, 166.4.

N-(7-Chloro-4-quinolyl)-N-[3-(6-hydroxypyridoyl]-1,3-diaminopropane, 62. Employing 0.1 g (0.43 mmol) of N-(7-chloro-4-quinolyl)-1,3-diaminopropane and 6-hydroxynicotinic acid (0.075 g, 0.52 mmol) in the procedure described above and purification by flash chromatography using EtOH:Et₃N (1:0.02 v/v) as mobile phase gave a colorless oil (0.038 g, 0.11 mmol, 25% yield). ¹H-NMR (300 MHz, CDCl₃) δ=1.82-1.98 (m, 2H), 3.62 (t, J=6.7 Hz, 2H), 3.79 (t, J=6.7 Hz, 2H), 6.42 (d, J=7.5 Hz, 1H), 7.15 (d, J=6.8 Hz, 1H), 7.24-7.38 (m, 2H), 8.21 (d, J=7.5 Hz, 1H), 8.32 (d, J=6.8 Hz, 1H), 8.42 (dd, J=2.0 Hz, J=5.4 Hz, 1H), 8.91 (d, J=2.0 Hz, 1H); ¹³C-NMR (75 MHz, CDCl₃) δ=28.4, 38.3, 39.6, 98.2, 110.9, 114.7, 119.4, 116.5, 124.6, 129.5, 134.0, 145.2, 149.8, 151.8, 154.1, 154.7, 166.7.

N-(7-Chloro-4-quinolyl)-N-(3-dimethylaminobenzoyl)-1,3-diaminopropane, 63. Employing 0.1 g (0.43 mmol) of N-(7-chloro-4-quinolyl)-1,3-diaminopropane and 3-dimethylaminobenzoic acid (0.086 g, 0.52 mmol) in the procedure described above and purification by flash chromatography using CH₂Cl₂:EtOH:Et₃N (1:1:0.02 v/v) as the mobile phase gave a colorless oil (0.042 g, 0.11 mmol, 25% yield). ¹H-NMR (300 MHz, CDCl₃) δ=1.95-2.18 (m, 2H), 3.47 (t, J=6.8 Hz, 2H), 3.55 (t, J=6.8 Hz, 2H), 3.96 (s, 6H), 6.55 (d, J=5.7 Hz, 1H), 7.36-7.42 (m, 2H), 7.53 (dd, J=6.3 Hz, J=7.5 Hz, 1H), 7.70 (dd, J=2.1 Hz, J=6.3 Hz, 1H), 7.75 (d, J=2.1 Hz, 2H), 8.12 (d, J=9 Hz, 1H), 8.32 (d, J=5.7 Hz, 1H); ¹³C-NMR (75 MHz, CDCl₃) δ=27.8, 39.4, 39.6, 40.7, 40.9, 98.7, 108.5, 116.3, 116.9, 120.6, 129.1, 134.4, 134.8, 148.5, 150.9, 151.7, 166.8.

N-(7-Chloro-4-quinolyl)-N-[3-(2-benzimidazol)propanoyl]-1,3-diaminopropane, 64. Employing 0.1 g (0.43 mmol) of N-(7-chloro-4-quinolyl)-1,3-diaminopropane and 2-benzimidazolepropionic acid (0.1 g, 0.52 mmol) in the procedure described above and purification by flash chromatography using EtOH:Et₃N (1:0.01 v/v) as mobile phase gave a colorless oil (0.061 g, 0.15 mmol, 35% yield). ¹H-NMR (300 MHz, MeOD) δ=1.78-1.95 (m, 2H), 2.27 (t, J=7.2 Hz, 2H), 3.21 (t, J=7.5 Hz, 2H), 3.25 (t, J=7.5 Hz, 2H), 3.27 (t, J=7.2 Hz, 2H), 6.38 (d, J=5.7 Hz, 1H), 7.10-7.18 (m, 2H), 7.37 (dd, J=2.1 Hz, J=9.0 Hz, 1H), 7.42-7.51 (m, 2H), 8.05 (d, J=9.0 Hz, 1H), 7.77 (d, J=2.1 Hz, 1H), 8.28 (d, J=5.7 Hz, 1H); ¹³C-NMR (75 MHz, CDCl₃) δ=24.5, 27.8, 33.5, 36.6, 39.9, 98.4, 117.4, 122.1, 123.3, 125.1, 125.4, 135.4, 135.7, 147.2, 150.1, 152.0, 154.3, 173.3.

N-(7-Chloro-4-quinolyl)-N-(2,5-diaminobenzoyl)-1,3-diaminopropane, 65. A mixture of 5-aminoisatoic anhydride (0.1 g, 0.56 mmol) and N-(7-chloro-4-quinolyl)-1,3-diaminopropane (0.15 g, 0.67 mmol) in ethanol was refluxed for 24 hours. After cooling to room temperature, the filtrate was concentrated under reduced pressure. The residue was purified using flash chromatography (MeOH:CH₂Cl₂ 3:7 v/v) to afford 0.13 g (0.34 mmol, 64% yield) of brown crystals. ¹H-NMR (300 MHz, CD₃OD) δ=1.95 (m, 2H), 3.36 (t, J=6.9 Hz, 1H), 3.43 (t, J=6.9 Hz, 1H), 6.42 (d, J=6.0 Hz, 1H), 6.65 (d, J=8.7 Hz, 1H), 6.74 (dd, J=2.4 Hz, J=8.7 Hz, 1H), 6.85 (d, J=2.4 Hz, 1H), 7.29 (dd, J=2.1 Hz, J=9.0 Hz, 1H), 7.70 (d, J=2.1 Hz, 1H), 8.01 (d, J=9.0 Hz, 1H), 8.26 (d, J=6.0 Hz, 1H); ¹³C-NMR (75 MHz, CD₃OD) δ=29.2, 38.1, 41.3, 99.5, 115.9, 118.6, 119.8, 120.0, 122.4, 124.2, 125.9, 127.2, 136.3, 139.0, 141.8, 149.1, 152.0, 152.5, 172.1.

N-[2-{(N′-2-(7-chloro-4-quinolyl)aminoethyl-N″-ethyl}aminoethyl]-2-benzylamino-4-fluorobenzamide, 66. Employing 0.034 g (0.14 mmol) of 4-fluoro-N-benzylanthranilic acid and N-(7-chloro-4-quinolyl)-N′-ethyl-N′-(2-aminoethyl)-1,2-diaminoethane 30 (0.04 g, 0.14 mmol) in the procedure described for the syntheses of 52-56 followed by flash chromatography using MeOH:CH₂Cl₂ (1:49 to 1:15 v/v) as the mobile phase gave 0.047 g (0.09 mmol, 66% yield) of a light yellow oil. ¹H-NMR (300 MHz, CD₃OD) δ=1.10 (t, J=6.9 Hz, 3H), 2.64-2.78 (m, 4H), 2.85 (t, J=6.3 Hz, 2H), 3.34-3.47 (m, 4H), 3.55 (t, J=6.9 Hz, 2H), 6.40 (d, J=5.7 Hz, 1H), 7.24 (dd, J=1.8 Hz, J=9.0 Hz, 1H), 7.66 (d, J=1.8 Hz, 1H), 7.96 (d, J=9.0 Hz, 1H), 8.08 (s, 1H), 8.24 (d, J=5.7 Hz, 1H), 8.38 (s, 2H); ¹⁹F-NMR (282 MHz, CDCl₃) δ=−62.9; ¹³C-NMR (75 MHz, CD₃OD) δ=12.2, 38.5, 41.5, 47.8, 52.4, 53.5, 98.8 (m), 99.8, 99.9, 102.5 (m), 113.1, 118.5, 123.9, 126.0, 127.2, 127.3, 128.2, 129.6, 131.2 (dd, J_{C-F}=6.8 Hz, J_{C-F}=22.6 Hz), 136.4, 136.9, 149.0, 152.0, 152.5, 152.6, 152.7, 165.4, 168.7, 171.3.

Representative Procedure for the Synthesis of Amide Analogs 67-70. To a solution of N-(7-chloro-4-quinolyl)-1,3-diaminopropane (0.11 g, 0.47 mmol) and Et₃N (0.13 mL, 0.94 mmol) in anhydrous DMF and CHCl₃ (1:1 v/v), 3,5-bis(trifluoromethyl)benzoyl chloride (0.092 mL, 0.51 mmol) was added at 0° C. The reaction mixture was stirred for 3 hours at room temperature and concentrated under reduced pressure. Saturated NaHCO₃ solution was added to the residue, which was then extracted with CH₂Cl₂, dried over anhydrous Na₂SO₄, and concentrated in vacuo. Flash chromatography using MeOH:CH₂Cl₂ (1:19 to 1:9 v/v) gave 0.224 g of the N-acyl quinolinium salt of 67. The crystalline residue was hydrolyzed in 1N NaOH solution, extracted with CH₂Cl₂, dried over anhydrous Na₂SO₄, and concentrated in vacuo to yield 0.13 g (0.27 mmol, 59% yield) of off-white crystals.

N-(7-Chloro-4-quinolyl)-N-{bis(trifluoromethyl)benzoyl}-1,3-diaminopropane, 67. ¹H-NMR (300 MHz, CD₃OD) δ=2.03 (m, 2H), 3.37 (t, J=6.9 Hz, 2H), 3.55 (t, J=6.9 Hz, 2H), 6.40 (d, J=5.7 Hz, 1H), 7.24 (dd, J=1.8 Hz, J=9.0 Hz, 1H), 7.66 (d, J=1.8 Hz, 1H), 7.96 (d, J=9.0 Hz, 1H), 8.08 (s, 1H), 8.24 (d, J=5.7 Hz, 1H), 8.38 (s, 2H); ¹⁹F-NMR (282 MHz, CDCl₃) δ=−62.9 (s, 6F); ¹³C-NMR (75 MHz, CD₃OD) δ=27.7, 37.9, 40.2, 98.3, 98.4, 117.4, 117.9, 121.5, 122.9, 124.7, 124.8, 125.1, 126.3, 126.4, 127.7, 128.7, 131.8 (q, J_{C-F}=126.0 Hz), 135.0, 136.7, 148.3, 151.0, 151.1, 151.2, 165.5.

N-(7-Chloro-4-quinolyl)-N-(pentafluorobenzoyl)-1,3-diaminopropane, 68. Using 0.11 g (0.47 mmol) of N-(7-chloro-4-quinolyl)-1,3-diaminopropane and 2,3,4,5,6-pentafluorobenzoyl chloride (0.07 mL, 0.51 mmol) in the procedure described above and purification by flash chromatography with MeOH:CH₂Cl₂ (1:19 to 1:9 v/v), followed by extraction with 1N NaOH gave 0.13 g (0.3 mmol, 65% yield) of off-white crystals. ¹H-NMR (300 MHz, CD₃OD) δ=2.03 (m, 2H), 3.46 (t, J=7.2 Hz, 2H), 3.54 (t, J=6.9 Hz, 2H), 6.53 (d, J=5.4 Hz, 1H), 7.38 (dd, J=2.1 Hz, J=9.0 Hz, 1H), 7.76 (d, J=2.1 Hz, 1H), 8.08 (d, J=9.0 Hz, 1H), 8.34 (d, J=5.4 Hz, 1H); ¹⁹F-NMR (282 MHz, CD₃OD) δ=−144.4 (m, 2F), −155.5 (tt, J=2.5 Hz, J=19.7 Hz, 1F), −164.0 (m, 2F); ¹³C-NMR (75 MHz, CD₃OD) δ=28.9, 38.7, 41.2, 99.5, 99.6, 113.4 (m), 118.7, 124.2, 126.0, 127.5, 127.6, 136.3, 138.9 (m), 143.3 (m), 145.0 (m), 149.5, 152.3, 152.5, 159.8.

N-(7-Chloro-4-quinolyl)-N-(heptafluorobutyryl)-1,3-diaminopropane, 69. Using 0.11 g (0.47 mmol) of N-(7-chloro-4-quinolyl)-1,3-diaminopropane and perfluorobutyryl chloride (0.08 mL, 0.51 mmol) in the procedure described above and purification by flash chromatography with MeOH:CH₂Cl₂ (1:16 v/v), followed by extraction with 1N NaOH gave 0.1 g (0.23 mmol, 49% yield) of off-white crystals. ¹H-NMR (300 MHz, CD₃OD) δ=1.98 (m, 2H), 3.39 (t, J=7.2 Hz, 2H), 3.45 (t, J=7.2 Hz, 2H), 6.50 (d, J=5.7 Hz, 1H), 7.39 (dd, J=2.1 Hz, J=9.0 Hz, 1H), 7.76 (d, J=2.1 Hz, 1H), 8.06 (d, J=9.0 Hz, 1H), 8.34 (d, J=5.7 Hz, 1H); ¹⁹F-NMR (282 MHz, CD₃OD) δ=−82.6 (t, J=9.0 Hz, 3F), −122.2 (q, J=9.0 Hz, 2F), −128.7 (s, 2F); ¹³C-NMR (75 MHz, CD₃OD) δ=28.6, 38.7, 41.1, 99.5, 99.6, 105.8-114.2 (m), 117.1 (t, J_{C-F}=141.0 Hz), 118.7, 120.9 (t, J_{C-F}=141.0 Hz), 124.1, 126.0, 126.0, 127.4, 127.5, 136.3, 149.4, 152.2, 152.5, 159.4 (t, J_{C-F}=95.9 Hz).

N-(7-Chloro-4-quinolyl)-N-(pentadecafluorooctanoyl)-1,3-diaminopropane, 70. Employing 0.15 g (0.64 mmol) of N-(7-chloro-4-quinolyl)-1,3-diaminopropane and pentadecafluorooctanoyl chloride (0.17 mL, 0.7 mmol) in the procedure described above and purification by flash chromatography using MeOH:CH₂Cl₂ (1:19 to 1:9 v/v), followed by extraction with 1N NaOH gave 0.1 g (0.16 mmol, 25% yield) of off-white crystals. ¹H-NMR (300 MHz, CD₃OD) δ=1.99 (m, 2H), 3.40 (t, J=7.2 Hz, 2H), 3.45 (t, J=6.9 Hz, 2H), 6.50 (d, J=6.0 Hz, 1H), 7.39 (dd, J=2.1 Hz, J=9.0 Hz, 1H), 7.77 (d, J=2.1 Hz, 1H), 8.07 (d, J=9.0 Hz, 1H), 8.34 (d, J=6.0 Hz, 1H); ¹⁹F-NMR (282 MHz, CDCl₃) δ=−78.8 (tt, J=2.5 Hz, J=9.9 Hz, 3F), −117.1 (t, J=12.5 Hz, 2F), −118.9 (m, 2F), −119.4 (m, 2F), −120.1 (m, 2F), −123.7 (m, 2F); ¹³C-NMR (75 MHz, CD₃OD) δ=28.6, 38.8, 41.2, 106.8-117.2 (m), 118.7, 120.3 (t, J_{C-F}=112.8 Hz), 124.1, 126.0, 126.1, 127.5, 127.6, 136.3, 149.6, 152.3, 152.4, 159.5 (t, J_{C-F}=96.9 Hz).

Representative Procedure for the Synthesis of Amide Analogs 71-74. To a solution of N-(7-chloro-4-quinolyl)-N′-ethyl-N′-(2-aminoethyl)-1,2-diaminoethane 30 (0.031 g, 0.11 mmol) and Et₃N (0.03 mL, 0.22 mmol) in anhydrous CH₂Cl₂, 3,5-bis(trifluoromethyl)benzoyl chloride (0.02 mL, 0.11 mmol) was added at 0° C. The reaction mixture was stirred for 1 hour at room temperature until saturated 1N NaOH solution was added. The mixture was extracted with CH₂Cl₂, and the combined organic layers were dried over anhydrous Na₂SO₄, and concentrated in vacuo. Flash chromatography using MeOH:CH₂Cl₂ (1:99 v/v) as the mobile phase gave 0.051 g of the N-acyl quinolinium salt. The residue was refluxed in 5 mL of methanol for 6 hours and concentrated under reduced pressure. Flash chromatography using MeOH:CH₂Cl₂ (1:49 v/v) gave 0.022 g (0.04 mmol, 39% yield) of a yellow oil.

N-[2-{(N′-2-(7-chloro-4-quinolyl)aminoethyl-N″-ethyl}aminoethyl]-3,5-(bistrifluoromethyl)benzamide, 71. ¹H-NMR (300 MHz, CD₃OD) δ=1.13 (t, J=7.2 Hz, 3H), 2.70-2.85 (m, 4H), 2.88 (t, J=6.3 Hz, 2H), 3.40 (t, J=6.0 Hz, 2H), 3.54 (t, J=6.0 Hz, 2H), 6.49 (d, J=5.7 Hz, 1H), 7.19 (dd, J=2.1 Hz, J=9.0 Hz, 1H), 7.67 (d, J=2.1 Hz, 1H), 7.82 (d, J=9.0 Hz, 1H), 7.99 (s, 1H), 8.24 (s, 2H), 8.29 (d, J=5.7 Hz, 1H); ¹⁹F-NMR (282 MHz, CDCl₃) δ=−63.3 (s, 6F); ¹³C-NMR (75 MHz, CD₃OD) δ=12.2, 39.2, 41.4, 52.5, 53.4, 99.7, 99.8, 118.3, 119.0, 122.6, 123.7, 126.0 (m), 127.3, 127.4, 128.6, 129.8, 132.8 (q, J_{C-F}=126.0 Hz), 136.3, 137.7, 149.1, 152.2, 152.3, 166.4.

N-[2-{(N′-2-(7-chloro-4-quinolyl)aminoethyl-N″-ethyl}aminoethyl]pentafluoro-benzamide, 72. Employing 0.047 g (0.16 mmol) of N-(7-chloro-4-quinolyl)-N′-ethyl-N′-(2-aminoethyl)-1,2-diaminoethane 30 and 2,3,4,5,6-pentafluorobenzoyl chloride (0.023 mL, 0.16 mmol) in the procedure described above and purification by flash chromatography using MeOH:EtOAc (1:24 v/v) as the mobile phase gave 0.018 g (0.04 mmol, 23% yield) of light yellow crystals. ¹H-NMR (300 MHz, CD₃OD+CDCl₃) δ=1.13 (t, J=6.9 Hz, 3H), 2.66-2.82 (m, 4H), 2.89 (t, J=6.3 Hz, 2H), 3.38 (t, J=5.7 Hz, 2H), 3.52 (t, J=6.0 Hz, 2H), 6.43 (d, J=5.7 Hz, 2H), 7.30 (dd, J=2.1 Hz, J=9.0 Hz, 1H), 7.82 (d, J=2.1 Hz, 1H), 7.83 (d, J=9.0 Hz, 1H), 8.38 (d, J=5.7 Hz, 1H); ¹⁹F-NMR (282 MHz, CDCl₃) δ=−142.0 (m, 2F), −152.3 (dt, J=3.1 Hz, J=20.6 Hz, 1F), −161.4 (m, 2F); ¹³C-NMR (75 MHz, CD₃OD) δ=11.0, 37.6, 39.9, 47.3, 51.1, 51.8, 98.4, 98.5, 111.0 (m), 116.7, 121.6, 124.8, 126.5, 126.6, 135.1, 136.9 (m, J_{C-F}=930.6 Hz), 141.6 (m, J_{C-F}=958.8 Hz), 143.4 (m, J_{C-F}=958.8 Hz), 147.6, 150.3, 150.7, 157.8.

N-[2-{(N′-2-(7-chloro-4-quinolyl)aminoethyl-N″-ethyl}aminoethyl]heptafluoro-butanamide, 73. Using 0.047 g (0.16 mmol) of N-(7-chloro-4-quinolyl)-N′-ethyl-N′-(2-aminoethyl)-1,2-diaminoethane 30 and perfluorobutyryl chloride (0.02 mL, 0.13 mmol) in the procedure described above and purification by flash chromatography with MeOH:EtOAc (1:24 v/v) gave 0.027 g (0.055 mmol, 42% yield) of a light yellow oil. ¹H-NMR (300 MHz, CD₃OD) δ=1.06 (t, J=7.2 Hz, 3H), 2.61-2.75 (m, 4H), 2.85 (t, J=6.6 Hz, 2H), 3.39-3.49 (m, 4H), 6.56 (d, J=5.7 Hz, 1H), 7.38 (dd, J=2.1 Hz, J=9.0 Hz, 1H), 7.78 (d, J=2.1 Hz, 1H), 8.09 (d, J=9.0 Hz, 1H), 8.36 (d, J=5.7 Hz, 1H); ¹⁹F-NMR (282 MHz, CDCl₃) δ=−82.6 (t, J=9.0 Hz, 3F), −122.1 (q, J=9.0 Hz, 2F), −128.7 (s, 2F); ¹³C-NMR (75 MHz, CD₃OD) δ=12.1, 39.0, 41.8, 48.8, 52.7, 53.5, 99.7, 99.8, 105.2-117.8 (m), 118.7, 120.8 (t, J_{C-F}=141.0 Hz), 124.2, 126.0, 127.5, 127.4, 136.4, 149.5, 152.3, 152.3, 152.6, 159.3 (t, J_{C-F}=94.0 Hz).

N-[2-{(N′-2-(7-chloro-4-quinolyl)aminoethyl-N″-ethyl}aminoethyl]pentadecafluoro-octanamide, 74. Employing 0.051 g (0.17 mmol) of N-(7-chloro-4-quinolyl)-N′-ethyl-N′-(2-aminoethyl)-1,2-diaminoethane 30 and pentadecafluorooctanoyl chloride (0.04 mL, 0.17 mmol) in the procedure described above and purification by flash chromatography using MeOH:EtOAc (1:24 v/v) gave 0.019 g (0.027 mmol, 16% yield) of a light yellow oil. ¹H-NMR (300 MHz, CD₃OD) δ=1.07 (t, J=7.2 Hz, 3H), 2.61-2.76 (m, 4H), 2.87 (t, J=6.6 Hz, 2H), 3.45 (q, J=6.6 Hz, 1H), 6.59 (d, J=6.0 Hz, 1H), 7.39 (dd, J=2.4 Hz, J=9.0 Hz, 1H), 7.79 (d, J=2.4 Hz, 1H), 8.12 (d, J=9.0 Hz, 1H), 8.37 (d, J=5.7 Hz, 1H); ¹⁹F-NMR (282 MHz, CDCl₃) δ=−82.7 (tt, J=2.3 Hz, J=9.9 Hz, 3F), −121.0 (t, J=13.0 Hz, 2F), −122.9 (m, 2F), −123.4 (m, 2F), −123.9 (m, 2F), −124.1 (m, 2F), −127.6 (m, 2F); ¹³C-NMR (75 MHz, CD₃OD) δ=12.1, 39.0, 41.9, 52.6, 53.5, 99.7, 118.6, 124.3, 126.1, 127.1, 136.6, 149.0, 151.9, 152.9.

Representative Procedure for the Synthesis of N-(7-chloro-4-quinolyl)-N′,N′-diethyl-1,(n)-diaminoalkanes and N-(7-chloro-4-quinolyl)-N′-ethyl-1,n-diaminoalkanes. To a solution of N-(7-chloro-4-quinolyl)-1,2-diaminoethane (3.8 g, 17.1 mmol) in anhydrous DMF was added Cs₂CO₃ (16.8 g, 51.4 mmol, 3 equiv.). The solution was stirred at 25° C. for 0.5 h and ethyl bromide (1.28 mL, 17.1 mmol, 1 equiv.) was added and stirred at 25° C. for 24 h. DMF was removed in vacuo. The residue was dissolved in CH₂Cl₂, extracted with water, dried over anhydrous Na₂SO₄, and the solvents were removed under reduced pressure. Flash chromatography (0.25%-1% Et₃N in EtOH) allowed isolation of 1.73 g (6.2 mmol, 26% yield) of N-(7-chloro-4-quinolyl)-N′,N′-diethyl-1,2-diaminoethane and 1.78 g (7.1 mmol, 31% yield) of N-(7-chloro-4-quinolyl)-N′-ethyl-1,2-diaminoethane as pale yellow crystals.

N-(7-Chloro-4-quinolyl)-N′,N′-diethyl-1,2-diaminoethane 86. See: Gallo, S.; Atifi, S.; Mahamoud, A.; Santelli-Rouvier, C.; Wolfart, K.; Molnar, J.; Barbe, J. Synthesis of aza mono, bi and tricyclic compounds. Evaluation of their anti MDR activity. Eur. J. Med. Chem. 2003, 38, 19-26; and De, D.; Byers, L. D.; Krogstad, D. J. Antimalarials: synthesis of 4-aminoquinolines that circumvent drug resistance in malaria parasites. J. Heterocycl. Chem. 1997, 34, 315-320. ¹H-NMR (300 MHz, CDCl₃) δ=1.07 (t, J=7.2 Hz, 6H), 2.60 (q, J=7.2 Hz, 4H), 2.81 (t, J=6.0 Hz, 2H), 3.20-3.30 (m, 2H), 6.09 (bs, 1H), 6.36 (d, J=5.4 Hz, 1H), 7.36 (dd, J=2.1 Hz, 9.0 Hz, 1H), 7.65 (d, J=9.0 Hz, 1H), 7.94 (d, J=2.1 Hz, 1H), 8.52 (d, J=5.4 Hz, 1H); ¹³C-NMR (75 MHz, CD₃OD) δ=11.6, 41.2, 47.9, 51.7, 99.6, 118.7, 124.0, 126.0, 127.6, 136.3, 149.5, 152.4.

N-(7-Chloro-4-quinolyl)-N′-ethyl-1,2-diaminoethane 81. See: Ridley, R. G.; Hofheinz, W.; Matile, H.; Jaquet, C.; Dorn, A.; Masciadri, R.; Jolidon, S.; Richter, W. F.; Guenzi, A.; Girometta, M-A.; Urwyler, H.; Huber, W.; Thaithong, S.; Peters, W.; 4-Aminoquinoline analogs of chloroquine with shortened side chains retain activity against chloroquine-resistant Plasmodium falciparum. Antimicrob. Agents Chemother. 1996, 40, 1846-1854. ¹H-NMR (300 MHz, CDCl₃) δ=1.14 (t, J=7.2 Hz, 3H), 1.24 (bs, 1H), 2.70 (q, J=7.2 Hz, 2H), 2.98-3.07 (m, 2H), 3.27-3.37 (m, 2H), 5.89 (bs, 1H), 6.38 (d, J=5.4 Hz, 1H), 7.34 (dd, J=2.1 Hz, 9.0 Hz, 1H), 7.70 (d, J=9.0 Hz, 1H), 7.93 (d, J=2.1 Hz, 1H), 8.51 (d, J=5.4 Hz, 1H); ¹³C-NMR (75 MHz, CDCl₃) δ=15.3, 41.9, 43.4, 47.1, 99.0, 117.2, 121.3, 124.9, 128.4, 134.5, 149.0, 149.8, 151.9.

N-(7-Chloro-4-quinolyl)-N′,N′-diethyl-1,3-diaminopropane 87. See: De, D.; Byers, L. D.; Krogstad, D. J. Antimalarials: synthesis of 4-aminoquinolines that circumvent drug resistance in malaria parasites. J. Heterocycl. Chem. 1997, 34, 315-320; and Madrid, P. B.; Sherrill, J.; Liou, A. P.; Weisman, J. L.; DeRisi, J. L.; Guy, R. K. Synthesis of ring-substituted 4-aminoquinolines and evaluation of their antimalarial activities. Bioorg. Med. Chem. Lett. 2005, 15, 1015-1018. Employing 3.0 g (12.7 mmol) of N-(7-chloro-4-quinolyl)-1,3-diaminopropane in the procedure described above and purification by flash chromatography (0.25%-1% Et₃N in EtOH) gave 0.85 g (2.9 mmol, 23% yield) of pale yellow crystals. ¹H-NMR (300 MHz, CDCl₃) δ=1.09 (t, J=7.2 Hz, 6H), 1.86-1.97 (m, 2H), 2.58-2.72 (m, 6H), 3.33-3.42 (m, 2H), 6.28 (d, J=5.4 Hz, 1H), 7.31 (dd, J=1.8 Hz, 8.7 Hz, 1H), 7.68 (d, J=8.7 Hz, 1H), 7.91 (d, J=1.8 Hz, 1H), 8.15 (bs, 1H), 8.49 (d, J=5.4 Hz, 1H); ¹³C-NMR (75 MHz, CDCl₃) δ=11.3, 24.0, 44.4, 46.8, 53.3, 98.0, 117.5, 122.0, 124.4, 128.3, 134.2, 149.0, 150.4, 151.9.

N-(7-Chloro-4-quinolyl)-N′-ethyl-1,3-diaminopropane 82. See: Ridley, R. G.; Hofheinz, W.; Matile, H.; Jaquet, C.; Dorn, A.; Masciadri, R.; Jolidon, S.; Richter, W. F.; Guenzi, A.; Girometta, M-A.; Urwyler, H.; Huber, W.; Thaithong, S.; Peters, W.; 4-Aminoquinoline analogs of chloroquine with shortened side chains retain activity against chloroquine-resistant Plasmodium falciparum. Antimicrob. Agents Chemother. 1996, 40, 1846-1854; and Tarbell, D. S.; Shakespeare, N.; Claus, C. J.; Bunnett, J. F. The synthesis of some 7-chloro-4-(3-alkylaminopropylamino)-quinolines. J. Am. Chem. Soc. 1946, 68, 1217-1219. Employing 3.0 g (12.7 mmol) of N-(7-chloro-4-quinolyl)-1,3-diaminopropane in the procedure described above and purification by flash chromatography (0.25%4% Et₃N in EtOH) gave 1.27 g (4.8 mmol, 37% yield) of pale yellow crystals. ¹H-NMR (300 MHz, CDCl₃) δ=1.22 (t, J=7.2 Hz, 3H), 1.35 (bs, 1H), 1.87-1.98 (m, 2H), 2.74 (q, J=7.2 Hz, 2H), 2.90-2.98 (m, 2H), 3.34-3.43 (m, 2H), 6.29 (d, J=5.4 Hz, 1H), 7.31 (dd, J=2.1 Hz, 9.3 Hz, 1H), 7.74 (d, J=9.3 Hz, 1H), 7.91 (d, J=2.1 Hz, 1H), 7.98 (bs, 1H), 8.49 (d, J=5.4 Hz, 1H); ¹³C-NMR (75 MHz, CDCl₃) δ=15.2, 27.0, 43.9, 44.0, 49.3, 98.0, 117.5, 122.2, 124.4, 128.2, 134.3, 149.0, 150.4, 151.9.

N-(7-Chloro-4-quinolyl)-N′,N′-diethyl-1,4-diaminobutane 88. See: De, D.; Byers, L. D.; Krogstad, D. J. Antimalarials: synthesis of 4-aminoquinolines that circumvent drug resistance in malaria parasites. J. Heterocycl. Chem. 1997, 34, 315-320; and Surrey, A. R.; Hammer, H. F. Some 7-substituted 4-aminoquinoline derivatives. J. Am. Chem. Soc. 1946, 68, 113-116. Employing 1.6 g (6.4 mmol) of N-(7-chloro-4-quinolyl)-1,4-diaminobutane in the procedure described above and purification by flash chromatography (0.25%-1% Et₃N in EtOH) gave 0.4 g (1.3 mmol, 20% yield) of pale yellow crystals. ¹H-NMR (300 MHz, CDCl₃) δ=1.03 (t, J=7.2 Hz, 6H), 1.61-1.74 (m, 2H), 1.78-1.90 (m, 2H), 2.50 (t, J=6.9 Hz, 2H), 2.57 (q, J=7.2 Hz, 4H), 3.25-3.34 (m, 2H), 5.96 (bt, 1H), 6.38 (d, J=5.4 Hz, 1H), 7.34 (dd, J=2.1 Hz, 9.0 Hz, 1H) 7.70 (d, J=9.0 Hz, 1H), 7.93 (d, J=2.1 Hz, 1H), 8.52 (d, J=5.4 Hz, 1H); ¹³C-NMR (75 MHz, CDCl₃) δ=11.1, 25.1, 26.7, 43.2, 46.6, 52.0, 98.8, 117.2, 121.6, 124.6, 128.4, 134.5, 149.0, 150.0, 151.9.

N-(7-Chloro-4-quinolyl)-N′-ethyl-1,4-diaminobutane 83. Employing 1.6 g (6.4 mmol) of N-(7-chloro-4-quinolyl)-1,4-diaminobutane in the procedure described above and purification by flash chromatography (0.25%-1% Et₃N in EtOH) gave 0.45 g (1.6 mmol, 26% yield) of pale yellow crystals. ¹H-NMR (300 MHz, CDCl₃) δ=1.16 (t, J=7.2 Hz, 3H), 1.60-1.74 (m, 3H), 1.77-1.89 (m, 2H), 2.64-2.77 (m, 4H), 3.30 (t, J=6.4 Hz, 2H), 6.18 (bs, 1H), 6.36 (d, J=5.4 Hz, 1H), 7.33 (dd, J=2.4 Hz, 9.0 Hz, 1H), 7.72 (d, J=9.0 Hz, 1H), 7.93 (d, J=2.4 Hz, 1H), 8.51 (d, J=5.4 Hz, 1H); ¹³C-NMR (75 MHz, CDCl₃) δ=15.0, 26.0, 27.7, 42.9, 43.9, 48.8, 98.5, 117.2, 121.7, 124.5, 128.1, 134.4, 148.9, 150.0, 151.7; MS (ESI) m/z calcd for C₁₅H₂₀ClN₃ 277.1. Found (M+H)⁺: 278.1.

N-(7-Chloro-4-quinolyl)-N′,N′-diethyl-1,5-diaminopentane 89. See: De, D.; Byers, L. D.; Krogstad, D. J. Antimalarials: synthesis of 4-aminoquinolines that circumvent drug resistance in malaria parasites. J. Heterocycl. Chem. 1997, 34, 315-320. Employing 2.48 g (9.4 mmol) of N-(7-chloro-4-quinolyl)-1,5-diaminopentane in the procedure described above and purification by flash chromatography (0.25%-1% Et₃N in EtOH) gave 0.99 g (3.1 mmol, 33% yield) of pale yellow crystals. ¹H-NMR (300 MHz, CDCl₃) δ=1.02 (t, J=7.2 Hz, 6H), 1.41-1.61 (m, 4H), 1.72-1.85 (m, 2H), 2.41-2.48 (m, 2H), 2.53 (q, J=7.2 Hz, 4H), 3.26-3.38 (m, 2H), 4.99 (bt, 1H), 6.41 (d, J=5.4 Hz, 1H), 7.36 (dd, J=2.4 Hz, 9.0 Hz, 1H), 7.65 (d, J=9.0 Hz, 1H), 7.95 (d, J=2.4 Hz, 1H), 8.53 (d, J=5.4 Hz, 1H); ¹³C-NMR (75 MHz, CD₃OD) δ=11.1, 26.0, 26.6, 28.9, 43.6, 47.3, 53.2, 99.2, 118.3, 123.8, 125.5, 127.4, 135.8, 149.2, 151.9, 152.1.

N-(7-Chloro-4-quinolyl)-N′-ethyl-1,5-diaminopentane 84. Employing 2.48 g (9.4 mmol) of N-(7-chloro-4-quinolyl)-1,5-diaminopentane in the procedure described above and purification by flash chromatography (0.25%-1% Et₃N in EtOH) gave 0.55 g (1.9 mmol, 20% yield) of pale yellow crystals. ¹H-NMR (300 MHz, CDCl₃) δ=1.10 (t, J=7.2 Hz, 3H), 1.22 (bs, 1H), 1.44-1.64 (m, 4H), 1.72-1.84 (m, 2H), 2.61-2.70 (m, 4H), 3.24-3.38 (m, 2H), 5.04 (bt, 1H), 6.39 (d, J=5.4 Hz, 1H), 7.34 (dd, J=2.1 Hz, 8.7 Hz, 1H), 7.65 (d, J=8.7 Hz, 1H), 7.94 (d, J=2.1 Hz, 1H), 8.52 (d, J=5.4 Hz, 1H); ¹³C-NMR (75 MHz, CDCl₃) δ=15.0, 24.6, 28.3, 29.6, 42.8, 43.9, 49.3, 98.6, 117.0, 121.3, 124.7, 128.1, 134.4, 148.8, 149.7, 151.6; MS (ESI) m/z calcd for C₁₆H₂₂ClN₃ 291.2. Found (M+H)⁺: 292.2.

N-(7-Chloro-4-quinolyl)-N′,N′-diethyl-1,6-diaminohexane 90. See: De, D.; Byers, L. D.; Krogstad, D. J. Antimalarials: synthesis of 4-aminoquinolines that circumvent drug resistance in malaria parasites. J. Heterocycl. Chem. 1997, 34, 315-320; and Drake, N. L.; Creech, H. J.; Garman, J. A.; Haywood, S. T.; Peck, R. M.; Van Hook, J. O.; Walton, E. Synthetic antimalarials. The preparation of certain 4-aminoquinolines. J. Am. Chem. Soc. 1946, 68, 1208-1213. Employing 4.0 g (14.4 mmol) of N-(7-chloro-4-quinolyl)-1,6-diaminohexane in the procedure described above and purification by flash chromatography (0.25%-1% Et₃N in EtOH) gave 0.96 g (2.9 mmol, 20% yield) of pale yellow crystals. ¹H-NMR (300 MHz, CDCl₃) δ=1.01 (t, J=7.2 Hz, 6H), 1.31-1.56 (m, 6H), 1.71-1.84 (m, 2H), 2.38-2.45 (m, 2H), 2.52 (q, J=7.2 Hz, 4H), 3.26-3.36 (m, 2H), 4.92 (bt, 1H), 6.41 (d, J=5.4 Hz, 1H), 7.36 (dd, J=2.1 Hz, 9.0 Hz, 1H), 7.65 (d, J=9.0 Hz, 1H) 7.95 (d, J=2.1 Hz, 1H), 8.53 (d, J=5.4 Hz, 1H); ¹³C-NMR (75 MHz, CDCl₃) δ=11.2, 26.4, 26.6, 26.8, 28.1, 42.6, 46.3, 52.2, 98.3, 116.9, 121.5, 124.3, 127.6, 134.1, 148.6, 149.7, 151.2.

N-(7-Chloro-4-quinolyl)-N′-ethyl-1,6-diaminohexane 85. Employing 4.0 g (14.4 mmol) of N-(7-chloro-4-quinolyl)-1,6-diaminohexane in the procedure described above and purification by flash chromatography (0.25%-1% Et₃N in EtOH) gave 1.28 g (4.2 mmol, 27% yield) of pale yellow crystals. ¹H-NMR (300 MHz, CDCl₃) δ=1.10 (t, J=7.2 Hz, 3H), 1.36-1.60 (m, 7H), 1.70-1.84 (m, 2H), 2.58-2.70 (m, 4H), 3.26-3.36 (m, 2H), 4.95 (bt, 1H), 6.40 (d, J=5.4 Hz, 1H), 7.35 (dd, J=2.1 Hz, 9.0 Hz, 1H), 7.65 (d, J=9.0 Hz, 1H), 7.95 (d, J=2.1 Hz, 1H), 8.53 (d, J=5.4 Hz, 1H); ¹³C-NMR (75 MHz, CDCl₃) δ=15.0, 26.8, 26.8, 28.3, 29.8, 42.8, 43.9, 49.4, 98.6, 117.0, 121.3, 124.6, 128.0, 134.3, 148.8, 149.7, 151.6; MS (ESI) m/z calcd for C₁₇H₂₄ClN₃ 305.2. Found (M+H)⁺: 306.2.

Representative Procedure for the Synthesis of α,ω)-(7-Chloro-4-quinolyl)alkanediols. To a solution of 4,7-dichloroquinoline (0.2 g, 1.0 mmol, 1 equiv.) in ethylene glycol (2.0 mL, 35.9 mmol, 35.5 equiv.) under inert atmosphere was added a 1.0 M solution of potassium t-butoxide in t-butyl alcohol (1.5 mL, 1.5 mmol, 1.5 equiv.). The reaction proceeded with good stirring at 80° C. for 18 h and was then quenched with saturated NaHCO₃. The mixture was extracted with CH₂Cl₂, dried over anhydrous MgSO₄, concentrated in vacuo, and purified by recrystallization from CHCl₃ to yield 0.21 g of white crystals (0.95 mmol, 94% yield).

O-(7-Chloro-4-quinolyl)ethylene glycol. ¹H-NMR (300 MHz, CDCl₃) δ=2.17 (bs, 1H), 4.16 (bt, 2H), 4.33 (t, J=4.5 Hz, 2H), 6.74 (d, J=5.1 Hz, 1H), 7.45 (dd, J=2.2 Hz, 8.9 Hz, 1H), 8.03 (d, J=2.2 Hz, 1H), 8.15 (d, J=8.9 Hz, 1H), 8.74 (d, J=5.1 Hz, 1H); ¹³C-NMR (75 MHz, CDCl₃) δ=61.4, 70.8, 101.6, 120.2, 124.0, 127.1, 128.1, 136.5, 150.0, 152.9, 162.1.

O-(7-Chloro-4-quinolyl)-1,3-propanediol. Employing 0.2 g (1.0 mmol) of 4,7-dichloroquinoline in the procedure described above and recrystallization from CHCl₃ gave 0.25 g (1.0 mmol, 99% yield) of white crystals. ¹H-NMR (300 MHz, CDCl₃) δ=2.18 (m, 2H), 3.03 (bs, 1H), 3.98 (t, J=5.9 Hz, 2H), 5.27 (t, J=5.9 Hz, 2H), 6.55 (d, J=5.5 Hz, 1H), 7.36 (dd, J=2.1 Hz, 8.7 Hz, 1H), 7.96 (d, J=2.1 Hz, 1H), 7.97 (d, J=8.7 Hz, 1H), 8.59 (d, J=5.5 Hz, 1H); ¹³C-NMR (75 MHz, CDCl₃) δ=31.7, 57.8, 64.8, 100.5, 119.3, 123.1, 126.2, 126.8, 135.7, 148.7, 151.8, 161.3.

O-(7-Chloro-4-quinolyl)-1,4-butanediol. Employing 0.2 g (1.0 mmol) of 4,7-dichloroquinoline in the procedure described above and recrystallization from CHCl₃ gave 0.17 g (0.67 mmol, 66% yield) of white crystals. ¹H-NMR (300 MHz, CDCl₃) δ=1.64 (bs, 1H), 1.80 (m, 2H), 2.06 (m, 2H), 3.79 (t, J=6.7 Hz, 2H), 4.24 (t, J=6.6 Hz, 2H), 6.72 (d, J=5.3 Hz, 1H), 7.44 (dd, J=2.0, 8.9 Hz, 1H), 8.02 (d, J=2.0 Hz, 1H,), 8.14 (d, J=8.9 Hz, 1H), 8.7 (d, J=5.3 Hz, 1H); ¹³C-NMR (75 MHz, CDCl₃) δ=25.8, 29.4, 62.5, 68.5, 100.9, 119.8, 123.3, 126.3, 127.8, 135.5, 149.5, 152.5, 161.6.

O-(7-Chloro-4-quinolyl)-1,5-pentanediol. Employing 0.2 g (1.0 mmol) of 4,7-dichloroquinoline in the procedure described above and recrystallization from CHCl₃ gave 0.3 g (1.1 mmol, 99% yield) of white crystals. ¹H-NMR (300 MHz, CDCl₃) δ=1.50 (bs, 1H), 1.68 (m, 4H), 1.99 (m, 2H), 3.73 (bt, 2H), 4.20 (t, J=6.8 Hz, 2H), 6.70 (d, J=5.3 Hz, 1H), 7.44 (dd, J=2.1, 9.0 Hz, 1H), 8.01 (d, J=2.1 Hz, 1H), 8.14 (d, J=9.0 Hz, 1H), 8.72 (d, J=5.3 Hz, 1H); ¹³C-NMR (75 MHz, CDCl₃) δ=22.4, 28.5, 32.3, 62.2, 68.4, 100.8, 119.7, 123.4, 126.3, 127.4, 135.6, 149.3, 152.3, 161.6.

O-(7-Chloro-4-quinolyl)-1,6-hexanediol. Employing 0.2 g (1.0 mmol) of 4,7-dichloroquinoline in the procedure described above and recrystallization from CHCl₃ gave 0.34 g (1.2 mmol, 92% yield) of white crystals. ¹H-NMR (300 MHz, CDCl₃) δ=1.40-1.69 (m, 6H), 1.99 (m, 2H), 3.68 (m, 3H), 4.24 (t, J=5.8 Hz, 2H), 6.71 (d, J=5.3 Hz, 1H), 7.44 (dd, J=2.1, 8.9 Hz, 1H), 8.01 (d, J=2.1 Hz, 1H), 8.14 (d, J=8.9 Hz, 1H), 8.72 (d, J=5.3 Hz, 1H); ¹³C-NMR (75 MHz, CDCl₃) δ=25.5, 25.8, 28.7, 32.5, 62.5, 68.5, 100.8, 119.8, 123.4, 126.3, 127.6, 135.6, 149.5, 152.4, 161.6.

Representative Procedure for the Synthesis of O-(7-Chloro-4-quinolyl)-N,N-diethylaminoalkanols. To a solution of O-(7-chloro-4-quinolyl)-1,4-butanediol (0.78 g, 3.1 mmol, 1 equiv.) and Et₃N (0.94 g, 9.3 mmol, 3 equiv.) in 20 mL of anhydrous THF at room temperature was added dropwise methansulfonyl chloride (1.07 g, 9.3 mmol, 3 equiv.). The reaction proceeded with good stirring for 10 minutes and was then quenched with saturated NaHCO₃. The mixture was extracted with CH₂Cl₂, dried over anhydrous MgSO₄, and concentrated in vacuo. The residue was dissolved in anhydrous CH₃CN (15.0 mL) under inert atmosphere and N,N-diisopropylethylamine (2.0 g, 15.5 mmol, 5 equiv.) and diethylamine (4.53 g, 62.0 mmol, 20 equiv.) were added. The reaction mixture was stirred at 40° C. for 48 h and was quenched with saturated NaHCO₃. The mixture was extracted with CH₂Cl₂, dried over anhydrous MgSO₄, and concentrated in vacuo. The product was purified by flash column chromatography using CH₂Cl₂:EtOH:Et₃N (5:1:0.005 v/v) as the mobile phase to give a light yellow oil (0.78 g, 2.5 mmol, 61% yield).

O-(7-Chloro-4-quinolyl)-2-(N,N-diethylamino)ethanol 91. See: Cheng, J.; Zeidan, R.; Mishra, S.; Liu, A.; Pun, S. H.; Kulkarni, R. P.; Jensen, G. S.; Bellocq, N.C.; Davis, M. E. Structure-Function Correlation of Chloroquine and Analogues as Transgene Expression Enhancers in Nonviral Gene Delivery. J. Med. Chem. 2006, 49, 6522-6531; and Clinton, R. O.; Suter, C. M. Some dialkylaminoalkyl sulfides and ethers derived from quinoline and acridine heterocycles. J. Am. Chem. Soc. 1948, 70, 491-494. Employing 0.07 g (0.3 mmol) of O-(7-chloro-4-quinolyl)ethylene glycol in the procedure described above and purification by flash chromatography using CH₂Cl₂:EtOH (5:1 v/v) containing 0.5% Et₃N as the mobile phase gave 0.07 g (0.27 mmol, 61% yield) of the desired product as a light yellow oil. ¹H-NMR (300 MHz, CDCl₃) δ=1.10 (t, J=7.1 Hz, 6H), 2.67 (q, J=7.1 Hz, 4H), 3.03 (t, J=5.9 Hz, 2H), 4.25 (t, J=5.9 Hz, 2H), 6.71 (d, J=5.1 Hz, 1H), 7.41 (dd, J=2.2 Hz, 8.9 Hz, 1H), 7.99 (d, J=2.2 Hz. 1H), 8.10 (d, J=8.9 Hz, 1H), 8.71 (d, J=5.1 Hz, 1H); ¹³C-NMR (75 MHz, CDCl₃) δ=12.6, 48.7, 51.9, 68.2, 101.7, 120.5, 124.1, 127.1, 128.5, 136.3, 150.4, 153.2, 162.2.

O-(7-Chloro-4-quinolyl)-3-(N,N-diethylamino)propanol 92. Employing 0.06 g (0.27 mmol) of O-(7-chloro-4-quinolyl)-1,3-propanediol in the procedure described above and purification by flash chromatography using CH₂Cl₂:EtOH (5:1 v/v) containing 0.5% Et₃N as the mobile phase gave 0.05 g (0.17 mmol, 69% yield) of a light yellow oil. ¹H-NMR (300 MHz, CDCl₃) δ=1.06 (t, J=7.2 Hz, 6H), 2.11 (m, 2H), 2.61 (q, J=7.2 Hz, 4H), 2.72 (t, J=6.8 Hz, 2H), 4.27 (t, J=6.2 Hz, 2H), 6.74 (d, J=5.3 Hz, 1H), 7.45 (dd, J=2.2 Hz, 8.8 Hz, 1H), 8.02 (d, J=2.2 Hz. 1H), 8.13 (d, J=8.8 Hz, 1H), 8.73 (d, J=5.3 Hz, 1H); ¹³C-NMR (75 MHz, CDCl₃) δ=11.7, 26.8, 47.0, 49.1, 66.9, 100.9, 119.8, 123.3, 126.3, 127.8, 135.5, 149.5, 152.5, 161.6; MS (ESI) m/z calcd for C₁₆H₂₁ClN₂O 292.1. Found (M+H)⁺: 293.1.

O-(7-Chloro-4-quinolyl)-4-(N,N-diethylamino)butanol 93. ¹H-NMR (300 MHz, CDCl₃) δ=1.03 (t, J=7.2 Hz, 6H), 1.85 (m, 2H), 2.06 (m, 2H), 2.55 (m, 6H), 4.17 (t, J=6.8 Hz, 2H), 6.72 (d, J=5.4 Hz, 1H), 7.44 (dd, J=2.1 Hz, 9.0 Hz, 1H), 8.01 (d, J=2.1 Hz, 1H), 8.15 (d, J=9.0 Hz, 1H), 8.72 (d, J=5.4 Hz, 1H); ¹³C-NMR (75 MHz, CDCl₃) δ=11.5, 23.7, 26.8, 46.7, 52.4, 68.4, 100.8, 119.8, 123.4, 126.3, 127.7, 135.5, 149.6, 152.4, 161.5; MS (ESI) m/z calcd for C₁₇H₂₃ClN₂O 306.2. Found (M+H)⁺: 307.2.

O-(7-Chloro-4-quinolyl)-5-(N,N-diethylamino)pentanol 94. Employing 0.09 g (0.35 mmol) of O-(7-chloro-4-quinolyl)-1,5-pentanediol in the procedure described above and purification by flash chromatography using CH₂Cl₂:EtOH (5:1 v/v) containing 0.5% Et₃N as the mobile phase gave 0.11 g (0.34 mmol, 96% yield) of a light yellow oil. ¹H-NMR (300 MHz, CDCl₃) δ=1.03 (t, J=7.1 Hz, 6H), 1.56 (m, 4H), 1.96 (m, 2H), 2.55 (m, 6H), 4.24 (t, J=6.4 Hz, 2H), 6.70 (d, J=5.3 Hz, 1H), 7.44 (dd, J=2.1 Hz, 9.0 Hz, 1H), 8.01 (d, J=2.1 Hz, 1H), 8.15 (d, J=9.0 Hz, 1H), 8.72 (d, J=5.3 Hz, 1H); ¹³C-NMR (75 MHz, CDCl₃) δ=11.2, 24.1, 26.4, 28.7, 46.7, 52.6, 68.4, 100.9, 119.9, 123.4, 126.4, 127.8, 135.6, 149.7, 152.5, 161.6; MS (ESI) m/z calcd for C₁₈H₂₅ClN₂O 320.2. Found (M+H)⁺: 321.1.

O-(7-Chloro-4-quinolyl)-6-(N,N-diethylamino)hexanol 95. Employing 0.07 g (0.25 mmol) of O-(7-chloro-4-quinolyl)-1,6-hexanediol in the procedure described above and purification by flash chromatography using CH₂Cl₂:EtOH (5:1 v/v) containing 0.5% Et₃N as the mobile phase gave 0.07 g (0.21 mmol, 83% yield) of a light yellow oil. ¹H-NMR (300 MHz, CDCl₃) δ=2.41 (t, J=7.2 Hz, 6H), 1.35-1.60 (m, 6H), 1.92 (m, 2H), 2.41 (t, J=7.5 Hz, 2H), 2.51 (q, J=7.2 Hz, 4H), 4.15 (t, J=6.9 Hz, 2H), 6.67 (d, J=5.3 Hz, 1H), 7.41 (dd, J=2.0 Hz, 9.0 Hz, 1H), 7.99 (d, J=2.0 Hz, 1H), 8.12 (d, J=9.0 Hz, 1H), 8.69 (d, J=5.3 Hz, 1H); ¹³C-NMR (75 MHz, CDCl₃) δ=11.5, 26.0, 26.9, 27.34, 28.7, 46.8, 52.7, 68.5, 100.8, 119.8, 123.4, 126.3, 127.8, 135.5, 149.6, 152.4, 161.6; GC-MS (CI) m/z calcd for C₁₉H₂₇ClN₂O 334.2. Found (M+H)⁺: 335.3.

Synthesis of S-(7-chloro-4-quinolyl)-2-(N,N-diethylamino)ethanethiol 96. See: Gallo, S.; Atifi, S.; Mahamoud, A.; Santelli-Rouvier, C.; Wolfart, K.; Molnar, J.; Barbe, J. Synthesis of aza mono, bi and tricyclic compounds. Evaluation of their anti MDR activity. Eur. J. Med. Chem. 2003, 38, 19-26; Cheng, J.; Zeidan, R.; Mishra, S.; Liu, A.; Pun, S. H.; Kulkarni, R. P.; Jensen, G. S.; Bellocq, N.C.; Davis, M. E. Structure-Function Correlation of Chloroquine and Analogues as Transgene Expression Enhancers in Nonviral Gene Delivery. J. Med. Chem. 2006, 49, 6522-6531; and Clinton, R. O.; Suter, C. M. Some dialkylaminoalkyl sulfides and ethers derived from quinoline and acridine heterocycles. J. Am. Chem. Soc. 1948, 70, 491-494. A solution of 1M potassium t-butoxide in t-butyl alcohol (6.0 mL, 6.0 mmol, 1.2 equiv.) was heated to 40° C. and 2-(diethylamino)ethanethiol (0.9 g, 6.0 mmol, 1.2 equiv.) was added dropwise. This mixture was refluxed under nitrogen for 5 minutes. A solution of 4,7-dichloroquinoline (1.0 g, 5.0 mmol, 1 equiv.) in ether was then added dropwise over a period of 10 minutes. The mixture was refluxed for an additional 12 h, cooled to room temperature and then filtered. Excess solvent was removed in vacuo and the yellow residue was purified by flash chromatography using CH₂Cl₂/MeOH/Et₃N (9:0.8:0.2 v/v) as the mobile phase to give a yellow oil (1.3 g, 4.4 mmol, 89% yield). ¹H-NMR (CDCl₃) δ=1.05 (t, J=7.2 Hz, 6H), 2.61 (q, J=7.2 Hz, 4H), 2.83 (t, J=6.6 Hz, 2H), 3.19 (t, J=6.6 Hz, 2H), 7.17 (d, J=5.1 Hz, 1H), 7.46 (dd, J=2.1 Hz, 9.9 Hz, 1H), 8.0-8.1 (m, 2H), 8.68 (d, J=5.1 Hz, 1H); ¹³C-NMR (75 MHz, CDCl₃) δ=29.9, 36.8, 51.3, 52.6, 116.2, 125.3, 125.3, 127.3, 129.1, 135.8, 148.2, 148.4, 150.4.

7-Chloroquinolyl-4-thiol. See: Surrey, A. R. Basic esters and amides of 4-quinolylmercaptoacetic acid derivatives. J. Am. Chem. Soc. 1948, 70, 2190-2193. A solution of 4,7-dichloroquinoline (3.0 g, 15.0 mmol, 1 equiv.) in 100 mL of EtOH was heated to 50° C. and thiourea (1.15 g, 15.0 mmol, 1 equiv.) was added at once. This mixture was shaken vigorously for 3 minutes and then left to cool slowly to room temperature. The white solid was filtered off, dissolved in water and Na₂CO₃ was added. A yellow-orange precipitate formed which was then filtered off and dissolved in 0.2 M NaOH solution. An insoluble solid, 7,7′-dichloro-4,4′-diquinolylsulfide, was filtered off. The filtrate was acidified with acetic acid to give 2.64 g of yellow crystals (13.5 mmol, 60% yield). ¹H-NMR (300 MHz, DMSO-d₆) δ=1.91 (s, 1H), 7.28 (d, J=6.6 Hz, 1H), 7.48 (dd, J=2.1 Hz, 9.0 Hz, 1H), 7.70 (d, J=2.1 Hz, 1H), 7.88 (d, J=6.6 Hz, 1H), 8.65 (d, J=8.7 Hz, 1H); ¹³C-NMR (75 MHz, DMSO-d6) δ=119.4, 125.4, 126.5, 131.5, 131.8, 135.0, 137.4, 137.7, 193.0.

Representative Procedure for the Synthesis of S-(7-Chloro-4-quinolyl)-n-(N,N-diethylamino)alkanethiols. A mixture of 7-chloroquinolyl-4-thiol (0.8 g, 4.1 mmol, 1 equiv.) and KOH (0.11 g, 4.1 mmol, 1 equiv.) in dry CH₃CN was stirred at 25° C. under inert atmosphere. 1,3-Dibromopropane (0.42 mL, 4.1 mmol, 1 equiv.) was added dropwise and the mixture was stirred at room temperature for 12 h. N,N-Diisopropylethylamine (0.7 mL, 4.1 mmol, 1 equiv.) followed by diethylamine (2.13 mL, 20.5 mmol, 5 equiv.) were added dropwise and the reaction was stirred for an additional 12 h. The reaction mixture was concentrated in vacuo, diluted with water (15.0 mL), and extracted with EtOAc. The combined organic layers were dried over anhydrous MgSO₄ and the solvents were removed under reduced pressure to give a light yellow oil. Purification was performed by flash chromatography using EtOAc/hexane/Et₃N (7:2.9:0.1 v/v) as the mobile phase to yield a yellow oil (0.8 g, 2.6 mmol, 64% yield).

S-(7-Chloro-4-quinolyl)-3-(N,N-diethylamino)propanethiol 97. See: Clinton, R. O.; Suter, C. M. Some dialkylaminoalkyl sulfides and ethers derived from quinoline and acridine heterocycles. J. Am. Chem. Soc. 1948, 70, 491-494. ¹H-NMR (300 MHz, CDCl₃) δ=1.11 (t, J=7.1 Hz, 6H), 1.95-2.07 (m, 2H), 2.55-2.76 (m, 6H), 3.18 (t, J=6.9 Hz, 2H), 7.17 (d, J=5.1 Hz, 1H), 7.51 (dd, J=2.1 Hz, 9.0 Hz, 1H), 8.05-8.09 (m, 2H), 8.72 (d, J=5.1 Hz, 1H); ¹³C-NMR (75 MHz, CDCl₃) δ=11.6, 25.8, 29.2, 47.2, 51.7, 116.3, 125.2, 127.4, 129.1, 135.8, 148.1, 148.2, 150.5.

S-(7-Chloro-4-quinolyl)-4-(N,N-diethylamino)butanethiol 98. See: Clinton, R. O.; Suter, C. M. Some dialkylaminoalkyl sulfides and ethers derived from quinoline and acridine heterocycles. J. Am. Chem. Soc. 1948, 70, 491-494. Employing 0.78 g (4.0 mmol) of 7-chloroquinolyl-4-thiol and 0.5 ml (4.0 mmol) of 1,4-dibromobutane in the procedure described above and purification by flash chromatography using EtOAc/hexane/Et₃N (7:2.9:0.1 v/v) as the mobile phase gave a yellow oil (0.91 g, 2.8 mmol, 69% yield). ¹H-NMR (300 MHz, CDCl₃) δ=1.04 (t, J=7.0 Hz, 6H), 1.62-1.76 (m, 2H), 1.78-1.92 (m, 2H), 2.45-2.60 (m, 6H), 3.14 (t, J=7.4 Hz, 2H), 7.20 (d, J=5.1 Hz, 1H), 7.50 (dd, J=2.3 Hz, 8.9 Hz, 1H), 8.02-8.10 (m, 2H), 8.71 (d, J=5.1 Hz, 1H); ¹³C-NMR (75 MHz, CDCl₃) δ=11.9, 26.8, 26.9, 31.3, 47.0, 52.4, 116.2, 125.2, 127.3, 129.1, 135.8, 148.2, 148.5, 150.4.

-(7-Chloro-4-quinolyl)-5-(N,N-diethylamino)pentanethiol 99. Employing 0.78 g (4.0 mmol) of 7-chloroquinolyl-4-thiol and 0.55 ml (4.0 mmol) of 1,5-dibromopentane in the procedure described above and purification by flash chromatography using EtOAc/hexane/Et₃N (7:2.9:0.1 v/v) as the mobile phase gave a yellow oil (0.81 g, 2.4 mmol, 59% yield). ¹H-NMR (300 MHz, CDCl₃) δ=1.13 (t, J=7.2 Hz, 6H), 1.50-1.70 (m, 4H), 1.80-2.0 (m, 2H), 2.50-2.80 (m, 6H), 3.14 (t, J=7.2 Hz, 2H), 7.18 (d, J=4.8 Hz, 1H), 7.51 (dd, J=1.5 Hz, 9.0 Hz, 1H), 8.00-8.10 (m, 2H), 8.72 (d, J=4.8 Hz, 1H); ¹³C-NMR (75 MHz, CDCl₃) δ=11.7, 26.8, 27.3, 28.4, 31.4, 47.1, 52.9, 116.2, 125.3, 127.4, 129.1, 135.8, 148.3, 148.5, 150.5; MS (ESI) m/z calcd for C₁₈H₂₅ClN₂S 336.1. Found (M+H)⁺: 337.1.

S-(7-Chloro-4-quinolyl)-6-(N,N-diethylamino)hexanethiol 100. Employing 0.78 g (4.0 mmol) of 7-chloroquinolyl-4-thiol and 0.6 ml (4.0 mmol) of 1,6-dibromohexane in the procedure described above and purification by flash chromatography using EtOAc/hexane/Et₃N (7:2.9:0.1 v/v) as the mobile phase gave a yellow oil (1.02 g, 2.9 mmol, 71% yield). ¹H-NMR (300 MHz, CDCl₃) δ=1.11 (t, J=7.2 Hz, 6H), 1.30-1.50 (m, 2H), 1.50-1.70 (m, 4H), 1.75-1.95 (m, 2H), 2.54 (t, J=7.4 Hz, 2H), 2.66 (q, J=7.2 Hz, 4H), 3.12 (t, J=7.4 Hz, 2H), 7.18 (d, J=4.8 Hz, 1H), 7.51 (dd, J=2.0 Hz, 8.9 Hz, 1H), 8.04-8.12 (m, 2H), 8.72 (d, J=4.8 Hz, 1H); ¹³C-NMR (75 MHz, CDCl₃) δ=13.3, 28.4, 29.6, 30.7, 31.4, 33.7, 49.4, 55.0, 118.6, 127.7, 129.7, 131.4, 138.2, 150.6, 150.8, 152.9; MS (ESI) m/z calcd for C₁₉H₂₇ClN₂S 350.2. Found (M+H)⁺: 351.2.

1,7-Bis(diethylamido)heptan-4-one. To a solution of 4-ketopimelic acid (0.2 g, 1.2 mmol) in CH₃CN was added diisopropylamine (0.5 mL, 2.9 mmol, 2.4 equiv.), PyBop (1.19 g, 2.3 mmol, 1.9 equiv.) and N,N-diisopropylethylamine (0.5 mL, 3.2 mmol, 2.7 equiv.). The reaction was refluxed at 80° C. for 48 h. The solvents were removed in vacuo and the residue was dissolved in CH₂Cl₂ and washed with 2M HCl and water. The organic layer was dried over anhydrous MgSO₄ and evaporated under reduced pressure to give 0.31 g (1.1 mmol, 98% yield) of a brown oil. ¹H-NMR (300 MHz, CDCl₃) δ=1.07 (t, J=7.2 Hz, 6H), 1.15 (t, J=7.2 Hz, 6H), 2.56 (t, J=6.6 Hz, 4H), 2.82 (t, J=6.6 Hz, 4H), 3.25-3.44 (m, 8H); ¹³C-NMR (75 MHz, CDCl₃) δ=13.2, 14.3, 27.1, 37.7, 40.4, 42.0, 171.0, 211.5.

1,7-Bis(diethylamino)heptan-4-ol. 1,7-Bis(diethylamido)heptan-4-one (0.1 g, 0.35 mmol) and lithium aluminum hydride (2.1 ml of a 1M solution in THF, 2.1 mmol, 6 equiv.) in 3 mL of anhydrous toluene were refluxed at 110° C. for 48 h. The reaction was quenched with 4M NaOH and extracted with CH₂Cl₂. The combined organic layers were dried over anhydrous MgSO₄ and evaporated under reduced pressure to afford 0.08 g (0.31 mmol, 85% yield) of a brown oil. ¹H-NMR (300 MHz, CDCl₃) δ=1.01 (t, J=7.2 Hz, 12H), 1.32-1.44 (m, 2H), 1.51-1.64 (m, 6H), 2.36-2.65 (m, 12H), 3.51-3.60 (m, 1H); ¹³C-NMR (75 MHz, CDCl₃) δ=11.3, 24.3, 37.0, 46.6, 53.5, 71.4.

7-Chloro-4-(1′,7′-bis(diethylamino)-4′-heptoxy)quinoline 102. A mixture of 4,7-dichloroquinoline (0.23 g, 1.2 mmol, 3 equiv.), 1,7-bis(diethylamino)heptan-4-ol (0.1 g, 0.39 mmol, 1 equiv.), and a 1.0 M solution of t-BuOK in t-BuOH (0.78 mL, 0.78 mmol, 2 equiv.) was heated under inert atmosphere to 120° C. for 72 h with good stirring in a closed vessel. Saturated NaHCO₃ was added to the cooled reaction mixture, which was extracted with CH₂Cl₂, dried over anhydrous MgSO₄, and concentrated in vacuo. Purification by flash chromatography using CH₂Cl₂:EtOH:Et₃N (2:1:0.02, v/v) as the mobile phase gave a yellow oil (0.09 g, 0.21 mmol, 54% yield, NMR yield >95%). ¹H-NMR (300 MHz, CDCl₃) δ=0.96 (t, J=7.1 Hz, 12H), 1.37-1.59 (m, 4H), 1.56-1.86 (m, 4H), 2.42-2.54 (overlapping t and q, 12H), 4.60 (sep, J=5.7 Hz, 1H), 6.77 (d, J=5.4 Hz, 1H), 7.40 (dd, J=1.9 Hz, 8.8 Hz, 1H), 7.98 (d, J=8.8 Hz, 1H), 8.12 (d, J=1.9 Hz, 1H), 8.69 (d, J=5.4 Hz, 1H); ¹³C-NMR (75 MHz, CDCl₃) δ=11.6, 23.0, 47.0, 52.8, 78.6, 101.9, 120.7, 123.9, 126.6, 128.2, 135.9, 150.3, 152.7, 161.3; MS (ESI) m/z calcd for C₂₄H₃₈ClN₃O 419.3. Found (M+H)⁺: 420.2.

1,9-Bis(diethylamido)nonan-5-one. To a mixture of 5-oxoazelaic acid (2.5 g, 12.4 mmol, 1 equiv.) and PyBop (15.4 g, 29.7 mmol, 2.4 equiv.) in anhydrous CH₃CN (18.0 mL) under inert atmosphere was added diethylamine (5.11 mL, 49.9 mmol, 4 equiv.) and N,N-diisopropylethylamine (6.0 ml, 34.2 mmol, 2.8 equiv.). The reaction proceeded with good stirring at 35° C. for 64 h and then solvents were removed in vacuo. The residue was dissolved in CH₂Cl₂, washed with a 2M HCl to remove N,N-diisopropylethylamine, dried over anhydrous MgSO₄, and concentrated in vacuo to produce a yellow oil (2.39 g, 7.7 mmol, 62% yield). ¹H-NMR (300 MHz, CDCl₃) δ=1.08 (t, J=7.1 Hz, 6H), 1.16 (t, J=7.2 Hz, 6H), 1.65-1.90 (m, 4H), 2.33 (t, J=7.5 Hz, 4H), 2.50 (t, J=7.1 Hz, 4H), 3.10-3.30 (m, 8H); ¹³C-NMR (75 MHz, CDCl₃) δ=14.9, 14.1, 19.3, 31.8, 40.0, 41.6, 41.9, 171.6, 210.5.

1,9-Bis(diethylamino)nonan-5-ol. 1,9-Bis(diethylamido)nonan-5-one (0.1 g, 0.32 mmol) and lithium aluminum hydride in 1M THF (2.1 ml, 2.1 mmol, 6.6 equiv.) were dissolved in 3 mL of anhydrous toluene and refluxed at 110° C. for 48 h. The reaction was quenched with 4M NaOH and extracted with CH₂Cl₂. The combined organic layers were dried over anhydrous MgSO₄ and evaporated under reduced pressure to 0.08 g (0.29 mmol, 90% yield) of a brown oil. ¹H-NMR (300 MHz, CDCl₃) δ=1.03 (t, J=6.9 Hz, 12H), 1.32-1.55 (m, 12H), 2.41 (t, J=6.6 Hz, 4H), 2.55 (q, J=6.9 Hz, 8H), 3.51-3.61 (m, 1H); ¹³C-NMR (75 MHz, CDCl₃) δ=11.6, 23.3, 26.9, 37.5, 46.9, 53.0, 71.2.

7-Chloro-4-(1′,9′-bis(diethylamino)-5′-nonoxy)quinoline 103. A mixture of 4,7-dichloroquinoline (0.21 g, 1.05 mmol, 3 equiv.), 1,9-bis(diethylamino)nonan-5-ol (0.1 g, 0.35 mmol, 1 equiv.), and a 1.0 M solution of t-BuOK in t-BuOH (0.70 mL, 0.7 mmol, 2 equiv.) was heated under inert atmosphere to 120° C. for 72 h in a closed vessel. Saturated NaHCO₃ solution was added to the cooled reaction mixture, which was extracted with CH₂Cl₂, dried over anhydrous MgSO₄, and concentrated in vacuo. Purification by flash chromatography using CH₂Cl₂:EtOH:Et₃N (2:1:0.02, v/v) as the mobile phase gave 0.06 g (0.12 mmol, 35% yield, NMR yield >95%) of a yellow oil. ¹H-NMR (300 MHz, CDCl₃) δ=0.98 (t, J=7.2 Hz, 12H), 1.43-1.95 (m, 12H), 2.40-2.59 (m, 8H), 4.55-4.70 (m, 1H), 6.69 (d, J=5.3 Hz, 1H), 7.41 (dd, J=1.8 Hz, 9.8 Hz, 1H), 8.00 (d, J=1.8 Hz, 1H), 8.15 (d, J=9.8 Hz, 1H), 8.70 (d, J=5.3 Hz, 1H); ¹³C-NMR (75 MHz, CDCl₃) δ=11.9, 23.6, 27.4, 33.8, 47.1, 53.0, 78.9, 101.7, 120.7, 123.9, 126.5, 128.1, 135.9, 150.3, 152.7, 161.5; GC-MS (CI) m/z calcd for C₂₆H₄₂ClN₃O 447.3. Found (M+H)⁺: 448.2.

7-Chloro-4-(1′,3′-bis(diethylamino)-2′-propoxy)quinoline 101. A mixture containing 4,7-dichloroquinoline (0.29 g, 1.48 mmol, 3 equiv.), 1,3-bis(diethylamino)propan-2-ol (0.1 g, 0.49 mmol, 1 equiv.), and t-BuOK in t-BuOH (1.0 mL, 1.0 mmol, 2 equiv.) was heated under inert atmosphere to 70° C. for 36 h in a closed vessel. The reaction mixture was allowed to cool to room temperature and saturated NaHCO₃ was added. The mixture was then extracted with CH₂Cl₂, dried over anhydrous MgSO₄, and concentrated in vacuo. Purification by flash chromatography using hexane:EtOH:Et₃N (2:1:0.01, v/v) as the mobile phase gave a yellow oil (0.16 g, 0.44 mmol, 90% yield). ¹H NMR (CDCl₃, ppm): ¹H-NMR (300 MHz, CDCl₃) δ=1.03 (t, J=6.9 Hz, 12H), 2.45-2.73 (m, 8H), 2.74-2.92 (m, 4H), 4.65-4.83 (m, 1H), 6.92 (d, J=5.1 Hz), 7.42 (dd, J=2.1 Hz, J=9.0 Hz, 1H), 8.05 (d, J=2.1 Hz, 1H), 8.13 (d, J=9.0 Hz, 1H), 8.72 (d, J=5.1 Hz); ¹³C-NMR (75 MHz, CDCl₃) δ=12.3, 48.2, 77.9, 102.2, 120.8, 123.9, 126.5, 128.1, 135.8, 150.3, 152.7, 161.5; MS (ESI) m/z calcd for C₂₀H₃₀ClN₃O 363.2. Found (M+H)⁺: 364.2.

### III. Heme Affinity Measurements

To measure affinity for monomeric heme, 1.2 mM stock solutions of hemin (sodium salt from Sigma-Aldrich) were prepared in DMSO and stored as 100 mL aliquots. 4.8 μM working solutions were prepared in 40% DMSO/phosphate buffer (fresh stocks prepared daily). Stock solutions of CQ and CQ analogues (diphosphate or dihydrochloride salts) were prepared in 40% DMSO/phosphate buffer and used for the titration experiments (all drugs dilutions were prepared in the same buffer).

1.5 mL cuvettes containing freshly prepared samples of 4.8 μM heme were titrated with increasing concentrations (0-50 μM) of drug. Following each addition, the sample was mixed and heme absorbance then recorded at 402 nm. Control experiments were performed by titrating 4.8 μM heme with similar volumes of solvent and no drug present. To measure affinity for μ-oxo dimeric heme, the procedure was similar except hemin was first converted to dimeric form in mild alkaline solution, followed by titration to pH 7.5 using Hepes buffer. The absorbance peak analysis was done using Microsoft excel and Kₐ were extracted from the ΔAbs₄₀₂ plots via Scatchard analysis using Microsoft Excel and Sigma plot 9.0.1 software. For each compound, Kₐ reported are the average of three separate determinations.

### IV. Quantification of In Vitro Hz Formation Inhibition

A stock solution of 5 mM hemin (Fluka) in 0.1M NaOH was prepared and stored as small aliquots at −20° C. Fresh aliquots were thawed daily to room temperature before use. Lecithin stock solutions were prepared by dissolving in distilled water to 10 mg/ml and similarly stored. 0.5 M propionate was used to buffer experiments in the pH range 5.2-5.6.

The assay mixture (1 mL volumes) contained: 200 μL lecithin solution (2 μg/ml final) 20 μL hematin (100 μM final concentration) 20 μl 0.1M HCl(Y) μl propionate buffer (X) μL drug (dependant on the concentration required 0-1000 μM). The addition sequence involved first adding the lecithin followed by heme, HCl, propionate buffer and finally the drug. Each sample was prepared in triplicate. Following addition of the reagents the samples were incubated at 37° C. with constant shaking for 18 h. After 18 h the assay was stopped by spinning the samples at 13,200 rpm for 10 minutes followed by carefully aspirating off the supernatant. The pellet was then resuspended in 50 mM bicarbonate buffer pH 9.0 (1 mL) and gently shaken at room temperature for 30 minutes to dissolve uncrystallized heme. The samples were then centrifuged as above and the supernatant removed. Following two additional bicarbonate washes the final pellet (Hz) was dried at 65° C. for ˜1 h. The samples were then dissolved in 0.1M NaOH to solubilize β-hematin to free heme and β-hematin formed was then quantified via heme absorbance at 402 nm. Calibration curves were prepared by titrating increasing amounts of heme in the same solvent vs. absorbance at 402 nm.

### V. pKa Determinations

SPARC pKa calculator is an online tool developed at the University of Georgia by S. W. Karickhoff, L. A. Carreira and S. H. Hilal. Experimental pKₐ were determined using an Accumet AB15 pH meter and a calomel electrode. 10 mM solutions of the drugs (as dibasic salts) were made in distilled H₂O and titrated at room temperature (23.0±2.0° C.) using 0.1 M NaOH. Titration plots were generated and pKₐ's extracted via inflection points from the second derivative plots; (Δ²pH/ΔV²) vs. V, where V represents the volume of the titrant added and ΔV is the volume increment.

### VI. Inversion Recovery and Distance Geometry Calculations

Relaxation rates of individual protons were converted into distances to the paramagnetic Fe center at one face of the μ-oxo dimer by applying the Solomon-Bloembergen equation:

$$R ( complex ) = ( 0.4 ) {( \frac{{μ}_{0}}{4 π} )}^{2} \frac{( {γ}_{N}^{2} {g}_{e}^{2} {μ}_{b}^{2} ) S ( S + 1 )}{{r}^{6}} {τ}_{c} , \qquad ( 1 )$$

where S is the total electron spin, r is the distance between the proton and the paramagnetic Fe, and γ_{N}, gₑ, μ₀, and μ_{b} are constants. Measurements of magnetic susceptibility for the samples used in the relaxation experiments indicate that the μ-oxo dimer has an effective spin state of ½ per Fe. Thus, S=½ is used in equation 1. The effective correlation time (τ_{c}) is defined via the relation 1/τ(effective))1/τ(rotation)+1/τ(exchange)+1/τ(electron relaxation). Since the electron relaxation time (7×10⁻¹² s) is the shortest among these time periods, it is essentially the effective correlation time. The factor 0.4 comes from simplifying the spectral density functions using (2π×500 MHz and 2π×329 GHz for the proton and electron angular frequencies, respectively). Using the distances derived from equation 1 as restraints, distance geometry/simulated annealing protocol is employed to solve the drug-μ-oxo dimer structures. The noncovalent complex is dynamic and the NMR spectrum is an average between free and complexed drug molecules. The distances 1/r⁶ are also time-averaged and since shorter distances are weighed more in this type of averaging, the r values obtained from the relaxation rates are used as minima in the distance geometry calculations. Further details are available from Leed, A.; DuBay, K.; Ursos, L. M.; Sears, D.; de Dios, A. C.; Roepe, P. D. Solution structures of antimalarial drug-heme complexes. Biochemistry 2002, 41, 10245-10255.

1. A compound of formula I-V: 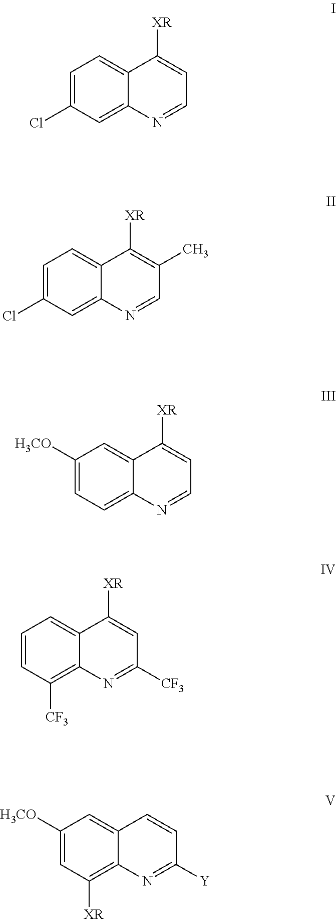
wherein, independently for each occurrence,
X is —N(H)—, —O— or —S—;
Y is hydrogen, alkyl, aryl or heteroaryl;
R is 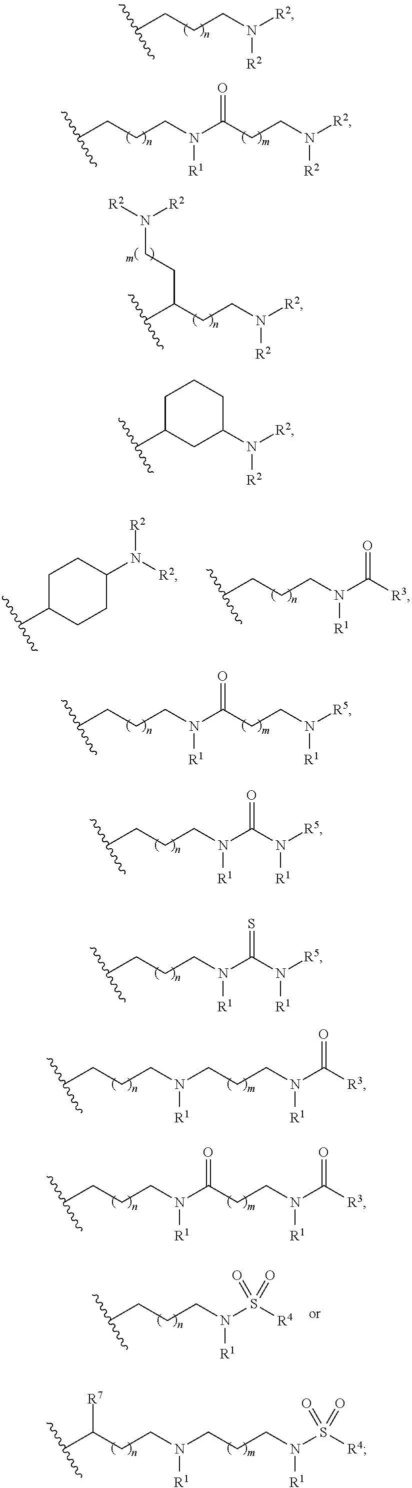
R¹ is hydrogen or alkyl;
R² is hydrogen or alkyl;
R³ is haloalkyl, aryl, aralkyl, heteroaryl, heteroaralkyl, 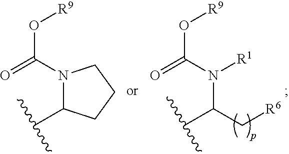
R⁴ is aryl or heteroaryl;
R⁵ is aryl or heteroaryl;
R⁶ is aryl, heteroaryl or 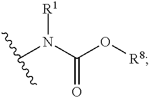
R⁷ is hydrogen or alkyl;
R⁸ is aryl, heteroaryl, aralkyl or heteroaralkyl;
R⁹ is hydrogen or alkyl;
n is 0-5 inclusive;
m is 0-5 inclusive;
p is 0-5 inclusive; and
each aryl and heteroaryl moiety, including those which are a part of an aralkyl or heteroaralkyl moiety, is optional substituted with 1-3 substitutents selected from the group consisting of alkyl, cycloalkyl, halo, perhaloalkyl, aralkyl, heteroaralkyl, alkenyl, alkynyl, carbonyl, ester, carboxyl, carboxylic acid, formyl, thiocarbonyl, thioester, thiocarboxylic acid, thioformyl, ketone, aldehyde, cyano, isocyano, amino, acylamino, amido, nitro, hydroxyl, alkoxy, aryloxy, heteroaryloxy, aralkyloxy, sulfhydryl, alkylthio, arylthio, heteroarylthio, aralkylthio, heteroaralkylthio, sulfoxido, sulfonyl, oxysulfonyl, sulfonylamino, sulfamoyl, carbocyclyl, polycyclyl, aryl, heteroaryl, and heterocyclyl.

2-5. (canceled)

6. The compound of claim 1, wherein the compound is represented by

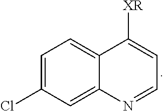

7. (canceled)

8. The compound of claim 1, wherein the compound is represented

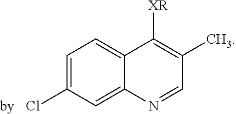

9. (canceled)

10. The compound of claim 1, wherein the compound is represented by

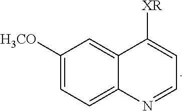

11. (canceled)

12. The compound of claim 1, wherein the compound is represented by

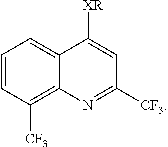

13. (canceled)

14. The compound of claim 1, wherein the compound is represented by

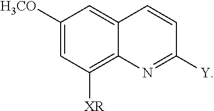

15-247. (canceled)

248. A pharmaceutical composition, comprising a compound of claim 1; and a pharmaceutically acceptable carrier or excipient.

249. A method for the therapeutic and/or prophylactic treatment of malaria in a subject in need of such treatment comprising administering to the subject a therapeutically effective amount of a compound of claim 1.

250. The method of claim 249, wherein the subject has been infected with Plasmodium falciparum.

251. The method of claim 249, wherein the subject has been infected with P. vivax.

252. The method of claim 249, wherein the subject has been infected with P. ovale.

253. The method of claim 249, wherein the subject has been infected with P. malariae.

254. The method of claim 249, wherein the compound is administered after the subject has been exposed to a malaria parasite.

255. The method of claim 249, wherein the malaria is associated with a malaria parasite that is a drug-resistant malarial strain.

256. The method of claim 255, wherein the drug-resistant malarial strain is resistant to one or more of chloroquine, mefloquine, halofantrine, artemisinin, atovaquone/proguanil, doxycycline or primaquine.

257. The method of claim 249, wherein the compound is administered before the subject travels to a country where malaria is endemic.

258. The method of claim 249, further comprising administering an antimalarial.

259. The method of claim 258, wherein the antimalarial is selected from the group consisting of quinolines, peroxide antimalarials, pyrimethamine-sulfadoxine antimalarials, hydroxynaphthoquinones, and acroline-type antimalarials.
